SEQUENCE LISTING (406 sequences)

SEQ ID NO: 1 - protein, 677 aa, Moloney murine leukemia virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 2 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 3 - protein, 1368 aa, Streptococcus pyogenes [sequence omitted]

SEQ ID NO: 4 - protein, 1052 aa, Staphylococcus lugdunensis
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKK

SEQ ID NO: 5 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1054 aa, synthetic construct
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 8 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 1054 aa, synthetic construct; identical to SEQ ID NO: 7

SEQ ID NO: 10 - protein, 1053 aa, synthetic construct
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 11 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 12 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 13 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 14 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 15 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 16 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 17 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 18 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 19 - protein, 1367 aa, synthetic construct
DKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 20 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 21 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 22 - protein, 1056 aa, synthetic construct
NQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 23 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 24 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 25 - protein, 1056 aa, synthetic construct
NQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 26 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 27 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 28 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 29 - protein, 30 aa, synthetic construct
MDSLLMNRRKFLYQFKNVRWAKGRRETYLC

SEQ ID NO: 30 - protein, 19 aa, synthetic construct
MKRTADGSEFESPKKKRKV

SEQ ID NO: 31 - protein, 17 aa, synthetic construct
KRTADGSEFEPKKKRKV

SEQ ID NO: 32 - protein, 19 aa, synthetic construct
NLSKRPAAIKKAGQAKKKK

SEQ ID NO: 33 - protein, 11 aa, synthetic construct
RQRRNELKRSF

SEQ ID NO: 34 - protein, 38 aa, synthetic construct
NQSSNFGPMKGGNFGGRSSGPYGGGGQYFAKPRNQGGY

SEQ ID NO: 35 - protein, 7 aa, synthetic construct
PKKKRKV

SEQ ID NO: 36 - protein, 16 aa, synthetic construct
KRXXXXXXXXXXKKKL

SEQ ID NO: 37 - protein, 16 aa, synthetic construct
KRPAATKKAGQAKKKK

SEQ ID NO: 38 - protein, 20 aa, synthetic construct
AVKRPAATKKAGQAKKKKLD

SEQ ID NO: 39 - protein, 25 aa, synthetic construct
MSRRRKANPTKLSENAKKLAKEVEN

SEQ ID NO: 40 - protein, 9 aa, synthetic construct
PAAKRVKLD

SEQ ID NO: 41 - protein, 9 aa, synthetic construct
KLKIKRPVK

SEQ ID NO: 42 - protein, 8 aa, synthetic construct
VSRKRPRP

SEQ ID NO: 43 - protein, 10 aa, synthetic construct
EGAPPAKRAR

SEQ ID NO: 44 - protein, 12 aa, synthetic construct
PPQPKKKPLDGE

SEQ ID NO: 45 - protein, 21 aa, synthetic construct
SGGSKRTADGSEFEPKKKRKV

SEQ ID NO: 46 - DNA, 33 bp, synthetic construct
  source 1..33: mol_type = other DNA
gccaacatgaggatcacccatgtctgcagggcc

SEQ ID NO: 47 - protein, 131 aa, synthetic construct
GSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIY

SEQ ID NO: 48 - protein, 150 aa, synthetic construct
  VARIANT 1..150: note = This sequence may encompass 1-30 GGGGS repeats
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 49 - protein, 30 aa, synthetic construct
  VARIANT 1..30: note = This sequence may encompass 1-30 residues
GGGGGGGGGGGGGGGGGGGGGGGGGGGGGG

SEQ ID NO: 50 - protein, 150 aa, synthetic construct
  VARIANT 1..150: note = This sequence may encompass 1-30 EAAAK repeats
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 51 - protein, 90 aa, synthetic construct
  VARIANT 1..90: note = This sequence may encompass 1-30 GGS repeats
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 52 - protein, 120 aa, synthetic construct
  VARIANT 1..120: note = This sequence may encompass 1-30 SGGS repeats
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 53 - protein, 60 aa, synthetic construct
  VARIANT 1..60: note = This sequence may encompass 1-30 XP repeats
XPXPXPXPXPXPXPXPXPXPXPXPXPXPXPXPXPXPXPXPXPXPXPXPXPXPXPXPXPXP

SEQ ID NO: 54 - protein, 16 aa, synthetic construct
SGSETPGTSESATPES

SEQ ID NO: 55 - protein, 32 aa, synthetic construct
SGGSSGGSSGSETPGTSESATPESSGGSSGGS

SEQ ID NO: 56 - protein, 33 aa, synthetic construct
SGGSSGGSSGSETPGTSESATPESSGGSSGGSS

SEQ ID NO: 57 - protein, 10 aa, synthetic construct
SGGSGGSGGS

SEQ ID NO: 58 - protein, 4 aa, synthetic construct
SGGS

SEQ ID NO: 59 - protein, 60 aa, synthetic construct
SGGSSGGSSGSETPGTSESATPESAGSYPYDVPDYAGSAAPAAKKKKLDGSGSGGSSGGS

SEQ ID NO: 60 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 61 - protein, 22 aa, synthetic construct
SGGSKRTADGSEFESPKKKRKV

SEQ ID NO: 62 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 63 - protein, 34 aa, synthetic construct
SGGSSGGSKRTADGSEFESPKKKRKVSGGSSGGS

SEQ ID NO: 64 - protein, 12 aa, synthetic construct
GSGPAAKRVKLD

SEQ ID NO: 65 - RNA, 21 nt, synthetic construct
  source 1..21: mol_type = other RNA
gtccaaaccgccttccaagtg

SEQ ID NO: 66 - RNA, 21 nt, synthetic construct
  source 1..21: mol_type = other RNA
gatccaaaccgccttccaagt

SEQ ID NO: 67 - RNA, 21 nt, synthetic construct
  source 1..21: mol_type = other RNA
gaatccaaaccgccttccaag

SEQ ID NO: 68 - RNA, 21 nt, synthetic construct
  source 1..21: mol_type = other RNA
gaaccaagtcccagacatgtc

SEQ ID NO: 69 - RNA, 21 nt, synthetic construct
  source 1..21: mol_type = other RNA
gagcggaacttgacacacttg

SEQ ID NO: 70 - RNA, 21 nt, synthetic construct
  source 1..21: mol_type = other RNA
gaaaagaaggtctagaagagg

SEQ ID NO: 71 - RNA, 21 nt, synthetic construct
  source 1..21: mol_type = other RNA
gaaagccatttctccaaaaga

SEQ ID NO: 72 - RNA, 21 nt, synthetic construct
  source 1..21: mol_type = other RNA
gaagtgcaacaagcagaaagg

SEQ ID NO: 73 - RNA, 21 nt, synthetic construct
  source 1..21: mol_type = other RNA
gagaaagtgcaacaagcagaa

SEQ ID NO: 74 - RNA, 21 nt, synthetic construct
  source 1..21: mol_type = other RNA
gtcagagcctgcaaaaagcaa

SEQ ID NO: 75 - skipped

SEQ ID NO: 76 - RNA, 10 nt, synthetic construct
  source 1..10: mol_type = other RNA
ttggaaggcg

SEQ ID NO: 77 - RNA, 13 nt, synthetic construct
  source 1..13: mol_type = other RNA
ttggaaggcggta

SEQ ID NO: 78 - skipped

SEQ ID NO: 79 - RNA, 11 nt, synthetic construct
  source 1..11: mol_type = other RNA
tggaaggcgga

SEQ ID NO: 80 - RNA, 13 nt, synthetic construct
  source 1..13: mol_type = other RNA
tggaaggcggttt

SEQ ID NO: 81 - skipped

SEQ ID NO: 82 - RNA, 11 nt, synthetic construct
  source 1..11: mol_type = other RNA
ggaaggcggta

SEQ ID NO: 83 - RNA, 13 nt, synthetic construct
  source 1..13: mol_type = other RNA
ggaaggcggtttg

SEQ ID NO: 84 - skipped

SEQ ID NO: 85 - RNA, 10 nt, synthetic construct
  source 1..10: mol_type = other RNA
atgtctggga

SEQ ID NO: 86 - RNA, 13 nt, synthetic construct
  source 1..13: mol_type = other RNA
atgtctgggacta

SEQ ID NO: 87 - skipped

SEQ ID NO: 88 - RNA, 11 nt, synthetic construct
  source 1..11: mol_type = other RNA
gtgtgtcaaga

SEQ ID NO: 89 - RNA, 13 nt, synthetic construct
  source 1..13: mol_type = other RNA
gtgtgtcaagttc

SEQ ID NO: 90 - skipped

SEQ ID NO: 91 - RNA, 11 nt, synthetic construct
  source 1..11: mol_type = other RNA
cttctagacca

SEQ ID NO: 92 - RNA, 14 nt, synthetic construct
  source 1..14: mol_type = other RNA
cttctagaccttca

SEQ ID NO: 93 - skipped

SEQ ID NO: 94 - RNA, 11 nt, synthetic construct
  source 1..11: mol_type = other RNA
tttggagaaaa

SEQ ID NO: 95 - RNA, 13 nt, synthetic construct
  source 1..13: mol_type = other RNA
tttggagaaatgg

SEQ ID NO: 96 - skipped

SEQ ID NO: 97 - RNA, 11 nt, synthetic construct
  source 1..11: mol_type = other RNA
ttctgcttgta

SEQ ID NO: 98 - RNA, 13 nt, synthetic construct
  source 1..13: mol_type = other RNA
ttctgcttgttgc

SEQ ID NO: 99 - skipped

SEQ ID NO: 100 - RNA, 10 nt, synthetic construct
  source 1..10: mol_type = other RNA
tgcttgttgc

SEQ ID NO: 101 - RNA, 13 nt, synthetic construct
  source 1..13: mol_type = other RNA
tgcttgttgcaca

SEQ ID NO: 102 - skipped

SEQ ID NO: 103 - RNA, 10 nt, synthetic construct
  source 1..10: mol_type = other RNA
ctttttgcag

SEQ ID NO: 104 - RNA, 13 nt, synthetic construct
  source 1..13: mol_type = other RNA
ctttttgcaggca

SEQ ID NO: 105 - RNA, 215 nt, synthetic construct
  source 1..215: mol_type = other RNA
gatgagtttggtgtaagatgcatttgtaaagcaaaataaaaagaatccacaaaacacacaaataaaatccaaaccgccttccaagtggggctctttcatgctgctgctgctgctgctcctcctcctcctcctccttctcctcctcctcctcctcttctagaccttcttttggagaaatggctttcggaagttttgccaggaaacgtagccctagg

SEQ ID NO: 106 - RNA, 195 nt, synthetic construct
  source 1..195: mol_type = other RNA
gtgtaagatgcatttgtaaagcaaaataaaaagaatccacaaaacacacaaataaaatccaaaccgccttccaagtggggctctttcatgctgctgctgctgctgctcctcctcctcctcctccttctcctcctcctcctcctcttctagaccttcttttggagaaatggctttcggaagttttgccaggaaacg

SEQ ID NO: 107 - RNA, 175 nt, synthetic construct
  source 1..175: mol_type = other RNA
catttgtaaagcaaaataaaaagaatccacaaaacacacaaataaaatccaaaccgccttccaagtggggctctttcatgctgctgctgctgctgctcctcctcctcctcctccttctcctcctcctcctcctcttctagaccttcttttggagaaatggctttcggaagttttg

SEQ ID NO: 108 - RNA, 155 nt, synthetic construct
  source 1..155: mol_type = other RNA
gcaaaataaaaagaatccacaaaacacacaaataaaatccaaaccgccttccaagtggggctctttcatgctgctgctgctgctgctcctcctcctcctcctccttctcctcctcctcctcctcttctagaccttcttttggagaaatggctttc

SEQ ID NO: 109 - RNA, 135 nt, synthetic construct
  source 1..135: mol_type = other RNA
aagaatccacaaaacacacaaataaaatccaaaccgccttccaagtggggctctttcatgctgctgctgctgctgctcctcctcctcctcctccttctcctcctcctcctcctcttctagaccttcttttggaga

SEQ ID NO: 110 - RNA, 115 nt, synthetic construct
  source 1..115: mol_type = other RNA
aaaacacacaaataaaatccaaaccgccttccaagtggggctctttcatgctgctgctgctgctgctcctcctcctcctcctccttctcctcctcctcctcctcttctagacctt

SEQ ID NO: 111 - RNA, 95 nt, synthetic construct
  source 1..95: mol_type = other RNA
aataaaatccaaaccgccttccaagtggggctctttcatgctgctgctgctgctgctcctcctcctcctcctccttctcctcctcctcctcctct

SEQ ID NO: 112 - RNA, 75 nt, synthetic construct
  source 1..75: mol_type = other RNA
aaaccgccttccaagtggggctctttcatgctgctgctgctgctgctcctcctcctcctcctccttctcctcctc

SEQ ID NO: 113 - RNA, 55 nt, synthetic construct
  source 1..55: mol_type = other RNA
ccaagtggggctctttcatgctgctgctgctgctgctcctcctcctcctcctcct

SEQ ID NO: 114 - RNA, 35 nt, synthetic construct
  source 1..35: mol_type = other RNA
ctctttcatgctgctgctgctgctgctcctcctcc

SEQ ID NO: 115 - RNA, 200 nt, synthetic construct
  source 1..200: mol_type = other RNA
gatgagtttggtgtaagatgcatttgtaaagcaaaataaaaagaatccacaaaacacacaaataaaatccaaaccgccttccaagtggggctctttcatgctcctcctcctcctcctccttctcctcctcctcctcctcttctagaccttcttttggagaaatggctttcggaagttttgccaggaaacgtagccctagg

SEQ ID NO: 116 - RNA, 180 nt, synthetic construct
  source 1..180: mol_type = other RNA
gtgtaagatgcatttgtaaagcaaaataaaaagaatccacaaaacacacaaataaaatccaaaccgccttccaagtggggctctttcatgctcctcctcctcctcctccttctcctcctcctcctcctcttctagaccttcttttggagaaatggctttcggaagttttgccaggaaacg

SEQ ID NO: 117 - RNA, 160 nt, synthetic construct
  source 1..160: mol_type = other RNA
catttgtaaagcaaaataaaaagaatccacaaaacacacaaataaaatccaaaccgccttccaagtggggctctttcatgctcctcctcctcctcctccttctcctcctcctcctcctcttctagaccttcttttggagaaatggctttcggaagttttg

SEQ ID NO: 118 - RNA, 140 nt, synthetic construct
  source 1..140: mol_type = other RNA
gcaaaataaaaagaatccacaaaacacacaaataaaatccaaaccgccttccaagtggggctctttcatgctcctcctcctcctcctccttctcctcctcctcctcctcttctagaccttcttttggagaaatggctttc

SEQ ID NO: 119 - RNA, 120 nt, synthetic construct
  source 1..120: mol_type = other RNA
aagaatccacaaaacacacaaataaaatccaaaccgccttccaagtggggctctttcatgctcctcctcctcctcctccttctcctcctcctcctcctcttctagaccttcttttggaga

SEQ ID NO: 120 - RNA, 100 nt, synthetic construct
  source 1..100: mol_type = other RNA
aaaacacacaaataaaatccaaaccgccttccaagtggggctctttcatgctcctcctcctcctcctccttctcctcctcctcctcctcttctagacctt

SEQ ID NO: 121 - RNA, 80 nt, synthetic construct
  source 1..80: mol_type = other RNA
aataaaatccaaaccgccttccaagtggggctctttcatgctcctcctcctcctcctccttctcctcctcctcctcctct

SEQ ID NO: 122 - RNA, 60 nt, synthetic construct
  source 1..60: mol_type = other RNA
aaaccgccttccaagtggggctctttcatgctcctcctcctcctcctccttctcctcctc

SEQ ID NO: 123 - RNA, 40 nt, synthetic construct
  source 1..40: mol_type = other RNA
ccaagtggggctctttcatgctcctcctcctcctcctcct

SEQ ID NO: 124 - RNA, 20 nt, synthetic construct
  source 1..20: mol_type = other RNA
ctctttcatgctcctcctcc

SEQ ID NO: 125 - RNA, 215 nt, synthetic construct
  source 1..215: mol_type = other RNA
cctagggctacgtttcctggcaaaacttccgaaagccatttctccaaaagaaggtctagaagaggaggaggaggaggagaaggaggaggaggaggaggagcagcagcagcagcagcatgaaagagccccacttggaaggcggtttggattttatttgtgtgttttgtggattctttttattttgctttacaaatgcatcttacaccaaactcatc

SEQ ID NO: 126 - RNA, 195 nt, synthetic construct
  source 1..195: mol_type = other RNA
cgtttcctggcaaaacttccgaaagccatttctccaaaagaaggtctagaagaggaggaggaggaggagaaggaggaggaggaggaggagcagcagcagcagcagcatgaaagagccccacttggaaggcggtttggattttatttgtgtgttttgtggattctttttattttgctttacaaatgcatcttacac

SEQ ID NO: 127 - RNA, 175 nt, synthetic construct
  source 1..175: mol_type = other RNA
caaaacttccgaaagccatttctccaaaagaaggtctagaagaggaggaggaggaggagaaggaggaggaggaggaggagcagcagcagcagcagcatgaaagagccccacttggaaggcggtttggattttatttgtgtgttttgtggattctttttattttgctttacaaatg

SEQ ID NO: 128 - RNA, 155 nt, synthetic construct
  source 1..155: mol_type = other RNA
gaaagccatttctccaaaagaaggtctagaagaggaggaggaggaggagaaggaggaggaggaggaggagcagcagcagcagcagcatgaaagagccccacttggaaggcggtttggattttatttgtgtgttttgtggattctttttattttgc

SEQ ID NO: 129 - RNA, 135 nt, synthetic construct
  source 1..135: mol_type = other RNA
tctccaaaagaaggtctagaagaggaggaggaggaggagaaggaggaggaggaggaggagcagcagcagcagcagcatgaaagagccccacttggaaggcggtttggattttatttgtgtgttttgtggattctt

SEQ ID NO: 130 - RNA, 115 nt, synthetic construct
  source 1..115: mol_type = other RNA
aaggtctagaagaggaggaggaggaggagaaggaggaggaggaggaggagcagcagcagcagcagcatgaaagagccccacttggaaggcggtttggattttatttgtgtgtttt

SEQ ID NO: 131 - RNA, 95 nt, synthetic construct
  source 1..95: mol_type = other RNA
agaggaggaggaggaggagaaggaggaggaggaggaggagcagcagcagcagcagcatgaaagagccccacttggaaggcggtttggattttatt

SEQ ID NO: 132 - RNA, 75 nt, synthetic construct
  source 1..75: mol_type = other RNA
gaggaggagaaggaggaggaggaggaggagcagcagcagcagcagcatgaaagagccccacttggaaggcggttt

SEQ ID NO: 133 - RNA, 55 nt, synthetic construct
  source 1..55: mol_type = other RNA
aggaggaggaggaggaggagcagcagcagcagcagcatgaaagagccccacttgg

SEQ ID NO: 134 - RNA, 35 nt, synthetic construct
  source 1..35: mol_type = other RNA
ggaggaggagcagcagcagcagcagcatgaaagag

SEQ ID NO: 135 - RNA, 200 nt, synthetic construct
  source 1..200: mol_type = other RNA
cctagggctacgtttcctggcaaaacttccgaaagccatttctccaaaagaaggtctagaagaggaggaggaggaggagaaggaggaggaggaggaggagcatgaaagagccccacttggaaggcggtttggattttatttgtgtgttttgtggattctttttattttgctttacaaatgcatcttacaccaaactcatc

SEQ ID NO: 136 - RNA, 180 nt, synthetic construct
  source 1..180: mol_type = other RNA
cgtttcctggcaaaacttccgaaagccatttctccaaaagaaggtctagaagaggaggaggaggaggagaaggaggaggaggaggaggagcatgaaagagccccacttggaaggcggtttggattttatttgtgtgttttgtggattctttttattttgctttacaaatgcatcttacac

SEQ ID NO: 137 - RNA, 160 nt, synthetic construct
  source 1..160: mol_type = other RNA
caaaacttccgaaagccatttctccaaaagaaggtctagaagaggaggaggaggaggagaaggaggaggaggaggaggagcatgaaagagccccacttggaaggcggtttggattttatttgtgtgttttgtggattctttttattttgctttacaaatg

SEQ ID NO: 138 - RNA, 140 nt, synthetic construct
  source 1..140: mol_type = other RNA
gaaagccatttctccaaaagaaggtctagaagaggaggaggaggaggagaaggaggaggaggaggaggagcatgaaagagccccacttggaaggcggtttggattttatttgtgtgttttgtggattctttttattttgc

SEQ ID NO: 139 - RNA, 120 nt, synthetic construct
  source 1..120: mol_type = other RNA
tctccaaaagaaggtctagaagaggaggaggaggaggagaaggaggaggaggaggaggagcatgaaagagccccacttggaaggcggtttggattttatttgtgtgttttgtggattctt

SEQ ID NO: 140 - RNA, 100 nt, synthetic construct
  source 1..100: mol_type = other RNA
aaggtctagaagaggaggaggaggaggagaaggaggaggaggaggaggagcatgaaagagccccacttggaaggcggtttggattttatttgtgtgtttt

SEQ ID NO: 141 - RNA, 80 nt, synthetic construct
  source 1..80: mol_type = other RNA
agaggaggaggaggaggagaaggaggaggaggaggaggagcatgaaagagccccacttggaaggcggtttggattttatt

SEQ ID NO: 142 - RNA, 60 nt, synthetic construct
  source 1..60: mol_type = other RNA
gaggaggagaaggaggaggaggaggaggagcatgaaagagccccacttggaaggcggttt

SEQ ID NO: 143 - RNA, 40 nt, synthetic construct
  source 1..40: mol_type = other RNA
aggaggaggaggaggaggagcatgaaagagccccacttgg

SEQ ID NO: 144 - RNA, 20 nt, synthetic construct
  source 1..20: mol_type = other RNA
ggaggaggagcatgaaagag

SEQ ID NO: 145 - RNA, 38 nt, synthetic construct
  source 1..38: mol_type = other RNA
tttccaagatttgcaaccggcaactgtatgctattacc

SEQ ID NO: 146 - RNA, 38 nt, synthetic construct
  source 1..38: mol_type = other RNA
ggtaatagcatacagttgccggttgcaaatcttggaaa

SEQ ID NO: 147 - RNA, 38 nt, synthetic construct
  source 1..38: mol_type = other RNA
tattgttccggtttatcacgaagcgtactcggattaca

SEQ ID NO: 148 - RNA, 38 nt, synthetic construct
  source 1..38: mol_type = other RNA
tgtaatccgagtacgcttcgtgataaaccggaacaata

SEQ ID NO: 149 - RNA, 38 nt, synthetic construct
  source 1..38: mol_type = other RNA
tatcgcaccaccctatgcgttccatatcaatacttatc

SEQ ID NO: 150 - RNA, 38 nt, synthetic construct
  source 1..38: mol_type = other RNA
gataagtattgatatggaacgcatagggtggtgcgata

SEQ ID NO: 151 - RNA, 38 nt, synthetic construct
  source 1..38: mol_type = other RNA
ttcgtctacagtatccttctttcaacctattcaccgca

SEQ ID NO: 152 - RNA, 38 nt, synthetic construct
  source 1..38: mol_type = other RNA
tgcggtgaataggttgaaagaaggatactgtagacgaa

SEQ ID NO: 153 - RNA, 38 nt, synthetic construct
  source 1..38: mol_type = other RNA
tgttatggtgtgaatacttcatggcggatgatttccac

SEQ ID NO: 154 - RNA, 38 nt, synthetic construct
  source 1..38: mol_type = other RNA
gtggaaatcatccgccatgaagtattcacaccataaca

SEQ ID NO: 155 - RNA, 38 nt, synthetic construct
  source 1..38: mol_type = other RNA
tatgtatacgaagcccatgcaaacatattgccgatcca

SEQ ID NO: 156 - RNA, 38 nt, synthetic construct
  source 1..38: mol_type = other RNA
tggatcggcaatatgtttgcatgggcttcgtatacata

SEQ ID NO: 157 - RNA, 38 nt, synthetic construct
  source 1..38: mol_type = other RNA
ttaccgtgtctacatggtatttggcatttgcgattacc

SEQ ID NO: 158 - RNA, 38 nt, synthetic construct
  source 1..38: mol_type = other RNA
ggtaatcgcaaatgccaaataccatgtagacacggtaa

SEQ ID NO: 159 - RNA, 38 nt, synthetic construct
  source 1..38: mol_type = other RNA
tatacaaggtacatataccaacgggcactccaacagaa

SEQ ID NO: 160 - RNA, 38 nt, synthetic construct
  source 1..38: mol_type = other RNA
ttctgttggagtgcccgttggtatatgtaccttgtata

SEQ ID NO: 161 - RNA, 38 nt, synthetic construct
  source 1..38: mol_type = other RNA
ttactgcgtatacttggaaatttacacgtccagaggga

SEQ ID NO: 162 - RNA, 38 nt, synthetic construct
  source 1..38: mol_type = other RNA
tccctctggacgtgtaaatttccaagtatacgcagtaa

SEQ ID NO: 163 - RNA, 38 nt, synthetic construct
  source 1..38: mol_type = other RNA
tgtatgtacagggtatacggtcaggatatggcaccgcc

SEQ ID NO: 164 - RNA, 38 nt, synthetic construct
  source 1..38: mol_type = other RNA
ggcggtgccatatcctgaccgtataccctgtacataca

SEQ ID NO: 165 - RNA, 38 nt, synthetic construct
  source 1..38: mol_type = other RNA
tcaccctcgacaagtacaggtactcgaatgcacggaca

SEQ ID NO: 166 - RNA, 38 nt, synthetic construct
  source 1..38: mol_type = other RNA
tgtccgtgcattcgagtacctgtacttgtcgagggtga

SEQ ID NO: 167 - RNA, 38 nt, synthetic construct
  source 1..38: mol_type = other RNA
ttccgaaagctttcgctctctcaggcgtattctggagc

SEQ ID NO: 168 - RNA, 38 nt, synthetic construct
  source 1..38: mol_type = other RNA
gctccagaatacgcctgagagagcgaaagctttcggaa

SEQ ID NO: 169 - RNA, 38 nt, synthetic construct
  source 1..38: mol_type = other RNA
tggcggagaaatcaattgctctcaacccgtgttgctcc

SEQ ID NO: 170 - RNA, 38 nt, synthetic construct
  source 1..38: mol_type = other RNA
ggagcaacacgggttgagagcaattgatttctccgcca

SEQ ID NO: 171 - RNA, 38 nt, synthetic construct
  source 1..38: mol_type = other RNA
tcacgttcctcatcttgttaccacgtgcaggcccatac

SEQ ID NO: 172 - RNA, 38 nt, synthetic construct
  source 1..38: mol_type = other RNA
gtatgggcctgcacgtggtaacaagatgaggaacgtga

SEQ ID NO: 173 - RNA, 38 nt, synthetic construct
  source 1..38: mol_type = other RNA
tctcgtcaagggaaccaaacacgagtatgtgtgggctc

SEQ ID NO: 174 - RNA, 38 nt, synthetic construct
  source 1..38: mol_type = other RNA
gagcccacacatactcgtgtttggttcccttgacgaga

SEQ ID NO: 175 - RNA, 38 nt, synthetic construct
  source 1..38: mol_type = other RNA
tatactgtgcgccgacatacgagggtttcctgtcactc

SEQ ID NO: 176 - RNA, 38 nt, synthetic construct
  source 1..38: mol_type = other RNA
gagtgacaggaaaccctcgtatgtcggcgcacagtata

SEQ ID NO: 177 - RNA, 38 nt, synthetic construct
  source 1..38: mol_type = other RNA
tctctggaatctttcgtttctcacgggtgtggtgtggc

SEQ ID NO: 178 - RNA, 38 nt, synthetic construct
  source 1..38: mol_type = other RNA
gccacaccacacccgtgagaaacgaaagattccagaga

SEQ ID NO: 179 - RNA, 38 nt, synthetic construct
  source 1..38: mol_type = other RNA
tacctatgtacaccagaaccagtcctcgtgtgctccac

SEQ ID NO: 180 - RNA, 38 nt, synthetic construct
  source 1..38: mol_type = other RNA
gtggagcacacgaggactggttctggtgtacataggta

SEQ ID NO: 181 - RNA, 38 nt, synthetic construct
  source 1..38: mol_type = other RNA
taccggagggcgacactcgaggttgtgacctcccacac

SEQ ID NO: 182 - RNA, 38 nt, synthetic construct
  source 1..38: mol_type = other RNA
gtgtgggaggtcacaacctcgagtgtcgccctccggta

SEQ ID NO: 183 - RNA, 38 nt, synthetic construct
  source 1..38: mol_type = other RNA
tcgtccctgtccgcggccgagagtattgtacgcccaca

SEQ ID NO: 184 - RNA, 38 nt, synthetic construct
  source 1..38: mol_type = other RNA
tgtgggcgtacaatactctcggccgcggacagggacga

SEQ ID NO: 185 - RNA, 38 nt, synthetic construct
  source 1..38: mol_type = other RNA
tcacgatcgatcgcgcggaactgggtatgctccaccgc

SEQ ID NO: 186 - RNA, 38 nt, synthetic construct
  source 1..38: mol_type = other RNA
gcggtggagcatacccagttccgcgcgatcgatcgtga

SEQ ID NO: 187 - RNA, 38 nt, synthetic construct
  source 1..38: mol_type = other RNA
ttctcggaggcatgcgggcatttgcatggctccgtccc

SEQ ID NO: 188 - RNA, 38 nt, synthetic construct
  source 1..38: mol_type = other RNA
gggacggagccatgcaaatgcccgcatgcctccgagaa

SEQ ID NO: 189 - RNA, 38 nt, synthetic construct
  source 1..38: mol_type = other RNA
tctctgccgctatgcgttcacgccatggcaaaggcgcc

SEQ ID NO: 190 - RNA, 38 nt, synthetic construct
  source 1..38: mol_type = other RNA
ggcgcctttgccatggcgtgaacgcatagcggcagaga

SEQ ID NO: 191 - RNA, 38 nt, synthetic construct
  source 1..38: mol_type = other RNA
tgcatgatggtccgatgctgcctgcctcggcctcatcc

SEQ ID NO: 192 - RNA, 38 nt, synthetic construct
  source 1..38: mol_type = other RNA
ggatgaggccgaggcaggcagcatcggaccatcatgca

SEQ ID NO: 193 - RNA, 38 nt, synthetic construct
  source 1..38: mol_type = other RNA
tgcgtcgacgcttgcgcatacatgcagaacgggcggtc

SEQ ID NO: 194 - RNA, 38 nt, synthetic construct
  source 1..38: mol_type = other RNA
gaccgcccgttctgcatgtatgcgcaagcgtcgacgca

SEQ ID NO: 195 - RNA, 38 nt, synthetic construct
  source 1..38: mol_type = other RNA
tcagctctgtcgaggcccaagccgtggtccttggtgac

SEQ ID NO: 196 - RNA, 38 nt, synthetic construct
  source 1..38: mol_type = other RNA
gtcaccaaggaccacggcttgggcctcgacagagctga

SEQ ID NO: 197 - RNA, 38 nt, synthetic construct
  source 1..38: mol_type = other RNA
tcttccggtacgcgtctcctgggtttcgcccgaaccga

SEQ ID NO: 198 - RNA, 38 nt, synthetic construct
  source 1..38: mol_type = other RNA
tcggttcgggcgaaacccaggagacgcgtaccggaaga

SEQ ID NO: 199 - RNA, 38 nt, synthetic construct
  source 1..38: mol_type = other RNA
tggcgcaccgcggcggtatctcgggccgagctctgtac

SEQ ID NO: 200 - RNA, 38 nt, synthetic construct
  source 1..38: mol_type = other RNA
gtacagagctcggcccgagataccgccgcggtgcgcca

SEQ ID NO: 201 - RNA, 38 nt, synthetic construct
  source 1..38: mol_type = other RNA
tgccaattgagccgaacagcgcctgcgggcctgccggc

SEQ ID NO: 202 - RNA, 38 nt, synthetic construct
  source 1..38: mol_type = other RNA
gccggcaggcccgcaggcgctgttcggctcaattggca

SEQ ID NO: 203 - RNA, 38 nt, synthetic construct
  source 1..38: mol_type = other RNA
tgtcacacatgctcccggcacctcgcccgacgcacggc

SEQ ID NO: 204 - RNA, 38 nt, synthetic construct
  source 1..38: mol_type = other RNA
gccgtgcgtcgggcgaggtgccgggagcatgtgtgaca

SEQ ID NO: 205 - RNA, 38 nt, synthetic construct
  source 1..38: mol_type = other RNA
tgcgggcgaacgctgctttcagcggcaccgggcacctc

SEQ ID NO: 206 - RNA, 38 nt, synthetic construct
  source 1..38: mol_type = other RNA
gaggtgcccggtgccgctgaaagcagcgttcgcccgca

SEQ ID NO: 207 - RNA, 38 nt, synthetic construct
  source 1..38: mol_type = other RNA
ttgccgcacgccgagccttctctacgggcccgggcgaa

SEQ ID NO: 208 - RNA, 38 nt, synthetic construct
  source 1..38: mol_type = other RNA
ttcgcccgggcccgtagagaaggctcggcgtgcggcaa

SEQ ID NO: 209 - RNA, 38 nt, synthetic construct
  source 1..38: mol_type = other RNA
tgcccgccacgctgcgtccagggacgggaaacgtcacc

SEQ ID NO: 210 - RNA, 38 nt, synthetic construct
  source 1..38: mol_type = other RNA
ggtgacgtttcccgtccctggacgcagcgtggcgggca

SEQ ID NO: 211 - RNA, 38 nt, synthetic construct
  source 1..38: mol_type = other RNA
tacggcggccggacctctccgcgttacgcctgcatggc

SEQ ID NO: 212 - RNA, 38 nt, synthetic construct
  source 1..38: mol_type = other RNA
gccatgcaggcgtaacgcggagaggtccggccgccgta

SEQ ID NO: 213 - RNA, 38 nt, synthetic construct
  source 1..38: mol_type = other RNA
tgctgcgagaaccgcctatcagccgggcgcacgctgcc

SEQ ID NO: 214 - RNA, 38 nt, synthetic construct
  source 1..38: mol_type = other RNA
ggcagcgtgcgcccggctgataggcggttctcgcagca

SEQ ID NO: 215 - RNA, 38 nt, synthetic construct
  source 1..38: mol_type = other RNA
tgtgcccaggctttatcggccgcctgccgcgcgttgcc

SEQ ID NO: 216 - RNA, 38 nt, synthetic construct
  source 1..38: mol_type = other RNA
ggcaacgcgcggcaggcggccgataaagcctgggcaca

SEQ ID NO: 217 - RNA, 38 nt, synthetic construct
  source 1..38: mol_type = other RNA
tgcatcgcggtgcggcccgagccgtcggcgggccttcc

SEQ ID NO: 218 - RNA, 38 nt, synthetic construct
  source 1..38: mol_type = other RNA
ggaaggcccgccgacggctcgggccgcaccgcgatgca

SEQ ID NO: 219 - RNA, 38 nt, synthetic construct
  source 1..38: mol_type = other RNA
tacgcacacccggcccgggagcgactcggccgcgtggc

SEQ ID NO: 220 - RNA, 38 nt, synthetic construct
  source 1..38: mol_type = other RNA
gccacgcggccgagtcgctcccgggccgggtgtgcgta

SEQ ID NO: 221 - RNA, 38 nt, synthetic construct
  source 1..38: mol_type = other RNA
tggccgactccggaccgtcacctcgcgcggagcgcggc

SEQ ID NO: 222 - RNA, 38 nt, synthetic construct
  source 1..38: mol_type = other RNA
gccgcgctccgcgcgaggtgacggtccggagtcggcca

SEQ ID NO: 223 - RNA, 38 nt, synthetic construct
  source 1..38: mol_type = other RNA
tgcgcgccaccacgcgcgcgtggcctccgcacgagacc

SEQ ID NO: 224 - RNA, 38 nt, synthetic construct
  source 1..38: mol_type = other RNA
ggtctcgtgcggaggccacgcgcgcgtggtggcgcgca

SEQ ID NO: 225 - RNA, 38 nt, synthetic construct
  source 1..38: mol_type = other RNA
tggcggatgagccgcccagaaggcggcgagggccggcc

SEQ ID NO: 226 - RNA, 38 nt, synthetic construct
  source 1..38: mol_type = other RNA
ggccggccctcgccgccttctgggcggctcatccgcca

SEQ ID NO: 227 - RNA, 38 nt, synthetic construct
  source 1..38: mol_type = other RNA
tgccaggtcgaccgaggccgcgggcccgggaagacggc

SEQ ID NO: 228 - RNA, 38 nt, synthetic construct
  source 1..38: mol_type = other RNA
gccgtcttcccgggcccgcggcctcggtcgacctggca

SEQ ID NO: 229 - RNA, 38 nt, synthetic construct
  source 1..38: mol_type = other RNA
tcgcccggcgccgggatgtgcgccgcggagcccggaaa

SEQ ID NO: 230 - RNA, 38 nt, synthetic construct
  source 1..38: mol_type = other RNA
tttccgggctccgcggcgcacatcccggcgccgggcga

SEQ ID NO: 231 - RNA, 38 nt, synthetic construct
  source 1..38: mol_type = other RNA
tcgaagccgcggctggcgcggacgtgcccggcgagcac

SEQ ID NO: 232 - RNA, 38 nt, synthetic construct
  source 1..38: mol_type = other RNA
gtgctcgccgggcacgtccgcgccagccgcggcttcga

SEQ ID NO: 233 - RNA, 38 nt, synthetic construct
  source 1..38: mol_type = other RNA
tccgtacgggcccagggtacgcccgcgtgccgggcagc

SEQ ID NO: 234 - RNA, 38 nt, synthetic construct
  source 1..38: mol_type = other RNA
gctgcccggcacgcgggcgtaccctgggcccgtacgga

SEQ ID NO: 235 - RNA, 23 nt, synthetic construct
  source 1..23: mol_type = other RNA
tccgcctcttatctgtgggccgc

SEQ ID NO: 236 - RNA, 23 nt, synthetic construct
  source 1..23: mol_type = other RNA
gcggcccacagataagaggcgga

SEQ ID NO: 237 - RNA, 23 nt, synthetic construct
  source 1..23: mol_type = other RNA
tgccaaccgtcgggtccatgcga

SEQ ID NO: 238 - RNA, 23 nt, synthetic construct
  source 1..23: mol_type = other RNA
tcgcatggacccgacggttggca

SEQ ID NO: 239 - RNA, 23 nt, synthetic construct
  source 1..23: mol_type = other RNA
ttcgagcgcacccgcaacaagcc

SEQ ID NO: 240 - RNA, 23 nt, synthetic construct
  source 1..23: mol_type = other RNA
ggcttgttgcgggtgcgctcgaa

SEQ ID NO: 241 - RNA, 23 nt, synthetic construct
  source 1..23: mol_type = other RNA
ttcgcgcagcagggttgcgaacc

SEQ ID NO: 242 - RNA, 23 nt, synthetic construct
  source 1..23: mol_type = other RNA
ggttcgcaaccctgctgcgcgaa

SEQ ID NO: 243 - RNA, 23 nt, synthetic construct
  source 1..23: mol_type = other RNA
tttggccggtgcgggacaggtac

SEQ ID NO: 244 - RNA, 23 nt, synthetic construct
  source 1..23: mol_type = other RNA
gtacctgtcccgcaccggccaaa

SEQ ID NO: 245 - RNA, 23 nt, synthetic construct
  source 1..23: mol_type = other RNA
tcgtttggcgatggcctcctccc

SEQ ID NO: 246 - RNA, 23 nt, synthetic construct
  source 1..23: mol_type = other RNA
gggaggaggccatcgccaaacga

SEQ ID NO: 247 - RNA, 23 nt, synthetic construct
  source 1..23: mol_type = other RNA
tctcggcgagccgtctacgtgtc

SEQ ID NO: 248 - RNA, 23 nt, synthetic construct
  source 1..23: mol_type = other RNA
gacacgtagacggctcgccgaga

SEQ ID NO: 249 - RNA, 23 nt, synthetic construct
  source 1..23: mol_type = other RNA
tcgtggtctcgggcatcagaccc

SEQ ID NO: 250 - RNA, 23 nt, synthetic construct
  source 1..23: mol_type = other RNA
gggtctgatgcccgagaccacga

SEQ ID NO: 251 - RNA, 23 nt, synthetic construct
  source 1..23: mol_type = other RNA
tgcccagtccgtgtctctgcgca

SEQ ID NO: 252 - RNA, 23 nt, synthetic construct
  source 1..23: mol_type = other RNA
tgcgcagagacacggactgggca

SEQ ID NO: 253 - RNA, 53 nt, synthetic construct
  source 1..53: mol_type = other RNA
tgcgtttgtgcaatgggcggcgcagtgggccgcgcatgtatcgccacagatca

SEQ ID NO: 254 - RNA, 53 nt, synthetic construct
  source 1..53: mol_type = other RNA
tgatctgtggcgatacatgcgcggcccactgcgccgcccattgcacaaacgca

SEQ ID NO: 255 - RNA, 53 nt, synthetic construct
  source 1..53: mol_type = other RNA
tcggaggccacggtcgccacagcaacgtggctgtttcactatggatgtcgggc

SEQ ID NO: 256 - RNA, 53 nt, synthetic construct
  source 1..53: mol_type = other RNA
gcccgacatccatagtgaaacagccacgttgctgtggcgaccgtggcctccga

SEQ ID NO: 257 - RNA, 53 nt, synthetic construct
  source 1..53: mol_type = other RNA
tctcggtcacgcacggcgcagctcggacaacccgacacgcattgctactccaa

SEQ ID NO: 258 - RNA, 53 nt, synthetic construct
  source 1..53: mol_type = other RNA
ttggagtagcaatgcgtgtcgggttgtccgagctgcgccgtgcgtgaccgaga

SEQ ID NO: 259 - RNA, 53 nt, synthetic construct
  source 1..53: mol_type = other RNA
ttgctccgtggtatcggtcccgagccatatcaggtttgccgccgcgcaaggga

SEQ ID NO: 260 - RNA, 53 nt, synthetic construct
  source 1..53: mol_type = other RNA
tcccttgcgcggcggcaaacctgatatggctcgggaccgataccacggagcaa

SEQ ID NO: 261 - RNA, 68 nt, synthetic construct
  source 1..68: mol_type = other RNA
ttgggtgtggcgccaacgggagtgcggcctgggtacctattattgcggccgttgtggtggagaggacc

SEQ ID NO: 262 - RNA, 68 nt, synthetic construct
  source 1..68: mol_type = other RNA
ggtcctctccaccacaacggccgcaataataggtacccaggccgcactcccgttggcgccacacccaa

SEQ ID NO: 263 - RNA, 68 nt, synthetic construct
  source 1..68: mol_type = other RNA
tcgctggcgtcgtacgcggagtgtgcaagagctgggtggcgaaagtcaagcagaccctggctgcacca

SEQ ID NO: 264 - RNA, 68 nt, synthetic construct
  source 1..68: mol_type = other RNA
tggtgcagccagggtctgcttgactttcgccacccagctcttgcacactccgcgtacgacgccagcga

SEQ ID NO: 265 - RNA, 68 nt, synthetic construct
  source 1..68: mol_type = other RNA
tcggctgtggcaggccgggaagctccatgtacacgctgcaaggcgaagctacccagccgtcaggaaca

SEQ ID NO: 266 - RNA, 68 nt, synthetic construct
  source 1..68: mol_type = other RNA
tgttcctgacggctgggtagcttcgccttgcagcgtgtacatggagcttcccggcctgccacagccga

SEQ ID NO: 267 - RNA, 68 nt, synthetic construct
  source 1..68: mol_type = other RNA
ttaccgggcagtgtcctttctgcgcagcagggtcgtaccggactacgcaggaggaaggcttccgcgtc

SEQ ID NO: 268 - RNA, 68 nt, synthetic construct
  source 1..68: mol_type = other RNA
gacgcggaagccttcctcctgcgtagtccggtacgaccctgctgcgcagaaaggacactgcccggtaa

SEQ ID NO: 269 - RNA, 68 nt, synthetic construct
  source 1..68: mol_type = other RNA
tacaacgcggatcagggcatccatccacagcgtgcgcgaacggcggtgtcccgagatgggtggtatgc

SEQ ID NO: 270 - RNA, 68 nt, synthetic construct
  source 1..68: mol_type = other RNA
gcataccacccatctcgggacaccgccgttcgcgcacgctgtggatggatgccctgatccgcgttgta

SEQ ID NO: 271 - RNA, 68 nt, synthetic construct
  source 1..68: mol_type = other RNA
tcctctcgcctgaatcggactggacagcttcgtgggatggctcgaagagcctccggaggcggtctacc

SEQ ID NO: 272 - RNA, 68 nt, synthetic construct
  source 1..68: mol_type = other RNA
ggtagaccgcctccggaggctcttcgagccatcccacgaagctgtccagtccgattcaggcgagagga

SEQ ID NO: 273 - RNA, 68 nt, synthetic construct
  source 1..68: mol_type = other RNA
ttgccgactgatactgcgtccttacgcccgattgcgttacaagggtgcaagcggacgcccgcgcagcc

SEQ ID NO: 274 - RNA, 68 nt, synthetic construct
  source 1..68: mol_type = other RNA
ggctgcgcgggcgtccgcttgcacccttgtaacgcaatcgggcgtaaggacgcagtatcagtcggcaa

SEQ ID NO: 275 - RNA, 68 nt, synthetic construct
  source 1..68: mol_type = other RNA
tcccggagcgaaaccggccaacgaagcgaaagggtggccctgcagtggatatgtggcggctgtcttcc

SEQ ID NO: 276 - RNA, 68 nt, synthetic construct
  source 1..68: mol_type = other RNA
ggaagacagccgccacatatccactgcagggccaccctttcgcttcgttggccggtttcgctccggga

SEQ ID NO: 277 - RNA, 68 nt, synthetic construct
  source 1..68: mol_type = other RNA
tccagcaagaggcaacccacccacgcgaagccaccacttgcgccgacgacggaacctcgctcgatttc

SEQ ID NO: 278 - RNA, 68 nt, synthetic construct
  source 1..68: mol_type = other RNA
gaaatcgagcgaggttccgtcgtcggcgcaagtggtggcttcgcgtgggtgggttgcctcttgctgga

SEQ ID NO: 279 - RNA, 83 nt, synthetic construct
  source 1..83: mol_type = other RNA
tccgaggaggacagagccaaggacagcccttcctcaatctactcacgatcgacgtgcgcggccttgaagcggcgaggtcacgc

SEQ ID NO: 280 - RNA, 83 nt, synthetic construct
  source 1..83: mol_type = other RNA
gcgtgacctcgccgcttcaaggccgcgcacgtcgatcgtgagtagattgaggaagggctgtccttggctctgtcctcctcgga

SEQ ID NO: 281 - RNA, 83 nt, synthetic construct
  source 1..83: mol_type = other RNA
tcccgccttcacaccctcgagatactgcgcgctcagccttcacacctatcgcgcacagtcggaccgggctgtactggatggga

SEQ ID NO: 282 - RNA, 83 nt, synthetic construct
  source 1..83: mol_type = other RNA
tcccatccagtacagcccggtccgactgtgcgcgataggtgtgaaggctgagcgcgcagtatctcgagggtgtgaaggcggga

SEQ ID NO: 283 - RNA, 83 nt, synthetic construct
  source 1..83: mol_type = other RNA
ttgcagccaattgtggcaacaccggaagccaaggcacaggatgccgatcgccggtgggcgcgccacttgctggccaagctatc

SEQ ID NO: 284 - RNA, 83 nt, synthetic construct
  source 1..83: mol_type = other RNA
gatagcttggccagcaagtggcgcgcccaccggcgatcggcatcctgtgccttggcttccggtgttgccacaattggctgcaa

SEQ ID NO: 285 - RNA, 83 nt, synthetic construct
  source 1..83: mol_type = other RNA
ttggccaaagacaccacgagaaatgcccaatcagggtggcagggactgccacgaagcggactgcgacgaaggcgcgcacagcc

SEQ ID NO: 286 - RNA, 83 nt, synthetic construct
  source 1..83: mol_type = other RNA
ggctgtgcgcgccttcgtcgcagtccgcttcgtggcagtccctgccaccctgattgggcatttctcgtggtgtctttggccaa

SEQ ID NO: 287 - RNA, 83 nt, synthetic construct
  source 1..83: mol_type = other RNA
ttactgagtggcccgtacgcgcagggccgtcggcagaactcagtgtcgaccgacggcagatggtccgtcttctggtcatccca

SEQ ID NO: 288 - RNA, 83 nt, synthetic construct
  source 1..83: mol_type = other RNA
tgggatgaccagaagacggaccatctgccgtcggtcgacactgagttctgccgacggccctgcgcgtacgggccactcagtaa

SEQ ID NO: 289 - RNA, 83 nt, synthetic construct
  source 1..83: mol_type = other RNA
tcgcggcctgcggttcggtacgttgcgtttcaaaccggtcttcctgcacgtctgcttgcggagcttcgaaagtcgccggagcc

SEQ ID NO: 290 - RNA, 83 nt, synthetic construct
  source 1..83: mol_type = other RNA
ggctccggcgactttcgaagctccgcaagcagacgtgcaggaagaccggtttgaaacgcaacgtaccgaaccgcaggccgcga

SEQ ID NO: 291 - RNA, 83 nt, synthetic construct
  source 1..83: mol_type = other RNA
tggcgcctgagccgtttgaaagaggcactcgtgccatattgcctcgacgcctctggcaacgccactccggtgtcccgtgtgga

SEQ ID NO: 292 - RNA, 83 nt, synthetic construct
  source 1..83: mol_type = other RNA
tccacacgggacaccggagtggcgttgccagaggcgtcgaggcaatatggcacgagtgcctctttcaaacggctcaggcgcca

SEQ ID NO: 293 - RNA, 83 nt, synthetic construct
  source 1..83: mol_type = other RNA
tcatacgcggcaatcgcgtccttctgtacggacccactacggttgccgctatgtcagagacggcggcgttgcacagccgtgcc

SEQ ID NO: 294 - RNA, 83 nt, synthetic construct
  source 1..83: mol_type = other RNA
ggcacggctgtgcaacgccgccgtctctgacatagcggcaaccgtagtgggtccgtacagaaggacgcgattgccgcgtatga

SEQ ID NO: 295 - RNA, 83 nt, synthetic construct
  source 1..83: mol_type = other RNA
tcccactaccgtcagactcgctcagaacagaccagatcacggcggcgggatccgccttgtcaaggagagcggagcgtacaccc

SEQ ID NO: 296 - RNA, 83 nt, synthetic construct
  source 1..83: mol_type = other RNA
gggtgtacgctccgctctccttgacaaggcggatcccgccgccgtgatctggtctgttctgagcgagtctgacggtagtggga

SEQ ID NO: 297 - RNA, 83 nt, synthetic construct
  source 1..83: mol_type = other RNA
tgggcgacttacgattggagcattctcctcggctttgttggctcagcgcgacccgctgggaggaggaggacacctgggagcga

SEQ ID NO: 298 - RNA, 83 nt, synthetic construct
  source 1..83: mol_type = other RNA
tcgctcccaggtgtcctcctcctcccagcgggtcgcgctgagccaacaaagccgaggagaatgctccaatcgtaagtcgccca

SEQ ID NO: 299 - RNA, 24 nt, synthetic construct
  source 1..24: mol_type = other RNA
acgatgatggtgatgatggtgtgc

SEQ ID NO: 300 - RNA, 24 nt, synthetic construct
  source 1..24: mol_type = other RNA
gcacaccatcatcaccatcatcgt

SEQ ID NO: 301 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 302 - RNA, 76 nt, synthetic construct
  source 1..76: mol_type = other RNA
gtttgagagctagaaatagcaagtttaaataaggctagtccgttatcaacttgaaaaagtgggaccgagtcggtcc

SEQ ID NO: 303 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 304 - RNA, 76 nt, synthetic construct
  source 1..76: mol_type = other RNA
gttttagtactctggaaacagaatctactaaaacaaggcaaaatgccgtgtttatctcgtcaacttgttggcgaga

SEQ ID NO: 305 - RNA, 76 nt, synthetic construct
  source 1..76: mol_type = other RNA
gttttagtactctggaaacagaatctactgaaacaagacaatatgtcgtgtttatcccatcaatttattggtggga

SEQ ID NO: 306 - RNA, 150 nt, synthetic construct
  source 1..150: mol_type = other RNA
gtccaaaccgccttccaagtggttttagagctagaaatagcaagttaaaataaggctagtccgttatcaacttgaaaaagtggcaccgagtcggtgcggcttgtcgacgacggcggtctccgtcgtcaggatcatttggaaggttttttt

SEQ ID NO: 307 - RNA, 152 nt, synthetic construct
  source 1..152: mol_type = other RNA
gtccaaaccgccttccaagtggttttagagctagaaatagcaagttaaaataaggctagtccgttatcaacttgaaaaagtggcaccgagtcggtgcggcttgtcgacgacggcggtctccgtcgtcaggatcatttggaaggcgttttttt

SEQ ID NO: 308 - RNA, 155 nt, synthetic construct
  source 1..155: mol_type = other RNA
gtccaaaccgccttccaagtggttttagagctagaaatagcaagttaaaataaggctagtccgttatcaacttgaaaaagtggcaccgagtcggtgcggcttgtcgacgacggcggtctccgtcgtcaggatcatttggaaggcggtattttttt

SEQ ID NO: 309 - RNA, 150 nt, synthetic construct
  source 1..150: mol_type = other RNA
gatccaaaccgccttccaagtgttttagagctagaaatagcaagttaaaataaggctagtccgttatcaacttgaaaaagtggcaccgagtcggtgcggcttgtcgacgacggcggtctccgtcgtcaggatcattggaaggcttttttt

SEQ ID NO: 310 - RNA, 153 nt, synthetic construct
  source 1..153: mol_type = other RNA
gatccaaaccgccttccaagtgttttagagctagaaatagcaagttaaaataaggctagtccgttatcaacttgaaaaagtggcaccgagtcggtgcggcttgtcgacgacggcggtctccgtcgtcaggatcattggaaggcggattttttt

SEQ ID NO: 311 - RNA, 155 nt, synthetic construct
  source 1..155: mol_type = other RNA
gatccaaaccgccttccaagtgttttagagctagaaatagcaagttaaaataaggctagtccgttatcaacttgaaaaagtggcaccgagtcggtgcggcttgtcgacgacggcggtctccgtcgtcaggatcattggaaggcggtttttttttt

SEQ ID NO: 312 - RNA, 150 nt, synthetic construct
  source 1..150: mol_type = other RNA
gaatccaaaccgccttccaaggttttagagctagaaatagcaagttaaaataaggctagtccgttatcaacttgaaaaagtggcaccgagtcggtgcggcttgtcgacgacggcggtctccgtcgtcaggatcatggaaggcgttttttt

SEQ ID NO: 313 - RNA, 153 nt, synthetic construct
  source 1..153: mol_type = other RNA
gaatccaaaccgccttccaaggttttagagctagaaatagcaagttaaaataaggctagtccgttatcaacttgaaaaagtggcaccgagtcggtgcggcttgtcgacgacggcggtctccgtcgtcaggatcatggaaggcggtattttttt

SEQ ID NO: 314 - RNA, 155 nt, synthetic construct
  source 1..155: mol_type = other RNA
gaatccaaaccgccttccaaggttttagagctagaaatagcaagttaaaataaggctagtccgttatcaacttgaaaaagtggcaccgagtcggtgcggcttgtcgacgacggcggtctccgtcgtcaggatcatggaaggcggtttgttttttt

SEQ ID NO: 315 - RNA, 150 nt, synthetic construct
  source 1..150: mol_type = other RNA
gaaccaagtcccagacatgtcgttttagagctagaaatagcaagttaaaataaggctagtccgttatcaacttgaaaaagtggcaccgagtcggtgcggcttgtcgacgacggcggtctccgtcgtcaggatcatatgtctggttttttt

SEQ ID NO: 316 - RNA, 152 nt, synthetic construct
  source 1..152: mol_type = other RNA
gaaccaagtcccagacatgtcgttttagagctagaaatagcaagttaaaataaggctagtccgttatcaacttgaaaaagtggcaccgagtcggtgcggcttgtcgacgacggcggtctccgtcgtcaggatcatatgtctgggattttttt

SEQ ID NO: 317 - RNA, 155 nt, synthetic construct
  source 1..155: mol_type = other RNA
gaaccaagtcccagacatgtcgttttagagctagaaatagcaagttaaaataaggctagtccgttatcaacttgaaaaagtggcaccgagtcggtgcggcttgtcgacgacggcggtctccgtcgtcaggatcatatgtctgggactattttttt

SEQ ID NO: 318 - RNA, 150 nt, synthetic construct
  source 1..150: mol_type = other RNA
gagcggaacttgacacacttggttttagagctagaaatagcaagttaaaataaggctagtccgttatcaacttgaaaaagtggcaccgagtcggtgcggcttgtcgacgacggcggtctccgtcgtcaggatcatgtgtgtcattttttt

SEQ ID NO: 319 - RNA, 153 nt, synthetic construct
  source 1..153: mol_type = other RNA
gagcggaacttgacacacttggttttagagctagaaatagcaagttaaaataaggctagtccgttatcaacttgaaaaagtggcaccgagtcggtgcggcttgtcgacgacggcggtctccgtcgtcaggatcatgtgtgtcaagattttttt

SEQ ID NO: 320 - RNA, 155 nt, synthetic construct
  source 1..155: mol_type = other RNA
gagcggaacttgacacacttggttttagagctagaaatagcaagttaaaataaggctagtccgttatcaacttgaaaaagtggcaccgagtcggtgcggcttgtcgacgacggcggtctccgtcgtcaggatcatgtgtgtcaagttcttttttt

SEQ ID NO: 321 - RNA, 150 nt, synthetic construct
  source 1..150: mol_type = other RNA
gaaaagaaggtctagaagagggttttagagctagaaatagcaagttaaaataaggctagtccgttatcaacttgaaaaagtggcaccgagtcggtgcatgatcctgacgacggagaccgccgtcgtcgacaagcccttctagattttttt

SEQ ID NO: 322 - RNA, 153 nt, synthetic construct
  source 1..153: mol_type = other RNA
gaaaagaaggtctagaagagggttttagagctagaaatagcaagttaaaataaggctagtccgttatcaacttgaaaaagtggcaccgagtcggtgcatgatcctgacgacggagaccgccgtcgtcgacaagcccttctagaccattttttt

SEQ ID NO: 323 - RNA, 156 nt, synthetic construct
  source 1..156: mol_type = other RNA
gaaaagaaggtctagaagagggttttagagctagaaatagcaagttaaaataaggctagtccgttatcaacttgaaaaagtggcaccgagtcggtgcatgatcctgacgacggagaccgccgtcgtcgacaagcccttctagaccttcattttttt

SEQ ID NO: 324 - RNA, 150 nt, synthetic construct
  source 1..150: mol_type = other RNA
gaaagccatttctccaaaagagttttagagctagaaatagcaagttaaaataaggctagtccgttatcaacttgaaaaagtggcaccgagtcggtgcatgatcctgacgacggagaccgccgtcgtcgacaagcctttggagattttttt

SEQ ID NO: 325 - RNA, 153 nt, synthetic construct
  source 1..153: mol_type = other RNA
gaaagccatttctccaaaagagttttagagctagaaatagcaagttaaaataaggctagtccgttatcaacttgaaaaagtggcaccgagtcggtgcatgatcctgacgacggagaccgccgtcgtcgacaagcctttggagaaaattttttt

SEQ ID NO: 326 - RNA, 155 nt, synthetic construct
  source 1..155: mol_type = other RNA
gaaagccatttctccaaaagagttttagagctagaaatagcaagttaaaataaggctagtccgttatcaacttgaaaaagtggcaccgagtcggtgcatgatcctgacgacggagaccgccgtcgtcgacaagcctttggagaaatggttttttt

SEQ ID NO: 327 - RNA, 150 nt, synthetic construct
  source 1..150: mol_type = other RNA
gaagtgcaacaagcagaaagggttttagagctagaaatagcaagttaaaataaggctagtccgttatcaacttgaaaaagtggcaccgagtcggtgcatgatcctgacgacggagaccgccgtcgtcgacaagccttctgcttttttttt

SEQ ID NO: 328 - RNA, 153 nt, synthetic construct
  source 1..153: mol_type = other RNA
gaagtgcaacaagcagaaagggttttagagctagaaatagcaagttaaaataaggctagtccgttatcaacttgaaaaagtggcaccgagtcggtgcatgatcctgacgacggagaccgccgtcgtcgacaagccttctgcttgtattttttt

SEQ ID NO: 329 - RNA, 155 nt, synthetic construct
  source 1..155: mol_type = other RNA
gaagtgcaacaagcagaaagggttttagagctagaaatagcaagttaaaataaggctagtccgttatcaacttgaaaaagtggcaccgagtcggtgcatgatcctgacgacggagaccgccgtcgtcgacaagccttctgcttgttgcttttttt

SEQ ID NO: 330 - RNA, 150 nt, synthetic construct
  source 1..150: mol_type = other RNA
gagaaagtgcaacaagcagaagttttagagctagaaatagcaagttaaaataaggctagtccgttatcaacttgaaaaagtggcaccgagtcggtgcatgatcctgacgacggagaccgccgtcgtcgacaagcctgcttgttttttttt

SEQ ID NO: 331 - RNA, 152 nt, synthetic construct
  source 1..152: mol_type = other RNA
gagaaagtgcaacaagcagaagttttagagctagaaatagcaagttaaaataaggctagtccgttatcaacttgaaaaagtggcaccgagtcggtgcatgatcctgacgacggagaccgccgtcgtcgacaagcctgcttgttgcttttttt

SEQ ID NO: 332 - RNA, 155 nt, synthetic construct
  source 1..155: mol_type = other RNA
gagaaagtgcaacaagcagaagttttagagctagaaatagcaagttaaaataaggctagtccgttatcaacttgaaaaagtggcaccgagtcggtgcatgatcctgacgacggagaccgccgtcgtcgacaagcctgcttgttgcacattttttt

SEQ ID NO: 333 - RNA, 150 nt, synthetic construct
  source 1..150: mol_type = other RNA
gtcagagcctgcaaaaagcaagttttagagctagaaatagcaagttaaaataaggctagtccgttatcaacttgaaaaagtggcaccgagtcggtgcatgatcctgacgacggagaccgccgtcgtcgacaagccctttttgcttttttt

SEQ ID NO: 334 - RNA, 152 nt, synthetic construct
  source 1..152: mol_type = other RNA
gtcagagcctgcaaaaagcaagttttagagctagaaatagcaagttaaaataaggctagtccgttatcaacttgaaaaagtggcaccgagtcggtgcatgatcctgacgacggagaccgccgtcgtcgacaagccctttttgcagttttttt

SEQ ID NO: 335 - RNA, 155 nt, synthetic construct
  source 1..155: mol_type = other RNA
gtcagagcctgcaaaaagcaagttttagagctagaaatagcaagttaaaataaggctagtccgttatcaacttgaaaaagtggcaccgagtcggtgcatgatcctgacgacggagaccgccgtcgtcgacaagccctttttgcaggcattttttt

SEQ ID NO: 336 - skipped

SEQ ID NO: 337 - DNA, 55 bp, synthetic construct
  source 1..55: mol_type = other DNA
gggtcaggagccccccccctgaacccaggataaccctcaaagtcggggggcaacc

SEQ ID NO: 338 - DNA, 56 bp, synthetic construct
  source 1..56: mol_type = other DNA
gggtcaggagccccccccctgaacccaggaaaaccctcaaagtcggggggcaaccc

SEQ ID NO: 339 - DNA, 56 bp, synthetic construct
  source 1..56: mol_type = other DNA
gggtcaggagccccccccctgcacccaggaaaaccctcaaagtcggggggcaaccc

SEQ ID NO: 340 - DNA, 56 bp, synthetic construct
  source 1..56: mol_type = other DNA
gggtcaggagccccccccctgcacccaggataaccctcaaagtcggggggcaaccc

SEQ ID NO: 341 - DNA, 60 bp, synthetic construct
  source 1..60: mol_type = other DNA
gtcagggtcaggagccccccccctgaacccaggaaaaccctcaaagtcggggggcaaccc

SEQ ID NO: 342 - DNA, 60 bp, synthetic construct
  source 1..60: mol_type = other DNA
gtcagggtcaggagccccccccctgcacccaggaaaaccctcaaagtcggggggcaaccc

SEQ ID NO: 343 - DNA, 51 bp, synthetic construct
  source 1..51: mol_type = other DNA
gggtcaggagccccccccctgaacccaggataaccctcaaagtcggggggc

SEQ ID NO: 344 - DNA, 13 bp, synthetic construct
  source 1..13: mol_type = other DNA
tggtggtggtggt

SEQ ID NO: 345 - DNA, 20 bp, synthetic construct
  source 1..20: mol_type = other DNA
gggacagggcagggacaggg

SEQ ID NO: 346 - DNA, 21 bp, synthetic construct
  source 1..21: mol_type = other DNA
gggtccgggtctgggtctggg

SEQ ID NO: 347 - DNA, 19 bp, synthetic construct
  source 1..19: mol_type = other DNA
gggcagggtctgggctggg

SEQ ID NO: 348 - DNA, 19 bp, synthetic construct
  source 1..19: mol_type = other DNA
gggctgggatgggaaaggg

SEQ ID NO: 349 - DNA, 19 bp, synthetic construct
  source 1..19: mol_type = other DNA
gggctctgggtgggccggg

SEQ ID NO: 350 - DNA, 18 bp, synthetic construct
  source 1..18: mol_type = other DNA
gggctgggctgggcaggg

SEQ ID NO: 351 - DNA, 17 bp, synthetic construct
  source 1..17: mol_type = other DNA
gggtgggctgggaaggg

SEQ ID NO: 352 - DNA, 17 bp, synthetic construct
  source 1..17: mol_type = other DNA
gggagggagggctaggg

SEQ ID NO: 353 - DNA, 17 bp, synthetic construct
  source 1..17: mol_type = other DNA
gggcagggctgggaggg

SEQ ID NO: 354 - DNA, 16 bp, synthetic construct
  source 1..16: mol_type = other DNA
gggtgggagggctggg

SEQ ID NO: 355 - DNA, 43 bp, synthetic construct
  source 1..43: mol_type = other DNA
gcgtaacctccatccgagttgcaagagagggaaacgcagtctc

SEQ ID NO: 356 - DNA, 160 bp, synthetic construct
  source 1..160: mol_type = other DNA
ggaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttca

SEQ ID NO: 357 - DNA, 42 bp, synthetic construct
  source 1..42: mol_type = other DNA
ttgacgcggttctatctagttacgcgttaaaccaactagaaa

SEQ ID NO: 358 - DNA, 42 bp, synthetic construct
  source 1..42: mol_type = other DNA
ttgacgcggttctatctacttacgcgttaaaccaactagaaa

SEQ ID NO: 359 - DNA, 44 bp, synthetic construct
  source 1..44: mol_type = other DNA
cgcgagtctaggggataacgcgttaaacttcctagaaggcggtt

SEQ ID NO: 360 - DNA, 44 bp, synthetic construct
  source 1..44: mol_type = other DNA
cgcggatctagattgtaacgcgttaaaccatctagaaggcggtt

SEQ ID NO: 361 - DNA, 44 bp, synthetic construct
  source 1..44: mol_type = other DNA
cgcgtcgctaccgcccggcgcgttaaacacactagaaggcggtt

SEQ ID NO: 362 - DNA, 37 bp, synthetic construct
  source 1..37: mol_type = other DNA
cgcggttctatctagttacgcgttaaaccaactagaa

SEQ ID NO: 363 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 364 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 365 - protein, 1052 aa, synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 366 - protein, 677 aa, Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 367 - protein, 6 aa, synthetic construct
GGSGGS

SEQ ID NO: 368 - RNA, 68 nt, synthetic construct
  source 1..68: mol_type = other RNA
ggccggcatggtcccagcctcctcgctggcgccggctgggcaacatgcttcggcatggcgaatgggac

SEQ ID NO: 369 - RNA, 75 nt, synthetic construct
  source 1..75: mol_type = other RNA
  variation 69..75: note = This region may be 3-7 nucleotides
ggccggcatggtcccagcctcctcgctggcgccggctgggcaacatgcttcggcatggcgaatgggacttttttt

SEQ ID NO: 370 - RNA, 18 nt, synthetic construct
  source 1..18: mol_type = other RNA
ggtgggagacgtcccacc

SEQ ID NO: 371 - RNA, 16 nt, synthetic construct
  source 1..16: mol_type = other RNA
tgggagacgtcccacc

SEQ ID NO: 372 - RNA, 23 nt, synthetic construct
  source 1..23: mol_type = other RNA
  variation 17..23: note = This region may be 3-7 nucleotides
tgggagacgtcccaccttttttt

SEQ ID NO: 373 - RNA, 24 nt, synthetic construct
  source 1..24: mol_type = other RNA
gagcagcatggcgtcgctgctcac

SEQ ID NO: 374 - RNA, 21 nt, synthetic construct
  source 1..21: mol_type = other RNA
ccatcagttgacaccctgagg

SEQ ID NO: 375 - RNA, 28 nt, synthetic construct
  source 1..28: mol_type = other RNA
  variation 22..28: note = This region may be 3-7 nucleotides
ccatcagttgacaccctgaggttttttt

SEQ ID NO: 376 - RNA, 28 nt, synthetic construct
  source 1..28: mol_type = other RNA
gcagacctaagtggtgacatatggtctg

SEQ ID NO: 377 - RNA, 24 nt, synthetic construct
  source 1..24: mol_type = other RNA
catgcgattagaaataatcgcatg

SEQ ID NO: 378 - RNA, 31 nt, synthetic construct
  source 1..31: mol_type = other RNA
  variation 25..31: note = This region may be 3-7 nucleotides
catgcgattagaaataatcgcatgttttttt

SEQ ID NO: 379 - RNA, 118 nt, synthetic construct
  source 1..118: mol_type = other RNA
tctgccatcaaagctgcgaccgtgctcagtctggtgggagacgtcccaccggccggcatggtcccagcctcctcgctggcgccggctgggcaacatgcttcggcatggcgaatgggac

SEQ ID NO: 380 - skipped

SEQ ID NO: 381 - RNA, 76 nt, synthetic construct
  source 1..76: mol_type = other RNA
gttttagagctatacgtagcaagttaaaataaggctagtccgttatcaactttacgaagtggcaccgagtcggtgc

SEQ ID NO: 382 - RNA, 76 nt, synthetic construct
  source 1..76: mol_type = other RNA
gttttagagctatacgtagcaagttaaaataaggctagtccgttatcaactttacgaagtgggaccgagtcggtcc

SEQ ID NO: 383 - RNA, 88 nt, synthetic construct
  source 1..88: mol_type = other RNA
gttttagagctagctcatgaaaatgagctagcaagttaaaataaggctagtccgttatcaacttgaaaaagtgggaccgagtcggtcc

SEQ ID NO: 384 - RNA, 76 nt, synthetic construct
  source 1..76: mol_type = other RNA
gtttaagagctagaaatagcaagtttaaataaggctagtccgttatcaacttgaaaaagtggcaccgagtcggtgc

SEQ ID NO: 385 - RNA, 86 nt, synthetic construct
  source 1..86: mol_type = other RNA
gttttagagctatgctggaaacagcatagcaagttaaaataaggctagtccgttatcaacttgaaaaagtggcaccgagtcggtgc

SEQ ID NO: 386 - protein, 9 aa, synthetic construct
GGSGGSGGS

SEQ ID NO: 387 - protein, 16 aa, Drosophila melanogaster
RQIKIWFQNRRMKWKK

SEQ ID NO: 388 - protein, 11 aa, Human immunodeficiency virus
YGRKKRRQRRR

SEQ ID NO: 389 - RNA, 60 nt, synthetic construct
  source 1..60: mol_type = other RNA
gtcagggtcaggagccccccccctgaacccaggataaccctcaaagtcggggggcaaccc

SEQ ID NO: 390 - DNA, 15 bp, synthetic construct
  source 1..15: mol_type = other DNA
ctgctgctgctgctg

SEQ ID NO: 391 - DNA, 150 bp, synthetic construct
  source 1..150: mol_type = other DNA
  variation 1..150: note = This sequence may encompass 35-50 CTG repeats
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 392 - DNA, 147 bp, synthetic construct
  source 1..147: mol_type = other DNA
  variation 1..147: note = This sequence may encompass 35-49 CTG repeats
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 393 - DNA, 450 bp, synthetic construct
  source 1..450: mol_type = other DNA
  variation 1..450: note = This sequence may encompass 50-150 CTG repeats
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 394 - DNA, 90 bp, synthetic construct
  source 1..90: mol_type = other DNA
ctgctgctgctgctgctgctgctgctgctgctgctgctgctgctgctgctgctgctgctgctgctgctgctgctgctgctgctgctgctg

SEQ ID NO: 395 - DNA, 60 bp, synthetic construct
  source 1..60: mol_type = other DNA
ctgctgctgctgctgctgctgctgctgctgctgctgctgctgctgctgctgctgctgctg

SEQ ID NO: 396 - DNA, 3000 bp, synthetic construct
  source 1..3000: mol_type = other DNA
  variation 1..3000: note = This sequence may encompass 50-1000 CTG repeats
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 397 - DNA, 3000 bp, synthetic construct; identical to SEQ ID NO: 396
  source 1..3000: mol_type = other DNA
  variation 1..3000: note = This sequence may encompass 100-1000 CTG repeats

SEQ ID NO: 398 - RNA, 99 nt, synthetic construct
  source 1..99: mol_type = other RNA
  variation 1..99: note = This sequence may encompass 1-33 CUG repeats
ctgctgctgctgctgctgctgctgctgctgctgctgctgctgctgctgctgctgctgctgctgctgctgctgctgctgctgctgctgctgctgctgctg

SEQ ID NO: 399 - RNA, 99 nt, synthetic construct
  source 1..99: mol_type = other RNA
  variation 1..99: note = This sequence may encompass 1-33 CAG repeats
cagcagcagcagcagcagcagcagcagcagcagcagcagcagcagcagcagcagcagcagcagcagcagcagcagcagcagcagcagcagcagcagcag

SEQ ID NO: 400 - DNA, 30 bp, synthetic construct
  source 1..30: mol_type = other DNA
ctgctgctgctgctgctgctgctgctgctg

SEQ ID NO: 401 - DNA, 72 bp, Homo sapiens
  source 1..72: mol_type = genomic DNA
ctgctgctgctgctgctgctgctgctgctgctgctgctgctgctgctgctgctgctgctgctgctgctgctg

SEQ ID NO: 402 - RNA, 114 nt, synthetic construct
  source 1..114: mol_type = other RNA
  variation 1..114: note = This sequence may encompass 1-38 CUG repeats
ctgctgctgctgctgctgctgctgctgctgctgctgctgctgctgctgctgctgctgctgctgctgctgctgctgctgctgctgctgctgctgctgctgctgctgctgctgctg

SEQ ID NO: 403 - RNA, 114 nt, synthetic construct
  source 1..114: mol_type = other RNA
  variation 1..114: note = This sequence may encompass 1-38 CAG repeats
cagcagcagcagcagcagcagcagcagcagcagcagcagcagcagcagcagcagcagcagcagcagcagcagcagcagcagcagcagcagcagcagcagcagcagcagcagcag

SEQ ID NO: 404 - protein, 9 aa, synthetic construct
RRRRRRRRR

SEQ ID NO: 405 - protein, 8 aa, synthetic construct
RRRRRRRR

SEQ ID NO: 406 - DNA, 102 bp, synthetic construct
  source 1..102: mol_type = other DNA
  variation 1..102: note = This sequence may encompass 5-34 CTG repeats
ctgctgctgctgctgctgctgctgctgctgctgctgctgctgctgctgctgctgctgctgctgctgctgctgctgctgctgctgctgctgctgctgctgctg

(19) United States
(12) Patent Application Publication
(10) Pub. No.: US 2025/0319202 A1
(43) Pub. Date: Oct. 16, 2025

(54) GENOME EDITING COMPOSITIONS AND METHODS FOR TREATMENT OF FUCHS ENDOTHELIAL CORNEAL DYSTROPHY

(71) Applicant: Prime Medicine, Inc., Cambridge, MA (US)

(72) Inventors: Tyler HARVEY, Somerville, MA (US); Yu ZHENG, Wellesley, MA (US)

(21) Appl. No.: 18/708,340

(22) PCT Filed: Nov. 9, 2022
(86) PCT No.: PCT/US2022/079578

§ 371 Date: May 8, 2024

Related U.S. Application Data
(60) Provisional application No. 63/277,355, filed on Nov. 9, 2021.

(51) Int. Cl. A61K 48/00; A61K 9/51; A61P 27/02; C12N 9/12; C12N 9/22; C12N 15/11; C12N 15/86; C12N 15/88

(52) U.S. Cl. CPC A61K 48/005; A61K 9/5123; A61P 27/02; C12N 9/1276; C12N 9/226; C12N 15/111; C12N 15/86; C12N 15/88; C07K 2319/80; C12N 2310/20; C12Y 207/07049

(57) ABSTRACT

Provided herein are prime editing methods and compositions for treatment of genetic disorders such as Fuchs endothelial corneal dystrophy type 3.

### CROSS REFERENCE TO RELATED APPLICATIONS

The present application claims priority to and the benefit of U.S. Provisional Application No. 63/277,355 entitled, filed Nov. 9, 2021, the contents of which are herein incorporated by reference in their entirety.

### REFERENCE TO THE SEQUENCE LISTING

The instant application contains a Sequence Listing which has been submitted electronically in XML format and which is hereby incorporated by reference in its entirety. Said XML copy, created on Nov. 9, 2022, is named 272059-517407_Sequence-Listing and is 420,825 kilobytes in size.

### BACKGROUND

The present invention describes dual prime editing as a genome editing approach for treating genetic diseases, for example, repeat expansion disorder Fuchs' endothelial corneal dystrophy type 3 (FECD)

FECD) is a late-onset disorder of the corneal endothelium, affecting about 4% of the over-40 population. Gradual accumulation of excrescences from Descemet's membrane eventually impairs the corneal epithelium, leading to stromal edema, corneal erosion, and vision loss.

FECD is associated with mutations in the TCF4 gene (OMIM #602272), encoded on the negative strand of human chromosome 18 at 18q21.2 (NC_000018.10: c55635957-55222185, GRCh38.p13). The TCF4 gene encodes Transcription Factor 4 (TCF4), a basic helix-loop-helix transcription factor expressed in most, if not all, human cell types. TCF4 is expressed in a wide variety of isoforms due to alternative splicing and alternative transcription start sites.

In individuals unaffected by FECD, intron 3 of the TCF4 gene contains a CTG trinucleotide repeat (TNR), known as CTG 18.1, with a small number of repeats, typically under 40. For example, there are 24 CTG repeats (SEQ ID NO: 401) in Genome Reference Consortium Human Build 38 patch release 13 (GRCh38.p13: NC_000018.10: c55586227-55586156). Individuals with more than 40 repeats are considered to have a trinucleotide expansion (Eghrari 2017, doi:10.1097/ico.0000000000001049), and an expansion of 50 or more repeats is associated with an increased risk of developing FECD (Sirp 2020, doi: 10.1038/s41598-020-75437-3). The severity of FECD is correlated with the number of abnormally expanded CTG repeats in the TCF4 gene, which have been reported to be into the hundreds in some cases (Eghrari 2017).

Definitive treatment for FECD includes corneal transplantation. More recently, rho-associated kinase inhibitors such as ripasudil have been investigated for restoration of corneal endothelial cells.

This disclosure provides dual prime editing methods and compositions for editing the TCF4 gene and removing pathogenic trinucleotide repeats associated with FECD and other diseases associated with the TCF4 intron-3 CTG repeat expansion, such as bipolar disorder.

### SUMMARY

The prime editing systems described herein comprise compositions, systems, and methods that relate to programmable editing of a double-stranded target DNA, e.g., a target gene such as a TCF4 gene, using two or more prime editing guide RNAs (PEgRNAs) each complexed with a prime editor (“dual prime editing”). The compositions, systems, and methods described herein may be used to incorporate one or more intended nucleotide edits into the double-stranded target DNA. In some embodiments, compositions, systems and methods relating to dual prime editing may be used to make alterations in a target sequence of a target gene, for example, a TCF4 gene.

In some embodiments, dual prime editing incorporates one or more intended nucleotide edits into the target DNA through excision of an endogenous DNA segment and/or replacement of the endogenous DNA segment with newly synthesized DNA via target-primed DNA synthesis. Dual prime editing involves two different PEgRNAs each complexed with a prime editor. Without wishing to be bound by any particular theory, each of the two PEgRNAs comprises a region of complementarity to a distinct search target sequence of a target DNA, for example, a TCF4 gene, wherein the two distinct search target sequences are on the two complementary strands of the target DNA. In some embodiments, the two PEgRNAs each can direct a prime editor to initiate the prime editing process on the two complementary strands of the target DNA, thereby incorporating one or more intended nucleotide edits into the target DNA, e.g., the TCF4 gene.

In one embodiment, a composition comprises a first prime editing guide RNA (PEgRNA) and a second PERNA, wherein: the first PEgRNA comprises a first spacer that is complementary to a first search target sequence on a first strand of a double-stranded TCF4 gene, a first gRNA core that associates with a first prime editor comprising a DNA binding domain and DNA polymerase domain, and a first editing template; and the second PEgRNA comprises a second spacer that is complementary to a second search target sequence on a second strand of the double-stranded TCF4 gene, a second gRNA core that associates with a second prime editor comprising a DNA binding domain and a DNA polymerase domain, and a second editing template, wherein the first strand and the second strand of the double-stranded TCF4 gene are complementary to each other, and wherein the first editing template and the second editing template each comprises a region of complementarity to each other.

In one embodiment, a composition comprises a first prime editing guide RNA (PEgRNA) and a second PEgRNA, wherein: the first PEgRNA comprises a first spacer that is complementary to a first search target sequence on a first strand of a double-stranded TCF4 gene, a first gRNA core that associates with a first prime editor comprising a DNA binding domain and DNA polymerase domain, and a first editing template; and the second PEgRNA comprises a second spacer that is complementary to a second search target sequence on a second strand of the double-stranded TCF4 gene, a second gRNA core that associates with a second prime editor comprising a DNA binding domain and a DNA polymerase domain, and a second editing template, wherein the first strand and the second strand of the double-stranded TCF4 gene are complementary to each other, wherein the first editing template comprises a region of identity to a sequence on the first strand of the TCF4 gene, and wherein the second editing template comprises a region of identity to a sequence on the second strand of the double-stranded TCF4 gene.

In some embodiments, the first PEgRNA directs the first prime editor to generate a first nick on the second strand of the TCF4 gene, wherein the second PEgRNA directs the second prime editor to generate a second nick on the first strand of the TCF4 gene, and wherein the TCF4 gene comprises an inter-nick duplex (IND) between the position of the first nick and the position of the second nick.

In some embodiments, the IND comprises an array of tri-nucleotide repeats.

In some embodiments, the double-stranded TCF4 gene comprises a mutation associated with FECD, the IND comprises the mutation associated with FECD, and/or the mutation is an increased number of tri-nucleotide repeats in the array of tri-nucleotide repeats compared to a wild-type TCF4 gene.

In some embodiments, the array of tri-nucleotide repeats comprises the sequence (CTG) n or a complementary sequence thereof, wherein n is any integer greater than 39, greater than 49, or greater than 99.

In some embodiments, the first editing template comprises an exogenous sequence compared to the TCF4 gene and/or the second editing template comprises an exogenous sequence compared to the TCF4 gene.

In some embodiments, the region of complementarity between the first editing template and the second editing template comprises an exogenous sequence compared to the TCF4 gene.

In some embodiments, the exogenous sequence comprises a marker, an expression tag, a barcode, or a regulatory sequence.

In some embodiments, the first editing template comprises a region of complementarity to the IND on the second strand of the TCF4 gene.

In some embodiments, the second editing template comprises a region of complementarity to the IND on the first strand of the TCF4 gene.

In some embodiments, the sequence of the region of complementarity between the first editing template and the second editing template is at least partially identical to a sequence in the IND.

In some embodiments, the first editing template comprises the sequence (CUG)ₙ, wherein n is any integer between 0 and 33, between 5 and 30, or between 10 and 25 (SEQ ID NO: 398).

In some embodiments, the second editing template comprises the sequence (CAG)ₘ, wherein m is any integer between 0 and 33, between 5 and 30, or between 10 and 25 (SEQ ID NO: 399).

In some embodiments, the region of complementarity between the first editing template and the second editing template comprises the sequence (CUG)_{w}, wherein w is any integer between 0 and 33, between 5 and 30, or between 10 and 25 (SEQ ID NO: 398).

In some embodiments, the (n+m−w) is an integer no greater than 38.

In some embodiments, the IND further comprises a sequence upstream of the array of tri-nucleotide repeats.

In some embodiments, the sequence upstream of the tri-nucleotide repeat sequence is at least 10 base pairs in length, is 5 to 25 base pairs in length, is 20 to 50 base pairs in length, or is 50 to 100 base pairs in length.

In some embodiments, the sequence upstream of the tri-nucleotide repeat sequence is 100, 200, 300, 400, or 500 base pairs in length.

In some embodiments, the IND further comprises a sequence downstream of the array of tri-nucleotide repeats.

In some embodiments, the sequence downstream of the tri-nucleotide repeat sequence is at least 10 base pairs in length, is 5 to 25 base pairs in length, is 20 to 50 base pairs in length, or is 50 to 100 base pairs in length.

In some embodiments, the sequence downstream of the tri-nucleotide repeat sequence is 100, 200, 300, 400, or 500 base pairs in length.

In some embodiments, the first editing template further comprises a region of complementarity to the sequence of the IND upstream of the array of tri-nucleotide repeats.

In some embodiments, the region of complementarity of the first editing template to the sequence of the IND upstream of the array of tri-nucleotide repeats is 5 to 25 nucleotides in length, 10 to 15 nucleotides in length, 20 to 50 nucleotides in length, or 50 to 100 nucleotides in length.

In certain embodiments, the first editing template comprises at its 5′ end, a sequence selected from the group consisting of: nucleotides 1-100 of SEQ ID NO: 115; nucleotides 1-90 of SEQ ID NO: 116; nucleotides 1-80 of SEQ ID NO: 117; nucleotides 1-70 of SEQ ID NO: 118; nucleotides 1-60 of SEQ ID NO: 119; nucleotides 1-50 of SEQ ID NO: 120; nucleotides 1-40 of SEQ ID NO: 121; nucleotides 1-30 of SEQ ID NO: 122; nucleotides 1-20 of SEQ ID NO: 123, nucleotides 1-10 of SEQ ID NO: 124, and SEQ ID NOs: 105-114 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, and 299.

In certain embodiments, the first editing template comprises at its 3′ end, a sequence selected from the group consisting of: the last 100 nucleotides of SEQ ID NO: 115; the last 90 nucleotides of SEQ ID NO: 116; the last 80 nucleotides of SEQ ID NO: 117; the last 70 nucleotides of SEQ ID NO: 118; the last 60 nucleotides of SEQ ID NO: 119; the last 50 nucleotides of SEQ ID NO: 120; the last 40 nucleotides of SEQ ID NO: 121; the last 30 nucleotides of SEQ ID NO: 122; the last 20 nucleotides of SEQ ID NO: 123; the last10 nucleotides of SEQ ID NO: 124, and SEQ ID NOs: 104-114 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, and 299.

In certain embodiments, the second editing template comprises at its 5′ end, a sequence selected from the group consisting of: nucleotides 1-100 of SEQ ID NO: 135; nucleotides 1-90 of SEQ ID NO: 136; nucleotides 1-80 of SEQ ID NO: 137; nucleotides 1-70 of SEQ ID NO: 138; nucleotides 1-60 of SEQ ID NO: 139; nucleotides 1-50 of SEQ ID NO: 140; nucleotides 1-40 of SEQ ID NO: 141; nucleotides 1-30 of SEQ ID NO: 142; nucleotides 1-20 of SEQ ID NO: 143, nucleotides 1-10 of SEQ ID NO: 144, and SEQ ID NOs: 125-134; and 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

In certain embodiments, the second editing template comprises at its 3′ end, a sequence selected from the group consisting of: the last 100 nucleotides of SEQ ID NO: 135; the last 90 nucleotides of SEQ ID NO: 136; the last 80 nucleotides of SEQ ID NO: 137; the last 70 nucleotides of SEQ ID NO: 138; the last 60 nucleotides of SEQ ID NO: 139; the last 50 nucleotides of SEQ ID NO: 140; the last 40 nucleotides of SEQ ID NO: 141; the last 30 nucleotides of SEQ ID NO: 142; the last 20 nucleotides of SEQ ID NO: 143, the last 10 nucleotides of SEQ ID NO: 144, and SEQ ID NOs: 125-134; 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

In some embodiments, the first editing template comprises nucleotides 1 to x of SEQ ID NO: a, wherein x is an integer from 10 to i, wherein i is the length of SEQ ID NO: a; wherein the second editing template comprises nucleotides 1 to y of SEQ ID NO: b, wherein y is an integer from (i+10−x) to i; wherein a is an integer from 105 to 124 or the integer 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, and wherein b is an integer that equals (a+99).

In some embodiments, the second editing template further comprises a region of complementarity to the sequence of the IND downstream of the array of tri-nucleotide repeats.

In some embodiments, the region of complementarity of the second editing template to the sequence of the IND downstream of the array of tri-nucleotide repeats is 5 to 25 nucleotides in length, 10 to 15 nucleotides in length, 20 to 50 nucleotides in length, or 50 to 100 nucleotides in length.

In some embodiments, the second editing template comprises a nucleotides 1 to x of SEQ ID NO: a, wherein x is an integer from 10 to i, wherein i is the length of SEQ ID NO: a; wherein the first editing template comprises nucleotides 1 to y of SEQ ID NO: b, wherein y is an integer from (i+10−x) to i; wherein a is an integer from 125 to 144 or the integer 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, or 300, and wherein b is an integer that equals (a+99).

In some embodiments, the first editing template and the second editing template are not complementary to each other.

In some embodiments, the first editing template and the second editing template comprise a region of complementarity to each other.

In some embodiments, the region of complementarity between the first editing template and the second editing template comprises an exogenous sequence compared to the double-stranded TCF4 gene.

In some embodiments, the exogenous sequence comprises a marker, an expression tag, a barcode, or a regulatory sequence.

In some embodiments, the first editing template comprises a region of identity or substantial identity to a sequence on the first strand of the double-stranded TCF4 gene immediately adjacent to and outside the IND.

In some embodiments, the region of identity or substantial identity of the first editing template to the sequence on the first strand of the double-stranded TCF4 gene immediately adjacent to and outside the IND is at least 10 nucleotides in length, or is 15 to 100 nucleotides in length.

In some embodiments, the second editing template comprises a region of identity or substantial identity to a sequence on the second strand of the TCF4 gene immediately adjacent to and outside the IND.

In some embodiments, the region of identity or substantial identity of the second editing template to the sequence on the second strand of the double-stranded TCF4 gene immediately adjacent to and outside the IND is at least 10 nucleotides in length, or is 15 to 100 nucleotides in length.

In some embodiments, the first PEgRNA comprises a first primer binding site (PBS) sequence that comprises a region of complementarity to the second strand of the double-stranded TCF4 gene.

In some embodiments, the second PEgRNA comprises a second PBS sequence that comprises a region of complementarity to the first strand of the double-stranded TCF4 gene.

In one embodiment, the first PEgRNA comprises a structure: 5′-[first spacer]-[first gRNA core]-[first editing template]-[first primer binding site sequence]-3′.

In another embodiment, the first PEgRNA comprises a structure: 5′-[first editing template]-[first primer binding site sequence]-[first spacer]-[first gRNA core]-3′.

In one embodiment, the second PEgRNA comprises a structure: 5′-[second spacer sequence]-[second gRNA core]-[second editing template]-[second primer binding site]-3′.

In another embodiment, the second PEgRNA comprises a structure: 5′-[second editing template]-[second primer binding site sequence]-[second spacer]-[second gRNA core]-3′.

In some embodiments, the first PBS is at least partially complementary to the first spacer sequence.

In some embodiments, the second PBS is at least partially complementary to the second spacer sequence.

In some embodiments, the first search target sequence is at least 10, 50, 100, 200, 300, 400 or 500 nucleotides upstream of the IND. In some embodiments, the first search target sequence is at least 100 nucleotides upstream of the IND. In some embodiments, the first search target sequence is at least 50 nucleotides upstream of the IND.

In some embodiments, the second search target sequence is at least 10, 50, 100, 200, 300, 400 or 500 nucleotides downstream of the IND. In some embodiments, the second search target sequence is at least 100 nucleotides downstream of the IND. In some embodiments, the second search target sequence is at least 50 nucleotides downstream of the IND.

In some embodiments, the first PBS is about 2 to 20 nucleotides in length or about 8 to 16 nucleotides in length.

In some embodiments, the second PBS is about 2 to 20 nucleotides in length or about 8 to 16 nucleotides in length.

In some embodiments, the first editing template is about 15-150 nucleotides in length, about 15-100 nucleotides in length, about 30-100 nucleotides in length, about 50-100 nucleotides in length, or about 15-50 nucleotides in length.

In some embodiments, the second editing template is about 15-150 nucleotides in length, about 15-100 nucleotides in length, about 30-100 nucleotides in length, about 50-100 nucleotides in length, or about 15-50 nucleotides in length.

In some embodiments, the first editing template and second editing template are of the same length.

In another embodiment, the first editing template and second editing template are of different lengths.

In some embodiments, the first and/or the second spacer is about 15 to 25 nucleotides in length, about 17 to 22 nucleotides in length, or about 20 to 22 nucleotides in length.

In some embodiments, the first spacer comprises a sequence selected from the group consisting of SEQ ID NOs: 65-69.

In some embodiments, the second spacer comprises a sequence selected from the group consisting of SEQ ID NOs: 70-74.

In some embodiments, the first PBS comprises a sequence selected from the group consisting of SEQ ID NOs: 75-89.

In some embodiments, the second PBS comprises a sequence selected from the group consisting of SEQ ID NOs: 90-104.

In some embodiments, the first editing template comprises a sequence selected from the group consisting of SEQ ID NOs: 105-124 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299.

In some embodiments, the second editing template comprises a sequence selected from the group consisting of SEQ ID NOs: 125-144 and 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, or 300.

In one embodiment, a composition comprises a first prime editing guide RNA (PEgRNA) and a second PEgRNA, wherein the first PEgRNA comprises a first spacer comprising a sequence selected from the group consisting of SEQ ID NOs: 65-69, a guide RNA core comprising a sequence selected from the group consisting of SEQ ID NOs: 301-305 and 318-385, a first PBS comprising a sequence selected from the group consisting of SEQ ID NOs: 75-87, and a first editing template comprising a sequence selected from the group consisting of SEQ ID NOs: 105-124 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, and wherein the second PEgRNA comprises a second spacer comprising a sequence selected from the group consisting of SEQ ID NOs: 70-74, a guide RNA core comprising a sequence selected from the group consisting of SEQ ID NOs: 301-305 and 381-385, a second PBS comprising a sequence selected from the group consisting of SEQ ID NOs: 90-104, and a second editing template comprising a sequence selected from the group consisting of SEQ ID NOs: 125-144 and 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, or 300.

In one embodiment, the editing pair is selected from the group comprising SEQ ID NOs: 317 and 335; SEQ ID NOs: 318 and 321; SEQ ID NOs: 318 and 322; SEQ ID NOs: 317 and 335, SEQ ID NOs: 318 and 321, SEQ ID NOs: 318 and 322, SEQ ID NOs: 315 and 321, SEQ ID NOs: 315 and 331, SEQ ID NOs: 319 and 321, SEQ ID NOs: 319 and 323, SEQ ID NOs: 319 and 324, SEQ ID NOs: 319 and 326, SEQ ID NOs: 315 and 332, SEQ ID NOs: 316 and 329, SEQ ID NOs: 316 and 332, SEQ ID NOs: 319 and 329, SEQ ID NOs: 319 and 335, SEQ ID NOs: 320 and 324, SEQ ID NOs: 320 and 325, SEQ ID NOs: 317 and 324, or combination thereof.

In one embodiment, the editing pair is selected from the group comprising SEQ ID NOs: 306 and 324, SEQ ID NOs: 306 and 329, SEQ ID NOs: 306 and 331, SEQ ID NOs: 306 and 332, SEQ ID NOs: 310 and 322, SEQ ID NOs: 310 and 323, SEQ ID NOs: 310 and 325, SEQ ID NOs: 310 and 326, SEQ ID NOs: 310 and 331, SEQ ID NOs: 310 and 332, SEQ ID NOs: 313 and 332, SEQ ID NOs: 314 and 326, SEQ ID NOs: 317 and 329, SEQ ID NOs: 317 and 331, SEQ ID NOs: 317 and 332, SEQ ID NOs: 318 and 323, SEQ ID NOs: 318 and 326, SEQ ID NOs: 307 and 322, SEQ ID NOs: 307 and 323, SEQ ID NOs: 307 and 326, SEQ ID NOs: 307 and 329, SEQ ID NOs: 307 and 331, SEQ ID NOs: 307 and 332, SEQ ID NOs: 311 and 322, SEQ ID NOs: 311 and 323, SEQ ID NOs: 311 and 329, SEQ ID NOs: 311 and 331, SEQ ID NOs: 311 and 332, SEQ ID NOs: 314 and 332, SEQ ID NOs: 315 and 322, SEQ ID NOs: 315 and 323, SEQ ID NOs: 315 and 325, SEQ ID NOs: 315 and 326, SEQ ID NOs: 315 and 328, SEQ ID NOs: 315 and 329, SEQ ID NOs: 318 and 329, SEQ ID NOs: 318 and 331, SEQ ID NOs: 318 and 332, SEQ ID NOs: 318 and 335, SEQ ID NOs: 319 and 322, SEQ ID NOs: 319 and 325, SEQ ID NOs: 319 and 328, SEQ ID NOs: 308 and 323, SEQ ID NOs: 308 and 326, SEQ ID NOs: 308 and 331, SEQ ID NOs: 308 and 332, SEQ ID NOs: 309 and 322, SEQ ID NOs: 309 and 323, SEQ ID NOs: 309 and 324, SEQ ID NOs: 312 and 322, SEQ ID NOs: 312 and 323, SEQ ID NOs: 312 and 326, SEQ ID NOs: 312 and 329, SEQ ID NOs: 312 and 331, SEQ ID NOs: 312 and 332, SEQ ID NOs: 316 and 322, SEQ ID NOs: 316 and 323, SEQ ID NOs: 316 and 325, SEQ ID NOs: 316 and 326, SEQ ID NOs: 316 and 328, SEQ ID NOs: 319 and 331, SEQ ID NOs: 319 and 332, SEQ ID NOs: 320 and 322, SEQ ID NOs: 320 and 323, SEQ ID NOs: 320 and 326, SEQ ID NOs: 320 and 329, SEQ ID NOs: 309 and 325, SEQ ID NOs: 309 and 326, SEQ ID NOs: 309 and 329, SEQ ID NOs: 309 and 331, SEQ ID NOs: 309 and 332, SEQ ID NOs: 313 and 322, SEQ ID NOs: 313 and 323, SEQ ID NOs: 317 and 321, SEQ ID NOs: 317 and 322, SEQ ID NOs: 317 and 323, SEQ ID NOs: 317 and 325, SEQ ID NOs: 317 and 326, SEQ ID NOs: 320 and 331, SEQ ID NOs: 320 and 332, or combination thereof.

In one embodiment, the present disclosure provides a dual prime editing system comprising the composition of any one of the preceding embodiments and a prime editor, wherein the prime editor comprises a DNA binding domain and a DNA polymerase domain.

In one embodiment, a dual prime editing system comprises the composition of any one of the preceding embodiments, and further comprises a first prime editor that comprises a DNA binding domain and a DNA polymerase domain and associates with the first PEgRNA, and a second prime editor that comprises a DNA binding domain and a DNA polymerase domain and associates with the second PEgRNA.

In some embodiments, the first prime editor and the second prime editor are the same.

In some embodiments, the DNA binding domain is a CRISPR associated (Cas) protein domain.

In some embodiments, the Cas protein domain has nickase activity.

In some embodiments, the Cas protein domain is a Cas9, Cas9 comprising a mutation in an HNH domain, Cas9 comprising a H840A mutation in the HNH domain, Cas12a, Cas12b, Cas12c, Cas12d, Cas12e, Cas14a, Cas14b, Cas14c, Cas14d, Cas14e, Cas14f, Cas14g, Cas14h, Cas14u, or a Casφ.

In some embodiments, the DNA polymerase domain is a reverse transcriptase.

In some embodiments, the reverse transcriptase is a retrovirus reverse transcriptase, or a Moloney murine leukemia virus (M-MLV) reverse transcriptase.

In some embodiments, the DNA polymerase domain and the DNA binding domain are fused or linked to form a fusion protein. In some embodiments, the DNA binding domain is a programmable DNA binding domain.

In one embodiment, a lipid nanoparticle (LNP) or ribonucleoprotein (RNP) comprises a dual prime editing system as disclosed herein, or a component thereof.

In one embodiment, a polynucleotide encodes a first PEgRNA and second PEgRNA of the compositions or dual prime editing systems disclosed herein, or components thereof.

In some embodiments, the polynucleotide is a mRNA.

In some embodiments, the polynucleotide is operably linked to a regulatory element.

In some embodiments, the regulatory element is an inducible regulatory element.

In one embodiment, a vector comprises a polynucleotide disclosed herein.

In one embodiment, a vector comprises a polynucleotide encoding a first PEgRNA and second PERNA of the compositions or dual prime editing systems disclosed herein.

In one embodiment, the vector is an AAV vector.

In one embodiment, an isolated cell comprises the first PEgRNA and second

PEgRNA of the compositions as disclosed or the dual prime editing system as disclosed herein.

In one embodiment, an isolated cell comprises the LNP or RNP as disclosed, the polynucleotide as disclosed, or the vector as disclosed herein.

In some embodiments, the cell is a human cell, a human eye cell, a human corneal cell, or a human corneal endothelial cell.

In some embodiments, the cell is a corneal endothelial cell, a corneal endothelial progenitor cell, a differentiated corneal endothelial cell, or a fibroblast.

In one embodiment, a pharmaceutical composition comprises (i) the composition(s), dual prime editing system(s), LNP or RNP, polynucleotide(s), vector(s), or cell(s) as disclosed and (ii) a pharmaceutically acceptable carrier.

In one embodiment, a method for editing a TCF4 gene comprises contacting the TCF4 gene with (i) a composition as disclosed herein, (ii) a first prime editor comprising a DNA binding domain and a DNA polymerase domain that associates with the first PEgRNA, and (iii) a second prime editor comprising a DNA binding domain and a DNA polymerase domain that associates with the second PEgRNA, wherein the first PEgRNA directs the first prime editor to generate a first nick on the second strand of the TCF4 gene, wherein the second PEgRNA directs the second prime editor to generate a second nick on the first strand of the TCF4 gene, and wherein the contacting results in excision of an inter-nick duplex (IND) between the position of the first nick and the position of the second nick of the TCF4 gene, thereby editing the TCF4 gene.

In one embodiment, a method for editing a TCF4 gene comprises contacting the TCF4 gene with the dual prime editing system as disclosed, wherein the first PEgRNA directs the first prime editor to generate a first nick on the second strand of the TCF4 gene, wherein the second PEgRNA directs the second prime editor to generate a second nick on the first strand of the TCF4 gene, and wherein the contacting results in excision of an inter-nick duplex (IND) between the position of the first nick and the position of the second nick of the TCF4 gene, thereby editing the TCF4 gene.

In some embodiments of a method disclosed herein, the first prime editor and the second prime editor are the same. In some embodiments, the first editing template encodes a first single-stranded DNA, and the first single-stranded DNA is incorporated in the TCF4 gene. In some embodiments, the second editing template encodes a second single-stranded DNA, and the second single-stranded DNA is incorporated in the TCF4 gene. In some embodiments, the contacting results in deletion of the array of tri-nucleotide repeats in the TCF4 gene. In some embodiments, the contacting results in a reduced number of tri-nucleotide repeats in the TCF4 gene. In some embodiments, the contacting results in deletion of the sequence (CTG) x in the TCF4 gene, wherein x is an integer no less than 1. In some embodiments, x is an integer between 5 and 30, greater than 50, greater than 100, and/or greater than 1000. In some embodiments, the contacting results in no greater than 33 CTG repeats in intron 3 of the TCF4 gene. In some embodiments, the contacting results in no CTG repeats in intron 3 of the TCF4 gene.

In one embodiment, a method for editing a TCF4 gene comprises contacting the TCF4 gene with (i) the compositions as disclosed and (ii) a prime editor comprising a DNA binding domain and a DNA polymerase domain, wherein the first PEgRNA and second PEgRNA direct the prime editor to incorporate the intended nucleotide edit in the TCF4 gene, thereby editing the TCF4 gene.

In one embodiment, a method for editing a TCF4 gene comprises contacting the TCF4 gene with the dual prime editing system as disclosed, wherein the first PEgRNA and second PEgRNA direct the prime editor to incorporate the intended nucleotide edit in the TCF4 gene, thereby editing the TCF4 gene.

In some embodiments, the TCF4 gene is in a cell, mammalian cell, human cell, primary cell, endothelial cell, or corneal endothelial cell.

In some embodiments, the cell is in a subject. In some embodiments, the cell is in a subject and the subject is a human.

In some embodiments, the cell is from a subject having FECD.

In some embodiments, a method disclosed herein further comprises administering the cell to the subject after the contacting.

In some embodiments, a method disclosed herein further comprises administering the cell to the subject after incorporation of the intended nucleotide edit.

In one embodiment, a method for treating FECD in a subject in need thereof comprises administering to the subject the composition(s), dual prime editing system(s), LNP or RNP, polynucleotide(s), vector(s), cell(s), or pharmaceutical composition(s) as disclosed, wherein the first PEgRNA and second PEgRNA direct the prime editor to incorporate the intended nucleotide edit in the TCF4 gene in the subject, thereby treating FECD in the subject.

In one embodiment, a method for treating FECD in a subject in need thereof comprises administering to the subject the composition(s), dual prime editing system(s), LNP or RNP, polynucleotide(s), vector(s), cell(s), or pharmaceutical composition(s) as disclosed, wherein the administration results in a reduced number of an array of CTG repeats in the TCF4 gene in the subject, thereby treating FECD in the subject.

In some embodiments, the subject is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic of a prime editing guide RNA (PEgRNA) binding to a double-stranded target DNA sequence.

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

FIG. 4A depicts an exemplary schematic of a dual prime editing system for editing both strands of a double-stranded target DNA. Same color/shading indicates complementarity or identity between sequences.

FIG. 4B depicts an exemplary schematic of dual prime editing with a replacement duplex (RD) comprising an overlap duplex (OD). Same color/shading indicates complementarity or identity between sequences.

FIG. 4C depicts an exemplary schematic of dual prime editing. Same color/shading indicates complementarity or identity between sequences.

FIG. 4D depicts an exemplary schematic of dual prime editing. Same color/shading indicates complementarity or identity between sequences.

FIG. 4E depicts an exemplary schematic of dual prime editing. Same color/shading indicates complementarity or identity between sequences.

FIG. 4F depicts an exemplary schematic of dual prime editing. Same color/shading indicates complementarity or identity between sequences.

FIG. 4G depicts an exemplary schematic of dual prime editing. Same color/shading indicates complementarity or identity between sequences.

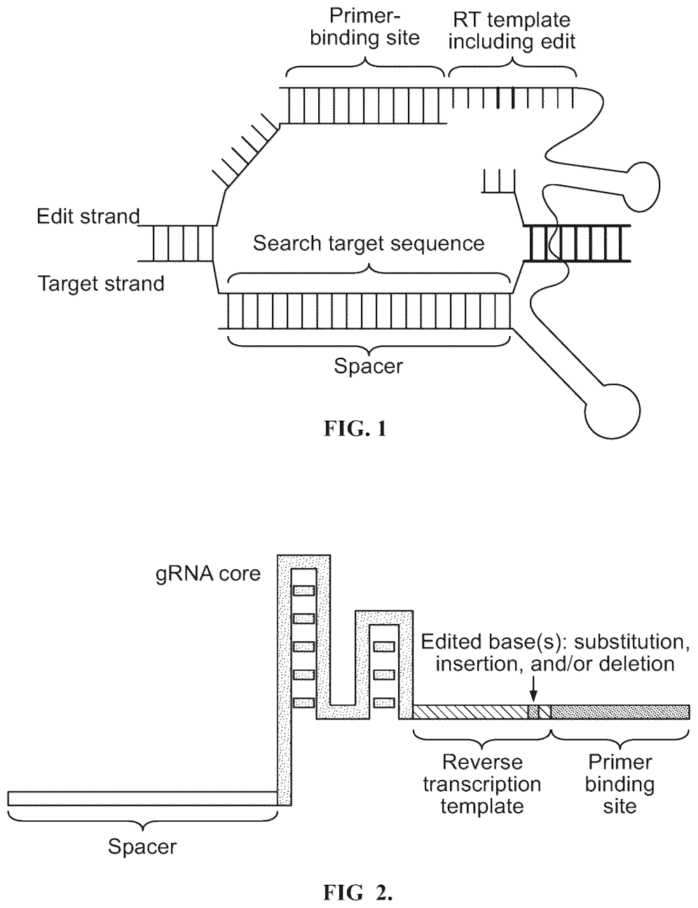

### DETAILED DESCRIPTION OF THE INVENTION

Provided herein, in some embodiments, are compositions and methods to edit the target gene TCF4 with dual prime editing. In certain embodiments, provided herein are compositions and methods for correction of mutations in the (TCF4) gene associated with FECD and/or bipolar disorder. Compositions provided herein can comprise prime editors (PEs) and engineered guide polynucleotides, e.g., prime editing guide RNAs (PEgRNAs), that can direct PEs to specific DNA targets and can encode DNA edits in the TCF4gene, including correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

This disclosure refers to position numbers in polynucleotides. Unless otherwise noted, nucleotide x, in a polynucleotide sequence, refers to the nucleotide at position number x in the polynucleotide sequence from a 5′ to 3′ order.

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used herein they mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” in relation to a numerical means, a range of values that fall within 10% greater than or less than the value. For example, about x means x±(10%*x).

The term “between” when used with reference to a range of numbers, means the range of numbers including the first and the last number in the range.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, and nematode), a cell from a vertebrate animal (e.g., fish, amphibian, reptile, bird, mammal), and a cell from a mammal (e.g., a pig, a cow, a goat, a sheep, a rodent, a rat, a mouse, a non-human primate, and a human). Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term primary cell means a cell isolated from an organism, e.g., a mammal, which is grown in tissue culture (i.e., in vitro) for the first time before subdivision and transfer to a subculture. In some non-limiting examples, mammalian primary cells can be modified through introduction of one or more polynucleotides, polypeptides, and/or prime editing compositions (e.g., through transfection, transduction, electroporation and the like) and further passaged. Such modified mammalian primary cells include muscle cells (e.g., cardiac muscle cells, smooth muscle cells, and myosatellite cells), endothelial cells (e.g., mammary endothelial cells, intestinal endothelial cells, and hepatocytes), endothelial cells, glial cells, neural cells, formed elements of the blood (e.g., lymphocytes, bone marrow cells), precursors of any of these somatic cell types, and stem cells. In some embodiments, the cell is a fibroblast. In some embodiments, the cell is a human fibroblast. In some embodiments, the cell is a cell that generates endothelium. In some embodiments, the cell is a cell that generates corneal endothelium. In some embodiments, the cell is a human fibroblast. In some embodiments, the cell is a cell that generates human endothelium. In some embodiments, the cell is a cell that generates human corneal endothelium. In some embodiments, the cell is a primary endothelial cell. In some embodiments, the cell is a human primary endothelial cell. In some embodiments, the cell is a stem cell. In some embodiments, the cell is an embryonic stem cell (ESC). In some embodiments, the cell is an induced human pluripotent stem cell (iPSC). In some embodiments, the cell is a human stem cell. In some embodiments, the cell is a human embryonic stem cell.

In some embodiments, a cell is not isolated from an organism but forms part of a tissue or organ of an organism, e.g., a mammal. In some non-limiting examples, mammalian cells include eye cells, corneal cells, muscle cells (e.g., cardiac muscle cells, smooth muscle cells, and myosatellite cells), endothelial cells (e.g., mammary endothelial cells, intestinal endothelial cells, and hepatocytes), endothelial cells, glial cells, neural cells, formed elements of the blood (e.g., lymphocytes, bone marrow cells), precursors of any of these somatic cell types, and stem cells. In some embodiments, the cell is a primary corneal endothelial cell. In some embodiments, the cell is a corneal endothelial cell. In some embodiments, the cell is a human corneal endothelial cell. In some embodiments, the cell is a stem cell. In some embodiments, the cell is a human stem cell.

In some embodiments, the cell is a progenitor cell. In some embodiments, the cell is a differentiated cell. In some embodiments, the cell is a fibroblast. In some embodiments, the cell is a corneal endothelial cell. In some embodiments, the corneal endothelial cell is differentiated from an iPSC, ESC or corneal endothelial progenitor cell. In some embodiments, the cell is a differentiated human cell. In some embodiments, a human corneal endothelial cell is differentiated from a human iPSC or human ESC.

In some embodiments, the cell comprises a prime editor or a prime editing composition. In some embodiments, the cell comprises a dual prime editing composition comprising a prime editor and at least two PEgRNAs that are different from each other. In some embodiments, the cell is from a human subject. In some embodiments, the human subject has a disease or condition associated with one or more mutations to be corrected by prime editing, for example, FECD. In some embodiments, the cell is from a human subject, and comprises a prime editor or a prime editing composition for correction of the one or more mutations. In some embodiments, the cell is from the human subject and the mutation has been edited or corrected by prime editing. In some embodiments, the cell is in a human subject, and comprises a prime editor or a prime editing composition for correction of the one or more mutations. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of Moloney murine leukemia virus. A protein that comprises amino acid sequences from different origins or naturally occurring proteins may be referred to as a fusion, or chimeric protein.

In some embodiments, a protein comprises a functional variant or functional fragment of a full-length wild-type protein. A “functional fragment” or “functional portion”, as used herein, refers to any portion of a reference protein (e.g., a wild-type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild-type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild-type Cas9, but retains its DNA binding ability and lacks its nuclease activity partially or completely.

A “functional variant”, as used herein, refers to any variant of a reference protein (e.g., a wild-type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild-type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional variant of a Cas9 may comprise one or more amino acid substitutions in a nuclease domain, e.g., an H840A amino acid substitution, compared to the amino acid sequence of a wild-type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptide includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

Gene homologs across species can be determined by sequence identity or similar function. Thus, the terms “homologous,” “homology,” or “percent homology” as used herein refer to the degree of sequence identity between an amino acid or polynucleotide sequence and a corresponding reference sequence or functional similarity. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote stable binding of a spacer, primer binding site or protospacer sequence to the complementary sequence of a genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding to the complementary sequence of a corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16:276-277), and the GGSEARCH program https://fasta.bioch.virginia.edu/fasta_www2/, which is part of the FASTA package (Pearson W and Lipman D, 1988, Proc. Natl. Acad. Sci. USA, 85:2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double-stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, a cytosine on one polynucleotide molecule will base pair to a guanine on a second polynucleotide molecule and an adenine on one polynucleotide molecule will base pair to a thymine or uracil on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, are translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA or transcript that is encoded by the gene after transcription of the gene. In some embodiments, expression of a polynucleotide, e.g., mRNA, is determined by the amount of the protein encoded by the mRNA after translation of the mRNA. In some embodiments, expression of a polynucleotide, e.g., mRNA or coding RNA, is determined by the amount of a functional form of the protein encoded by the polypeptide after translation of the polynucleotide.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a nucleic acid that in its native state or when manipulated by methods well known to those skilled in the art, contains the information that can be used as a template for synthesis of another nucleic acid, amino acid, or polypeptide. For example, a DNA sequence can encode a RNA sequence, i.e., serve as the template for transcription of the RNA sequence. A RNA sequence can encode a DNA sequence, serving as the template for reverse transcription of RNA into a cDNA sequence. A DNA or RNA sequence can encode a polypeptide sequence and can contain the information for the translation template of the polypeptide sequence. In some embodiments, one or more or a few nucleotides can be said to specifically encode a mutation or a desired nucleotide edit. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” or “variant” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. A mutation in a polynucleotide may be insertion or expansion of one or more nucleotides, or example, expansion of three nucleotides (tri-nucleotide expansion). In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of a polypeptide or the mutation in the nucleic acid sequence of a polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective” means having the ability to produce a biological response. For example, “effective amount” or “therapeutically effective amount” may refer to a quantity of a composition, for example a composition comprising a construct, that can be sufficient to result in a desired activity upon introduction into a subject as disclosed herein. An effective amount of the prime editing compositions can be provided to the target gene or cell, whether the cell is in vitro, ex vivo or in vivo.

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro, ex vivo or in vivo).

An effective amount can be the amount to induce, when administered to a population of cells, at least about a 2-fold decrease in the number of cells that have expanded CTG repeat number in the TCF4 gene. An effective amount can be the amount to induce, when administered to a population of cells, at least about a 2-fold decrease in the number of cells that have 40 or more CTG repeats in a TCF4 mRNA encoded by the TCF4 gene, or at least about a 2-fold decrease in the number of cells that have 50 or more CTG repeats in a TCF4 mRNA encoded by the TCF4 gene.

In some embodiments, an effective amount can be the amount to induce, for example, at least about 1.1-fold decrease, about 1.5 fold decrease, about 2-fold decrease, about 3-fold decrease, about 4-fold decrease, about 5-fold decrease, about 6-fold decrease, about 7-fold decrease, about 8-fold decrease, about 9-fold decrease, about 10-fold decrease, about 25-fold decrease, about 50-fold decrease, about 100-fold decrease, about 200-fold decrease, about 500-fold decrease, about 700-fold decrease, about 1000-fold decrease, about 5000-fold decrease, or about 10,000-fold decrease in the amount of TCF4 transcripts having 40 or more CTG repeats. In some embodiments, an effective amount can be the amount to induce, for example, at least about 2-fold decrease, about 3-fold decrease, about 4-fold decrease, about 5-fold decrease, about 6-fold decrease, about 7-fold decrease, about 8-fold decrease, about 9-fold decrease, about 10-fold decrease, about 25-fold decrease, about 50-fold decrease, about 100-fold decrease, about 200-fold decrease, about 500-fold decrease, about 700-fold decrease, about 1000-fold decrease, about 5000-fold decrease, or about 10,000-fold decrease in the amount of TCF4 transcripts having 50 or more CTG repeats.

The term “construct” refers to a polynucleotide or a portion of a polynucleotide, comprising one or more nucleic acid sequences encoding one or more transcriptional products and/or proteins. A construct may be a recombinant nucleic acid molecule or a part thereof. In some embodiments, the one or more nucleic acid sequences of a construct are operably linked to one or more regulatory sequences, for example, transcriptional initiation regulatory sequences. In some embodiments, a construct is a vector, a plasmid, or a portion thereof. In some embodiments, a construct comprises DNA. In some embodiments, a construct comprises RNA. In some embodiments, a construct is double-stranded. In some embodiments, a construct is single-stranded. In some embodiments, a construct comprises an expression cassette. An expression cassette means a polynucleotide comprising a nucleic acid sequence that encodes one or more transcriptional products and is operably linked to at least one transcriptional regulatory sequence, e.g., a promoter.

The term “exogenous” when used in reference to a biomolecule, e.g., a polynucleotide sequence or a polypeptide sequence refers to a biomolecule that is not native to a specific biological context, e.g., a gene, a particular chromosome, a particular cell or chromosomal site of the cell, tissue, or organism, or, if from the same source, is modified from its original form or is present in a non-native location, e.g., a chromosome location.

The term “endogenous” when used in reference to a biomolecule, e.g., a polynucleotide sequence or a polypeptide sequence refers to a biomolecule that is native to or naturally occurring in a specific biological context, e.g., a gene, a particular chromosome, a particular cell or chromosomal site of the cell, tissue, or organism. For example, an endogenous sequence may be a wild-type sequence or may comprise one or more mutations compared to a wild-type sequence. In some embodiments, an endogenous sequence is mutated compared to a wild-type sequence and may cause or be associated with a disease or disorder in a subject. As used herein, in some embodiments, a wild-type sequence, with respect to a specific gene and a specific disease, is a gene sequence found in healthy individuals, wherein the wild-type sequence does not include a mutation causative of the specific disease. In some embodiments, in the context of repeat expansion disease, a wild-type sequence may be used to refer to a sequence that harbors an array of a specific tri-nucleotide repeats within a normal range. For example, in some embodiments, the tri-nucleotide repeat is a CTG repeat in a TCF4 gene, and a wild-type sequence of a TCF4 gene may have 5 to 34 CTG repeats (SEQ ID NO: 406). For example, repeat lengths greater than 40 are often considered pathogenic, causing FECD.

A “repeat expansion disorder” or “trinucleotide repeat disorder” or “expansion repeat disorder” refers to a set of genetic disorders which are caused by “trinucleotide repeat expansion,” which is a kind of mutation characterized by expanded number of repeats of an contiguous array of three nucleotides each having the same sequence, referred to as “trinucleotide repeat” or “triplet repeat”. As used herein, an array of tri-nucleotide repeats means at least two contiguous tri-nucleotides that are the same. In some embodiments, an array of tri-nucleotide repeats comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 repeats of the same tri-nucleotides. In some embodiments, an array of tri-nucleotide repeats comprises at least 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 repeats of the same tri-nucleotides. In some embodiments, an array of tri-nucleotide repeats comprises about 50 to 100, about 100 to 150, about 150 to 200, about 200 to 250, about 250 to 300, about 300 to 400, about 400 to 500, about 500 to 600, about 600 to 700, about 700 to 800, about 800 to 900, about 900 to 1000 repeats of the same tri-nucleotides. In some embodiments, an array of tri-nucleotide repeats comprises more than 1000 repeats of the same tri-nucleotides. In some embodiments, an array of tri-nucleotide repeats comprises about 5 to 10, 5 to 15, 5 to 20, 5 to 25, 5 to 30, 5 to 35, 5 to 40, 5 to 45, 5 to 50, 10 to 15, 10 to 20, 10 to 25, 10 to 30, 10 to 35, 10 to 40, 10 to 45, 10 to 50, 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 repeats of the same tri-nucleotides. In some embodiments, an array of tri-nucleotide repeats is in a non-coding region of a gene, for example, a 3′ UTR, a 5′ UTR, or an intron of a gene. In some embodiments, an array of tri-nucleotide repeats is in a regulatory sequence, e.g., a promoter of a gene. In some embodiments, an array of tri-nucleotide repeats is in an upstream sequence or a downstream sequence of a gene. In some embodiments, an array of tri-nucleotide repeats is in a coding region of a gene. In some embodiments, an array of tri-nucleotide repeats encodes an array of amino acid repeats. In some embodiments, the number of repeats of the same tri-nucleotides in an array of tri-nucleotide repeats is altered as compared to the number of repeats in a reference array of tri-nucleotide repeats, for example, the number of repeats in a wild-type gene sequence. In some embodiments, the number of repeats of the same tri-nucleotides in an array of tri-nucleotide repeats is increased as compared to the number of repeats in a reference array of tri-nucleotide repeats, for example, the number of repeats in a wild-type gene sequence. In some embodiments, the altered or increased number of repeats in an array of tri-nucleotide repeats compared to the number of the same repeats in a wild-type gene sequence is associated with a disease.

### Dual Prime Editing

In some embodiments, prime editing may comprise programmable editing of a target DNA using one or more prime editors each complexed with a PEgRNA (“dual prime editing”). Dual prime editing refers to programmable editing of a double-stranded target DNA using two or more PEgRNAs, each of which is complexed with a prime editor for incorporating one or more intended nucleotide edits into the double-stranded target DNA. In some embodiments, dual prime editing incorporates one or more intended nucleotide edits into a double-stranded target DNA through excision of an endogenous DNA segment and/or replacement of the endogenous DNA segment with newly synthesized DNA via target-primed DNA synthesis. In some embodiments, dual prime editing may be used to edit a target DNA that is or is part of a target gene. In some embodiments, the target gene is a disease-associated gene. In some embodiments, the target gene is a monogenic disease-associated gene. In some embodiments, the target gene is a polygenic disease-associated gene. In some embodiments, the target gene is mutated compared to a wild-type sequence of the same gene and may cause or be associated with a disease or disorder in a subject. In some embodiments, the mutated target gene causes a disease or a disorder in a human subject.

The term “prime editing” refers to programmable editing of a target DNA using a prime editor complexed with a PEgRNA to incorporate an intended nucleotide edit into the target DNA through target-primed DNA synthesis. In prime editing, a target DNA may comprise a double-stranded DNA molecule having two complementary strands. When viewed in the context of each specific PEgRNA, the two complementary strands of a double-stranded target DNA may comprise a strand that may be referred to as a “target strand” or a “non-edit strand”, and a complementary strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a PAM sequence. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise uracil (U) and the protospacer sequence may comprise thymine (T).

In some embodiments, dual prime editing involves using two different PEgRNAs each complexed with a prime editor, wherein each of the two PEgRNAs comprises a spacer complementary or substantially complementary to a separate search target sequence. In some embodiments, each of the two PEgRNAs anneals with a separate search target sequence through its spacer. Accordingly, references to a “PAM strand”, a “non-PAM strand”, a “target strand’, a “non-target strand”, an “edit strand” or a “non-edit strand” are relative in the context of a specific PEgRNA, e.g., one of the two PEgRNAs in dual prime editing.

In some embodiments, dual prime editing involves two PEgRNAs, different from one another, each complexed with a prime editor. In some embodiments, each of the two PEgRNAs comprises a region of complementarity to a distinct search target sequence of the target DNA, wherein the two distinct search target sequences are on the two complementary strands of the target DNA. The terms “region”, “portion”, and “segment” are used interchangeably to refer to a proportion of a molecule, for example, a polynucleotide or a polypeptide. For example, a region of a polynucleotide may be 5%, 10%, 15%, 20%, 25%, 30%, 35%, 40%, 45%, 50%, 55%, 60%, 65%, 70%, 75%, 80%, 85%, 90%, 95% or 100% of the polynucleotide. In some embodiments, the two PEgRNAs each can direct a prime editor to initiate the prime editing process on the two complementary strands of the target DNA.

In some embodiments, dual prime editing involves two PEgRNAs each complexed with a prime editor. In some embodiments, a first PEgRNA comprises a first spacer complementary to a first search target sequence on a first strand of a double-stranded target DNA, e.g., a double-stranded target gene. In the context of the first PEgRNA, the first strand of the double-stranded target DNA may be referred to as a first target strand, and the complementary strand referred to as the first PAM strand.

In some embodiments, a second PEgRNA comprises a second spacer complementary to a second search target sequence on a second strand of the double-stranded target DNA. In some embodiments, the first strand and the second strand of the double-stranded target DNA, e.g., a double-stranded target gene, are complementary to each other. Accordingly, in some embodiments, the second PEgRNA and the first PEgRNA bind opposite strands of the double-stranded target DNA. In the context of the second PERNA, the second strand of the double-stranded target DNA may be referred to as a second target strand, and the complementary strand referred to as the second PAM strand. In some embodiments, the first target strand is the same strand as the second PAM strand of the double-stranded target DNA. In some embodiments, the second target strand is the same strand as the first PAM strand of the double-stranded target DNA.

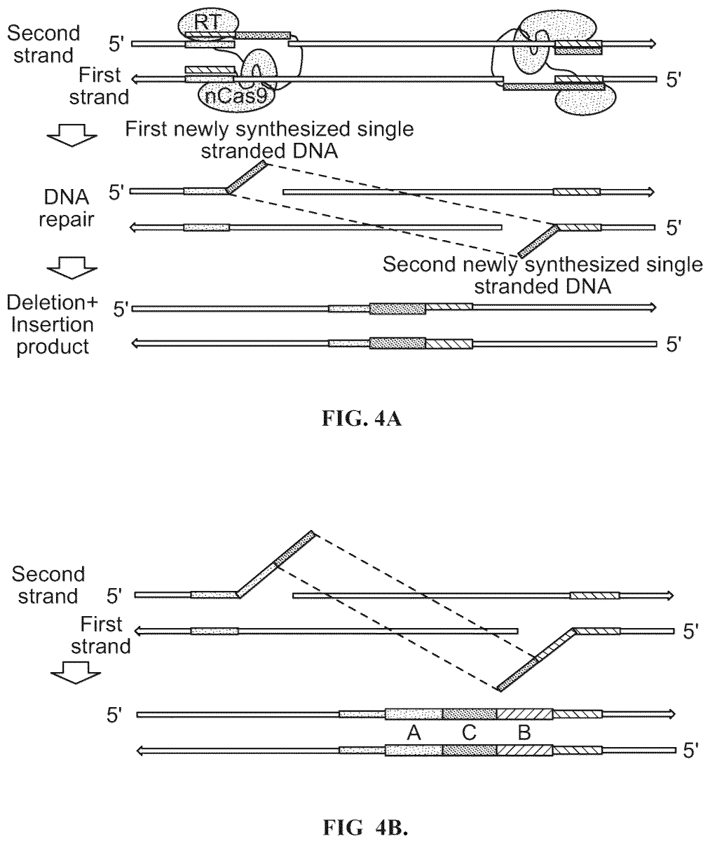

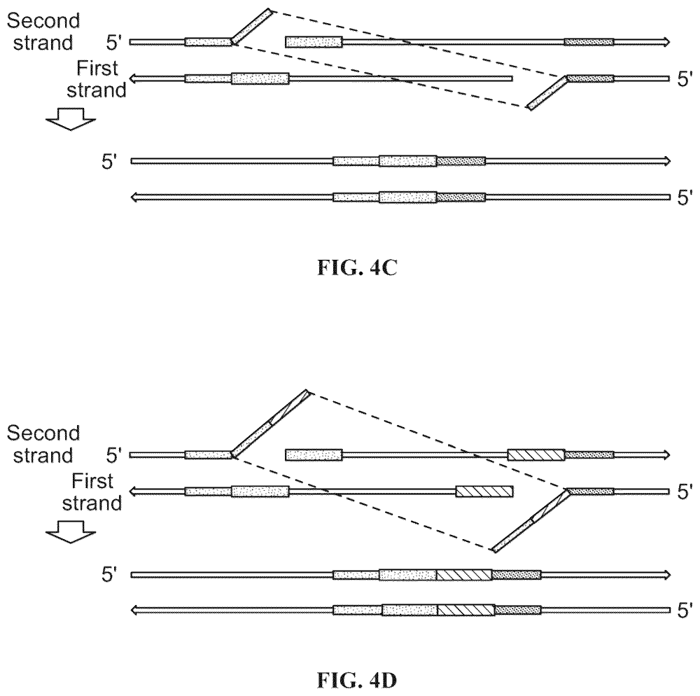

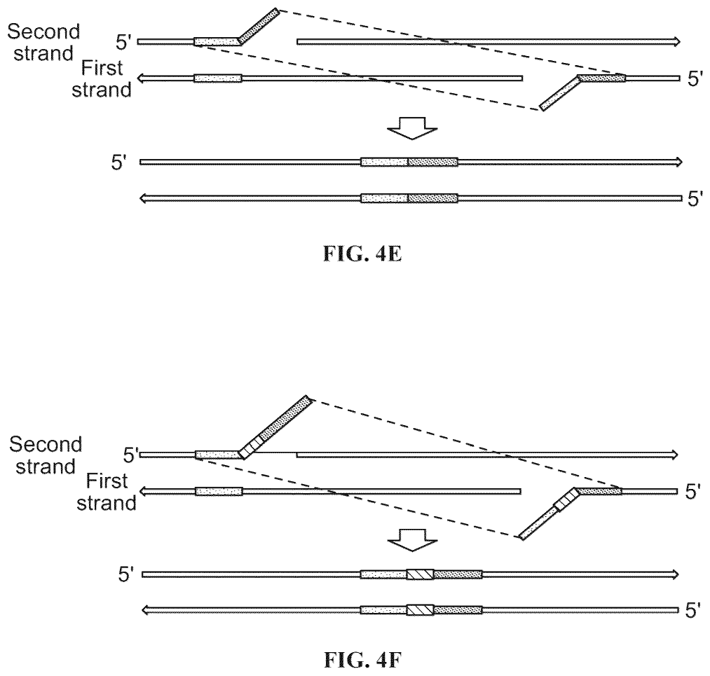

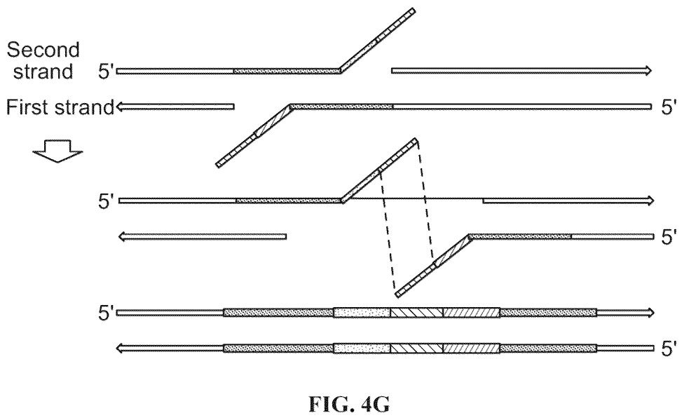

As used herein for editing of the TCF4 gene, the first PEgRNA may also be referred to as the “5′ PEgRNA”, and the second PEgRNA may be referred to as the “3′ PEgRNA”. Specifically, the 5′ to 3′ orientation of the TCF4 gene refers to the 5′ to 3′ orientation of the coding strand (i.e. sense strand) of the TCF4 gene. The first PERNA (5′ PEgRNA) comprises a first spacer having complementarity to a first search target sequence on the non-coding strand of TCF4, and is capable of directing a prime editor to nick the coding strand at a first nick site that is 5′ to the CTG repeats. The second PEgRNA (3′ PERNA) comprises a second spacer having complementarity to a second search target sequence on the coding strand of TCF4, and is capable of directing a prime editor to nick the non-coding strand at a second nick site that is 5′ to the CAG repeats (that is, the position corresponding to the second nick site on the coding strand is 3′ to the CTG repeats). An exemplary dual prime editing strategy for editing the TCF4 gene is provided in FIG. 4A, where the first strand (bottom) is the non-coding strand, and the second strand (top) is the coding strand. The (CTG) repeats are accordingly in the coding strand, and the non-coding strand contains the complementary (CAG) repeats. A 5′ PERNA complexed with a prime editor is at the left side of the figure, and a 3′ PEgRNA complexed with a prime editor is at the right side.

In some embodiments, the first PEgRNA anneals with the first target strand of the double-stranded target DNA, through the first spacer of the first PEgRNA. In some embodiments, the first PERNA complexes with and directs a first prime editor to bind the double-stranded target DNA at the position corresponding to the first search target sequence. In some embodiments, the second PEgRNA anneals with the second search target sequence on the second target strand of the double-stranded target DNA, through a second spacer of the second PERNA. In some embodiments, the second PEgRNA complexes with and directs a second prime editor to bind the double-stranded target DNA at the position corresponding to the second search target sequence. In some embodiments, the first prime editor and the second prime editor are the same. In some embodiments, the first prime editor and the second prime editor are different.

In some embodiments, the first search target sequence recognized by the spacer of the first PEgRNA and the second search target sequence recognized by the spacer of the second PEgRNA have a region of complementarity to each other. In some embodiments, the region of complementarity is 2 to 20 nucleotides in length. In some embodiments, the region of complementarity is 5 to 15 nucleotides in length.

In some embodiments, the first search target sequence recognized by the spacer of the first PEgRNA and the second search target sequence recognized by the spacer of the second PEgRNA do not have a region of complementarity to each other. In some embodiments, the positions of the first and second search target sequences relative to each other may be determined by their positions in the double-stranded target DNA prior to editing. In some embodiments, the positions of the first and second search target sequences relative to each other may be determined by their positions in a reference double-stranded target DNA.

In some embodiments, the 3′ end of the first search target sequence and the position corresponding to the 3′ end of the second search target sequence are 2, 3, 4, 5, 6, 7, 8, 9, 10, 11, 12, 13, 14, 15, 16, 17, 18, 19, 20, 21,,22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides apart from each other. In some embodiments, the 3′ end of the first search target sequence and the position corresponding to the 3′ end of the second search target sequence are about 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nucleotides apart from each other. In some embodiments, the 3′ end of the first search target sequence and the position corresponding to the 3′ end of the second search target sequence are about 500, 600, 700, 800, 900, 1000, 1100, 1200, 1300, 1400, 1500, 1600, 1700, 1800, 1900, 2000 or more nucleotides apart from each other. In some embodiments, the 3′ end of the first search target sequence and the position corresponding to the 3′ end of the second search target sequence are about 150 to about 450 nucleotides apart from each other. In some embodiments, the 3′ end of the first search target sequence and the position corresponding to the 3′ end of the second search target sequence are about 105 to about 145 nucleotides apart from each other. In some embodiments, the 3′ end of the first search target sequence and the position corresponding to the 3′ end of the second search target sequence are about 300 to about 3000 nucleotides apart from each other. In some embodiments, the 3′ end of the first search target sequence and the position corresponding to the 3′ end of the second search target sequence at least about 3000 nucleotides apart from each other.

In some embodiments, the bound first prime editor generates a first nick on the first PAM strand of the double-stranded target DNA. In some embodiments, a first PEgRNA comprises a first primer binding site (PBS, also referred to herein as “primer binding site sequence”) that is complementary to the sequence of the first PAM strand of the double-stranded target DNA that is immediately upstream of the first nick site, and can anneal with the sequence of the first PAM strand at a free 3′ end formed at the first nick site. In some embodiments, a first PEgRNA comprises a first primer binding site (PBS) that anneals to a free 3′ end formed at the first nick site and the first prime editor initiates DNA synthesis from the nick site, using the free 3′ end as a primer. In some embodiments, the first prime editor generates a first newly synthesized single-stranded DNA encoded by a first editing template of the first PEgRNA.

In some embodiments, the bound second prime editor generates a second nick on the second PAM strand of the double-stranded target DNA. In some embodiments, the double-stranded target DNA, e.g., a target gene, comprises a double-stranded DNA sequence between the first nick generated by the first prime editor on the second target strand (also referred to as the first PAM strand) and the second nick generated by the second prime editor on the first target strand (also referred to as the second PAM strand), which may be referred to as an inter-nick duplex (IND). In some embodiments, the two strands of an IND are perfectly complementary to each other. In some embodiments, the two strands of an IND are partially complementary to each other. In some embodiments, the IND is subsequently excised from the double-stranded target DNA, e.g., the target gene.

In some embodiments, the IND is 1, 2, 3, 4, 5, 6, 7, 8, 9, 10, 11, 12, 13, 14, 15, 16, 17, 18, 19, 20 or more base pairs in length. In some embodiments, the IND is up to 5, up to 10, up to 15, up to 20, up to 25, up to 30, up to 40, or up to 50 base pairs in length. In some embodiments, the IND is 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 base pairs in length. In some embodiments, the IND is 500-3000, 500-2500, 500-2000, 500-1500, 500-1000, 500-900, 500-800, 500-700, or 500-600 base pairs in length. In some embodiments, the IND is 30-300, 30-250, 30-200, 30-150, 30-100, 30-75, 30-50, 50-200, 50-150, 50-100, 50-75, 75-100, 75-150, 75-200, 75-250, 75-300 base pairs in length. In some embodiments, the IND is 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 base pairs in length. In some embodiments, the IND is 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 base pairs in length. In some embodiments, the IND is 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 base pairs in length. In some embodiments, the IND is about 150 to about 450 base pairs in length. In some embodiments, the IND is about 105 to about 145 base pairs in length. In some embodiments, the IND is about 300 to about 3000 base pairs in length. In some embodiments, the IND is more than about 3000 base pairs in length.

In some embodiments, the double-stranded target DNA is a double-stranded target gene or a part of a double-stranded target gene, and the IND comprises a part of a coding sequence of the target gene. In some embodiments, the IND comprises a part of a non-coding sequence of the target gene. In some embodiments, the IND comprises a part of an exon. In some embodiments, the IND comprises an entire exon. In some embodiments, the IND comprises a part of an intron. In some embodiments, the IND comprises an entire intron. In some embodiments, the IND comprises a 3′ UTR sequence of the target gene. In some embodiments, the IND comprises a 5′ UTR sequence of the target gene. In some embodiments, the IND comprises a whole or a part of an ORF of the target gene. In some embodiments, the IND comprises both coding and non-coding sequences of the target gene. In some embodiments, the IND comprises both intron and exon sequences of the target gene. For example, in some embodiments, the IND comprises the sequence of an exon flanked by an intronic sequence at the 5′ end, the 3′ end, or both ends. In some embodiments, the IND comprises one or more exons and intervening introns. In some embodiments, the IND comprises two or more exons and intervening introns. In some embodiments, the IND comprises all of the coding regions of a target gene, regulatory sequences of a target gene, or the entire target gene comprising its exons, introns and regulatory sequences. In some embodiments, the double-stranded DNA comprises a gene or a part of a gene, and the IND comprises one or more mutations compared to a wild-type reference sequence of the same gene. In some embodiments, the one or more mutations are associated with a disease. In some embodiments, the IND comprises an array of three nucleotide repeats (or tri-nucleotide repeats). In some embodiments, the IND comprises an array of tri-nucleotide repeats, wherein the number of the tri-nucleotide repeats is associated with a disease. As used herein, an array of tri-nucleotide repeats means at least two tri-nucleotides that are the same. In some embodiments, an array of tri-nucleotide repeats comprises at least 10, 20, 30, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 repeats of the same tri-nucleotides. In some embodiments, an array of tri-nucleotide repeats comprises at least 34 tri-nucleotide repeats. In some embodiments, an array of tri-nucleotide repeats comprises at least 50 tri-nucleotide repeats. In some embodiments, an array of tri-nucleotide repeats comprises at least 100 tri-nucleotide repeats. In some embodiments, an array of tri-nucleotide repeats comprises at least 1000 tri-nucleotide repeats. In some embodiments, an array of tri-nucleotide repeats is in a non-coding region of a gene, for example, a 3′ UTR, of a gene. In some embodiments, the array of tri-nucleotide repeats is an array of CTG (or the reverse complement CAG) repeats.

In some embodiments, a first PEgRNA comprises a first primer binding site (PBS) that is complementary to a free 3′ end of the second strand of the double-stranded target DNA formed at the first nick site. In some embodiments, the first PBS anneals with the free 3′ end formed at the first nick site, and the first prime editor initiates DNA synthesis from the first nick site, using the free 3′ end at the first nick site as a primer. In some embodiments, the first prime editor synthesizes a first new single-stranded DNA encoded by the first editing template of the first PEgRNA. In some embodiments, the second PEgRNA comprises a second PBS that is complementary to a free 3′ end of the first strand of the double-stranded target DNA formed at the second nick site. In some embodiments, the second PBS anneals with the free 3′ end formed at the second nick site, and the second prime editor initiates DNA synthesis from the nick site, using the free 3′ end at the second nick site as a primer. In some embodiments, the second prime editor synthesizes a second newly synthesized single-stranded DNA encoded by a second editing template of the second PEgRNA.

In some embodiments, through DNA repair, the sequence of the first newly synthesized single-stranded DNA encoded by the first editing template and/or the sequence of the second newly synthesized single-stranded DNA encoded by the second editing template is incorporated into the double-stranded target DNA, e.g., a target gene, thereby incorporating one or more intended nucleotide edits in the double-stranded target DNA, e.g., the target gene.

As used herein, a “nucleotide edit” or an “intended nucleotide edit” refers to a specified edit of a double-stranded target DNA. A nucleotide edit or intended nucleotide edit refers to a (i) deletion of one or more contiguous nucleotides at one specific position, (ii) insertion of one or more contiguous nucleotides at one specific position, (iii) substitution of one or more contiguous nucleotides, or (iv) a combination of contiguous nucleotide substitutions, insertions and/or deletions of two or more contiguous nucleotides at one specific position, or other alterations at one specific position to be incorporated into the sequence of the double-stranded target DNA. An intended nucleotide edit may refer to the edit on an editing template (e.g., a first editing template or a second editing template) as compared to the sequence of the double-stranded target gene, or may refer to the edit encoded by an editing template in the newly synthesized single-stranded DNA that is incorporated in the double-stranded target DNA, e.g., the TCF4 gene, as compared to endogenous sequence of the double-stranded target DNA, e.g., the TCF4 gene. In some embodiments, an intended nucleotide edit may also refer to the edit that results from incorporation of the newly synthesized DNA encoded by an editing template, or incorporation of the two newly synthesized single-stranded DNA encoded by each of the first PERNA and the second PEgRNA in dual prime editing.

In some embodiments, the sequence of the first newly synthesized single-stranded DNA and/or the sequence of the second newly synthesized single-stranded DNA are incorporated into the double-stranded target DNA, e.g., the target gene. In some embodiments, the first and/or the second newly synthesized single-stranded DNAs comprises one or more intended nucleotide edits compared to the endogenous sequence of the double-stranded target DNA, e.g., the target gene, which are incorporated in the double-stranded target DNA, e.g., the target gene. In some embodiments, the sequence of the first newly synthesized single-stranded DNA encoded by the first editing template is incorporated in the double-stranded target DNA, e.g., the target gene, thereby incorporating one or more intended nucleotide edits in the double-stranded target DNA, e.g., the target gene. In some embodiments, the sequence of the second newly synthesized single-stranded DNA encoded by the second editing template is incorporated in the double-stranded target DNA, e.g., the target gene, thereby incorporating one or more intended nucleotide edits in the double-stranded target DNA, e.g., the target gene. In some embodiments, the sequence of the first newly synthesized single-stranded DNA encoded by the first editing template and the sequence of the second newly synthesized single-stranded DNA encoded by the second editing template are incorporated in the double-stranded target DNA, e.g., the target gene, thereby incorporating one or more intended nucleotide edits in the double-stranded target DNA, e.g., the target gene.

In some embodiments, the intended nucleotide edit comprises an insertion, deletion, nucleotide substitution, inversion, or any combination thereof compared to the endogenous sequence of the double-stranded target DNA, e.g., the target gene. In some embodiments, the intended nucleotide edit comprises 1, 2, 3, 4, 5, 6, 7, 8, 9, 10, 11, 12, 13, 14, 15, 16, 17, 18, 19, 20 or more nucleotide substitutions compared to the endogenous sequence of the double-stranded target DNA, e.g., the target gene. In some embodiments, the intended nucleotide edit comprises up to 5, up to 10, up to 15, up to 20, up to 25, up to 30, up to 40, or up to 50 nucleotide substitutions compared to the endogenous sequence of the double-stranded target DNA, e.g., the target gene. In some embodiments, the intended nucleotide edit comprises 1-50, 1-40, 1-30, 1-25, 1-20, 1-15, 1-10, or 1-5 nucleotide substitutions compared to the endogenous sequence of the double-stranded target DNA, e.g., the target gene. In some embodiments, the intended nucleotide edit comprises 3-50, 3-40, 3-30, 3-25, 3-20, 3-15, 3-10, or 3-5 nucleotide substitutions compared to the endogenous sequence of the double-stranded target DNA, e.g., the target gene. In some embodiments, the intended nucleotide edit comprises 5-50, 5-40, 5-30, 5-25, 5-20, 5-15, or 5-10 nucleotide substitutions compared to the endogenous sequence of the double-stranded target DNA, e.g., the target gene.

In some embodiments, the intended nucleotide edit comprises 1, 2, 3, 4, 5, 6, 7, 8, 9, 10, 11, 12, 13, 14, 15, 16, 17, 18, 19, 20 or more nucleotide insertions compared to the endogenous sequence of the double-stranded target DNA, e.g., the target gene. In some embodiments, the intended nucleotide edit comprises up to 5, up to 10, up to 15, up to 20, up to 25, up to 30, up to 40, or up to 50 nucleotide insertions compared to the endogenous sequence of the double-stranded target DNA, e.g., the target gene. In some embodiments, the intended nucleotide edit comprises single nucleotide insertions at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises nucleotide insertions of greater than one nucleotide at each site in the double-stranded target DNA compared to the endogenous sequence of the double-stranded target DNA, e.g., the target gene. As used herein, “site” refers to a specific position in the sequence of a target DNA, e.g., a target gene. In some embodiments, the specific position in the sequence of the double-stranded target DNA, e.g., a target gene, can be referred to by specific positions in a reference sequence, e.g., a wild-type gene sequence. In some embodiments, a nucleotide insertion at position x refers to insertion of one or more nucleotides between position x and position x+1 as set forth by numbering in a reference sequence. In some embodiments, a nucleotide deletion at position x refers to deletion of the specific nucleotide at position x as set forth by numbering in a reference sequence. In some embodiments, a nucleotide deletion of positions x to x+n refers to deletion of the specific nucleotides starting at nucleotide x to nucleotide x+n, including nucleotide x and nucleotide x+n, as set forth by numbering in a reference sequence. In some embodiments, a nucleotide inversion of positions x to x+n refers to inversion of the specific nucleotides starting at nucleotide x to nucleotide x+n, including nucleotide x and nucleotide x+n, as set forth by numbering in a reference sequence.

In some embodiments, the intended nucleotide edit comprises nucleotide insertions of greater than one nucleotide at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 nucleotide insertions compared to the endogenous sequence of the double-stranded target DNA, e.g., the target gene. In some embodiments, the intended nucleotide edit comprises 500-3000, 500-2500, 500-2000, 500-1500, 500-1000, 500-900, 500-800, 500-700, or 500-600 nucleotide insertions compared to the endogenous sequence of the double-stranded target DNA, e.g., the target gene. In some embodiments, the intended nucleotide edit comprises 30-300, 30-250, 30-200, 30-150, 30-100, 30-75, 30-50, 50-200, 50-150, 50-100, 50-75, 75-100, 75-150, 75-200, 75-250, or 75-300 nucleotide insertions compared to the endogenous sequence of the double-stranded target DNA, e.g., the target gene. In some embodiments, the intended nucleotide edit comprises nucleotide insertions of 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 nucleotides at each site in the double-stranded target DNA compared to the endogenous sequence of the double-stranded target DNA, e.g., the target gene.

In some embodiments, the intended nucleotide edit comprises 1, 2, 3, 4, 5, 6, 7, 8, 9, 10, 11, 12, 13, 14, 15, 16, 17, 18, 19, 20 or more nucleotide deletions compared to the endogenous sequence of the double-stranded target DNA, e.g., the target gene. In some embodiments, the intended nucleotide edit comprises up to 5, up to 10, up to 15, up to 20, up to 25, up to 30, up to 40, or up to 50 nucleotide deletions compared to the endogenous sequence of the double-stranded target DNA, e.g., the target gene. In some embodiments, the intended nucleotide edit comprises single nucleotide deletions at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises nucleotide deletions of greater than one nucleotide at each site in the double-stranded target DNA compared to the endogenous sequence of the double-stranded target DNA, e.g., the target gene. In some embodiments, the intended nucleotide edit comprises nucleotide deletions of greater than one nucleotide at 1, 2, 3, 4, 5, 6, 7, 8, 9, 10, 11, 12, 13, 14, 15, 16, 17, 18, 19, 20 or more sites in the double-stranded target DNA compared to the endogenous sequence of the double-stranded target DNA, e.g., the target gene. In some embodiments, the intended nucleotide edit comprises 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, or 1-5 nucleotide deletions compared to the endogenous sequence of the double-stranded target DNA, e.g., the target gene. In some embodiments, the intended nucleotide edit comprises 500-3000, 500-2500, 500-2000, 500-1500, 500-1000, 500-900, 500-800, 500-700, or 500-600 nucleotide deletions compared to the endogenous sequence of the double-stranded target DNA, e.g., the target gene. In some embodiments, the intended nucleotide edit comprises 30-300, 30-250, 30-200, 30-150, 30-100, 30-75, 30-50, 50-200, 50-150, 50-100, 50-75, 75-100, 75-150, 75-200, 75-250, 75-300 nucleotide deletions compared to the endogenous sequence of the double-stranded target DNA, e.g., the target gene. In some embodiments, the intended nucleotide edit comprises deletion of about 105-145 nucleotides at a site of the target gene, e.g., the TCF4 gene. In some embodiments, the intended nucleotide edit comprises deletion of about 150-450 nucleotides at a site of the target gene, e.g., the TCF4 gene. In some embodiments, the intended nucleotide edit comprises deletion of about 300-3000 nucleotides at a site of the target gene, e.g., the TCF4 gene. In some embodiments, the intended nucleotide edit comprises deletion of more than about 3000 nucleotides at a site of the target gene, e.g., the TCF4 gene.

In some embodiments, the intended nucleotide edit comprises 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 nucleotide deletions compared to the endogenous sequence of the double-stranded target DNA, e.g., the target gene. In some embodiments, the intended nucleotide edit comprises nucleotide deletions of 1-3000, 1-2500, 1-2000, 1-1500, 1-1000, 1-900, 1-800, 1-700, 1-600, 1-500, 1-400, 1-300, 1-200, 1-100, 1-50, 1-40, 1-30, 1-25, 1-20, 1-15, 1-10, 1-5, 500-3000, 500-2500, 500-2000, 500-1500, 500-1000, 500-900, 500-800, 500-700, 500-600, 30-300, 30-250, 30-200, 30-150, 30-100, 30-75, 30-50, 50-200, 50-150, 50-100, 50-75, 75-100, 75-150, 75-200, 75-250, or 75-300 nucleotides at each site in the double-stranded target DNA compared to the endogenous sequence of the double-stranded target DNA, e.g., the target gene. In some embodiments, the intended nucleotide edit comprises nucleotide deletions of 1-3, 1-6, 1-9, 1-12, 1-15, 1-18, 1-21, 1-24, 1-27, 1-30, 1-36, 1-45, 1-60, 1-72, 1-90, 3-6, 3-9, 3-12, 3-15, 3-18, 3-21, 3-24, 3-27, 3-30, 3-36, 3-45, 3-60, 3-72, 3-90, 6-9, 6-12, 6-15, 6-18, 6-21, 6-24, 6-27, 6-30, 6-36, 6-45, 6-60, 6-72, 6-90, 9-12, 9-15, 9-18, 9-21, 9-24, 9-27, 9-30, 9-36, 9-45, 9-60, 9-72, 9-90, 12-15, 12-18, 12-21, 12-24, 12-27, 12-30, 12-36, 12-45, 12-60, 12-72, 12-90, 15-18, 15-21, 15-24, 15-27, 15-30, 15-36, 15-45, 15-60, 15-72, 15-90, 18-21, 18-24, 18-27, 18-30, 18-36, 18-45, 18-60, 18-72, 18-90, 21-24, 21-27, 21-30, 21-36, 21-45, 21-60, 21-72, 21-90, 24-27, 24-30, 24-36, 24-45, 24-60, 24-72, 24-90, 27-30, 27-36, 27-45, 27-60, 27-72, 27-90, 30-36, 30-45, 30-60, 30-72, 30-90, 45-60, 45-72, 60-72, 60-90, or 72-90 nucleotides at each site in the double-stranded target DNA compared to the endogenous sequence of the double-stranded target DNA, e.g., the target gene.

In some embodiments, the intended nucleotide edits, e.g., nucleotide substitutions, insertions, or deletions, are in consecutive or contiguous nucleotides in the double-stranded target DNA sequence compared to the endogenous sequence of the double-stranded target DNA, e.g., the target gene. In some embodiments, the intended nucleotide edits, e.g., nucleotide substitutions, insertions, or deletions are in non-consecutive or non-contiguous nucleotides in the double-stranded target DNA sequence compared to the endogenous sequence of the double-stranded target DNA, e.g., the target gene.

In some embodiments, the intended nucleotide edit comprises an inversion as compared to the endogenous sequence of the double-stranded target DNA, e.g., the target gene. In some embodiments, a segment of 2, 3, 4, 5, 6, 7, 8, 9, 10, 20, 30, 40, 50, 60, 70, 80, 90, 100, 110, 120, 130, 140, 150, 200, 250, 300 or more nucleotides of the endogenous sequence of the double-stranded target DNA is inverted. In some embodiments, a segment of 1-50, 1-40, 1-30, 1-25, 1-20, 1-15, 1-10, or 1-5 nucleotides of the endogenous sequence of the double-stranded target DNA is inverted. In some embodiments, a segment of 3-50, 3-40, 3-30, 3-25, 3-20, 3-15, 3-10, 3-5, 5-50, 5-40, 5-30, 5-25, 5-20, 5-15, or 5-10 nucleotides of the endogenous sequence of the double-stranded target DNA is inverted.

In some embodiments, the intended nucleotide edit comprises more than one nucleotide edit in the double-stranded target DNA sequence compared to the endogenous sequence of the double-stranded target DNA, e.g., the target gene. In some embodiments, the intended nucleotide edit comprises a combination of one or more of nucleotide substitutions, one or more of nucleotide insertions, one or more of nucleotide deletions and one or more of nucleotide inversions compared to the endogenous sequence of the double-stranded target DNA, e.g., the target gene. In some embodiments, the intended nucleotide edit comprises one or more nucleotide substitutions and one or more nucleotide insertions. In some embodiments, the intended nucleotide edit comprises one or more nucleotide substitutions and one or more nucleotide deletions. In some embodiments, the intended nucleotide edit comprises one or more nucleotide substitutions and one or more nucleotide inversions. In some embodiments, the intended nucleotide edit comprises one or more nucleotide insertions and one or more nucleotide deletions. In some embodiments, the intended nucleotide edit comprises one or more nucleotide insertions and one or more nucleotide inversions. In some embodiments, the intended nucleotide edit comprises one or more nucleotide deletions and one or more nucleotide inversions. In some embodiments, the intended nucleotide edit comprises one or more nucleotide substitutions, one or more nucleotide insertions and one or more nucleotide deletions. In some embodiments, the intended nucleotide edit comprises one or more nucleotide substitutions, one or more nucleotide insertions and one or more nucleotide inversions. In some embodiments, the intended nucleotide edit comprises one or more nucleotide substitutions, one or more nucleotide deletions and one or more nucleotide inversions. In some embodiments, the intended nucleotide edit comprises one or more nucleotide insertions, one or more nucleotide deletions and one or more nucleotide inversions. In some embodiments, the intended nucleotide edit comprises one or more nucleotide substitutions, one or more nucleotide insertions, one or more nucleotide deletions and one or more nucleotide inversions.

In some embodiments, the first newly synthesized single-stranded DNA and the second newly synthesized single-stranded DNA have a region of complementarity to each other. In some embodiments, the first newly synthesized single-stranded DNA has a region of complementarity to an endogenous sequence of the double-stranded target DNA, e.g., the target gene, adjacent to or near a nick site. In some embodiments, the first newly synthesized single-stranded DNA has a region of complementarity to an endogenous sequence of the double-stranded target DNA, e.g., the target gene, on the first strand adjacent to the second nick site. In some embodiments, the first newly synthesized single-stranded DNA has a region of complementarity to an endogenous sequence of the double-stranded target DNA, e.g., the target gene, on the first strand adjacent to and downstream of the second nick site. In some embodiments, the first newly synthesized single-stranded DNA has a region of identity to an endogenous sequence of the double-stranded target DNA e.g., the target gene, on the second strand adjacent to and downstream of the first nick site. In some embodiments, the first newly synthesized single-stranded DNA has a region of identity to an endogenous sequence of the double-stranded target on the second strand adjacent to and upstream of the second nick site.

In some embodiments, the second newly synthesized single-stranded DNA has a region of complementarity to an endogenous sequence of the double-stranded target DNA, e.g., the target gene, adjacent to or near a nick site. In some embodiments, the second newly synthesized single-stranded DNA has a region of complementarity to an endogenous sequence of the double-stranded target DNA, e.g., the target gene, on the second strand adjacent to the first nick site. In some embodiments, the second newly synthesized single-stranded DNA has a region of complementarity to an endogenous sequence of the double-stranded target DNA, e.g., the target gene, on the second strand adjacent to and downstream of the first nick site. In some embodiments, the second newly synthesized single-stranded DNA has a region of identity to an endogenous sequence of the double-stranded target DNA, e.g., the target gene, on the first strand adjacent to and upstream of the second nick site.

As used herein, reference for positioning in a chromosome or a double-stranded polynucleotide, e.g., a double-stranded target DNA, includes the position on either strand of the two strands, unless otherwise specified. For example, a position of a first nick site may be used refer to the first nick site on the first edit strand and/or the corresponding position on the second edit strand.

By “upstream” and “downstream” it is intended to define relative positions of at least two nucleotides, regions or sequences in a nucleic acid molecule oriented in a 5′-to-3′ direction according to a reference strand of the nucleic acid molecule. For example, a first nucleotide is upstream of a second nucleotide when the first nucleotide is 5′ to the second nucleotide. A first sequence is upstream of a second sequence in a DNA molecule where the first sequence is positioned 5′ to the second sequence according to a reference strand of the DNA molecule. Accordingly, the second sequence is downstream, that is, 3′, of the first sequence.

In some embodiments, each of the first newly synthesized single-stranded DNA and the second newly synthesized single-stranded DNA has a region of complementarity to an endogenous sequence of the double-stranded target DNA, e.g., the target gene, adjacent to or near a nick site. In some embodiments, each of the first newly synthesized single-stranded DNA and the second newly synthesized single-stranded DNA has a region of identity to an endogenous sequence of the double-stranded target DNA, e.g., the target gene, adjacent to or near a nick site. In some embodiments, the first newly synthesized single-stranded DNA has a region of complementarity to an endogenous sequence of the double-stranded target DNA, e.g., the target gene, on the first strand adjacent to and upstream of the second nick site, and the second newly synthesized single-stranded DNA has a region of complementarity to an endogenous sequence of the double-stranded target DNA, e.g., the target gene, on the second strand adjacent to and upstream of the first nick site. In some embodiments, the first newly synthesized single-stranded DNA has a region of identity to an endogenous sequence of the double-stranded target DNA on the second strand adjacent to and downstream of the first nick site and/or a region of identity to an endogenous sequence of the double-stranded target DNA on the second strand adjacent to and downstream of the position corresponding to the second nick site, and the second newly synthesized single-stranded DNA has a region of identity to an endogenous sequence of the double-stranded target DNA on the first strand adjacent to and downstream of the first nick site and/or a region of identity to an endogenous sequence of the double-stranded target DNA on the first strand adjacent to and downstream of the position corresponding to the first snick site.

In some embodiments, the first newly synthesized single-stranded DNA encoded by the first editing template and the second newly synthesized single-stranded DNA encoded by the second editing template have a region of complementarity to each other. The complementary region between the first newly synthesized single-stranded DNA and the second newly synthesized single-stranded DNA may be referred to as an overlap duplex (OD).

In some embodiments, the first newly synthesized single-stranded DNA encoded by the first editing template and the second newly synthesized single-stranded DNA encoded by the second editing template are substantially complementary to each other. In some embodiments, the OD is incorporated in the double-stranded target DNA, e.g., the target gene, thereby incorporating one or more intended nucleotide edits encoded by the first editing template and the second editing template into the double-stranded target DNA, e.g., the target gene. In some embodiments, the OD replaces all or a portion of the IND, thereby incorporating one or more intended nucleotide edits in the double-stranded target DNA, e.g., the target gene. In some embodiments, the IND is excised or degraded, and the OD is incorporated at the place of the IND excision, followed by ligation of the nicks on both strands of the double-stranded target DNA, e.g., the target gene, thereby incorporating the one or more intended nucleotide edits in the double-stranded target DNA. In some embodiments, the sequence of the OD comprises partial identity compared to the sequence of the IND. In some embodiments, the sequence of the OD comprises no identity compared to the sequence of the IND. In some embodiments, the sequence of the OD comprises a sequence exogenous to the double-stranded target DNA.

In some embodiments, the first editing template and the second editing template comprise a region of complementarity or substantial complementarity to each other, and do not have complementarity to either strand of the double-stranded target DNA, e.g., the target gene. Accordingly, in some embodiments, the first newly synthesized single-stranded DNA encoded by the first editing template and the second newly synthesized single-stranded DNA encoded by the second editing template can anneal to each other to form an OD that does not have substantial sequence identity with the endogenous sequence of double-stranded target DNA, e.g., the target gene. In some embodiments, the sequence of the OD comprises a sequence exogenous to the double-stranded target DNA, e.g., the target gene. In some embodiments, the sequence of the OD consists of a sequence exogenous to the double-stranded target DNA, e.g., the target gene. In some embodiments, the IND is excised, and the OD is incorporated at the place of the IND excision, followed by ligation of the nicks on both strands of the target DNA, thereby incorporating the sequence of the OD in the double-stranded target DNA.

In some embodiments, the OD comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more contiguous complementary or substantially complementary base pairs. In some embodiments, the OD comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, 045 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 contiguous complementary or substantially complementary base pairs. In some embodiments, the OD comprise 10, 11, 12, 13, 14, 15, 16, 17, 18, 19, 20, 21, 22, 23, 24 or 25 contiguous complementary or substantially complementary base pairs. In some embodiments, the OD comprise 30, 35, 40, 50, 60, 70, 80, 90, or 100 contiguous complementary or substantially complementary base pairs. In some embodiments, the OD comprise no greater than 20, 25, 30, 35, 40, 50, 60, 70, 80, 90, or 100 contiguous complementary or substantially complementary base pairs. In some embodiments, the OD comprises a sufficient number of contiguous complementary base pairs to form a sufficiently stable duplex for replacement of the IND. In some embodiments, the OD comprises at least 10 contiguous complementary or substantially complementary base pairs. In some embodiments, the OD comprises at least 15 contiguous complementary or substantially complementary base pairs. In some embodiments, the OD comprises about 20 contiguous complementary or substantially complementary base pairs.

In some embodiments, the OD contains about 20 to 40 contiguous complementary or substantially complementary base pairs. In some embodiments, the OD contains about 20, about 30, about 40, about 50, about 60, about 70, or about 80 contiguous complementary or substantially complementary base pairs. In some embodiments, the OD contains about 10 to 19 contiguous complementary or substantially complementary base pairs. In some embodiments, the OD contains about 20 to 30 contiguous complementary or substantially complementary base pairs. In some embodiments, the OD contains about 30 to 40 contiguous complementary or substantially complementary base pairs. In some embodiments, the OD contains at least 40contiguous complementary or substantially complementary base pairs. In some embodiments, the OD consists of about 20 to 40 contiguous complementary or substantially complementary base pairs. In some embodiments, the OD consists of about 20, about 30, about 40, about 50, about 60, about 70, or about 80 contiguous complementary or substantially complementary base pairs. In some embodiments, the OD consists of about 10 to 19 contiguous complementary or substantially complementary base pairs. In some embodiments, the OD consists of about 20 to 30 contiguous complementary or substantially complementary base pairs. In some embodiments, the OD consists of about 30 to 40 contiguous complementary or substantially complementary base pairs. In some embodiments, the OD consists of 23, 38, 53, 68, or 83 contiguous complementary or substantially complementary base pairs. In some embodiments, the OD consists of 38 contiguous complementary or substantially complementary base pairs.

The sequence of the OD can comprises any exogenous sequence or any endogenous sequence of the TCF4 gene. The GC content of the OD may vary. In some embodiments, the GC content of the OD is less than about 45%. In some embodiments, the GC content of the OD is about 45%-60%. In some embodiments, the GC content of the OD is about 60%-75%. In some embodiments, the GC content of the OD is at least about 75%. In some embodiments, the GC content of the OD is about 40%-80%. In some embodiments, the GC content of the OD is about 50%-60%. In some embodiments, the GC content of the OD is about 60%-80%. In some embodiments, the GC content of the OD is about 60%-70%. In some embodiments, the GC content of the OD is about 70%-80%. In some embodiments, the GC content of the OD is about 10%-20%, about 20%-30%, about 30%-40%, about 40%-50%, about 50%-60%, about 60%-70%, about 70%-80%, about 80%-90% or about 90%-100%. In some embodiments, the GC content of the OD is about 42%. In some embodiments, the GC content of the OD is about 53%. In some embodiments, the GC content of the OD is about 63%. In some embodiments, the GC content of the OD is about 71%. In some embodiments, the GC content of the OD is about 79%. In some embodiments, the GC content of the OD is about 63%.

In some embodiments, the OD replaces the IND of a target DNA, wherein the double-stranded target DNA is an entire target gene or is part of a target gene. In some embodiments, the OD replaces part of an exon or an entire exon, part of an intron or an entire intron, one or more exons and intervening introns, all of the coding regions of a target gene, regulatory sequences of a target gene, or the entire target gene comprising its exons, introns and regulatory sequences. In some embodiments, the OD comprises a region of identity to an endogenous sequence of the double-stranded target DNA. In some embodiments, the OD does not have sequence identity to an endogenous sequence of the double-stranded target DNA. In some embodiments, the OD is exogenous to the double-stranded target DNA, e.g., the target gene.

In some embodiments, the OD has a biological function or encodes a polypeptide having a biological function, or a portion thereof. In some embodiments, the OD comprises an expression cassette. In some embodiments, the OD comprises a nucleotide sequence that encodes an expression tag, for example, an affinity tag, a His tag, a V5 tag, or a FLAG tag. In some embodiments, the OD comprises a nucleotide sequence that encodes a His tag. In some embodiments, the OD comprises a nucleotide sequence that encodes a FLAG tag. In some embodiments, the OD comprises a nucleotide sequence that encodes an attB or an attP sequence. In some embodiments, the OD comprises a nucleotide sequence that encodes a reporter protein, for example, a green fluorescence protein, a blue fluorescence protein, a cyan fluorescence protein, a yellow fluorescence protein, an auto fluorescent protein, or a luciferase. In some embodiments, the OD comprises a recognition site of an enzyme, for example, a recombinase recognition sequence. In some embodiments, the OD comprises nucleotide sequence that encodes a selectable marker, for example, an antibiotic resistance marker. In some embodiments, the OD comprises a regulatory sequence, for example, a promoter, an enhancer, or an insulator. In some embodiments, the OD comprises a trackable sequence, for example, a barcode. In some embodiments, replacement of the IND by the OD restores or partially restores the function of the target gene. In some embodiments, the target gene is a disease-associated gene. In some embodiments, the target gene is a monogenic disease-associated gene. In some embodiments, the target gene is a polygenic disease-associated gene. In some embodiments, the target gene is a disease-associated gene containing one or more disease-causing mutations, wherein replacement of the IND by the OD corrects the mutations, thereby restoring or partially restoring the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the OD corrects the mutated gene, thereby restoring or partially restoring the function of the target gene. In some embodiments, the target gene is a disease-associated gene containing one or more disease-causing mutations, wherein replacement of the IND by the OD modifies the target gene to restore or partially restore the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the OD modifies the mutated gene to restore or partially restore the function of the target gene.

In some embodiments, the first editing template and/or the second editing template comprises a nucleotide sequence that encodes a polypeptide sequence that is the same as, or a portion of, the polypeptide sequence encoded by the IND, but does not have substantial nucleotide sequence complementarity or identity to the sequence of the IND. In some embodiments, the second newly synthesized single-stranded DNA encoded by the second editing template comprises a nucleotide sequence that encodes a polypeptide sequence that is the same as, or a portion of, the polypeptide sequence encoded by the IND, but does not have substantial nucleotide sequence complementarity or identity to the sequence of the IND. Accordingly, in some embodiments, the OD comprises a sequence that encodes a polypeptide sequence that is the same as the polypeptide sequence or a portion of the same polypeptide sequence encoded by the IND, wherein the OD does not have substantial nucleotide sequence identity to the sequence of the IND.

In some embodiments, the first newly synthesized single-stranded DNA encoded by the first editing template and the second newly synthesized single-stranded DNA encoded by the second editing template comprises a region of complementarity with each other, and can anneal with each other to form an OD. In some embodiments, the first newly synthesized single-stranded DNA encoded by the first editing template further comprises a region that does not have complementarity with the second newly synthesized single-stranded DNA encoded by the second editing template (see exemplary schematic in FIG. 4B). In some embodiments, the second newly synthesized single-stranded DNA encoded by the second editing template further comprises a region that does not have complementarity with the first newly synthesized single-stranded DNA encoded by the first editing template. Accordingly, in some embodiments, the first newly synthesized single-stranded DNA encoded by the first editing template and the second newly synthesized single-stranded DNA encoded by the second editing template can anneal to each other through the partially complementary sequences to form an OD that is linked to a 5′ overhang and/or a 3′overhang. In some embodiments, the IND is removed, the OD, along with the 5′ overhang and/or the 3′ overhang, is incorporated at the place of the IND excision in the double-stranded target DNA, e.g., the target gene. Through DNA repair, the gaps corresponding to the positions of the 5′ overhang and/or the 3′ overhangs are filled and ligated, thereby incorporating the one or more intended nucleotide edits in the double-stranded target DNA, e.g., the target gene.

Accordingly, in some embodiments, the IND is replaced by the sequence of (A+C), (B+C), or (A+B+C), wherein A is the region, and its complementary strand, of the first newly synthesized single-stranded DNA that is not complementary to the second newly synthesized single-stranded DNA, wherein B is the region, and its complementary strand, of the second newly synthesized single-stranded DNA that is not complementary to the first newly synthesized single-stranded DNA, and wherein C is the OD. The double-stranded sequence of (A+C), (B+C), or (A+B+C) that replaces the IND may be referred to as the “replacement duplex (RD)”.

Accordingly, in some embodiments, the RD comprises the OD. In some embodiments, as exemplified in FIG. 4A, the first editing template and the second editing template are substantially complementary to each other. Accordingly, in some embodiments, the OD comprises the entirety or substantially the entirety of the first newly synthesized single-stranded DNA encoded by the first editing template and the second newly synthesized single-stranded DNA encoded by the second editing template. In some embodiments, the RD consists of the OD. In some embodiments, as exemplified in FIG. 4B, the RD comprises the OD, the non-complementary region of the first newly synthesized DNA compared to the second newly synthesized DNA and complement thereof, and/or the non-complementary region of the second newly synthesized DNA compared to the first newly synthesized DNA and complement thereof.

In some embodiments, the RD comprises 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200 or more base pairs. In some embodiments, the RD comprises about 5 to 10, 5 to 15, 5 to 20, 5 to 25, 5 to 30, 5 to 35, 5 to 40, 5 to 45, 5 to 50, to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 base pairs. In some embodiments, the RD comprise 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs. In some embodiments, the RD comprise at least 30, 35, 40, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 base pairs. In some embodiments, the RD comprise no greater than 20, 25, 30, 35, 40, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 base pairs.

In some embodiments, the RD replaces the IND of a target DNA, wherein the IND is an entire target gene or is part of a target gene. In some embodiments, the RD replaces part of an exon or an entire exon, part of an intron or an entire intron, one or more exons and intervening introns, all of the coding regions of a target gene, regulatory sequences of a target gene, or the entire target gene comprising its exons, introns and regulatory sequences, thereby incorporating the one or more intended nucleotide edits compared to the endogenous sequence of the double-stranded target DNA, e.g., the TCF4 gene. In some embodiments, the RD comprises a region of identity to an endogenous sequence of the double-stranded target DNA. In some embodiments, the RD does not have sequence identity to an endogenous sequence of the double-stranded target DNA. In some embodiments, the RD is exogenous to the double-stranded target DNA, e.g., the target gene. Accordingly, in some embodiments, the intended nucleotide edit(s) comprises replacement of an endogenous sequence of the double-stranded target DNA, e.g., the TCF4 gene, in its entirety, by the sequence of the RD.

In some embodiments, the RD has a biological function or encodes a polypeptide having a biological function. In some embodiments, the RD comprises an expression cassette. In some embodiments, the RD comprises a nucleotide sequence that encodes an expression tag, for example, an affinity tag, a His tag, a V5 tag, or a FLAG tag. In some embodiments, the RD comprises a nucleotide sequence that encodes a His tag. In some embodiments, the RD comprises a nucleotide sequence that encodes a FLAG tag. In some embodiments, the RD comprises a nucleotide sequence that encodes an attB or an attP sequence. In some embodiments, the RD comprises a nucleotide sequence that encodes a reporter protein, for example, a green fluorescence protein, a blue fluorescence protein, a cyan fluorescence protein, a yellow fluorescence protein, an auto fluorescent protein, or a luciferase. In some embodiments, the RD comprises a recognition site of an enzyme, for example, a recombinase recognition sequence. In some embodiments, the RD comprises a nucleotide sequence that encodes a selectable marker, for example, an antibiotic resistance marker. In some embodiments, the RD comprises a regulatory sequence, for example, a promoter, an enhancer, or an insulator. In some embodiments, the RD comprises a trackable sequence, for example, a barcode. In some embodiments, replacement of the IND by the RD restores or partially restores the function of the target gene. In some embodiments, the target gene is a disease-associated gene. In some embodiments, the target gene is a monogenic disease-associated gene. In some embodiments, the target gene is a polygenic disease-associated gene. In some embodiments, the target gene is a disease-associated gene containing one or more disease-causing mutations, wherein replacement of the IND by the RD corrects the mutations, thereby restoring or partially restoring the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the RD corrects the mutated gene, thereby restoring or partially restoring the function of the target gene. In some embodiments, the target gene is a disease-associated gene containing one or more disease-causing mutations, wherein replacement of the IND by the RD modifies the target gene to restore or partially restore the function of the target gene. In some embodiments, the disease-associated gene containing one or more disease-causing mutations is in a human subject in need of treatment. In some embodiments, the target gene is a mutated gene causing a disease or disorder in a human subject, wherein replacement of the IND by the RD modifies the mutated gene to restore or partially restore the function of the target gene.

In some embodiments, the first editing template and the second editing template are partially complementary to each other. As used herein, the first editing template is partially complementary to the second editing template when the first and the second editing templates have complementary or substantially complementary region(s) over part of the length of both editing templates. The partially complementary region(s) in the first editing template and the second editing template can be in any position within the first editing template and the second editing template. Accordingly, in some embodiments, the first newly synthesized single-stranded DNA encoded by the first editing template and the second newly synthesized single-stranded DNA encoded by the second editing template are partially complementary to each other, at any position within the first newly synthesized single-stranded DNA encoded by the first editing template and the second newly synthesized single-stranded DNA encoded by the second editing template. In some embodiments, the first newly synthesized single-stranded DNA comprises a region of complementarity to the second newly synthesized single-stranded DNA, at or near the 3′ end of the first newly synthesized single-stranded DNA. In some embodiments, the first newly synthesized single-stranded DNA comprises a region of complementarity to the second newly synthesized single-stranded DNA, at or near the 5′ end of the first newly synthesized single-stranded DNA. In some embodiments, the first newly synthesized single-stranded DNA comprises a region of complementarity to the second newly synthesized single-stranded DNA, in the middle of the first newly synthesized single-stranded DNA.

In some embodiments, the second newly synthesized single-stranded DNA comprises a region of complementarity to the first newly synthesized single-stranded DNA, at or near the 3′ end of the second newly synthesized single-stranded DNA. In some embodiments, the second newly synthesized single-stranded DNA comprises a region of complementarity to the first newly synthesized single-stranded DNA, at or near the 5′ end of the second newly synthesized single-stranded DNA. In some embodiments, the second newly synthesized single-stranded DNA comprises a region of complementarity to the first newly synthesized single-stranded DNA, in the middle of the second newly synthesized single-stranded DNA.

In some embodiments, the first newly synthesized single-stranded DNA and the second newly synthesized single-stranded DNA each comprises a region of complementarity to each other at the 3′ end of each of the first newly synthesized single-stranded DNA and the second newly synthesized single-stranded DNA.

In some embodiments, the first editing template and the second editing template are of the same length. In some embodiments, the first editing template and the second editing template are of different lengths.

In some embodiments, the first editing template comprises a region that has complementarity or substantial complementarity to the second editing template (the OD encoding region), and further comprises a region that does not have complementarity to the second editing template. In some embodiments, the first editing template comprises a region that has complementarity or substantial complementarity to the second editing template (the OD encoding region), wherein the region is flanked by one or more regions that do not have complementarity to the second editing template. In some embodiments, the entirety of the first editing template has complementarity or substantial complementarity to a region of the second editing template, wherein the second editing template comprises a region that does not have complementarity to the first editing template.

In some embodiments, the first editing template comprises a region that does not have complementarity to the second editing template, wherein the region is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the first editing template comprises a region that does not have complementarity to the second editing template, wherein the region is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the second editing template comprises a region that has complementarity or substantial complementarity to the first editing template, and further comprises a region that does not have complementarity to the first editing template. In some embodiments, the second editing template comprises a region that has complementarity or substantial complementarity to the first editing template, and is flanked by one or more regions that do not have complementarity to the first editing template. The region(s) in the first editing template and the second editing template may have same or different lengths. In some embodiments, the entirety of the second editing template has complementarity or substantial complementarity to a region of the first editing template, wherein the first editing template comprises a region that does not have complementarity to the second editing template.

In some embodiments, the second editing template comprises a region that does not have complementarity to the first editing template, wherein the region is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the second editing template comprises a region that does not have complementarity to the first editing template, wherein the region is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the RD comprises a region (or a subset) of the sequence of the IND. In some embodiments, the RD consists of a region of the sequence of the IND. In some embodiments, the RD comprises one or more intended nucleotide edits compared to the IND. In some embodiments, the RD comprises a region(s) that has substantial sequence identity to the sequence of the IND, wherein the region(s) comprises one or more nucleotide edits compared to the sequence of the IND. For example, the RD may comprise a region that has substantial sequence identity to the sequence of the IND, wherein the region comprises one or more nucleotide substitutions, insertions, or deletions. In some embodiments, the RD comprises a region of the sequence of the IND, and further comprises a region that does not have sequence identity or complementary to the IND. In some embodiments, the RD comprises a region that has substantial identity to the sequence of the IND comprising one or more nucleotide edits, and further comprises a region that does not have sequence identity or complementary to the IND. In some embodiments, the region that does not have sequence identity or complementary to the IND has a biological function or encodes a polypeptide or a portion thereof having a biological function. In some embodiments, the RD comprises one or more intended nucleotide edits compared to the IND and encodes a polypeptide or a portion thereof.

In some embodiments, the OD comprises a region (or a subset) of the sequence of the IND. In some embodiments, the OD consists of a region of the sequence of the IND. In some embodiments, the OD comprises one or more intended nucleotide edits compared to the IND. In some embodiments, the OD comprises a region(s) that has substantial sequence identity to the sequence of the IND, wherein the region(s) comprise one or more nucleotide edits compared to the sequence of the IND. For example, the OD may comprise a region that has substantial sequence identity to the sequence of the IND, wherein the region comprises one or more nucleotide substitutions, insertions, or deletions. In some embodiments, the OD comprises a region of the sequence of the IND, and further comprises a region that does not have sequence identity or complementary to the IND. In some embodiments, the OD comprises a region that has substantial identity to the sequence of the IND comprising one or more nucleotide edits, and further comprises a region that does not have sequence identity or complementarity to the IND. In some embodiments, the region that does not have sequence identity or complementarity to the IND has a biological function or encodes a polypeptide or a portion thereof having a biological function. In some embodiments, the OD comprises one or more intended nucleotide edits compared to the IND and encodes a polypeptide or a portion thereof.

In some embodiments, the IND comprises an array of nucleotide motifs. In some embodiments, the IND has an array of three nucleotide repeats (or tri-nucleotide repeats). In some embodiments, the IND has an array of 5-10, 5-15, 5-20, 5-25, 5-30, 5-35, 5-40, 5-50, 5-60, 5-70, 5-80, 5-90, 5-100, 20-30, 20-40, 20-50, 20-60, 20-70, 20-80, 20-90, 20-100, 35-40, 35-50, 35-60, 35-70, 35-80, 35-90, 35-100, 50-70, 50-80, 50-90, 50-100, 50-150, 50-200, 50-250, 50-300, 50-350, 50-400, 50-450, 50-500, 50-550, 50-600, 50-650, 50-700, 50-750, 50-800, 50-850, 50-900, 50-950, 50-1000, 50-1050, 50-1100, 50-1150, 50-1200, 50-1250, 50-1300, 50-1350, 50-1400, 50-1450, 50-1500, 100-150, 100-200, 100-250, 100-300, 100-350, 100-400, 100-450, 100-500, 100-550, 100-600, 100-650, 100-700, 100-750, 100-800, 100-850, 100-900, 100-950, 100-1000, 100-1050, 100-1100, 100-1150, 100-1200, 100-1250, 100-1300, 100-1350, 100-1400, 100-1450, 100-1500, 150-200, 150-250, 150-300, 150-350, 150-400, 150-450, 150-500, 150-550, 150-600, 150-650, 150-700, 150-750, 150-800, 150-850, 150-900, 150-950, 150-1000, 150-1050, 150-1100, 150-1150, 150-1200, 150-1250, 150-1300, 150-1350, 150-1400, 150-1450, 150-1500, 200-250, 200-300, 200-350, 200-400, 200-450, 200-500, 200-550, 200-600, 200-650, 200-700, 200-750, 200-800, 200-850, 200-900, 200-950, 200-1000, 200-1050, 200-1100, 200-1150, 200-1200, 200-1250, 200-1300, 200-1350, 200-1400, 200-1450, 200-1500, 250-300, 250-350, 250-400, 250-450, 250-500, 250-550, 250-600, 250-650, 250-700, 250-750, 250-800, 250-850, 250-900, 250-950, 250-1000, 250-1050, 250-1100, 250-1150, 250-1200, 250-1250, 250-1300, 250-1350, 250-1400, 250-1450, 250-1500, 300-350, 300-400, 300-450, 300-500, 300-550, 300-600, 300-650, 300-700, 300-750, 300-800, 300-850, 300-900, 300-950, 300-1000, 300-1050, 300-1100, 300-1150, 300-1200, 300-1250, 300-1300, 300-1350, 300-1400, 300-1450, 300-1500, 400-450, 400-500, 400-550, 400-600, 400-650, 400-700, 400-750, 400-800, 400-850, 400-900, 400-950, 400-1000, 400-1050, 400-1100, 400-1150, 400-1200, 400-1250, 400-1300, 400-1350, 400-1400, 400-1450, 400-1500, 500-550, 500-600, 500-650, 500-700, 500-750, 500-800, 500-850, 500-900, 500-950, 500-1000, 500-1050, 500-1100, 500-1150, 500-1200, 500-1250, 500-1300, 500-1350, 500-1400, 500-1450, 500-1500, 600-650, 600-700, 600-750, 600-800, 600-850, 600-900, 600-950, 600-1000, 600-1050, 600-1100, 600-1150, 600-1200, 600-1250, 600-1300, 600-1350, 600-1400, 600-1450, 600-1500, 700-750, 700-800, 700-850, 700-900, 700-950, 700-1000, 700-1050, 700-1100, 700-1150, 700-1200, 700-1250, 700-1300, 700-1350, 700-1400, 700-1450, 700-1500, 800-850, 800-900, 800-950, 800-1000, 800-1050, 800-1100, 800-1150, 800-1200, 800-1250, 800-1300, 800-1350, 800-1400, 800-1450, 800-1500, 900-950, 900-1000, 900-1050, 900-1100, 900-1150, 900-1200, 900-1250, 900-1300, 900-1350, 900-1400, 900-1450, 900-1500, 1000-1050, 1000-1100, 1000-1150, 1000-1200, 1000-1250, 1000-1300, 1000-1350, 1000-1400, 1000-1450, 1000-1500, 1100-1200, 1100-1300, 1100-1400, 1100-1500, 1200-1300, 1200-1400, 1200-1500, 1300-1400, 1300-1500, or 1400-1500 tri-nucleotide repeats. In some embodiments, the IND has an array of more than 100, 200, 300, 400, 500, 600, 700, 800, 900, or 1000 repeats. In some embodiments, the IND has an array of more than 1000 repeats. In some embodiments, the IND has an array of more than 1500 repeats. In some embodiments, the IND has an array of 35-50 CTG repeats (SEQ ID NO: 391). In some embodiments, the IND has an array of 35-49 CTG repeats (SEQ ID NO: 392). In some embodiments, the IND has an array of 50-1000 CTG repeats (SEQ ID NO: 396). In some embodiments, the IND has an array of 100-1000 CTG repeats (SEQ ID NO: 397). In some embodiments, the IND has an array of 50-150 CTG repeats (SEQ ID NO: 393). In some embodiments, the IND has an array of more than 1000 CTG repeats.

In some embodiments, the first editing template comprises a region of identity to a sequence adjacent to the second nick site on the second PAM strand of the double-stranded target DNA, wherein the sequence is outside the IND. In some embodiments, the second editing template comprises a region of identity to a sequence adjacent to the first nick site on the first PAM strand of the double-stranded target DNA, wherein the sequence is outside the IND. Accordingly, in some embodiments, the first newly synthesized single-stranded DNA encoded by the first editing template comprises a region of complementarity to a sequence adjacent to the second nick site on the second PAM strand of the double-stranded target DNA, wherein the sequence is outside the IND. In some embodiments, the second newly synthesized single-stranded DNA encoded by the second editing template comprises a region of complementarity to a sequence adjacent to the first nick site on the first PAM strand of the double-stranded target DNA, wherein the sequence is outside the IND.

In some embodiments, the first newly synthesized single-stranded DNA comprises a region of complementarity to a sequence immediately adjacent to the second nick site on the second PAM strand of the double-stranded target DNA, wherein the sequence is outside the IND. In some embodiments, the second newly synthesized single-stranded DNA encoded by the second editing template comprises a region of complementarity to a sequence immediately adjacent to the first nick site on the first PAM strand of the double-stranded target DNA, wherein the sequence is outside the IND (see e.g., FIG. 4F).

In some embodiments, the first newly synthesized single-stranded DNA comprises a region of complementarity to a sequence adjacent to the second nick site on the second PAM strand of the double-stranded target DNA, wherein the sequence is outside the IND, and is 1, 2, 3, 4, 5, 6, 7, 8, 9, or 10 nucleotides apart from the second nick site. In some embodiments, the second newly synthesized single-stranded DNA encoded by the second editing template comprises a region of complementarity to a sequence adjacent to the first nick site on the first PAM strand of the double-stranded target DNA, wherein the sequence is outside the IND, and is 1, 2, 3, 4, 5, 6, 7, 8, 9, or 10 nucleotides apart from the first nick site.

In some embodiments, the IND consists of all tri-nucleotide repeats of the double-stranded target DNA, e.g. the TCF4 gene. In some embodiments, through prime editing and DNA repair, the IND is excised, and the tri-nucleotide repeats are deleted from the double-stranded target DNA, e.g., the TCF4 gene.

In some embodiments, the IND comprises the tri-nucleotide repeats of the double-stranded target DNA, and further comprises one or more base pairs upstream and/or downstream of the tri-nucleotide repeats of the double-stranded target DNA, e.g., the TCF4 gene. In some embodiments, through prime editing and DNA repair, the IND is excised, and the array of tri-nucleotide repeats, along with the one or more base pairs upstream and/or downstream of the array of tri-nucleotide repeats are deleted from the double-stranded target DNA, e.g., the TCF4 gene. Accordingly, in some embodiments, incorporation of the first newly synthesized single-stranded DNA and the second newly synthesized single-stranded DNA results in the incorporation of one or more intended nucleotide edits, which comprise deletion of the array of the tri-nucleotide repeat sequence. In some embodiments, incorporation of the first newly synthesized single-stranded DNA and the second newly synthesized single-stranded DNA results incorporation of one or more intended nucleotide edits, which comprise deletion of the array of the tri-nucleotide repeat sequence and deletion of the one or more base pairs upstream and/or downstream of the trinucleotide repeat array sequence.

In some embodiments, the first editing template and the second editing template each comprises a region of complementarity or substantial complementarity to each other. In some embodiments, the first editing template comprises a sequence that is exogenous to the double-stranded target DNA. In some embodiments, the second editing template comprise a sequence that is exogenous to the double-stranded target DNA. In some embodiments, the sequence in the first editing template that is exogenous to the double-stranded target DNA comprises a region of complementarity or substantial complementarity to the sequence in the second editing template that is exogenous to the double-stranded target DNA. In some embodiments, the sequence in the first editing template that is exogenous to the double-stranded target DNA further comprises a region that is not complementary to the sequence in the second editing template that is exogenous to the double-stranded target DNA. In some embodiments, the sequence in the second editing template that is exogenous to the double-stranded target DNA comprises a region of complementarity or substantial complementarity to the sequence in the first editing template that is exogenous to the double-stranded target DNA. In some embodiments, the sequence in the second editing template that is exogenous to the double-stranded target DNA further comprises a region that is not complementary to the sequence in the first editing template that is exogenous to the double-stranded target DNA. In some embodiments, the first editing template comprises a sequence exogenous to the double-stranded target DNA, wherein the sequence exogenous to the double-stranded target DNA comprises a polynucleotide sequence that encodes an expression tag, for example, an affinity tag, a His tag, a V5 tag, or a FLAG tag. In some embodiments, the first editing template comprises a sequence exogenous to the double-stranded target DNA, wherein the sequence exogenous to the double-stranded target DNA comprises an attB or an attP sequence. In some embodiments, the second editing template comprises a sequence exogenous to the double-stranded target DNA, wherein the sequence exogenous to the double-stranded target DNA comprises a polynucleotide sequence that encodes an expression tag, for example, an affinity tag, a His tag, a V5 tag, or a FLAG tag.

Accordingly, in some embodiments, the first newly synthesized single-stranded DNA and the second newly synthesized single-stranded DNA each comprises a region of complementarity or substantial complementarity to each other. In some embodiments, the first newly synthesized single-stranded DNA comprise a sequence that is exogenous to the double-stranded target DNA. In some embodiments, the second newly synthesized single-stranded DNA comprise a sequence that is exogenous to the double-stranded target DNA. In some embodiments, the sequence in the first newly synthesized single-stranded DNA that is exogenous to the double-stranded target DNA comprises a region of complementarity or substantial complementarity to the sequence in the second newly synthesized single-stranded DNA that is exogenous to the double-stranded target DNA. In some embodiments, the sequence in the first newly synthesized single-stranded DNA that is exogenous to the double-stranded target DNA further comprises a region that is not complementary to the sequence in the second newly synthesized single-stranded DNA that is exogenous to the double-stranded target DNA. In some embodiments, the sequence in the second newly synthesized single-stranded DNA that is exogenous to the double-stranded target DNA comprises a region of complementarity or substantial complementarity to the sequence in the first newly synthesized single-stranded DNA that is exogenous to the double-stranded target DNA. In some embodiments, the sequence in the second newly synthesized single-stranded DNA that is exogenous to the double-stranded target DNA further comprises a region that is not complementary to the sequence in the first newly synthesized single-stranded DNA that is exogenous to the double-stranded target DNA.

Accordingly, in some embodiments, the first newly synthesized single-stranded DNA and the second newly synthesized single-stranded DNA form an OD that comprises a sequence that is exogenous to the double-stranded target DNA, e.g. the TCF4 gene. In some embodiments, the first newly synthesized single-stranded DNA and the second newly synthesized single-stranded DNA form an RD that comprises a sequence that is exogenous to the double-stranded target DNA, e.g. the TCF4 gene. In some embodiments, the IND comprises substantially all or all tri-nucleotide repeats of the double-stranded target DNA, e.g. the TCF4 gene. Through prime editing, in some embodiments, the IND is excised and is replaced by the RD. In some embodiments, the IND is excised and is replaced by the RD. Accordingly, in some embodiments, substantially all or all tri-nucleotide repeats of the double-stranded target DNA, e.g., the TCF4 gene, are deleted and replaced by the sequence exogenous to the double-stranded target DNA, e.g., the TCF4 gene. In some embodiments, the sequence exogenous to the double-stranded target DNA comprises a polynucleotide sequence that encodes an expression tag, for example, an affinity tag, a His tag, a V5 tag, or a FLAG tag. In some embodiments, the sequence exogenous to the double-stranded target DNA comprises an attB or an attP sequence. Accordingly, in some embodiments, incorporation of the one or more intended nucleotide edits comprises deletion of array of the tri-nucleotide repeat sequence and incorporation of one or more exogenous sequences encoded by the first editing template and/or the second editing template.

In some embodiments, the first editing template comprises a sequence that has complementarity or substantial complementarity to an endogenous sequence of the double-stranded target DNA, e.g., the TCF4 gene. In some embodiments, the second editing template comprises a sequence that has complementarity or substantial complementarity to an endogenous sequence of the double-stranded target DNA, e.g., the TCF4 gene. In some embodiments, the first and/or the second editing template comprises a sequence that has complementarity or substantial complementarity to an endogenous sequence of the double-stranded target gene, e.g., the TCF4 gene, wherein the endogenous sequence of the double-stranded target DNA, e.g., the TCF4 gene does not comprise the array of CTG tri-nucleotide repeat of the double-stranded target DNA, e.g., the TCF4 gene. In some embodiments, the endogenous sequence of the double-stranded target DNA, e.g., the TCF4 gene, has complementarity or substantial complementarity to the first and/or the second editing template does not comprise an array of tri-nucleotide repeat or any nucleotide repeat structure. In some embodiments, the first editing template comprises a sequence that has complementarity or substantial complementarity to an endogenous sequence on the second strand of the double-stranded target DNA, e.g., the TCF4 gene, wherein the endogenous sequence of the double-stranded target DNA, e.g., the TCF4 gene, is upstream of the array of tri-nucleotide repeats. In some embodiments, the first editing template comprises a sequence that has complementarity or substantial complementarity to an endogenous sequence on the second strand of the double-stranded target DNA, e.g., the TCF4 gene, wherein the endogenous sequence of the double-stranded target DNA, e.g., the TCF4 gene, is downstream of the array of tri-nucleotide repeats.

In some embodiments, the second editing template comprises a sequence that has complementarity or substantial complementarity to an endogenous sequence on the first strand of the double-stranded target DNA, e.g., the TCF4 gene, wherein the endogenous sequence of the double-stranded target DNA, e.g., the TCF4 gene, is upstream of the array of tri-nucleotide repeats. In some embodiments, the second editing template comprises a sequence that has complementarity or substantial complementarity to an endogenous sequence on the first strand of the double-stranded target DNA, e.g., the TCF4 gene, wherein the endogenous sequence of the double-stranded target DNA, e.g., the TCF4 gene, is downstream of the array of tri-nucleotide repeats.

In some embodiments, the first editing template comprises a sequence that has complementarity or substantial complementarity to an endogenous sequence on the second strand of the double-stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the first editing template comprises a sequence that has complementarity or substantial complementarity to an endogenous sequence on the second strand of the double-stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the first editing template comprises a sequence that has complementarity or substantial complementarity to an endogenous sequence on the second strand of the double-stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides upstream of the array of the tri-nucleotide repeats as measured at the 5′ ends. In some embodiments, the first editing template comprises a sequence that has complementarity or substantial complementarity to an endogenous sequence on the second strand of the double-stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides downstream of the array of the tri-nucleotide repeats as measured at the 5′ ends.

In some embodiments, the second editing template comprises a sequence that has complementarity or substantial complementarity to an endogenous sequence on the first strand of the double-stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the second editing template comprises a sequence that has complementarity or substantial complementarity to an endogenous sequence on the first strand of the double-stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the second editing template comprises a sequence that has complementarity or substantial complementarity to an endogenous sequence on the first strand of the double-stranded target DNA that is upstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides upstream of the array of the tri-nucleotide repeats as measured at the 5′ ends.

In some embodiments, the second editing template comprises a sequence that has complementarity or substantial complementarity to an endogenous sequence on the first strand of the double-stranded target DNA that is downstream of the array of the trinucleotide-repeats, wherein the endogenous sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more nucleotides downstream of the array of the tri-nucleotide repeats as measured at the 5′ ends.

In some embodiments, the sequence of the first editing template that has complementarity or substantial complementarity to an endogenous sequence of the double-stranded target DNA comprises a region of complementarity or substantial complementarity to the sequence of the second editing template that has complementarity or substantial complementarity to an endogenous sequence of the double-stranded target DNA. In some embodiments, the sequence of the first editing template that complementarity or substantial complementarity to an endogenous sequence of the double-stranded target DNA further comprises a region that is not complementary to the sequence of the second editing template that has complementarity or substantial complementarity to an endogenous sequence of the double-stranded target DNA. In some embodiments, the sequence of the second editing template that has complementarity or substantial complementarity to an endogenous sequence of the double-stranded target DNA comprises a region of complementarity or substantial complementarity to the sequence of the first editing template that has complementarity or substantial complementarity to an endogenous sequence of the double-stranded target DNA. In some embodiments, the sequence of the second editing template that has complementarity or substantial complementarity to an endogenous sequence of the double-stranded target DNA further comprises a region that is not complementary to the sequence of the first editing template that has identity or substantial identity to an endogenous sequence of the double-stranded target DNA.

Accordingly, in some embodiments, the first newly synthesized single-stranded DNA comprises a sequence that has identity or substantial identity to an endogenous sequence of the double-stranded target DNA, e.g., the TCF4 gene. In some embodiments, the second newly synthesized single-stranded DNA comprises a sequence that has identity or substantial identity to an endogenous sequence of the double-stranded target DNA, e.g., the TCF4 gene. In some embodiments, the first newly synthesized single-stranded DNA and/or the second newly synthesized single-stranded DNA comprises a sequence that has identity or substantial identity to an endogenous sequence of the double-stranded target DNA, e.g., the TCF4 gene, wherein the endogenous sequence of the double-stranded target DNA, e.g., the TCF4 gene does not comprise the array of tri-nucleotide repeat of the double-stranded target DNA, e.g., the TCF4 gene. In some embodiments, the first newly synthesized single-stranded DNA comprises a sequence that has identity or substantial identity to an endogenous sequence on the second strand of the double-stranded target DNA, e.g., the TCF4 gene, wherein the endogenous sequence of the double-stranded target DNA, e.g., the TCF4 gene, is upstream of the array of tri-nucleotide repeats. In some embodiments, the first newly synthesized single-stranded DNA comprises a sequence that has identity or substantial identity to an endogenous sequence on the second strand of the double-stranded target DNA, e.g., the TCF4 gene, wherein the endogenous sequence of the double-stranded target DNA, e.g., the TCF4 gene, is downstream of the array of tri-nucleotide repeats. In some embodiments, the second newly synthesized single-stranded DNA comprises a sequence that has identity or substantial identity to an endogenous sequence on the first strand of the double-stranded target DNA, e.g., the TCF4 gene, wherein the endogenous sequence of the double-stranded target DNA, e.g., the TCF4 gene, is upstream of the array of tri-nucleotide repeats. In some embodiments, the second newly synthesized single-stranded DNA comprises a sequence that has identity or substantial identity to an endogenous sequence on the first strand of the double-stranded target DNA, e.g., the TCF4 gene, wherein the endogenous sequence of the double-stranded target DNA, e.g., the TCF4 gene, is downstream of the array of tri-nucleotide repeats.

In some embodiments, the array of tri-nucleotide repeats of the TCF4 gene is an array of CTG repeats on the coding strand (the second strand) or the reverse complement CAG repeats on the non-coding strand (the first strand).

In some embodiments, the sequence of the first newly synthesized single-stranded DNA that has identity or substantial identity to an endogenous sequence of the double-stranded target DNA comprises a region of complementarity or substantial complementarity to the sequence of the second newly synthesized single-stranded DNA that has identity or substantial identity to an endogenous sequence of the double-stranded target DNA. In some embodiments, the sequence of the first newly synthesized single-stranded DNA that has identity or substantial identity to an endogenous sequence of the double-stranded target DNA further comprises a region that is not complementary to the sequence of the second newly synthesized single-stranded DNA that has identity or substantial identity to an endogenous sequence of the double-stranded target DNA. In some embodiments, the sequence of the second newly synthesized single-stranded DNA that has identity or substantial identity to an endogenous sequence of the double-stranded target DNA comprises a region of complementarity or substantial complementarity to the sequence of the first newly synthesized single-stranded DNA that has identity or substantial identity to an endogenous sequence of the double-stranded target DNA. In some embodiments, the sequence of the second newly synthesized single-stranded DNA that has identity or substantial identity to an endogenous sequence of the double-stranded target DNA further comprises a region that is not complementary to the sequence of the first newly synthesized single-stranded DNA that has identity or substantial identity to an endogenous sequence of the double-stranded target DNA.

Accordingly, in some embodiments, the first newly synthesized single-stranded DNA and the second newly synthesized single-stranded DNA form an OD that comprises an endogenous sequence of the double-stranded target DNA, e.g. the TCF4 gene. In some embodiments, the first newly synthesized single-stranded DNA and the second newly synthesized single-stranded DNA form an RD that comprises an endogenous sequence of the double-stranded target DNA, e.g. the TCF4 gene. In some embodiments, the RD or the OD comprises an endogenous sequence of the double-stranded target DNA, e.g., the TCF4 gene, upstream of the array of tri-nucleotide repeats. In some embodiments, the RD or the OD comprises an endogenous sequence of the double-stranded target DNA, e.g., the TCF4 gene, downstream of the array of tri-nucleotide repeats. In some embodiments, the RD or the OD comprises a sequence that is endogenous compared to the double-stranded target DNA, e.g., the TCF4 gene, wherein the sequence comprises two regions: a) a region that is identical or substantially identical to an endogenous sequence upstream of the array of the tri-nucleotide repeats, and b) a region that is identical or substantially identical to an endogenous sequence downstream of the array of the tri-nucleotide repeats. In some embodiments, the region identical or substantially identical to the endogenous sequence upstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs in length. In some embodiments, the region identical or substantially identical to the endogenous sequence downstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs in length. In some embodiments, the region identical or substantially identical to the endogenous sequence upstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more base pairs upstream of the array of the tri-nucleotide repeats as measured at the 5′ ends. In some embodiments, the region identical or substantially identical to the endogenous sequence upstream of the array of the trinucleotide-repeats is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500 or more base pairs downstream of the array of the tri-nucleotide repeats as measured at the 5′ ends. In some embodiments, the array of tri-nucleotide repeats of the TCF4 gene is an array of CTG (or the reverse complement CAG) repeats.

In some embodiments, the IND comprises all tri-nucleotide repeats of the double-stranded target DNA, e.g. the entire array of CTG (or the reverse complement CAG) repeats of the TCF4 gene. In some embodiments, the IND comprises all tri-nucleotide repeats of the double-stranded target DNA, e.g. the entire array of CTG (or CAG) repeats of the TCF4 gene, and further comprises one or more base pairs upstream and/or downstream of the array of tri-nucleotide repeats. Through prime editing, the IND is excised and is replaced by the RD or the OD. Accordingly, in some embodiments, all tri-nucleotide repeats of the double-stranded target DNA, e.g., the entire array of CTG (or CAG) repeats of the TCF4 gene, are deleted, and the endogenous sequence upstream of the array of tri-nucleotide repeats is retained. In some embodiments, all tri-nucleotide repeats of the double-stranded target DNA, e.g. the entire array of CTG (or CAG) repeats of the TCF4 gene are deleted, and the endogenous sequence downstream of the array of tri-nucleotide repeats is retained. In some embodiments, the IND comprises all tri-nucleotide repeats of the double-stranded target DNA, e.g. the TCF4 gene. In some embodiments, the first editing template has a different number of tri-nucleotide repeats compared to the number of tri-nucleotide repeats in the IND. In some embodiments, the first editing template has a reduced number of tri-nucleotide repeats compared to the number of tri-nucleotide repeats in the IND. In some embodiments, the second editing template has a different number of tri-nucleotide repeats compared to the number of tri-nucleotide repeats in the IND. In some embodiments, the second editing template has a reduced number of tri-nucleotide repeats compared to the number of tri-nucleotide repeats in the IND.

Accordingly, in some embodiments, the first newly synthesized single-stranded DNA encoded by the first editing template has a different number of tri-nucleotide repeats compared to the number of tri-nucleotide repeats in the IND. In some embodiments, the first newly synthesized single-stranded DNA encoded by the first editing template has a reduced number of tri-nucleotide repeats compared to the number of tri-nucleotide repeats in the IND. In some embodiments, the second newly synthesized single-stranded DNA encoded by the second editing template has a different number of tri-nucleotide repeats compared to the number of tri-nucleotide repeats in the IND. In some embodiments, the second newly synthesized single-stranded DNA encoded by the second editing template has a reduced number of tri-nucleotide repeats compared to the number of tri-nucleotide repeats in the IND. In some embodiments, the first newly synthesized single-stranded DNA and the second newly synthesized single-stranded DNA can form an OD or a RD that comprises a reduced number of tri-nucleotide repeats compared to the number of tri-nucleotide repeats in the IND.

In some embodiments, the OD has an array of the same nucleotide repeat motifs, for example, CTG repeats, but of a different number compared to the number of the tri-nucleotide repeats in the IND. In some embodiments, the RD has an array of the same nucleotide repeat motifs, for example, CTG repeats, but of a different number compared to the number of the tri-nucleotide repeats in the IND. In some embodiments, the OD has a reduced number of the tri-nucleotide repeats, e.g., CTG repeats compared to the endogenous number of tri-nucleotide repeats of the double-stranded target DNA, e.g., the TCF4 gene. In some embodiments, the RD has a reduced number of the tri-nucleotide repeats, e.g., CTG repeats, compared to the endogenous number of tri-nucleotide repeats of the double-stranded target DNA, e.g., the TCF4 gene. In some embodiments, the RD contains at most 30, 20, 10, or 5 CTG tri-nucleotide repeats (SEQ ID NOs: 394, 395, 400 or 390, respectively). In some embodiments, the RD contains 30, 20, 10, or 5 CTG tri-nucleotide repeats (SEQ ID NOs: 394, 395, 400 or 390, respectively). In some embodiments, the OD contains at most 30, 20, 10, or 5 CTG tri-nucleotide repeats (SEQ ID NOs: 394, 395, 400 or 390, respectively). In some embodiments, the OD contains 30, 20, 10, or 5 CTG tri-nucleotide repeats (SEQ ID NOS 394, 395, 400 or 390, respectively). In some embodiments, the RD contains 5 CTG repeats (SEQ ID NO: 390). In some embodiments, the OD contains 5 CTG repeats (SEQ ID NO: 390). In some embodiments, the RD or the OD contains the same number of tri-nucleotide repeats as a reference gene, for example, a wild-type TCF4 gene.

Accordingly, in some embodiments, excision of the IND and incorporation of the RD results in deletion of a portion of the nucleotide repeats sequences of the IND from the double-stranded target DNA, e.g., the target gene.

In some embodiments, excision of the IND and incorporation of the OD results in deletion of a portion of the nucleotide repeats sequences of the IND from the double-stranded target DNA, e.g., the target gene. In some embodiments, the deletion comprises at least 1, 2, 3, 4, 5, 6, 7, 8, 9, 10, 11, 12, 13, 14, 15, 16, 17, 18, 19, 20 or more tri-nucleotide repeats. In some embodiments, the deletion comprises 1-3, 1-5, 1-10, 1-15, 1-20, 1-25, 1-30, 1-35, 1-40, 1-45, 1-50, 1-55, 1-60, 1-75 or more tri-nucleotide repeats. In some embodiments, the deletion comprises deletion of at least 1, 2, 3, 4, 5, 6, 7, 8, 9, 10, 11, 12, 13, 14, 15, 16, 17, 18, 19, 20 tri-nucleotide repeats from the double-stranded target DNA, e.g., the target gene. In some embodiments, the deletion comprises deletion of 1-3, 1-5, 1-10, 1-15, 1-20, 1-25, 1-30, 1-35, 1-40, 1-45, 1-50, 1-55, 1-60, 1-75 or more tri-nucleotide repeats from the double-stranded target DNA, e.g., the target gene. In some embodiments, the deletion comprises deletion of 5-10, 5-15, 5-20, 5-25, 5-30, 5-35, 5-40, 5-50, 5-60, 5-70, 5-80, 5-90, 5-100, 20-30, 20-40, 20-50, 20-60, 20-70, 20-80, 20-90, 20-100, 35-40, 35-50, 35-60, 35-70, 35-80, 35-90, 35-100, 50-70, 50-80, 50-90, 50-100, 50-150, 50-200, 50-250, 50-300, 50-350, 50-400, 50-450, 50-500, 50-550, 50-600, 50-650, 50-700, 50-750, 50-800, 50-850, 50-900, 50-950, 50-1000, 50-1050, 50-1100, 50-1150, 50-1200, 50-1250, 50-1300, 50-1350, 50-1400, 50-1450, 50-1500, 100-150, 100-200, 100-250, 100-300, 100-350, 100-400, 100-450, 100-500, 100-550, 100-600, 100-650, 100-700, 100-750, 100-800, 100-850, 100-900, 100-950, 100-1000, 100-1050, 100-1100, 100-1150, 100-1200, 100-1250, 100-1300, 100-1350, 100-1400, 100-1450, 100-1500, 150-200, 150-250, 150-300, 150-350, 150-400, 150-450, 150-500, 150-550, 150-600, 150-650, 150-700, 150-750, 150-800, 150-850, 150-900, 150-950, 150-1000, 150-1050, 150-1100, 150-1150, 150-1200, 150-1250, 150-1300, 150-1350, 150-1400, 150-1450, 150-1500, 200-250, 200-300, 200-350, 200-400, 200-450, 200-500, 200-550, 200-600, 200-650, 200-700, 200-750, 200-800, 200-850, 200-900, 200-950, 200-1000, 200-1050, 200-1100, 200-1150, 200-1200, 200-1250, 200-1300, 200-1350, 200-1400, 200-1450, 200-1500, 250-300, 250-350, 250-400, 250-450, 250-500, 250-550, 250-600, 250-650, 250-700, 250-750, 250-800, 250-850, 250-900, 250-950, 250-1000, 250-1050, 250-1100, 250-1150, 250-1200, 250-1250, 250-1300, 250-1350, 250-1400, 250-1450, 250-1500, 300-350, 300-400, 300-450, 300-500, 300-550, 300-600, 300-650, 300-700, 300-750, 300-800, 300-850, 300-900, 300-950, 300-1000, 300-1050, 300-1100, 300-1150, 300-1200, 300-1250, 300-1300, 300-1350, 300-1400, 300-1450, 300-1500, 400-450, 400-500, 400-550, 400-600, 400-650, 400-700, 400-750, 400-800, 400-850, 400-900, 400-950, 400-1000, 400-1050, 400-1100, 400-1150, 400-1200, 400-1250, 400-1300, 400-1350, 400-1400, 400-1450, 400-1500, 500-550, 500-600, 500-650, 500-700, 500-750, 500-800, 500-850, 500-900, 500-950, 500-1000, 500-1050, 500-1100, 500-1150, 500-1200, 500-1250, 500-1300, 500-1350, 500-1400, 500-1450, 500-1500, 600-650, 600-700, 600-750, 600-800, 600-850, 600-900, 600-950, 600-1000, 600-1050, 600-1100, 600-1150, 600-1200, 600-1250, 600-1300, 600-1350, 600-1400, 600-1450, 600-1500, 700-750, 700-800, 700-850, 700-900, 700-950, 700-1000, 700-1050, 700-1100, 700-1150, 700-1200, 700-1250, 700-1300, 700-1350, 700-1400, 700-1450, 700-1500, 800-850, 800-900, 800-950, 800-1000, 800-1050, 800-1100, 800-1150, 800-1200, 800-1250, 800-1300, 800-1350, 800-1400, 800-1450, 800-1500, 900-950, 900-1000, 900-1050, 900-1100, 900-1150, 900-1200, 900-1250, 900-1300, 900-1350, 900-1400, 900-1450, 900-1500, 1000-1050, 1000-1100, 1000-1150, 1000-1200, 1000-1250, 1000-1300, 1000-1350, 1000-1400, 1000-1450, 1000-1500, 1100-1200, 1100-1300, 1100-1400, 1100-1500, 1200-1300, 1200-1400, 1200-1500, 1300-1400, 1300-1500, or 1400-1500 tri-nucleotide repeats from the double-stranded target DNA, e.g., the target gene. In some embodiments, the deletion comprises deletion of 100, 200, 300, 400, 500, 600, 700, 800, 900, or 1000 trinucleotide repeats from the double-stranded target DNA, e.g., the target gene. In some embodiments, the deletion comprises deletion of more than 1000 trinucleotide repeats from the double-stranded target DNA, e.g., the target gene.

In some embodiments, the first editing template has a reduced number of tri-nucleotide repeats compared to the number of tri-nucleotide repeats in the IND, and further comprises a region that is complementary or substantially complementary to a sequence on the second strand of the double-stranded target DNA that is downstream of the array of tri-nucleotide repeats. In some embodiments, the first editing template has a reduced number of tri-nucleotide repeats compared to the number of tri-nucleotide repeats in the IND, and further comprises a) a region that is complementary or substantially complementary to a sequence on the second strand of the double-stranded target DNA that is downstream of the array of tri-nucleotide repeats, and b) a region that is complementary or substantially complementary to a sequence on the second strand of the double-stranded target DNA that is upstream of the array of tri-nucleotide repeats.

In some embodiments, the second editing template has a reduced number of tri-nucleotide repeats compared to the number of tri-nucleotide repeats in the IND, and further comprises a region that is complementary or substantially complementary to a sequence on the first strand of the double-stranded target DNA that is upstream of the array of tri-nucleotide repeats. In some embodiments, the second editing template has a reduced number of tri-nucleotide repeats compared to the number of tri-nucleotide repeats in the IND, and further comprises a) a region that is complementary or substantially complementary to a sequence on the first strand of the double-stranded target DNA that is upstream of the array of tri-nucleotide repeats, and b) a region that is complementary or substantially complementary to a sequence on the first strand of the double-stranded target DNA that is downstream of the array of tri-nucleotide repeats.

Accordingly, in some embodiments, the first newly synthesized single-stranded DNA encoded by the first editing template has a reduced number of tri-nucleotide repeats compared to the number of tri-nucleotide repeats in the IND, and further comprises a region that is identical or substantially identical to a sequence on the second strand of the double-stranded target DNA that is downstream of the array of tri-nucleotide repeats. In some embodiments, the first newly synthesized single-stranded DNA encoded by the first editing template has a reduced number of tri-nucleotide repeats compared to the number of tri-nucleotide repeats in the IND, and further comprises a) a region that is identical or substantially identical to a sequence on the second strand of the double-stranded target DNA that is downstream of the array of tri-nucleotide repeats, and b) a region that is identical or substantially identical to a sequence on the second strand of the double-stranded target DNA that is upstream of the array of tri-nucleotide repeats.

In some embodiments, the second newly synthesized single-stranded DNA encoded by the second editing template has a reduced number of tri-nucleotide repeats compared to the number of tri-nucleotide repeats in the IND, and further comprises a region that is identical or substantially identical to a sequence on the first strand of the double-stranded target DNA that is upstream of the array of tri-nucleotide repeats. In some embodiments, the second newly synthesized single-stranded DNA encoded by the second editing template has a reduced number of tri-nucleotide repeats compared to the number of tri-nucleotide repeats in the IND, and further comprises a) a region that is identical or substantially identical to a sequence on the first strand of the double-stranded target DNA that is upstream of the array of tri-nucleotide repeats, and b) a region that is identical or substantially identical to a sequence on the first strand of the double-stranded target DNA that is downstream of the array of tri-nucleotide repeats.

Accordingly, in some embodiments, the RD or the OD has a reduced number of tri-nucleotide repeats compared to the number of tri-nucleotide repeats in the IND, and further comprises a double-stranded sequence of the double-stranded target DNA that is upstream or downstream of the IND. In some embodiments, the RD or the OD has a reduced number of tri-nucleotide repeats compared to the number of tri-nucleotide repeats in the IND, and further comprises a double-stranded sequence of the double-stranded target DNA that is upstream of the IND, and a double-stranded sequence of the double-stranded target DNA that is downstream of the IND.

In some embodiments, through prime editing, the IND is removed, and the sequence of the RD or the OD is incorporated into the double-stranded target DNA. As a result, a portion of the trinucleotide repeats of the double-stranded target DNA is deleted from the double-stranded target DNA, e.g., the target gene, and the sequences flanking the array of tri-nucleotide repeats are retained.

In some embodiments, the first editing template and/or the second editing template is partially complementary, substantially complementary, or identical to the sequence of the IND. In some embodiments, for example, the first editing template comprises a region that is complementary or identical to a region of a sequence of the IND. In some embodiments, the first editing template comprises a region of complementarity to the sequence on the first PAM strand of the IND. In some embodiments, the first editing template further comprises a region of complementarity to the second editing template. In some embodiments, the first editing template is partially complementary, substantially complementary or identical to a sequence of the IND, and is also substantially complementary to the second editing template. In some embodiments, the second editing template comprises a region that is complementary or identical to a region of a sequence of the IND. In some embodiments, the second editing template comprises a region of complementarity to the sequence on the second PAM strand of the IND. In some embodiments, the second editing template further comprises a region of complementarity to the first editing template. In some embodiments, the second editing template is partially complementary, substantially complementary or identical to a sequence of the IND, and is also substantially complementary to the first editing template. In some embodiments, the first editing template and the second editing template each comprises a region of complementarity to a sequence of the IND.

The partially complementary region(s) in the first editing template and the second editing template can be in any position within the first editing template and the second editing template. Accordingly, in some embodiments, the first newly synthesized single-stranded DNA encoded by the first editing template and the second newly synthesized single-stranded DNA encoded by the second editing template are partially complementary to each other, at any position within the first newly synthesized single-stranded DNA encoded by the first editing template and the second newly synthesized single-stranded DNA encoded by the second editing template. In some embodiments, the first newly synthesized single-stranded DNA comprises a region of complementarity to the first strand of the IND, at or near the 3′ end of the first newly synthesized single-stranded DNA. In some embodiments, the first newly synthesized single-stranded DNA comprises a region of complementarity to the first strand of the IND, at or near the 5′ end of the first newly synthesized single-stranded DNA. In some embodiments, the first newly synthesized single-stranded DNA comprises a region of complementarity to the first strand of the IND, in the middle of the first newly synthesized single-stranded DNA. In some embodiments, the second newly synthesized single-stranded DNA comprises a region of complementarity to the second strand of the IND, at or near the 3′ end of the second newly synthesized single-stranded DNA. In some embodiments, the second newly synthesized single-stranded DNA comprises a region of complementarity to the second strand of the IND, at or near the 5′ end of the second newly synthesized single-stranded DNA. In some embodiments, the second newly synthesized single-stranded DNA comprises a region of complementarity to the second strand of the IND, in the middle of the second newly synthesized single-stranded DNA. In some embodiments, the first newly synthesized single-stranded DNA and the second newly synthesized single-stranded DNA each comprises a region of complementarity to each other at the 3′ end.

Accordingly, as exemplified in FIG. 4C-FIG. 4D, in some embodiments, the first newly synthesized single-stranded DNA encoded by the first editing template comprises a region that is identical to a region of the sequence on the first PAM strand of the IND. In some embodiments, the second newly synthesized single-stranded DNA encoded by the second editing template comprises a region that is identical to a region of the sequence on the second PAM strand of the IND. In some embodiments, the first newly synthesized single-stranded DNA comprises two or more sub regions, each of which is identical to a sub region of the sequence on the first PAM strand of the IND (as exemplified in FIG. 4D). The sub regions on the first newly synthesized single-stranded DNA and/or the first PAM strand of the IND may or may not be consecutive. For example, the first newly synthesized single-stranded DNA may comprise 2 sub regions each identical to a sub region of the sequence on the first PAM strand of the IND, wherein the two sub regions of the sequence on the first PAM strand of the IND are separated by a region that does not have identity or substantial identity to the first newly synthesized single-stranded DNA. In some embodiments, the second newly synthesized single-stranded DNA comprises two or more sub regions, each of which is identical to a sub region of the sequence on the second PAM strand of the IND (as exemplified in FIG. 4D). The sub regions on the second newly synthesized single-stranded DNA and/or the second PAM strand of the IND may or may not be consecutive. For example, the second newly synthesized single-stranded DNA may comprise 2 sub regions each identical to a sub region of the sequence on the second PAM strand of the IND, wherein the two sub regions of the sequence on the second PAM strand of the IND are separated by a region that does not have identity or substantial identity to the second newly synthesized single-stranded DNA.

In some embodiments, the region of the sequence on the first PAM strand of the IND and the region of the sequence on the second PAM strand of the IND are complementary to each other. In some embodiments, the first newly synthesized single-stranded DNA encoded by the first editing template and the second newly synthesized single-stranded DNA encoded by the second editing template are at least partially complementary to each other and can anneal to each other to form an OD. In some embodiments, the first newly synthesized single-stranded DNA encoded by the first editing template and the second newly synthesized single-stranded DNA encoded by the second editing template are substantially complementary or complementary to each other and can anneal to each other to form an OD. In some embodiments, the IND is excised, and the OD is incorporated in the double-stranded target DNA at the place of the IND excision. As a result, the portion in the IND that is not complementary or identical to the first editing template or the second editing template is deleted from the double-stranded target DNA. In some embodiments, the deletion is at the 3′ end of the IND. In some embodiments, the deletion is at the 5′ end of the IND. In some embodiments, the deletion is in the middle of the IND.

In some embodiments, the first editing template of the first PERNA is at least partially complementary, substantially complementary, at least partially identical, or identical to a sequence of the double-stranded target DNA outside the IND. “Outside the IND” refers to sequences or positions of the double-stranded target DNA that are not in between the two nick sites generated by the first prime editor and the second prime editor. In some embodiments, the first editing template comprises a region of identity to a sequence outside the IND on the second PAM strand (or the first strand) of the double-stranded target DNA. In some embodiments, the first editing template comprises a region of identity to a sequence on the first strand of the double-stranded target DNA adjacent to the second nick site generated by the second prime editor complexed with the second PEgRNA, wherein the sequence is outside the IND. In some embodiments, the first editing template comprises a region of identity to a sequence on the first strand of the double-stranded target DNA immediately adjacent to the second nick site generated by the second prime editor complexed with the second PEgRNA, wherein the sequence is outside the IND

In some embodiments, the region of identity is at least 10, at least 15, at least 20, at least 25, at least 30, at least 35, at least 40, at least 45, at least 50, at least 55, at least 60, at least 65, at least 70, or at least 75 nucleotides in length. In some embodiments, the region of identity is about 10 to 15, about 10 to 20, about 10 to 25, about 10 to 30, about 10 to 35 about 10 to 40, about 15 to 20, about 15 to 25, about 15 to 30, about 15 to 35, about 15 to 40, about 20 to 25, about 20 to 30, about 20 to 35, about 20 to 40, about 25 to 30, about 25 to 35, about 25 to 40, about 30 to 35, or about 35 to 40 nucleotides in length. In some embodiments, the region of identity is no more than 25, no more than 30, no more than 35, no more than 40, or no more than 45 nucleotides in length. In some embodiments, the first editing template comprises a region of identity to the second spacer. In some embodiments, the first editing template comprises nucleotides m to (n−3) of the second spacer, wherein n is the length of the second spacer, and m is any integer between 1 and (n−12). In some embodiments, n is an integer from 16 to 22. For example, for a 20 nucleotide second spacer, the first editing template can contain nucleotides 1-17, 2-17, 3-17, 4-17, 5-17, 6-17, 7-7, or 8-17 of the second spacer.

Accordingly, as exemplified in FIG. 4E, in some embodiments, the first newly synthesized single-stranded DNA encoded by the first editing template comprises a region of complementarity to a sequence on the first strand of the double-stranded target DNA adjacent or immediately adjacent to the second nick site generated by the second prime editor complexed with the second PEgRNA. In some embodiments, the first newly synthesized single-stranded DNA encoded by the first editing template comprises a region of complementarity to a sequence on the first strand of the double-stranded target DNA immediately adjacent to the second nick site generated by the second prime editor complexed with the second PEgRNA, wherein the sequence is outside the IND. In some embodiments, the first newly synthesized single-stranded DNA anneals with the sequence on the first strand of the double-stranded target DNA adjacent or immediately adjacent to the second nick site generated by the second prime editor. In some embodiments, through DNA repair, the IND is excised and deleted from the double-stranded target DNA, e.g., the target gene.

In some embodiments, the second editing template of the second PEgRNA is at least partially complementary, substantially complementary, at least partially identical, or identical to a sequence of the double-stranded target DNA outside the IND. In some embodiments, the second editing template of the second PEgRNA comprises a region of identity to a sequence outside the IND on the first PAM strand (or the second strand) of the double-stranded target DNA. In some embodiments, the second editing template of the second PEgRNA comprises a region of identity to a sequence on the second strand of the double-stranded target DNA adjacent to the first nick site generated by the first prime editor complexed with the first PEgRNA, wherein the sequence is outside the IND. In some embodiments, the second editing template of the second PEgRNA comprises a region of identity to a sequence on the second strand of the double-stranded target DNA immediately adjacent to the first nick site generated by the first prime editor complexed with the first PEgRNA, wherein the sequence is outside the IND. In some embodiments, the region of identity is at least 10, at least 15, at least 20, at least 25, at least 30, at least 35, at least 40, at least 45, at least 50, at least 55, at least 60, at least 65, at least 70, or at least 75 nucleotides in length. In some embodiments, the region of identity is about 10 to 15, about 10 to 20, about 10 to 25, about 10 to 30, about 10 to 35 about 10 to 40, about 15 to 20, about 15 to 25, about 15 to 30, about 15 to 35, about 15 to 40, about 20 to 25, about 20 to 30, about 20 to 35, about 20 to 40, about 25 to 30, about 25 to 35, about 25 to 40, about 30 to 35, or about 35 to 40 nucleotides in length. In some embodiments, the region of identity is no more than 25, no more than 30, no more than 35, no more than 40, or no more than 45 nucleotides in length. In some embodiments, the second editing template comprises a region of identity to the second spacer. In some embodiments, the second editing template comprises nucleotides m to (n−3) of the first spacer, wherein n is the length of the first spacer, and m is any integer between 1 and (n−12). In some embodiments, n is an integer from 16 to 22. For example, for a 20 nucleotide first spacer, the second editing template can contain nucleotides 1-17, 2-17, 3-17, 4-17, 5-17, 6-17, 7-7, or 8-17 of the second spacer.

Accordingly, as exemplified in FIG. 4E, in some embodiments, the second newly synthesized single-stranded DNA encoded by the second editing template comprises a region of complementarity to a sequence on the second strand of the double-stranded target DNA adjacent or immediately adjacent to the first nick site generated by the first prime editor complexed with the first PERNA. In some embodiments, the second newly synthesized single-stranded DNA encoded by the second editing template comprises a region of complementarity to a sequence on the second strand of the double-stranded target DNA adjacent or immediately adjacent to the first nick site generated by the first prime editor complexed with the first PEgRNA, wherein the sequence is outside the IND. In some embodiments, the second newly synthesized single-stranded DNA anneals with the sequence on the second strand of the double-stranded target DNA adjacent or immediately adjacent to the first nick site generated by the first prime editor. In some embodiments, through DNA repair, the IND is excised and deleted from the double-stranded target DNA, e.g., the target gene.

In some embodiments, the first editing template of the first PEgRNA comprises a region at least partially identical to a sequence on the first strand of the double-stranded target DNA immediately adjacent to the second nick site generated by the second prime editor complexed with the second PEgRNA, wherein the sequence is outside the IND. In some embodiments, the second editing template of the second PEgRNA comprises a region at least partially identical to a sequence on the second strand of the double-stranded target DNA immediately adjacent to the first nick site generated by the first prime editor complexed with the first PEgRNA, wherein the sequence is outside the IND. In some embodiments, the first editing template and the second editing template further comprise a region of complementarity or substantial complementarity to each other.

Accordingly, as exemplified in FIG. 4F, in some embodiments, the first newly synthesized single-stranded DNA encoded by the first editing template comprises a region of complementarity to a sequence on the first strand of the double-stranded target DNA, wherein the sequence is immediately adjacent to the second nick site generated by the second prime editor complexed with the second PEgRNA and is outside the IND. In some embodiments, the second newly synthesized single-stranded DNA encoded by the second editing template comprises a region of complementarity or substantial complementarity to a sequence on the second strand of the double-stranded target DNA, wherein the sequence is immediately adjacent to the first nick site generated by the first prime editor complexed with the first PEgRNA and is outside the IND. In some embodiments, the first newly synthesized single-stranded DNA encoded by the first editing template and the second newly synthesized single-stranded DNA encoded by the second editing template further comprise a region of complementarity or substantial complementarity to each other, and can anneal to each other to form an OD.

In some embodiments, the first newly synthesized single-stranded DNA encoded by the first editing template further comprises a region that is not complementary to the second newly synthesized single-stranded DNA encoded by the second editing template and does not have complementarity or identity to the double-stranded target DNA. In some embodiments, the second newly synthesized single-stranded DNA encoded by the second editing template further comprises a region that is not complementary to the first newly synthesized single-stranded DNA encoded by the first editing template and does not have complementarity or identity to the double-stranded target DNA, e.g., the target gene.

Accordingly, in some embodiments, the RD comprises (i) the OD, (ii) the region of the first newly synthesized single-stranded DNA that is not complementary to the second newly synthesized single-stranded DNA and does not have complementarity or identity to the double-stranded target DNA, and a complementary sequence thereof, and (iii) the region of the second newly synthesized single-stranded DNA that is not complementary to the first newly synthesized single-stranded DNA and does not have complementarity or identity to the double-stranded target DNA, and a complementary sequence thereof. In some embodiments, through DNA repair, the IND is excised from the double-stranded target DNA, e.g., the TCF4 gene, and the RD is incorporated into the double-stranded target DNA.

In some embodiments, the IND is excised and deleted from the target gene, and the RD is incorporated at the place of excision of the IND. In some embodiments, the IND is excised and deleted from the target gene, and the OD is incorporated at the place of excision of the IND. In some embodiments, the RD comprises a region of identity to an endogenous sequence of the double-stranded target DNA. In some embodiments, the OD comprises a region of identity to an endogenous sequence of the double-stranded target DNA. In some embodiments, the RD does not have sequence identity to an endogenous sequence of the double-stranded target DNA. In some embodiments, the RD is exogenous to the double-stranded target DNA, e.g., the target gene. In some embodiments, the RD has a biological function or encodes a polypeptide having a biological function. In some embodiments, the OD does not have sequence identity to an endogenous sequence of the double-stranded target DNA. In some embodiments, the OD is exogenous to the double-stranded target DNA, e.g., the target gene. In some embodiments, the OD has a biological function or encodes a polypeptide having a biological function.

In some embodiments, the first editing template of the first PEgRNA comprises a region at least partially identical to a sequence of the double-stranded target DNA that is outside the IND and is not immediately adjacent to (also referred to as “distal to”) the second nick site on the second PAM strand of the double-stranded target DNA. In some embodiments, the first editing template of the first PEgRNA comprises a region of identity to a sequence of double-stranded target DNA on the second PAM strand that is outside the IND and is at least 1, 2, 3, 4, 5, 6, 7, 8, 9, 10, 11, 12, 13, 14, 15, 16, 17, 18, 19, 20 or more nucleotides upstream of the second nick site.

In some embodiments, the second editing template of the second PEgRNA comprises a region at least partially identical to a sequence of the double-stranded target DNA that is outside the IND and is not immediately adjacent to the first nick site on the first PAM strand of the double-stranded target DNA. In some embodiments, the second editing template of the second PEgRNA comprises a region of identity to a sequence of the double-stranded target DNA on the first PAM strand that is outside the IND and is at least 1, 2, 3, 4, 5, 6, 7, 8, 9, 10, 11, 12, 13, 14, 15, 16, 17, 18, 19, 20 or more nucleotides upstream of the first nick site.

Accordingly, in some embodiments, the first newly synthesized single-stranded DNA encoded by the first editing template comprises a region of complementarity to a sequence of the double-stranded target DNA that is outside the IND and is not immediately adjacent (i.e., distal) to the second nick site on the second PAM strand. In some embodiments, the first newly synthesized DNA encoded by the first editing template can anneal with the sequence that is outside the IND and is not immediately adjacent to the second nick site on the second PAM strand of the double-stranded target DNA. In some embodiments, the first newly synthesized DNA encoded by the first editing template comprises a region of complementarity to, and can anneal with a sequence of the double-stranded target DNA that is outside the IND and is at least 1, 2, 3, 4, 5, 6, 7, 8, 9, 10, 11, 12, 13, 14, 15, 16, 17, 18, 19, 20 or more nucleotides downstream upstream of the second nick site. In some embodiments, the second newly synthesized single-stranded DNA encoded by the second editing template comprises a region of complementarity to a sequence of the first PAM strand of the double-stranded target DNA that is outside the IND and is not immediately adjacent to (also referred to as “distal to”) the first nick site on the first PAM strand. In some embodiments, the second newly synthesized DNA encoded by the second editing template can anneal with the sequence that is outside the IND and is not immediately adjacent to the first nick site on the first PAM strand of the double-stranded target DNA. In some embodiments, the second newly synthesized DNA encoded by the second editing template comprises a region of complementarity to, and can anneal with a sequence of the double-stranded target DNA that is outside the IND and is at least 1, 2, 3, 4, 5, 6, 7, 8, 9, 10, 11, 12, 13, 14, 15, 16, 17, 18, 19, 20 or more nucleotides upstream of the first nick site.

In some embodiments, through DNA repair, the IND is excised and deleted from the double-stranded target DNA, e.g., the target gene. In some embodiments, the endogenous sequence of the double-stranded target DNA between the 3′ end of the sequence that is outside the IND and is distal to the second nick site on the second PAM strand and the 3′ end of the sequence of the double-stranded target DNA that is outside the IND and is distal to the first nick site on the first PAM strand of the double-stranded target DNA is excised and deleted from the double-stranded target DNA.

In some embodiments, as exemplified in FIG. 4G, the first search target sequence protospacer is downstream of the second search target sequence. In some embodiments, the first editing template comprises a region that has complementarity or substantial complementarity to the second editing template, and optionally further comprises a region that does not have a complementarity to the second editing template. In some embodiments, the second editing template comprises a region that has complementarity or substantial complementarity to the first editing template, and optionally further comprises a region that does not have complementarity to the first editing template.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the polypeptide domain having DNA binding activity is a polypeptide domain having programmable DNA binding activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides or polypeptide domains involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide and a reverse transcriptase polypeptide that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PERNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

The term “prime editor complex” is used interchangeably with the term “prime editing complex” and refers to a complex comprising one or more prime editor components (e.g., a polypeptide domain having DNA binding activity and a polypeptide domain having DNA polymerase activity) complexed with a PEgRNA.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single-stranded DNA using a PEgRNA editing template that comprises a DNA sequence as a template. In such cases, the PEgRNA is a chimeric or hybrid PERNA, and comprises an extension arm comprising a DNA strand. As used herein, an “extension arm” is a polynucleotide portion of a PERNA that comprises an editing template and a primer binding site sequence (PBS). In some embodiments, an extension arm further comprises additional components, for example, a 3′ modifier. The chimeric or hybrid PEgRNA may comprise an RNA portion (including the spacer and the gRNA core) and a DNA portion (the extension arm comprising the editing template that includes a strand of DNA).

The DNA polymerases can be wild-type polymerases from eukaryotic, prokaryotic, archael, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol IV family DNA polymerase. In some embodiments, the DNA polymerase is a E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLDI DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol III family DNA polymerase. In some embodiments, the DNA polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). A RT or an RT domain may be a wild-type RT domain, a full-length RT domain, or may be a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MLV) RT; human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus RT, Avian Myelocytomatosis Virus MC29 Helper Virus (MCAV) RT, Avian Reticuloendotheliosis Virus (REV-T; REV-A) Helper Virus RT, Avian Sarcoma Virus UR2 Helper Virus (UR2AV) RT, Avian Sarcoma Virus Y73 Helper Virus (YAV) RT, Rous Associated Virus (RAV) RT, and Myeloblastosis Associated Virus (MAV) RT, all of which may be suitably used in the methods and composition described herein.

In some embodiments, the prime editor comprises a wild-type M-MLV RT. An exemplary sequence of a reference M-MLV RT is provided in SEQ ID NO: 366.

In some embodiments, the prime editor comprises a M-MLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 366, where X is any amino acid other than the wild-type amino acid. In some embodiments, the prime editor comprises a M-MLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the wild-type M-MLV RT as set forth in SEQ ID NO: 366. In some embodiments, the prime editor comprises a M-MLV RT comprising one or more amino acid substitutions D200N, T330P, L603W, T306K, and W313F as compared to the reference M-MLV RT as set forth in SEQ ID NO: 366. In some embodiments, the prime editor comprises a M-MLV RT comprising amino acid substitutions D200N, T330P, L603W, T306K, and W313F as compared to the reference M-MLV RT as set forth in SEQ ID NO: 366. In some embodiments, a prime editor comprises a M-MLV RT variant having the sequence as set forth in SEQ ID NO: 2.

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., a wild-type RT. In some embodiments, the RT variant comprises a fragment of a reference RT, e.g., a wild-type RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the reference RT. In some embodiments, the fragment is 30%, 35%, 40%, 45%, 50%, 55%, 60%, 65%, 70%, 75%, 80%, 85%, 90%, 95%, 96%, 97%, 98%, 99%, or 99.5% of the amino acid length of a corresponding reference RT (M-MLV reverse transcriptase) (e.g., SEQ ID NO: 366).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT. In some embodiments, the reference RT has the sequence as set forth in SEQ ID NO: 366. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT. In some embodiments, the reference RT comprises the sequence set forth in SEQ ID NO: 366. In still other embodiments, the RT truncated variant has a truncation at the N-terminal and the C-terminal end compared to a reference RT. In some embodiments, the reference RT consist of the sequence as set forth in SEQ ID NO: 366. In some embodiments, the N-terminal truncation and the C-terminal truncation are of the same length. In some embodiments, the N-terminal truncation and the C-terminal truncation are of different lengths. For example, the prime editors disclosed herein may include a functional variant of a wild-type M-MLV reverse transcriptase. In some embodiments, the prime editor comprises a functional variant of a reference M-MLV RT, wherein the functional variant of M-MLV RT is truncated after amino acid position 502 compared to a reference M-MLV RT as set forth in SEQ ID NO: 366. In some embodiments, the functional variant of M-MLV RT further comprises a D200X, T306X, W313X, and/or T330X amino acid substitution compared to a reference M-MLV RT as set forth in SEQ ID NO: 366, wherein X is any amino acid other than the original amino acid. In some embodiments, the functional variant of M-MLV RT further comprises a D200N, T306K, W313F, and/or T330P amino acid substitution compared to a reference M-MLV RT as set forth in SEQ ID NO: 366, wherein X is any amino acid other than the original amino acid. A DNA sequence encoding a prime editor comprising this truncated RT is 522 bp smaller than the DNA encoding the reference MLV RT (e.g., the RT as set forth in SEQ ID NO: 366), and therefore makes it potentially useful for applications where delivery of the DNA sequence is challenging due to its size (i.e., adeno-associated virus and lentivirus delivery).

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.12) RT. In some embodiments, the prime editor comprises a retron RT. Table 20 provides sequences of the reference MLV RT (SEQ ID NO: 366) and MLV RT variant (SEQ ID NO: 2).

### Programmable DNA Binding Domain

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprise a nuclease activity. In some embodiments, the DNA-binding domain of a prime editor comprises an endonuclease domain having single-strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild-type endonuclease domain. For example, the endonuclease domain may comprise one or more amino acid substitutions as compared to a wild-type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild-type Cas protein, the Cas nickase comprises one or more amino acid substitutions in a nuclease domain that reduces or abolishes its double-strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, Cas @, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

A Cas protein, e.g., Cas9, can be a wild-type or a modified form of a Cas protein. A Cas protein, e.g., Cas9, can be a nuclease active variant, nuclease inactive variant, a nickase, or a functional variant or functional fragment of a wild-type Cas protein. A Cas protein, e.g., Cas9, can comprise an amino acid change such as a deletion, insertion, substitution, fusion, chimera, or any combination thereof relative to a wild-type version of the Cas protein. A Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild-type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain that can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand (i.e., the PAM strand) or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain. In some embodiments, the Cas9 nickase comprises a D10X amino acid substitution compared to a wild-type S. pyogenes Cas9, as set forth in SEQ ID NO: 3, wherein X is any amino acid other than D. In some embodiments, a prime editor comprises a Cas9 nickase having an amino acid substitution in the HNH domain. In some embodiments, the Cas9 nickase comprises a H840X amino acid substitution compared to a wild-type S. pyogenes Cas9, as set forth in SEQ ID NO: 3, wherein X is any amino acid other than H.

In some embodiments, a prime editor comprises a Cas protein that can bind to the target gene in a sequence-specific manner but lacks or has abolished nuclease activity and may not cleave either strand of a double-stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild-type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild-type reference Cas9 protein (e.g., Cas9 from S. pyogenes). In some embodiments, the Cas9 comprises an amino acid change such as a deletion, insertion, substitution, fusion, chimera, or any combination thereof as compared to a wild-type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or those known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

In some embodiments, a Cas9 is a chimeric Cas9, e.g., modified Cas9; e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa).

An exemplary wild-type Streptococcus pyogenes Cas9 (SpCas9) amino acid sequence is provided in SEQ ID NO: 3.

In some embodiments, a prime editor comprises a Cas9 protein from Staphylococcus lugdunensis (Slu Cas9). An exemplary amino acid sequence of a wild-type Slu Cas9 is provided in SEQ ID NO: 4.

In some embodiments, a Cas9 protein comprises a variant Cas9 protein containing one or more amino acid substitutions. In some embodiments, a wild-type Cas9 protein comprises a RuvC domain and an HNH domain. In some embodiments, a prime editor comprises a nuclease active Cas9 protein that may cleave both strands of a double-stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double-stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double-stranded target DNA sequence. In some embodiments, a prime editor comprises a Cas9 having a non-functional RuvC domain that can cleave the target strand (i.e., the non-PAM strand), but not the edit strand of a double-stranded target DNA sequence. In some embodiments, a prime editor comprises a Cas9 that has neither a functional RuvC domain nor a functional HNH domain, which may not cleave any strand of a double-stranded target DNA sequence.

In some embodiments, a prime editor comprises a Cas9 having a mutation in the RuvC domain that reduces or abolishes the nuclease activity of the RuvC domain. In some embodiments, the Cas9 comprise a mutation at amino acid D10 as compared to a wild-type SpCas9 as set forth in SEQ ID NO: 3, or a corresponding mutation thereof. In some embodiments, the Cas9 comprise a D10A mutation as compared to a wild-type SpCas9 as set forth in SEQ ID NO: 3, or a corresponding mutation thereof. In some embodiments, the Cas9 polypeptide comprise a mutation at amino acid D10, G12, and/or G17 as compared to a wild-type SpCas9 as set forth in SEQ ID NO: 3, or a corresponding mutation thereof. In some embodiments, the Cas9 polypeptide comprise a D10A mutation, a G12A mutation, and/or a G17A mutation as compared to a wild-type SpCas9 as set forth in SEQ ID NO: 3, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild-type SpCas9 as set forth in SEQ ID NO: 3, or a corresponding mutation thereof. In some embodiments, the Cas9 polypeptide comprise a H840A mutation as compared to a wild-type SpCas9 as set forth in SEQ ID NO: 3, or a corresponding mutation thereof. In some embodiments, the Cas9 polypeptide comprises a mutation at amino acid E762, D839, H840, N854, N856, N863, H982, H983, A984, D986, and/or a A987 as compared to a wild-type SpCas9 as set forth in SEQ ID NO: 3, or a corresponding mutation thereof. In some embodiments, the Cas9 polypeptide comprise a E762A, D839A, H840A, N854A, N856A, N863A, H982A, H983A, A984A, and/or a D986A mutation as compared to a wild-type SpCas9 as set forth in SEQ ID NO: 3, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild-type SpCas9 as set forth in SEQ ID NO: 3 or corresponding mutations thereof, wherein X is any amino acid other than H for the H840X substitution and any amino acid other than D for the D10X substitution. In some embodiments, the dead Cas9 comprises a H840A and a D10A mutation as compared to a wild-type SpCas9 as set forth in SEQ ID NO: 3, or corresponding mutations thereof.

In some embodiments, the N-terminal methionine is removed from a Cas9 nickase, or from any Cas9 variant, ortholog, or equivalent disclosed or contemplated herein. For example, methionine-minus Cas9 nickases, or a variant thereof having an amino acid sequence that has at least 80%, at least 85%, at least 90%, at least 95%, or at least 99% sequence identity thereto.

In some embodiments, a prime editor comprises a Streptococcus pyogenes Cas9 (SpCas9) having a nuclease inactivating mutation in the HNH domain (a SpCas9 nickase). In some embodiments, the SpCas9 nickase lacks the N-terminus methionine relative to a corresponding reference SpCas9 (e.g., wild-type SpCas9). In some embodiments, a prime editor comprises a SpCas9 nickase having the sequences as provide in SEQ ID NO: 5 (SpCas9 H840A nickase including the N-terminal methionine). In some embodiments, a prime editor comprises a SpCas9 nickase having the sequences as provide in SEQ ID NO: 6 (SpCas9 H840A nickase lacking the N-terminal methionine).

In some embodiments, the SpCas9 nickase further comprises a R221K and/or a N394K amino acid substitution compared to a reference SpCas9 sequence set forth in SEQ ID NO: 3. In some embodiments, the SpCas9 nickase comprises a sequence as set forth in SEQ ID NO: 3.

In some embodiments, a prime editor comprises a Staphylococcus lugdunensis (SluCas9) having a nuclease inactivating mutation in the HNH domain (a SluCas9 nickase). In some embodiments, the SluCas9 nickase lacks the N-terminus methionine relative to a corresponding reference SluCas9 (e.g., wild-type SluCas9). In some embodiments, a prime editor comprises a SluCas9 nickase having the sequences as provide in SEQ ID NO: 9 (SluCas9 H840A nickase including the N-terminal methionine). In some embodiments, a prime editor comprises a SluCas9 nickase having the sequences as provide in SEQ ID NO: 10 (SluCas9 H840A nickase lacking the N-terminal methionine).

Besides dead Cas9 and Cas9 nickase variants, the Cas9 proteins used herein may also include other Cas9 variants having at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to any reference Cas9 protein, including any wild-type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild-type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild-type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild-type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif”, known as a PAM sequence, or PAM-like motif, may be used to refer to a short DNA sequence immediately following the protospacer on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 1 below. It should be appreciated that for each of the variants provided, the Cas protein comprises one or more of the amino acid substitutions as indicated compared to a wild-type Cas protein sequence, for example, the SpCas9 as set forth in SEQ ID NO: 3.

The PAM motifs as shown in Table 1 below are in the order of 5′ to 3′. The nucleotides listed in Table 1 are represented by the base codes as provided in the Handbook on Industrial Property Information and Documentation, World Intellectual Property Organization (WIPO) Standard ST.26, Version 1.4. For example, an “R” in Table 1 represents the nucleotide A or G; “W” in Table 1 represents A or T; and “V” in Table 1 represents A or C or G.

TABLE 1
Cas protein variants and corresponding PAM sequences

| Variant | PAM |
|---|---|
| spCas9 (wild-type) | NGG, NGA, NAG |
| spCas9- VRVRFRR (also referred to as SpCas9-NG) | NG |
| R1335V/L1111R/D1135V/G1218R/E1219F/A1322R/T1337R | |
| spCas9-VQR (D1135V/R1335Q/T1337R) | NGA |
| spCas9-EQR (D1135E/R1335Q/T1337R) | NGA |
| spCas9-VRER (D1135V/G1218R/R1335E/T1337R) | NGCG |
| xCas9 (E480K, E543D, E1219V, K294R, Q1256K, A262T, | NGN |
| S409I, M694I) | |
| SluCa9 | NNGG |
| saCas9 | NNGRRT, |
| | NNGRRN |
| saCas9-KKH (E782K, N968K, R1015H) | NNNRRT |
| spCas9-MQKSER (D1135M, S1136Q, G1218K, E1219S, | NGCG/NGCN |
| R1335E, T1337R) | |
| spCas9-LRKIQK (D1135L, S1136R, G1218K, E1219I, | NGTN |
| R1335Q, T1337K) | |
| spCas9-LRVSQK (D1135L, S1136R, G1218V, E1219S, | NGTN |
| R1335Q, T1337K) | |
| spCas9-LRVSQL(D1135L, S1136R, G1218V, E1219S, | NGTN |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |
| spCas9-VRQR (D1135V, G1218R, R1335Q, T1337R) | NGA |
| SpG Cas9 (D1135L, S1136W, G1218K, E1219Q, R1335Q, | NGN |
| T1337R) | |
| SyRY Cas9 | NRN |
| (A61R, L1111R, N1317R, A1322R, and R1333P) | |
| SRGN1, sRGN2, sRGN4, sRGN3.1, sRGN3.3 | NNGG |

Exemplary Cas9s that allow alternative PAM recognition are provided in SEQ ID NO: 11 (SpCas9-NG), SEQ ID NO: 12 (SpCas9-NG H840A nickase), SEQ ID NO: 13 (SpCas9-NG H839A nickase lacking N-terminal methionine), SEQ ID NO: 14 (SpCas9-VRQR), SEQ ID NO: 15 (SpCas9-VRQR H840A nickase), SEQ ID NO: 16 (SpCas9-VRQR H839A nickase lacking N-terminal methionine), SEQ ID NO: 17 (SpRY Cas9), SEQ ID NO: 18 (SpRY Cas9 H840A nickase), SEQ ID NO: 19 (SpRY Cas9 H839A nickase lacking N-terminal methionine), SEQ ID NO: 20 (sRGN3.1), SEQ ID NO: 21 (sRGN3.1 N585A nickase), SEQ ID NO: 22 (sRNA3.1 N584A nickase lacking N-terminal methionine), SEQ ID NO: 23 (sRGN3.3), SEQ ID NO: 24 (sRGN3.3 N585A nickase), SEQ ID NO: 25 (sRGN3.3 N584A nickase lacking N-terminal methionine), SEQ ID NO: 26 (SpG Cas9), SEQ ID NO: 27 (SpG Cas9 H840A nikcase), and SEQ ID NO: 28 (SpG Cas9 H839A nickase lacking N-terminal methionine). Table 20 provides the sequences of the referenced Cas9 wild-type and variant homologs.

In some embodiments, a prime editor comprises a Cas9 polypeptide comprising one or more mutations selected from the group consisting of: A61R, L111R, D1135V, R221K, A262T, R324L, N394K, S409I, S409I, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wild-type SpCas9 polypeptide as set forth in SEQ ID NO: 3.

In some embodiments, a prime editor comprises a SaCas9 polypeptide. In some embodiments, the SaCas9 polypeptide comprises one or more of mutations E782K, N968K, and R1015H as compared to a wild-type SaCas9. In some embodiments, a prime editor comprises a FnCas9 polypeptide, for example, a wild-type FnCas9 polypeptide or a FnCas9 polypeptide comprising one or more of mutations E1369R, E1449H, or R1556A as compared to the wild-type FnCas9. In some embodiments, a prime editor comprises a Sc Cas9, for example, a wild-type ScCas9 or a ScCas9 polypeptide comprises one or more of mutations I367K, G368D, I369K, H371L, T375S, T376G, and T1227K as compared to the wild-type ScCas9. In some embodiments, a prime editor comprises a St1 Cas9 polypeptide, a St3 Cas9 polypeptide, or a Slu Cas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild-type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a SpCas9, may have the following structure: N-terminus-[original C-terminus]-[optional linker]-[original N-terminus]-C-terminus. In some embodiments, a circular permutant SpCas9 comprises any one of the following structures (amino acid positions as set forth in SEQ ID NO: 3.
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 3:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus;
N-terminus-[1300-1368]-[optional linker]-[1-1299]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 3:
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus;
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the C-terminal 95% or more of the amino acids of a Cas9 (e.g., amino acids about 1300-1368 as set forth in SEQ ID NO: 3 or corresponding amino acid positions thereof), or the C-terminal 90%, 85%, 80%, 75%, 70%, 65%, 60%, 55%, 50%, 45%, 40%, 35%, 30%, 25%, 20%, 15%, 10%, or 5% or more of a Cas9. The N-terminal portion may correspond to the N-terminal 95% or more of the amino acids of a Cas9 (e.g., amino acids about 1-1300 as set forth in SEQ ID NO: 3 or corresponding amino acid positions thereof), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., as set forth in SEQ ID NO: 3 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID NO: 3 or corresponding amino acid positions thereof). In some embodiments, the C-terminal fragment that is rearranged to the N-terminus, includes or corresponds to the C-terminal 30%, 29%,28%,27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID NO: 3 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID NO: 3 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., as set forth in SEQ ID NO: 3 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID NO: 3 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 3: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID NO: 3 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰ Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 3, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild-type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Csel, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild-type Cas9 polypeptide of SEQ ID NO: 3). In various other embodiments, the polypeptide domain having DNA binding activity can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof. Exemplary Cas proteins and nomenclature are shown in Table 2 below:

TABLE 2
Exemplary Cas proteins and nomenclature

| | |
|---|---|
| Legacy nomenclature | Current nomenclature |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas 12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a CasΦ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas @ protein. In some embodiments, the Cas protein is a Cas12e, Cas12d, Cas13, or Cas @ nickase.

### Flap Endonuclease

In some embodiments, a prime editor further comprises additional polypeptide components, for example, a flap endonuclease (FEN), e.g., FEN1. In some embodiments, the flap endonuclease excises the 5′ single-stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN.

In some embodiments, a prime editor or prime editing composition comprises a flap nuclease. In some embodiments, the flap nuclease is a FEN1, or any FEN1 functional variant, functional mutant, or functional fragment thereof. In some embodiments, the flap nuclease is a TREX2, EXO1, or any other flap nuclease known in the art, or any functional variant, functional mutant, or functional fragment thereof. In some embodiments, the flap nuclease has an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, for example, a fusion protein comprising a DNA binding domain and a DNA polymerase that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing composition comprises at least one NLS. In some embodiments, a prime editor or prime editing composition comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise one NLS. In some cases, a prime editor may further comprise two NLSs. In other cases, a prime editor may further comprise three NLSs. In one case, a primer editor may further comprise more than 4, 5, 6, 7, 8, 9 or 10 NLSs.

In addition, the NLSs may be expressed as part of a prime editor or prime editing composition. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is a fusion protein that comprises an NLS at the N terminus. In some embodiments, a prime editor is a fusion protein that comprises an NLS at the C terminus. In some embodiments, a prime editor is a fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence

(SEQ ID NO: 29)
MDSLLMNRRKFLYQFKNVRWAKGRRETYLC,
(SEQ ID NO: 30)
MKRTADGSEFESPKKKRKV,
(SEQ ID NO: 31)
KRTADGSEFEPKKKRKV,
(SEQ ID NO: 32)
NLSKRPAAIKKAGQAKKKK,
(SEQ ID NO: 33)
RQRRNELKRSF,
or
(SEQ ID NO: 34)
NQSSNFGPMKGGNFGGRSSGPYGGGGQYFAKPRNQGGY.

In some embodiments, a NLS is a monopartite NLS. For example, in some embodiments, a NLS is a SV40 large T antigen NLS PKKKRKV (SEQ ID NO: 35). In some embodiments, a NLS is a bipartite NLS. In some embodiments, a bipartite NLS comprises two basic domains separated by a linker comprising a variable number of amino acids. In some embodiments, a NLS is a bipartite NLS. In some embodiments, a bipartite NLS consists of two basic domains separated by a linker comprising a variable number of amino acids. In some embodiments, the linker amino acid sequence comprises the sequence KRXXXXXXXXXXKKKL (SEQ ID NO: 36), wherein X is any amino acid. In some embodiments, the NLS comprises a nucleoplasmin NLS sequence KRPAATKKAGQAKKKK (SEQ ID NO: 37). In some embodiments, a NLS is a noncanonical sequences such as M9 of the hnRNP Alprotein, the influenza virus nucleoprotein NLS, and the yeast Gal4 protein NLS.

Other non-limiting examples of NLS sequences are provided in Table 3 below.

TABLE 3
Exemplary nuclear localization sequences

| SEQ ID | | |
|---|---|---|
| NO: | Description | Sequence |
| 35 | NLS of SV40 | PKKKRKV |
| | Large T-AG | |
| 30 | NLS | MKRTADGSEFESPKKKRKV |
| 29 | NLS | MDSLLMNRRKFLYQFKNVRWAKGRRETYLC |
| 38 | NLS of | AVKRPAATKKAGQAKKKKLD |
| | Nucleoplasmin | |
| 39 | NLS of EGL-13 | MSRRRKANPTKLSENAKKLAKEVEN |
| 40 | NLS of C-Myc | PAAKRVKLD |
| 41 | NLS of Tus-protein | KLKIKRPVK |
| 42 | NLS of polyoma | VSRKRPRP |
| | large T-AG | |
| 43 | NLS of Hepatitis D | EGAPPAKRAR |
| | virus antigen | |
| 44 | NLS of murine p53 | PPQPKKKPLDGE |
| 45 | C-terminal NLS | SGGSKRTADGSEFEPKKKRKV |
| | and linker for | |
| | exemplary PE | |
| | fusion protein | |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a trans-splicing reaction. In some embodiments, the trans-splicing reaction excises the two intein sequences and links the two extein sequences with a peptide bond. As a result, the intein-N and the intein-C are spliced out, and a protein domain linked to the intein-N is fused to a protein domain linked to the intein-C essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild-type intein-N or wild-type intein-C, for example, amino acid substitutions that enhances the trans-splicing activity of the split intein. In some embodiments, the intein-C comprises 4 to 7 contiguous amino acid residues, wherein at least 4 amino acids of which are from the last β-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-S-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

Polypeptides comprising components of a prime editor may be fused via peptide linkers, or may be provided in trans relative to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Corn polypeptide and a Corn RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer is fused or linked to a portion of the PEgRNA. For example, an MS2 coat protein may be fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP or MS2cp), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 46). In some embodiments, the amino acid sequence of the MCP is:

(SEQ ID NO: 47)
GSASNFTQFVLVDNGGTGDVTVAPSNFANGVAEWISSNSRSQAYKVTC
SVRQSSAQNRKYTIKVEVPKVATQTVGGEELPVAGWRSYLNMELTIPI
FATNSDCELIVKAMQGLLKDGNPIPSAIAANSGIY.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, and carbon-heteroatom bond).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)ₙ (SEQ ID NO: 48), (G)ₙ (SEQ ID NO: 49), (EAAAK)ₙ (SEQ ID NO:50), (GGS)ₙ (SEQ ID NO: 51), (SGGS)ₙ (SEQ ID NO: 52), (XP)ₙ (SEQ ID NO: 53), or any combination thereof, wherein n is independently an integer between 1 and 30, and wherein X is any amino acid. In some embodiments, the linker comprises the amino acid sequence (GGS) n (SEQ ID NO: 51), wherein n is 1, 3, or 7. In some embodiments, the linker comprises the amino acid sequence SGSETPGTSESATPES (SEQ ID NO: 54). In some embodiments, the linker comprises the amino acid sequence SGGSSGGSSGSETPGTSESATPESSGGSSGGS (SEQ ID NO: 55). In some embodiments, the linker comprises the amino acid sequence SGGSSGGSSGSETPGTSESATPESSGGSSGGSS (SEQ ID NO: 56). In some embodiments, the linker comprises the amino acid sequence SGGSGGSGGS (SEQ ID NO: 57). In some embodiments, the linker comprises the amino acid sequence SGGS (SEQ ID NO: 58). In other embodiments, the linker comprises the amino acid sequence SGGSSGGSSGSETPGTSESATPESAGSYPYDVPDYAGSAAPAAKKKKLDGSGSGGS SGGS (SEQ ID NO: 59).

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, or combination thereof). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, or combination thereof). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[DNA polymerase]-COOH; or NH2-[DNA polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans-splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell.

The amino acid sequence of an exemplary prime editor (PE) fusion protein and its individual components is shown in Table 4. Table 5 also includes the amino acid sequence of an exemplary prime editor (PE) fusion protein and its individual components.

In some embodiments, a prime editing composition comprises a fusion protein comprising a DNA binding domain (e.g., Cas9 (H840A)) and a reverse transcriptase (e.g., a variant M-MLV RT) having the following structure: [NLS]-[Cas9 (H840A)]-[linker]-[M-MLV_RT (D200N) (T330P) (L603W) (T306K) (W313F)], and one or more desired PERNAs. In some embodiments, the prime editing composition comprises a prime editor fusion protein that has the amino acid sequence of SEQ ID NO: 60.

In various embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the exemplary PE fusion protein provided below, or any of the prime editor fusion sequences described herein or known in the art.

TABLE 4
Amino acid sequence of exemplary PE and components

| SEQ ID | | |
|---|---|---|
| NO: | Description | Sequence |
| 60 | PE FUSION | MKRTADGSEFESPKKKRKVDKKYSIGLDIGTNSVGW |
| | PROTEIN | AVITDEYKVPSKKFKVLGNTDRHSIKKNLIGALLFD |
| | CAS9(H840A)- | SGETAEATRLKRTARRRYTRRKNRICYLQEIFSNEM |
| | MMLV_RT | AKVDDSFFHRLEESFLVEEDKKHERHPIFGNIVDEV |
| | D200N T330P | AYHEKYPTIYHLRKKLVDSTDKADLRLIYLALAHMI |
| | L603W T306K | KFRGHFLIEGDLNPDNSDVDKLFIQLVQTYNQLFEE |
| | W313F | NPINASGVDAKAILSARLSKSRRLENLIAQLPGEKKN |
| | | GLFGNLIALSLGLTPNFKSNFDLAEDAKLQLSKDTY |
| | | DDDLDNLLAQIGDQYADLFLAAKNLSDAILLSDILR |
| | | VNTEITKAPLSASMIKRYDEHHQDLTLLKALVRQQ |
| | | LPEKYKEIFFDQSKNGYAGYIDGGASQEEFYKFIKPI |
| | | LEKMDGTEELLVKLNREDLLRKQRTFDNGSIPHQI |
| | | HLGELHAILRRQEDFYPFLKDNREKIEKILTFRIPYY |
| | | VGPLARGNSRFAWMTRKSEETITPWNFEEVVDKGA |
| | | SAQSFIERMTNFDKNLPNEKVLPKHSLLYEYFTVYN |
| | | ELTKVKYVTEGMRKPAFLSGEQKKAIVDLLFKTNR |
| | | KVTVKQLKEDYFKKIECFDSVEISGVEDRFNASLGT |
| | | YHDLLKIIKDKDFLDNEENEDILEDIVLTLTLFEDRE |
| | | MIEERLKTYAHLFDDKVMKQLKRRRYTGWGRLSR |
| | | KLINGIRDKQSGKTILDFLKSDGFANRNFMQLIHDD |
| | | SLTFKEDIQKAQVSGQGDSLHEHIANLAGSPAIKKGI |
| | | LQTVKVVDELVKVMGRHKPENIVIEMARENQTTQ |
| | | KGQKNSRERMKRIEEGIKELGSQILKEHPVENTQL |
| | | QNEKLYLYYLQNGRDMYVDQELDINRLSDYDVDAI |
| | | VPQSFLKDDSIDNKVLTRSDKNRGKSDNVPSEEVVK |
| | | KMKNYWRQLLNAKLITQRKFDNLTKAERGGLSEL |
| | | DKAGFIKRQLVETRQITKHVAQILDSRMNTKYDEN |
| | | DKLIREVKVITLKSKLVSDFRKDFQFYKVREINNYH |
| | | HAHDAYLNAVVGTALIKKYPKLESEFVYGDYKVYD |
| | | VRKMIAKSEQEIGKATAKYFFYSNIMNFFKTEITLA |
| | | NGEIRKRPLIETNGETGEIVWDKGRDFATVRKVLS |
| | | MPQVNIVKKTEVQTGGFSKESILPKRNSDKLIARKK |
| | | DWDPKKYGGFDSPTVAYSVLVVAKVEKGKSKKLK |
| | | SVKELLGITIMERSSFEKNPIDFLEAKGYKEVKKDLI |
| | | IKLPKYSLFELENGRKRMLASAGELQKGNELALPS |
| | | KYVNFLYLASHYEKLKGSPEDNEQKQLFVEQHKHY |
| | | LDEIIEQISEFSKRVILADANLDKVLSAYNKHRDKPI |
| | | REQAENIIHLFTLTNLGAPAAFKYFDTTIDRKRYTST |
| | | KEVLDATLIHQSITGLYETRIDLSQLGGDSGGSSGGS |
| | | SGSETPGTSESATPESSGGSSGGSSTLNIEDEYRLHETS |
| | | KEPDVSLGSTWLSDFPQAWAETGGMGLAVRQAPLIIPLKA |
| | | TSTPVSIKQYPMSQEARLGIKPHIQRLLDQGILVPCQSPWN |
| | | TPLLPVKKPGTNDYRPVQDLREVNKRVEDIHPTVPNPYNL |
| | | LSGLPPSHOWYTVLDLKDAFFCLRLHPTSQPLFAFEWRD |
| | | PEMGISGQLTWTRLPQGFKNSPTLFNEALHRDLADFRIQ |
| | | HPDLILLQYVDDLLLAATSELDCQQGTRALLQTLGNLGYR |
| | | ASAKKAQICQKQVKYLGYLLKEGQRWLTEARKETVMGQP |
| | | TPKTPROLREFLGKAGFCRLFIPGFAEMAAPLYPLTKPGT |
| | | LFNWGPDQQKAYQEIKQALLTAPALGLPDLTKPFELFVD |
| | | EKQGYAKGVLTQKLGPWRRPVAYLSKKLDPVAAGWPPCL |
| | | RMVAAIAVLTKDAGKLTMGQPLVILAPHAVEALVKQPPDR |
| | | WLSNARMTHYQALLLDTDRVQFGPVVALNPATLLPLPEE |
| | | GLOHNCLDILAEAHGTRPDLTDQPLPDADHTWYTDGSSL |
| | | LQEGORKAGAAVTTETEVIWAKALPAGTSAQRAELIALTQ |
| | | ALKMAEGKKLNVYTDSRYAFATAHIHGEIYRRRGWLTSEG |
| | | KEIKNKDEILALLKALFLPKRLSIIHCPGHQKGHSAEARGN |
| | | RMADQAARKAAITETPDTSTLLIENSSPSGGSKRTADGSE |
| | | FEPKKKRKV |
| | | KEY: |
| | | NUCLEAR LOCALIZATION SEQUENCE (NLS) |
| | | CAS9(H840A) |
| | | 33-AMINO ACID LINKER |
| | | M-MLV REVERSE TRANSCRIPTASE |
| 30 | N-TERMINAL | MKRTADGSEFESPKKKRKV |
| | NLS | |
| 6 | CAS9 (H840A) | DKKYSIGLDIGTNSVGWAVITDEYKVPSKKFKVLGNT |
| | (MET MINUS) | DRHSIKKNLIGALLFDSGETAEATRLKRTARRRYTRRK |
| | | NRICYLQEIFSNEMAKVDDSFFHRLEESFLVEEDKKHE |
| | | RHPIFGNIVDEVAYHEKYPTIYHLRKKLVDSTDKADLR |
| | | LIYLALAHMIKFRGHFLIEGDLNPDNSDVDKLFIQLVQT |
| | | YNQLFEENPINASGVDAKAILSARLSKSRRLENLIAQLP |
| | | GEKKNGLFGNLIALSLGLTPNFKSNFDLAEDAKLQLSK |
| | | DTYDDDLDNLLAQIGDQYADLFLAAKNLSDAILLSDIL |
| | | RVNTEITKAPLSASMIKRYDEHHQDLTLLKALVRQQLP |
| | | EKYKEIFFDQSKNGYAGYIDGGASQEEFYKFIKPILEK |
| | | MDGTEELLVKLNREDLLRKQRTFDNGSIPHQIHLGELH |
| | | AILRRQEDFYPFLKDNREKIEKILTFRIPYYVGPLARGN |
| | | SRFAWMTRKSEETITPWNFEEVVDKGASAQSFIERMT |
| | | NFDKNLPNEKVLPKHSLLYEYFTVYNELTKVKYVTEG |
| | | MRKPAFLSGEQKKAIVDLLFKTNRKVTVKQLKEDYFK |
| | | KIECFDSVEISGVEDRFNASLGTYHDLLKIIKDKDFLDN |
| | | EENEDILEDIVLTLTLFEDREMIEERLKTYAHLFDDKV |
| | | MKQLKRRRYTGWGRLSRKLINGIRDKQSGKTILDFLKS |
| | | DGFANRNFMQLIHDDSLTFKEDIQKAQVSGQGDSLHE |
| | | HIANLAGSPAIKKGILQTVKVVDELVKVMGRHKPENIV |
| | | IEMARENQTTQKGQKNSRERMKRIEEGIKELGSQILKE |
| | | HPVENTQLQNEKLYLYYLQNGRDMYVDQELDINRLS |
| | | DYDVDAIVPQSFLKDDSIDNKVLTRSDKNRGKSDNVPS |
| | | EEVVKKMKNYWRQLLNAKLITQRKFDNLTKAERGGL |
| | | SELDKAGFIKRQLVETRQITKHVAQILDSRMNTKYDEN |
| | | DKLIREVKVITLKSKLVSDFRKDFQFYKVREINNYHHA |
| | | HDAYLNAVVGTALIKKYPKLESEFVYGDYKVYDVRK |
| | | MIAKSEQEIGKATAKYFFYSNIMNFFKTEITLANGEIRK |
| | | RPLIETNGETGEIVWDKGRDFATVRKVLSMPQVNIVK |
| | | KTEVQTGGFSKESILPKRNSDKLIARKKDWDPKKYGG |
| | | FDSPTVAYSVLVVAKVEKGKSKKLKSVKELLGITIMER |
| | | SSFEKNPIDFLEAKGYKEVKKDLIIKLPKYSLFELENGR |
| | | KRMLASAGELQKGNELALPSKYVNFLYLASHYEKLKG |
| | | SPEDNEQKQLFVEQHKHYLDEIIEQISEFSKRVILADAN |
| | | LDKVLSAYNKHRDKPIREQAENIIHLFTLTNLGAPAAF |
| | | KYFDTTIDRKRYTSTKEVLDATLIHQSITGLYETRIDLS |
| | | QLGGD |
| 56 | LINKER | SGGSSGGSSGSETPGTSESATPESSGGSSGGSS |
| | BETWEEN | |
| | CAS9 | |
| | DOMAIN AND | |
| | RT DOMAIN | |
| | (33 AMINO | |
| | ACIDS) | |
| 2 | MMLV_RT | TLNIEDEYRLHETSKEPDVSLGSTWLSDFPQAWAETGG |
| | D200N | MGLAVRQAPLIIPLKATSTPVSIKQYPMSQEARLGIKPH |
| | T330P | IQRLLDQGILVPCQSPWNTPLLPVKKPGTNDYRPVQDL |
| | L603W | REVNKRVEDIHPTVPNPYNLLSGLPPSHQWYTVLDLK |
| | T306K | DAFFCLRLHPTSQPLFAFEWRDPEMGISGQLTWTRLPQ |
| | W313F | GFKNSPTLFNEALHRDLADFRIQHPDLILLQYVDDLLL |
| | | AATSELDCQQGTRALLQTLGNLGYRASAKKAQICQKQ |
| | | VKYLGYLLKEGQRWLTEARKETVMGQPTPKTPRQLR |
| | | EFLGKAGFCRLFIPGFAEMAAPLYPLTKPGTLFNWGPD |
| | | QQKAYQEIKQALLTAPALGLPDLTKPFELFVDEKQGY |
| | | AKGVLTQKLGPWRRPVAYLSKKLDPVAAGWPPCLRM |
| | | VAAIAVLTKDAGKLTMGQPLVILAPHAVEALVKQPPD |
| | | RWLSNARMTHYQALLLDTDRVQFGPVVALNPATLLPL |
| | | PEEGLOHNCLDILAEAHGTRPDLTDQPLPDADHTWYT |
| | | DGSSLLQEGQRKAGAAVTTETEVIWAKALPAGTSAQR |
| | | AELIALTQALKMAEGKKLNVYTDSRYAFATAHIHGEI |
| | | YRRRGWLTSEGKEIKNKDEILALLKALFLPKRLSIIHCP |
| | | GHQKGHSAEARGNRMADQAARKAAITETPDTSTLLIE |
| | | NSSP |
| 45 | C-TERMINAL | SGGSKRTADGSEFEPKKKRKV |
| | NLS | |

TABLE 5
Amino acid sequence of exemplary PE and its individual components

| SEQ ID | | |
|---|---|---|
| NO: | Description | Sequence |
| 62 | Exemplary prime | MKRTADGSEFESPKKKRKVDKKYSIGLDIGTNSVGWAV |
| | editor [NLS]- | ITDEYKVPSKKFKVLGNTDRHSIKKNLIGALLFDSGET |
| | [Cas9((R220K) | AEATRLKRTARRRYTRRKNRICYLQEIFSNEMAKVDD |
| | (R393K) | SFFHRLEESFLVEEDKKHERHPIFGNIVDEVAYHEKYP |
| | (H839A)]- | TIYHLRKKLVDSTDKADLRLIYLALAHMIKFRGHFLIE |
| | [linker]- | GDLNPDNSDVDKLFIQLVQTYNQLFEENPINASGVDAK |
| | [MMLV_RT | AILSARLSKSRKLENLIAQLPGEKKNGLFGNLIALSLG |
| | (D200N)(T330P) | LTPNFKSNFDLAEDAKLQLSKDTYDDDLDNLLAQIGD |
| | (L603W)(T306K) | QYADLFLAAKNLSDAILLSDILRVNTEITKAPLSASMIK |
| | (W313F)]-[NLS] | RYDEHHQDLTLLKALVRQQLPEKYKEIFFDQSKNGYA |
| | | GYIDGGASQEEFYKFIKPILEKMDGTEELLVKLKREDL |
| | | LRKQRTFDNGSIPHQIHLGELHAILRRQEDFYPFLKDN |
| | | REKIEKILTFRIPYYVGPLARGNSRFAWMTRKSEETIT |
| | | PWNFEEVVDKGASAQSFIERMTNFDKNLPNEKVLPKH |
| | | SLLYEYFTVYNELTKVKYVTEGMRKPAFLSGEQKKAI |
| | | VDLLFKTNRKVTVKQLKEDYFKKIECFDSVEISGVEDR |
| | | FNASLGTYHDLLKIIKDKDFLDNEENEDILEDIVLTLTL |
| | | FEDREMIEERLKTYAHLFDDKVMKQLKRRRYTGWG |
| | | RLSRKLINGIRDKQSGKTILDFLKSDGFANRNFMQLIH |
| | | DDSLTFKEDIQKAQVSGQGDSLHEHIANLAGSPAIKKG |
| | | ILQTVKVVDELVKVMGRHKPENIVIEMARENQTTQK |
| | | GQKNSRERMKRIEEGIKELGSQILKEHPVENTQLQNE |
| | | KLYLYYLQNGRDMYVDQELDINRLSDYDVDAIVPQSF |
| | | LKDDSIDNKVLTRSDKNRGKSDNVPSEEVVKKMKNY |
| | | WRQLLNAKLITQRKFDNLTKAERGGLSELDKAGFIKR |
| | | QLVETRQITKHVAQILDSRMNTKYDENDKLIREVKVIT |
| | | LKSKLVSDFRKDFQFYKVREINNYHHAHDAYLNAVVG |
| | | TALIKKYPKLESEFVYGDYKVYDVRKMIAKSEQEIGK |
| | | ATAKYFFYSNIMNFFKTEITLANGEIRKRPLIETNGETG |
| | | EIVWDKGRDFATVRKVLSMPQVNIVKKTEVQTGGFS |
| | | KESILPKRNSDKLIARKKDWDPKKYGGFDSPTVAYSV |
| | | LVVAKVEKGKSKKLKSVKELLGITIMERSSFEKNPIDF |
| | | LEAKGYKEVKKDLIIKLPKYSLFELENGRKRMLASAG |
| | | ELQKGNELALPSKYVNFLYLASHYEKLKGSPEDNEQK |
| | | QLFVEQHKHYLDEIIEQISEFSKRVILADANLDKVLSAY |
| | | NKHRDKPIREQAENIIHLFTLTNLGAPAAFKYFDTTID |
| | | RKRYTSTKEVLDATLIHQSITGLYETRIDLSQLGGDSG |
| | | GSSGGSKRTADGSEFESPKKKRKVSGGSSGGSTLNIEDE |
| | | YRLHETSKEPDVSLGSTWLSDFPQAWAETGGMGLAVRQAPL |
| | | IIPLKATSTPVSIKQYPMSQEARLGIKPHIQRLLDQGILVPCQS |
| | | PWNTPLLPVKKPGTNDYRPVQDLREVNKRVEDIHPTVPNPY |
| | | NLLSGLPPSHOWYTVLDLKDAFFCLRLHPTSQPLFAFEWRD |
| | | PEMGISGOLTWTRLPQGFKNSPTLFNEALHRDLADFRIQHP |
| | | DLILLQYVDDLLLAATSELDCQQGTRALLQTLGNLGYRASAK |
| | | KAQICQKQVKYLGYLLKEGQRWLTEARKETVMGQPTPKTPR |
| | | QLREFLGKAGFCRLFIPGFAEMAAPLYPLTKPGTLFNWGPD |
| | | QQKAYQEIKQALLTAPALGLPDLTKPFELFVDEKQGYAKGV |
| | | LTQKLGPWRRPVAYLSKKLDPVAAGWPPCLRMVAAIAVLTK |
| | | DAGKLTMGQPLVILAPHAVEALVKOPPDRWLSNARMTHYQA |
| | | LLLDTDRVQFGPVVALNPATLLPLPEEGLOHNCLDILAEAHG |
| | | TRPDLTDQPLPDADHTWYTDGSSLLQEGORKAGAAVTTETE |
| | | VIWAKALPAGTSAQRAELIALTQALKMAEGKKLNVYTDSRYA |
| | | FATAHIHGEIYRRRGWLTSEGKEIKNKDEILALLKALFLPKRL |
| | | SIIHCPGHQKGHSAEARGNRMADQAARKAAITETPDTSTLLIE |
| | | NSSPSGGSKRTADGSEFESPKKKRKVGSGPAAKRVKLD |
| | | KEY: |
| | | N-terminal bipartiteSV40NLS |
| | | CAS9(R221K N394K H840A) |
| | | SGGSx2-met-bpSV40NLS-SGGSx2 LINKER |
| | | M-MLV D200N T306K W313F T330P L603W REVERSE |
| | | TRANSCRIPTASE |
| | | C-terminal linker-NLS1 |
| | | C-terminal linker-NLS2 |
| 30 | -N-terminal | MKRTADGSEFESPKKKRKV |
| | bpSV40NLS | |
| 8 | -CAS9 (R221K | DKKYSIGLDIGTNSVGWAVITDEYKVPSKKFKVLGNTDR |
| | N394K H840A) | HSIKKNLIGALLFDSGETAEATRLKRTARRRYTRRKNRIC |
| | | YLQEIFSNEMAKVDDSFFHRLEESFLVEEDKKHERHPIFG |
| | | NIVDEVAYHEKYPTIYHLRKKLVDSTDKADLRLIYLALA |
| | | HMIKFRGHFLIEGDLNPDNSDVDKLFIQLVQTYNQLFEEN |
| | | PINASGVDAKAILSARLSKSRKLENLIAQLPGEKKNGLFG |
| | | NLIALSLGLTPNFKSNFDLAEDAKLQLSKDTYDDDLDNLL |
| | | AQIGDQYADLFLAAKNLSDAILLSDILRVNTEITKAPLSAS |
| | | MIKRYDEHHQDLTLLKALVRQQLPEKYKEIFFDQSKNGY |
| | | AGYIDGGASQEEFYKFIKPILEKMDGTEELLVKLKREDLL |
| | | RKQRTFDNGSIPHQIHLGELHAILRRQEDFYPFLKDNREKI |
| | | EKILTFRIPYYVGPLARGNSRFAWMTRKSEETITPWNFEE |
| | | VVDKGASAQSFIERMTNFDKNLPNEKVLPKHSLLYEYFT |
| | | VYNELTKVKYVTEGMRKPAFLSGEQKKAIVDLLFKTNRK |
| | | VTVKQLKEDYFKKIECFDSVEISGVEDRFNASLGTYHDLL |
| | | KIIKDKDFLDNEENEDILEDIVLTLTLFEDREMIEERLKTY |
| | | AHLFDDKVMKQLKRRRYTGWGRLSRKLINGIRDKQSGK |
| | | TILDFLKSDGFANRNFMQLIHDDSLTFKEDIQKAQVSGQG |
| | | DSLHEHIANLAGSPAIKKGILQTVKVVDELVKVMGRHKP |
| | | ENIVIEMARENQTTQKGQKNSRERMKRIEEGIKELGSQIL |
| | | KEHPVENTQLQNEKLYLYYLQNGRDMYVDQELDINRLS |
| | | DYDVDAIVPQSFLKDDSIDNKVLTRSDKNRGKSDNVPSE |
| | | EVVKKMKNYWRQLLNAKLITQRKFDNLTKAERGGLSEL |
| | | DKAGFIKRQLVETRQITKHVAQILDSRMNTKYDENDKLIR |
| | | EVKVITLKSKLVSDFRKDFQFYKVREINNYHHAHDAYLN |
| | | AVVGTALIKKYPKLESEFVYGDYKVYDVRKMIAKSEQEI |
| | | GKATAKYFFYSNIMNFFKTEITLANGEIRKRPLIETNGETG |
| | | EIVWDKGRDFATVRKVLSMPQVNIVKKTEVQTGGFSKES |
| | | ILPKRNSDKLIARKKDWDPKKYGGFDSPTVAYSVLVVAK |
| | | VEKGKSKKLKSVKELLGITIMERSSFEKNPIDFLEAKGYK |
| | | EVKKDLIIKLPKYSLFELENGRKRMLASAGELQKGNELAL |
| | | PSKYVNFLYLASHYEKLKGSPEDNEQKQLFVEQHKHYLD |
| | | EIIEQISEFSKRVILADANLDKVLSAYNKHRDKPIREQAEN |
| | | IIHLFTLTNLGAPAAFKYFDTTIDRKRYTSTKEVLDATLIH |
| | | QSITGLYETRIDLSQLGGD |
| 63 | -SGGSx2- | SGGSSGGSKRTADGSEFESPKKKRKVSGGSSGGS |
| | bpSV40NLS- | |
| | SGGSx2 linker | |
| 2 | -MMLV RT | TLNIEDEYRLHETSKEPDVSLGSTWLSDFPQAWAETGGM |
| | D200N T330P | GLAVRQAPLIIPLKATSTPVSIKQYPMSQEARLGIKPHIQR |
| | L603W T306K | LLDQGILVPCQSPWNTPLLPVKKPGTNDYRPVQDLREVN |
| | W313F | KRVEDIHPTVPNPYNLLSGLPPSHQWYTVLDLKDAFFCLR |
| | (pentamutant) | LHPTSQPLFAFEWRDPEMGISGQLTWTRLPQGFKNSPTLF |
| | | NEALHRDLADFRIQHPDLILLQYVDDLLLAATSELDCQQ |
| | | GTRALLQTLGNLGYRASAKKAQICQKQVKYLGYLLKEG |
| | | QRWLTEARKETVMGQPTPKTPRQLREFLGKAGFCRLFIP |
| | | GFAEMAAPLYPLTKPGTLFNWGPDQQKAYQEIKQALLT |
| | | APALGLPDLTKPFELFVDEKQGYAKGVLTQKLGPWRRPV |
| | | AYLSKKLDPVAAGWPPCLRMVAAIAVLTKDAGKLTMGQ |
| | | PLVILAPHAVEALVKQPPDRWLSNARMTHYQALLLDTDR |
| | | VQFGPVVALNPATLLPLPEEGLQHNCLDILAEAHGTRPDL |
| | | TDQPLPDADHTWYTDGSSLLQEGQRKAGAAVTTETEVI |
| | | WAKALPAGTSAQRAELIALTQALKMAEGKKLNVYTDSR |
| | | YAFATAHIHGEIYRRRGWLTSEGKEIKNKDEILALLKALF |
| | | LPKRLSIIHCPGHQKGHSAEARGNRMADQAARKAAITET |
| | | PDTSTLLIENSSP |
| 61 | C-terminal linker- | SGGSKRTADGSEFESPKKKRKV |
| | NLS | |
| 64 | C-terminal linker- | GSGPAAKRVKLD |
| | NLS2 | |

### PEgRNA For Editing of TCF4 Gene

The term “prime editing guide RNA”, or “PEgRNA”, refers to a guide polynucleotide that comprises one or more intended nucleotide edits for incorporation into the target double-stranded DNA. In some embodiments, the PEgRNA associates with and directs a prime editor to incorporate the one or more intended nucleotide edits into the target gene via prime editing.

In some embodiments, a PEgRNA comprises a spacer that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA comprises an editing template. In some embodiments, the PEgRNA comprises a primer binding site (PBS). In some embodiments, a PEgRNA comprises an extension arm that comprises an editing template and a primer binding site (PBS).

Dual prime editing involves two different PEgRNAs each complexed with a prime editor. In some embodiments, the prime editor is the same for each of the PEgRNA-prime editor complexes. In some embodiments, the prime editor is different for each of the PEgRNA-prime editor complexes. In some embodiments, each of the two PEgRNAs comprises a region of complementarity to a distinct search target sequence of the double-stranded target DNA, wherein the two distinct search target sequences are on the two complementary strands of the double-stranded target DNA. In some embodiments, the two PEgRNAs each can direct a prime editor to initiate the prime editing process on the two complementary strands of the double-stranded target DNA. In some embodiments, each of the two PEgRNAs comprises a spacer complementary to a separate search target sequence. In some embodiments, each of the two PEgRNAs anneals with a separate search target sequence through its spacer.

In some embodiments, a first PEgRNA comprises a first spacer complementary to a first search target sequence on a first strand of a double-stranded target DNA, e.g., a double-stranded target gene. In the context of the first PEgRNA, the first strand of the double-stranded target DNA may be referred to as a first target strand, and the complementary strand referred to as the first PAM strand. In some embodiments, a first PEgRNA comprises a first gRNA core. In some embodiments, a first PEgRNA comprises a first editing template. In certain embodiments, a first PEgRNA comprises a first primer binding site (PBS) that is complementary to a free 3′ end formed at the first nick site.

In some embodiments, a second PEgRNA comprises a second spacer complementary to a second search target sequence on a second strand of a double-stranded target DNA, e.g., a double-stranded target gene. In the context of the second PEgRNA, the second strand of the double-stranded target DNA may be referred to as a second target strand, and the complementary strand referred to as the second PAM strand. In some embodiments, a second PEgRNA comprises a second gRNA core. In some embodiments, a second PEgRNA comprises a second editing template. In some embodiments, a second PEgRNA comprises a second primer binding site (PBS) that is complementary to a free 3′ end formed at the second nick site.

In certain embodiments, the first editing template of a first PEgRNA and the second editing template of a second PEgRNA comprise a region of complementarity to each other. In certain embodiments, the region of complementarity between the first editing template and the second editing template comprises a nucleotide sequence that is exogenous to the double-stranded target DNA or target gene. In certain embodiments, the exogenous sequence may be a marker, expression tag, barcode or regulatory sequence. In certain embodiments, the region of complementarity between the first editing template and the second editing template comprises a nucleotide sequence that is at least partially identical to a sequence in the double-stranded target DNA or target gene. In certain embodiments, the region of complementarity between the first editing template and the second editing template comprises a nucleotide sequence that is at least partially identical to a sequence in the IND.

In certain embodiments, the first editing template of a first PEgRNA and the second editing template of a second PEgRNA do not comprise a region of complementarity to each other. In certain embodiments, the first editing template of a first PEgRNA comprises region of identity to a sequence on the first target strand (or the first strand), and the second editing template comprises a region of identity to a sequence on the second target strand (or the second strand). In certain embodiments, the first editing template of a first PERNA comprises a region of identity to a sequence on the first target strand immediately adjacent to and outside the IND. In certain embodiments, the second editing template of a second PEgRNA comprises a region of identity to a sequence on the second target strand immediately adjacent to and outside the IND. In some embodiments, an editing template comprises one or more intended nucleotide edits to be incorporated in the double-stranded target DNA, e.g., the TCF4 gene, by prime editing. In some embodiments, incorporation of the newly synthesized single-stranded DNA encoded by the editing template results in incorporation of one or more intended nucleotide edit in the double-stranded target DNA, e.g., the TCF4 gene, compared to the endogenous sequence of the double-stranded target gene. For example, in some embodiments, the one or more intended nucleotide edits comprises deletion, insertion, and/or substitution of one or more nucleotides compared to the endogenous sequence of the double-stranded target gene, e.g., the TCF4 gene. In some embodiments, the one or more intended nucleotide edits comprises deletion of an array of tri-nucleotide repeats compared to the endogenous sequence of the double-stranded target gene, e.g., the TCF4 gene. In some embodiments, the one or more intended nucleotide edits comprises deletion of an array of CTG repeats compared to the endogenous sequence of the double-stranded target gene, e.g., the TCF4 gene. In some embodiments, the one or more intended nucleotide edits comprises deletion of an array of tri-nucleotide repeats, e.g., an array of CTG repeats, and insertion of one or more exogenous sequences compared to the endogenous sequence of the double-stranded target gene, e.g., the TCF4 gene. In some embodiments, the one or more intended nucleotide edits comprises deletion of a portion of an array of tri-nucleotide repeats, e.g., an array of CTG repeats, and optionally insertion of one or more exogenous sequences compared to the endogenous sequence of the double-stranded target gene, e.g., the TCF4 gene. In some embodiments, the one or more intended nucleotide edits comprises deletion of 1-3, 1-5, 1-10, 1-15, 1-20, 1-25, 1-30, 1-35, 1-40, 1-45, 1-50, 1-55, 1-60, 1-75 or more tri-nucleotide repeats compared to the endogenous sequence of the double-stranded target gene, e.g., the TCF4 gene. In some embodiments, the one or more intended nucleotide edits comprises deletion of 1-3, 1-5, 1-10, 1-15, 1-20, 1-25, 1-30, 1-35, 1-40, 1-45, 1-50, 1-55, 1-60, 1-75 or more tri-nucleotide repeats compared to the endogenous sequence of the double-stranded target gene, e.g., the TCF4 gene. In some embodiments, the one or more intended nucleotide edits comprises deletion of 5-10, 5-15, 5-20, 5-25, 5-30, 5-35, 5-40, 5-50, 5-60, 5-70, 5-80, 5-90, 5-100, 20-30, 20-40, 20-50, 20-60, 20-70, 20-80, 20-90, 20-100, 35-40, 35-50, 35-60, 35-70, 35-80, 35-90, 35-100, 50-70, 50-80, 50-90, 50-100, 50-150, 50-200, 50-250, 50-300, 50-350, 50-400, 50-450, 50-500, 50-550, 50-600, 50-650, 50-700, 50-750, 50-800, 50-850, 50-900, 50-950, 50-1000, 50-1050, 50-1100, 50-1150, 50-1200, 50-1250, 50-1300, 50-1350, 50-1400, 50-1450, 50-1500, 100-150, 100-200, 100-250, 100-300, 100-350, 100-400, 100-450, 100-500, 100-550, 100-600, 100-650, 100-700, 100-750, 100-800, 100-850, 100-900, 100-950, 100-1000, 100-1050, 100-1100, 100-1150, 100-1200, 100-1250, 100-1300, 100-1350, 100-1400, 100-1450, 100-1500, 150-200, 150-250, 150-300, 150-350, 150-400, 150-450, 150-500, 150-550, 150-600, 150-650, 150-700, 150-750, 150-800, 150-850, 150-900, 150-950, 150-1000, 150-1050, 150-1100, 150-1150, 150-1200, 150-1250, 150-1300, 150-1350, 150-1400, 150-1450, 150-1500, 200-250, 200-300, 200-350, 200-400, 200-450, 200-500, 200-550, 200-600, 200-650, 200-700, 200-750, 200-800, 200-850, 200-900, 200-950, 200-1000, 200-1050, 200-1100, 200-1150, 200-1200, 200-1250, 200-1300, 200-1350, 200-1400, 200-1450, 200-1500, 250-300, 250-350, 250-400, 250-450, 250-500, 250-550, 250-600, 250-650, 250-700, 250-750, 250-800, 250-850, 250-900, 250-950, 250-1000, 250-1050, 250-1100, 250-1150, 250-1200, 250-1250, 250-1300, 250-1350, 250-1400, 250-1450, 250-1500, 300-350, 300-400, 300-450, 300-500, 300-550, 300-600, 300-650, 300-700, 300-750, 300-800, 300-850, 300-900, 300-950, 300-1000, 300-1050, 300-1100, 300-1150, 300-1200, 300-1250, 300-1300, 300-1350, 300-1400, 300-1450, 300-1500, 400-450, 400-500, 400-550, 400-600, 400-650, 400-700, 400-750, 400-800, 400-850, 400-900, 400-950, 400-1000, 400-1050, 400-1100, 400-1150, 400-1200, 400-1250, 400-1300, 400-1350, 400-1400, 400-1450, 400-1500, 500-550, 500-600, 500-650, 500-700, 500-750, 500-800, 500-850, 500-900, 500-950, 500-1000, 500-1050, 500-1100, 500-1150, 500-1200, 500-1250, 500-1300, 500-1350, 500-1400, 500-1450, 500-1500, 600-650, 600-700, 600-750, 600-800, 600-850, 600-900, 600-950, 600-1000, 600-1050, 600-1100, 600-1150, 600-1200, 600-1250, 600-1300, 600-1350, 600-1400, 600-1450, 600-1500, 700-750, 700-800, 700-850, 700-900, 700-950, 700-1000, 700-1050, 700-1100, 700-1150, 700-1200, 700-1250, 700-1300, 700-1350, 700-1400, 700-1450, 700-1500, 800-850, 800-900, 800-950, 800-1000, 800-1050, 800-1100, 800-1150, 800-1200, 800-1250, 800-1300, 800-1350, 800-1400, 800-1450, 800-1500, 900-950, 900-1000, 900-1050, 900-1100, 900-1150, 900-1200, 900-1250, 900-1300, 900-1350, 900-1400, 900-1450, 900-1500, 1000-1050, 1000-1100, 1000-1150, 1000-1200, 1000-1250, 1000-1300, 1000-1350, 1000-1400, 1000-1450, 1000-1500, 1100-1200, 1100-1300, 1100-1400, 1100-1500, 1200-1300, 1200-1400, 1200-1500, 1300-1400, 1300-1500, or 1400-1500 tri-nucleotide repeats compared to the endogenous sequence of the double-stranded target gene, e.g., the TCF4 gene.

In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT.

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the first PEgRNA comprises a first PBS that comprises a region of complementarity to the second strand of the double-stranded target DNA or target gene. In some embodiments, the second PEgRNA comprises a second PBS that comprises a region of complementarity to the first strand of the double-stranded target DNA or target gene. In some embodiments, the first PEgRNA comprises a first PBS that comprises a region of complementarity to the first spacer of the first PEgRNA. In some embodiments, the first PEgRNA comprises a first PBS that is at least partially complementary to the first spacer of the first PEgRNA. In some embodiments, the second PEgRNA comprises a second PBS that comprises a region of complementarity to the second spacer of the second PEgRNA. In some embodiments, the second PEgRNA comprises a second PBS that is at least partially complementary to the second spacer of the second PEgRNA.

In some embodiments, a PEgRNA includes only RNA nucleotides and forms an RNA polynucleotide. In some embodiments, a PERNA is a chimeric polynucleotide that includes both RNA and DNA nucleotides. For example, a PEgRNA can include DNA in the spacer, the gRNA core, or the extension arm. In some embodiments, a PERNA comprises DNA in the spacer. In some embodiments, the entire spacer of a PEgRNA is a DNA sequence. In some embodiments, the PERNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer, the primer binding site sequence (PBS) and the editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm (i.e., the PBS and editing template) of the PEgRNA. In some embodiments, the gRNA core of a PERNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, a PEgRNA comprises, from 5′ to 3′: a spacer, a gRNA core, and an extension arm. In some embodiments, a PEgRNA comprises, from 5′ to 3′: a spacer, a gRNA core, an editing template, and a PBS. In some embodiments, a PEgRNA comprises, from 5′ to 3′: an extension arm, a spacer, and a gRNA core. In some embodiments, the PEgRNA comprises, from 5′ to 3′: an editing template a PBS, a spacer, and a gRNA core. In some embodiments, a first PERNA comprises a structure: 5′-[first spacer]-[first gRNA core]-[first editing template]-[first primer binding site sequence]-3′. In some embodiments, a first PEgRNA comprises a structure: 5′-[first editing template]-[first primer binding site sequence]-[first spacer]-[first gRNA core]-3′. In some embodiments, a second PEgRNA comprises a structure: 5′-[second spacer]-[second gRNA core]-[second editing template]-[second primer binding site sequence]-3′. In some embodiments, a second PERNA comprises a structure: 5′-[second editing template]-[second primer binding site sequence]-[second spacer]-[second gRNA core]-3′.

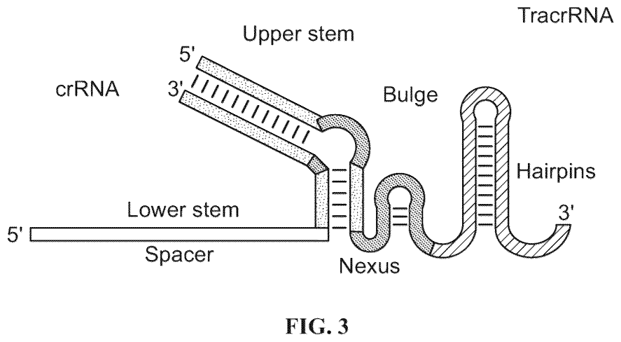

In some embodiments, a PEgRNA comprises a single polynucleotide molecule that comprises the spacer, the gRNA core, and the editing template. In some embodiments, a PEgRNA comprises a single polynucleotide molecule that comprises the spacer, the gRNA core, and the extension arm (i.e., a PBS and editing template). In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a first spacer comprises a region that has substantial complementarity to a first search target sequence on a first target strand, or first strand, of a double-stranded target DNA, e.g., a TCF4 gene. In some embodiments, the first spacer of a PEgRNA is identical or substantially identical to a protospacer sequence on the second strand of the target gene (except that the protospacer sequence comprises thymine and the spacer may comprise uracil). In some embodiments, the first spacer is at least about 70%, 75%, 80%, 85%, 90%, 95%, or 100% complementary to a first search target sequence in the target gene. In some embodiments, the first spacer is substantially complementary to the first search target sequence. In some embodiments, a second spacer comprises a region that has substantial complementarity to a second search target sequence on the second target strand, or the second strand, of a double-stranded target DNA, e.g., a TCF4 gene. In some embodiments, the second spacer of a PERNA is identical or substantially identical to a protospacer on the first strand of the target gene (except that the protospacer comprises thymine and the spacer may comprise uracil). In some embodiments, the second spacer is at least about 70%, 75%, 80%, 85%, 90%, 95%, or 100% complementary to a second search target sequence in the target gene. In some embodiments, the second spacer is substantially complementary to the second search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 17 to 23 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length. In some embodiments, the spacer is 21 to 22 nucleotides in length.

As used herein in a PEgRNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

Exemplary sequences for PEgRNA spacers are provided in Tables 6 and 7 (SEQ ID NOs: 65-69 and 70-74).

TABLE 6
TCF4 PEgRNA 5′ spacer (“First PEgRNA spacer”)
sequences

| SEQ ID NO: | TCF4 PEgRNA 5′ Spacer Sequence |
|---|---|
| 65 | GTCCAAACCGCCTTCCAAGTG |
| 66 | GATCCAAACCGCCTTCCAAGT |
| 67 | GAATCCAAACCGCCTTCCAAG |
| 68 | GAACCAAGTCCCAGACATGTC |
| 69 | GAGCGGAACTTGACACACTTG |

TABLE 7
TCF4 PEgRNA 3′ spacer (“Second PEgRNA spacer”)
sequences

| SEQ ID NO: | TCF4 PEgRNA 3′ Spacer Sequence |
|---|---|
| 70 | GAAAAGAAGGTCTAGAAGAGG |
| 71 | GAAAGCCATTTCTCCAAAAGA |
| 72 | GAAGTGCAACAAGCAGAAAGG |
| 73 | GAGAAAGTGCAACAAGCAGAA |
| 74 | GTCAGAGCCTGCAAAAAGCAA |

Each of these PEgRNA spacer sequences has had a “G” residue added to the 5′ end in order to facilitate production of PEgRNA by transcription from a plasmid, because the endogenous sequence on which the spacer is based does not have a G in what would be the 5′-most position. The leading G residue, if added for plasmid production, should be omitted if the PEgRNA is being chemically synthesized.

The extension arm of a first PEgRNA may be partially complementary to the spacer of the first PEgRNA. In some embodiments, the editing template (e.g., RTT) of a first PEgRNA is partially complementary to the spacer of the first PEgRNA. In some embodiments, the RTT and the primer binding site (PBS) of the first PEgRNA are each partially complementary to the spacer of the first PEgRNA. The extension arm of a PEgRNA may comprise a PBS and an RTT. The extension arm of a second PERNA may be partially complementary to the spacer of the second PEgRNA. In some embodiments, the RTT of a second PEgRNA is partially complementary to the spacer of the second PEgRNA. In some embodiments, the RTT and the PBS of the second PEgRNA are each partially complementary to the spacer of the second PEgRNA.

An extension arm of a PEgRNA may comprise a primer binding site sequence (also referred to as a primer binding site, PBS, or PBS sequence) that hybridizes with a free 3′ end of a single-stranded DNA in the target gene (e.g., the TCF4 gene) generated by nicking with a prime editor. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 4 nucleotides in length. In some embodiments, the PBS is at least 6 nucleotides in length. In some embodiments, the PBS is about 4 to 12 nucleotides, about 6 to 12 nucleotides, about 8 to 12 nucleotides, about 10 to 12 nucleotides, 4 to 14 nucleotides, about 6 to 14 nucleotides, about 8 to 14 nucleotides, about 10 to 14 nucleotides, about 12 to 14 nucleotides, 4 to 16 nucleotides, about 6 to 16 nucleotides, about 8 to 16 nucleotides, about 10 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length. In some embodiments, the PBS is 3 to 14 nucleotides in length. In some embodiments, the PBS is 3 to 14 nucleotides in length. In some embodiments, the PBS is about 8 to 14 nucleotides in length. In some embodiments, the PBS is 8 to 9, 8 to 10, 8 to 11, or 8 to 12 nucleotides in length.

The PBS of a first PEgRNA may be complementary or substantially complementary to a DNA sequence in the second strand of the target gene. The PBS of a second PEgRNA may be complementary or substantially complementary to a DNA sequence in the first strand of the target gene. By annealing with the edit strand at a free hydroxy group, e.g., a free 3′ end generated by prime editor nicking, a PBS may initiate synthesis of a new single-stranded DNA encoded by the editing template at the nick site. In some embodiments, a PBS is at least about 70%, 75%, 80%, 85%, 90%, 95%, or 100% complementary to a region of the edit strand of the target gene (e.g., the TCF4 gene). In some embodiments, a PBS is perfectly complementary, or 100% complementary, to a region of the edit strand of the target gene (e.g., the TCF4 gene).

Exemplary sequences for PBS are provided in Tables 8 and 9 (SEQ ID NOs: 52-69 and 85-99). As used herein in a PEgRNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PERNA or guide RNA or any chemically modified uracil nucleobase known in the art, such as 5-methoxyuracil. The sequences for PEgRNA PBSs are provided in Tables 8 and 9 (SEQ ID NOs: 75-89; 90-104).

TABLE 8
TCF4 PEgRNA 5′ PBS Sequence

| SEQ ID | |
|---|---|
| NOs: | 5′ PBS Sequence |
| 75 | TTGGAAGG |
| 76 | TTGGAAGGCG |
| 77 | TTGGAAGGCGGTA |
| 78 | TGGAAGGC |
| 79 | TGGAAGGCGGA |
| 80 | TGGAAGGCGGTTT |
| 81 | GGAAGGCG |
| 82 | GGAAGGCGGTA |
| 83 | GGAAGGCGGTTTG |
| 84 | ATGTCTGG |
| 85 | ATGTCTGGGA |
| 86 | ATGTCTGGGACTA |
| 87 | GTGTGTCA |
| 88 | GTGTGTCAAGA |
| 89 | GTGTGTCAAGTTC |

TABLE 9
TCF4 PEgRNA 3′ PBS Sequence

| SEQ ID | |
|---|---|
| NOs: | 3′ PBS Sequence |
| 90 | CTTCTAGA |
| 91 | CTTCTAGACCA |
| 92 | CTTCTAGACCTTCA |
| 93 | TTTGGAGA |
| 94 | TTTGGAGAAAA |
| 95 | TTTGGAGAAATGG |
| 96 | TTCTGCTT |
| 97 | TTCTGCTTGTA |
| 98 | TTCTGCTTGTTGC |
| 99 | TGCTTGTT |
| 100 | TGCTTGTTGC |
| 101 | TGCTTGTTGCACA |
| 102 | CTTTTTGC |
| 103 | CTTTTTGCAG |
| 104 | CTTTTTGCAGGCA |

SEQ ID NOs: 77, 79, 82, 86, 88, 91, 92, 94, 97, 101, and 104 have an “A” added to the 3′ end because the next residue in the endogenous sequence would have been T. If a PEgRNA incorporating one of these PBS sequences is made by chemical synthesis, the 3′-most A, if added for plasmid-based production, should be omitted.

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PERNA. In some embodiments, the editing template serves as a DNA synthesis template for a reverse transcriptase, and the editing template is referred to as a reverse transcription editing template or simply reverse transcriptase template (RTT).

In some embodiments, the editing template (e.g., RTT) is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the editing template is 12, 13, 14, 15, 16, 17, 18, 19, 20, 21, 22, 23, 24, or 25 nucleotides in length. In some embodiments, the editing template is 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the editing template comprises 2, 3, 4, 5, 6, 7, 8, 9, 10, 11, 12, 13, 14, 15, 16, 17, 18, 19, 20, 21, 22, 23, 24, 25, 30, 35, 40, 45, 50 or more nucleotides. In some embodiments, the editing template comprises about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, 045 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 nucleotides in length.

In some embodiments, the editing template comprises 10, 11, 12, 13, 14, 15, 16, 17, 18, 19, 20, 21, 22, 23, 24 or 25 nucleotides in length. In some embodiments, the editing template comprises 30, 35, 40, 50, 60, 70, 80, 90, or 100 nucleotides in length. In some embodiments, the editing template comprises no greater than 20, 25, 30, 35, 40, 50, 60, 70, 80, 90, or 100 nucleotides in length. In some embodiments, the editing template comprises a sufficient number of nucleotides to form a sufficiently stable duplex with a sequence on the double-stranded target DNA. In some embodiments, the editing template comprises at least 10 polynucleotides. In some embodiments, the editing template comprises at least 15 polynucleotides. In some embodiments, the editing template comprises at least 20 polynucleotides.

Exemplary sequences for editing templates of the first PEgRNA and the second PEgRNA are provided in Tables 10 and 11 (SEQ ID NOs: 105-144). SEQ ID NO: 105 through 114 include 100, 90, 80, 70, 60, 50, 40, 30, 20, and 10 bases of endogenous sequence upstream and downstream of the CTG repeat, respectively, flanking 5 CTG (CUG) repeats (SEQ ID NO: 390). SEQ ID NO: 115 through 124 include 100, 90, 80, 70, 60, 50, 40, 30, 20, and 10 bases of endogenous sequence upstream and downstream of the CTG repeat, respectively, concatenated to one another with no CTG/CUG repeats. Table 11 lists the reverse complements of each sequence in Table 10. Use of a first PEgRNA containing an editing template sequence from Table 10 in combination with a second PEgRNA containing an editing template sequence from Table 11 that is the reverse complement of the editing sequence from Table 10 will result in an edit in which the number of genomic repeats is reduced to the number of repeats in the editing template pair. Editing templates containing any suitable number of CTG/CUG repeats (e.g., 5, 10, 15, 20, 25, 30, or 35) and any suitable length of upstream/downstream endogenous sequence (e.g., 10 to 100) may be used in this fashion. When used with corresponding spacers (i.e., designed for the appropriate strand) that result in nicks at the correct position, these template pairs may result in “seamless” edits, i.e., edits that reduce the number of repeats without altering the surrounding endogenous DNA. Other spacers may result in some alteration to surrounding endogenous sequence, but such alterations would not be expected to alter gene expression, because the repeat and the surrounding endogenous sequence is located in an intron. Sequences should be selected such that they do not introduce or disturb splice sites.

TABLE 10
First editing template (RTT) sequence

| SEQ | |
|---|---|
| ID | |
| NO: | First editing template (RTT) sequence |
| 105 | GATGAGTTTGGTGTAAGATGCATTTGTAAAGCAAAATAA |
| | AAAGAATCCACAAAACACACAAATAAAATCCAAACCGCC |
| | TTCCAAGTGGGGCTCTTTCATGCTGCTGCTGCTGCTGCTC |
| | CTCCTCCTCCTCCTCCTTCTCCTCCTCCTCCTCCTCTTCTA |
| | GACCTTCTTTTGGAGAAATGGCTTTCGGAAGTTTTGCCAG |
| | GAAACGTAGCCCTAGG |
| 106 | GTGTAAGATGCATTTGTAAAGCAAAATAAAAAGAATCCA |
| | CAAAACACACAAATAAAATCCAAACCGCCTTCCAAGTGG |
| | GGCTCTTTCATGCTGCTGCTGCTGCTGCTCCTCCTCCTCCT |
| | CCTCCTTCTCCTCCTCCTCCTCCTCTTCTAGACCTTCTTTT |
| | GGAGAAATGGCTTTCGGAAGTTTTGCCAGGAAACG |
| 107 | CATTTGTAAAGCAAAATAAAAAGAATCCACAAAACACAC |
| | AAATAAAATCCAAACCGCCTTCCAAGTGGGGCTCTTTCAT |
| | GCTGCTGCTGCTGCTGCTCCTCCTCCTCCTCCTCCTTCTCC |
| | TCCTCCTCCTCCTCTTCTAGACCTTCTTTTGGAGAAATGGC |
| | TTTCGGAAGTTTTG |
| 108 | GCAAAATAAAAAGAATCCACAAAACACACAAATAAAAT |
| | CCAAACCGCCTTCCAAGTGGGGCTCTTTCATGCTGCTGCT |
| | GCTGCTGCTCCTCCTCCTCCTCCTCCTTCTCCTCCTCCTCC |
| | TCCTCTTCTAGACCTTCTTTTGGAGAAATGGCTTTC |
| 109 | AAGAATCCACAAAACACACAAATAAAATCCAAACCGCCT |
| | TCCAAGTGGGGCTCTTTCATGCTGCTGCTGCTGCTGCTCC |
| | TCCTCCTCCTCCTCCTTCTCCTCCTCCTCCTCCTCTTCTAG |
| | ACCTTCTTTTGGAGA |
| 110 | AAAACACACAAATAAAATCCAAACCGCCTTCCAAGTGGG |
| | GCTCTTTCATGCTGCTGCTGCTGCTGCTCCTCCTCCTCCTC |
| | CTCCTTCTCCTCCTCCTCCTCCTCTTCTAGACCTT |
| 111 | AATAAAATCCAAACCGCCTTCCAAGTGGGGCTCTTTCATG |
| | CTGCTGCTGCTGCTGCTCCTCCTCCTCCTCCTCCTTCTCCT |
| | CCTCCTCCTCCTCT |
| 112 | AAACCGCCTTCCAAGTGGGGCTCTTTCATGCTGCTGCTGC |
| | TGCTGCTCCTCCTCCTCCTCCTCCTTCTCCTCCTC |
| 113 | CCAAGTGGGGCTCTTTCATGCTGCTGCTGCTGCTGCTCCT |
| | CCTCCTCCTCCTCCT |
| 114 | CTCTTTCATGCTGCTGCTGCTGCTGCTCCTCCTCC |
| 115 | GATGAGTTTGGTGTAAGATGCATTTGTAAAGCAAAATAA |
| | AAAGAATCCACAAAACACACAAATAAAATCCAAACCGCC |
| | TTCCAAGTGGGGCTCTTTCATGCTCCTCCTCCTCCTCCTCC |
| | TTCTCCTCCTCCTCCTCCTCTTCTAGACCTTCTTTTGGAGA |
| | AATGGCTTTCGGAAGTTTTGCCAGGAAACGTAGCCCTAG |
| | G |
| 116 | GTGTAAGATGCATTTGTAAAGCAAAATAAAAAGAATCCA |
| | CAAAACACACAAATAAAATCCAAACCGCCTTCCAAGTGG |
| | GGCTCTTTCATGCTCCTCCTCCTCCTCCTCCTTCTCCTCCT |
| | CCTCCTCCTCTTCTAGACCTTCTTTTGGAGAAATGGCTTTC |
| | GGAAGTTTTGCCAGGAAACG |
| 117 | CATTTGTAAAGCAAAATAAAAAGAATCCACAAAACACAC |
| | AAATAAAATCCAAACCGCCTTCCAAGTGGGGCTCTTTCAT |
| | GCTCCTCCTCCTCCTCCTCCTTCTCCTCCTCCTCCTCCTCT |
| | TCTAGACCTTCTTTTGGAGAAATGGCTTTCGGAAGTTTTG |
| 118 | GCAAAATAAAAAGAATCCACAAAACACACAAATAAAAT |
| | CCAAACCGCCTTCCAAGTGGGGCTCTTTCATGCTCCTCCT |
| | CCTCCTCCTCCTTCTCCTCCTCCTCCTCCTCTTCTAGACCT |
| | TCTTTTGGAGAAATGGCTTTC |
| 119 | AAGAATCCACAAAACACACAAATAAAATCCAAACCGCCT |
| | TCCAAGTGGGGCTCTTTCATGCTCCTCCTCCTCCTCCTCCT |
| | TCTCCTCCTCCTCCTCCTCTTCTAGACCTTCTTTTGGAGA |
| 120 | AAAACACACAAATAAAATCCAAACCGCCTTCCAAGTGGG |
| | GCTCTTTCATGCTCCTCCTCCTCCTCCTCCTTCTCCTCCTC |
| | CTCCTCCTCTTCTAGACCTT |
| 121 | AATAAAATCCAAACCGCCTTCCAAGTGGGGCTCTTTCATG |
| | CTCCTCCTCCTCCTCCTCCTTCTCCTCCTCCTCCTCCTCT |
| 122 | AAACCGCCTTCCAAGTGGGGCTCTTTCATGCTCCTCCTCC |
| | TCCTCCTCCTTCTCCTCCTC |
| 123 | CCAAGTGGGGCTCTTTCATGCTCCTCCTCCTCCTCCTCCT |
| 124 | CTCTTTCATGCTCCTCCTCC |

TABLE 11
Second editing template sequences

| SEQ | |
|---|---|
| ID | |
| NO: | Second editing template (RTT) sequence |
| 125 | CCTAGGGCTACGTTTCCTGGCAAAACTTCCGAAAGCCATT |
| | TCTCCAAAAGAAGGTCTAGAAGAGGAGGAGGAGGAGGA |
| | GAAGGAGGAGGAGGAGGAGGAGCAGCAGCAGCAGCAGC |
| | ATGAAAGAGCCCCACTTGGAAGGCGGTTTGGATTTTATTT |
| | GTGTGTTTTGTGGATTCTTTTTATTTTGCTTTACAAATGCA |
| | TCTTACACCAAACTCATC |
| 126 | CGTTTCCTGGCAAAACTTCCGAAAGCCATTTCTCCAAAAG |
| | AAGGTCTAGAAGAGGAGGAGGAGGAGGAGAAGGAGGAG |
| | GAGGAGGAGGAGCAGCAGCAGCAGCAGCATGAAAGAGC |
| | CCCACTTGGAAGGCGGTTTGGATTTTATTTGTGTGTTTTGT |
| | GGATTCTTTTTATTTTGCTTTACAAATGCATCTTACAC |
| 127 | CAAAACTTCCGAAAGCCATTTCTCCAAAAGAAGGTCTAG |
| | AAGAGGAGGAGGAGGAGGAGAAGGAGGAGGAGGAGGA |
| | GGAGCAGCAGCAGCAGCAGCATGAAAGAGCCCCACTTGG |
| | AAGGCGGTTTGGATTTTATTTGTGTGTTTTGTGGATTCTTT |
| | TTATTTTGCTTTACAAATG |
| 128 | GAAAGCCATTTCTCCAAAAGAAGGTCTAGAAGAGGAGGA |
| | GGAGGAGGAGAAGGAGGAGGAGGAGGAGGAGCAGCAG |
| | CAGCAGCAGCATGAAAGAGCCCCACTTGGAAGGCGGTTT |
| | GGATTTTATTTGTGTGTTTTGTGGATTCTTTTTATTTTGC |
| 129 | TCTCCAAAAGAAGGTCTAGAAGAGGAGGAGGAGGAGGA |
| | GAAGGAGGAGGAGGAGGAGGAGCAGCAGCAGCAGCAGC |
| | ATGAAAGAGCCCCACTTGGAAGGCGGTTTGGATTTTATTT |
| | GTGTGTTTTGTGGATTCTT |
| 130 | AAGGTCTAGAAGAGGAGGAGGAGGAGGAGAAGGAGGAG |
| | GAGGAGGAGGAGCAGCAGCAGCAGCAGCATGAAAGAGC |
| | CCCACTTGGAAGGCGGTTTGGATTTTATTTGTGTGTTTT |
| 131 | AGAGGAGGAGGAGGAGGAGAAGGAGGAGGAGGAGGAG |
| | GAGCAGCAGCAGCAGCAGCATGAAAGAGCCCCACTTGGA |
| | AGGCGGTTTGGATTTTATT |
| 132 | GAGGAGGAGAAGGAGGAGGAGGAGGAGGAGCAGCAGC |
| | AGCAGCAGCATGAAAGAGCCCCACTTGGAAGGCGGTTT |
| 133 | AGGAGGAGGAGGAGGAGGAGCAGCAGCAGCAGCAGCAT |
| | GAAAGAGCCCCACTTGG |
| 134 | GGAGGAGGAGCAGCAGCAGCAGCAGCATGAAAGAG |
| 135 | CCTAGGGCTACGTTTCCTGGCAAAACTTCCGAAAGCCATT |
| | TCTCCAAAAGAAGGTCTAGAAGAGGAGGAGGAGGAGGA |
| | GAAGGAGGAGGAGGAGGAGGAGCATGAAAGAGCCCCAC |
| | TTGGAAGGCGGTTTGGATTTTATTTGTGTGTTTTGTGGATT |
| | CTTTTTATTTTGCTTTACAAATGCATCTTACACCAAACTCA |
| | TC |
| 136 | CGTTTCCTGGCAAAACTTCCGAAAGCCATTTCTCCAAAAG |
| | AAGGTCTAGAAGAGGAGGAGGAGGAGGAGAAGGAGGAG |
| | GAGGAGGAGGAGCATGAAAGAGCCCCACTTGGAAGGCG |
| | GTTTGGATTTTATTTGTGTGTTTTGTGGATTCTTTTTATTT |
| | TGCTTTACAAATGCATCTTACAC |
| 137 | CAAAACTTCCGAAAGCCATTTCTCCAAAAGAAGGTCTAG |
| | AAGAGGAGGAGGAGGAGGAGAAGGAGGAGGAGGAGGA |
| | GGAGCATGAAAGAGCCCCACTTGGAAGGCGGTTTGGATT |
| | TTATTTGTGTGTTTTGTGGATTCTTTTTATTTTGCTTTACA |
| | AATG |
| 138 | GAAAGCCATTTCTCCAAAAGAAGGTCTAGAAGAGGAGGA |
| | GGAGGAGGAGAAGGAGGAGGAGGAGGAGGAGCATGAA |
| | AGAGCCCCACTTGGAAGGCGGTTTGGATTTTATTTGTGTG |
| | TTTTGTGGATTCTTTTTATTTTGC |
| 139 | TCTCCAAAAGAAGGTCTAGAAGAGGAGGAGGAGGAGGA |
| | GAAGGAGGAGGAGGAGGAGGAGCATGAAAGAGCCCCAC |
| | TTGGAAGGCGGTTTGGATTTTATTTGTGTGTTTTGTGGATT |
| | CTT |
| 140 | AAGGTCTAGAAGAGGAGGAGGAGGAGGAGAAGGAGGAG |
| | GAGGAGGAGGAGCATGAAAGAGCCCCACTTGGAAGGCG |
| | GTTTGGATTTTATTTGTGTGTTTT |
| 141 | AGAGGAGGAGGAGGAGGAGAAGGAGGAGGAGGAGGAG |
| | GAGCATGAAAGAGCCCCACTTGGAAGGCGGTTTGGATTT |
| | TATT |
| 142 | GAGGAGGAGAAGGAGGAGGAGGAGGAGGAGCATGAAA |
| | GAGCCCCACTTGGAAGGCGGTTT |
| 143 | AGGAGGAGGAGGAGGAGGAGCATGAAAGAGCCCCACTT |
| | GG |
| 144 | GGAGGAGGAGCATGAAAGAG |

RTT's may also include sequences unrelated to the endogenous sequence, that is, essentially random. This may be done, for example, to insert a readily-identifiable sequence to permit rapid determination of successful editing, or to improve editing efficiency by controlling insert length or GC content. Exemplary random or quasi-random RTT sequences are presented in Table 12, in which consecutive even- and odd-numbered sequences are reverse complements of one another. In some embodiments, the first editing template is selected from the group consisting of SEQ ID NO: 300+2n, where n is any number from 0 to 77. In some embodiments, the second editing template is selected from the group consisting of SEQ ID NO: 146+2n, where n is any number from 0 to 77. In some embodiments, the first editing template is selected from the group consisting of SEQ ID NO: 145+2n and the second editing template is selected from the group consisting of SEQ ID NO: 146+2n, where n is any number from 0 to 77. In some embodiments, such as SEQ ID NOs: 145-162, the editing template has a GC content of 42%. In some embodiments, such as SEQ IDO NOs: 163-180, the editing template has a GC content of 53%. In some embodiments, such as SEQ ID NOs: 181-198 and 235-298, the editing template has a GC content of 63%. In some embodiments, such as SEQ ID NOs: 199-216, the editing template has a GC content of 71%. In some embodiments, such as SEQ ID NOs: 217-234, the editing template has a GC content of 79%.

TABLE 12
Exemplary random or quasi-random RTT sequences

| First RTT | | Second RTT | |
|---|---|---|---|
| SEQ ID | | SEQ ID | |
| NOs: | RTT Sequence | NOs: | RTT Sequence |
| 145 | TTTCCAAGATTTGCAACCG | 146 | GGTAATAGCATACAGTTGCCGGTT |
| | GCAACTGTATGCTATTACC | | GCAAATCTTGGAAA |
| 147 | TATTGTTCCGGTTTATCACG | 148 | TGTAATCCGAGTACGCTTCGTGAT |
| | AAGCGTACTCGGATTACA | | AAACCGGAACAATA |
| 149 | TATCGCACCACCCTATGCG | 150 | GATAAGTATTGATATGGAACGCA |
| | TTCCATATCAATACTTATC | | TAGGGTGGTGCGATA |
| 151 | TTCGTCTACAGTATCCTTCT | 152 | TGCGGTGAATAGGTTGAAAGAAG |
| | TTCAACCTATTCACCGCA | | GATACTGTAGACGAA |
| 153 | TGTTATGGTGTGAATACTTC | 154 | GTGGAAATCATCCGCCATGAAGT |
| | ATGGCGGATGATTTCCAC | | ATTCACACCATAACA |
| 155 | TATGTATACGAAGCCCATG | 156 | TGGATCGGCAATATGTTTGCATGG |
| | CAAACATATTGCCGATCCA | | GCTTCGTATACATA |
| 157 | TTACCGTGTCTACATGGTAT | 158 | GGTAATCGCAAATGCCAAATACC |
| | TTGGCATTTGCGATTACC | | ATGTAGACACGGTAA |
| 159 | TATACAAGGTACATATACC | 160 | TTCTGTTGGAGTGCCCGTTGGTAT |
| | AACGGGCACTCCAACAGAA | | ATGTACCTTGTATA |
| 161 | TTACTGCGTATACTTGGAA | 162 | TCCCTCTGGACGTGTAAATTTCCA |
| | ATTTACACGTCCAGAGGGA | | AGTATACGCAGTAA |
| 163 | TGTATGTACAGGGTATACG | 164 | GGCGGTGCCATATCCTGACCGTAT |
| | GTCAGGATATGGCACCGCC | | ACCCTGTACATACA |
| 165 | TCACCCTCGACAAGTACAG | 166 | TGTCCGTGCATTCGAGTACCTGTA |
| | GTACTCGAATGCACGGACA | | CTTGTCGAGGGTGA |
| 167 | TTCCGAAAGCTTTCGCTCTC | 168 | GCTCCAGAATACGCCTGAGAGAG |
| | TCAGGCGTATTCTGGAGC | | CGAAAGCTTTCGGAA |
| 169 | TGGCGGAGAAATCAATTGC | 170 | GGAGCAACACGGGTTGAGAGCAA |
| | TCTCAACCCGTGTTGCTCC | | TTGATTTCTCCGCCA |
| 171 | TCACGTTCCTCATCTTGTTA | 172 | GTATGGGCCTGCACGTGGTAACA |
| | CCACGTGCAGGCCCATAC | | AGATGAGGAACGTGA |
| 173 | TCTCGTCAAGGGAACCAAA | 174 | GAGCCCACACATACTCGTGTTTGG |
| | CACGAGTATGTGTGGGCTC | | TTCCCTTGACGAGA |
| 175 | TATACTGTGCGCCGACATA | 176 | GAGTGACAGGAAACCCTCGTATG |
| | CGAGGGTTTCCTGTCACTC | | TCGGCGCACAGTATA |
| 177 | TCTCTGGAATCTTTCGTTTC | 178 | GCCACACCACACCCGTGAGAAAC |
| | TCACGGGTGTGGTGTGGC | | GAAAGATTCCAGAGA |
| 179 | TACCTATGTACACCAGAAC | 180 | GTGGAGCACACGAGGACTGGTTC |
| | CAGTCCTCGTGTGCTCCAC | | TGGTGTACATAGGTA |
| 181 | TACCGGAGGGCGACACTCG | 182 | GTGTGGGAGGTCACAACCTCGAG |
| | AGGTTGTGACCTCCCACAC | | TGTCGCCCTCCGGTA |
| 183 | TCGTCCCTGTCCGCGGCCG | 184 | TGTGGGCGTACAATACTCTCGGCC |
| | AGAGTATTGTACGCCCACA | | GCGGACAGGGACGA |
| 185 | TCACGATCGATCGCGCGGA | 186 | GCGGTGGAGCATACCCAGTTCCG |
| | ACTGGGTATGCTCCACCGC | | CGCGATCGATCGTGA |
| 187 | TTCTCGGAGGCATGCGGGC | 188 | GGGACGGAGCCATGCAAATGCCC |
| | ATTTGCATGGCTCCGTCCC | | GCATGCCTCCGAGAA |
| 189 | TCTCTGCCGCTATGCGTTCA | 190 | GGCGCCTTTGCCATGGCGTGAAC |
| | CGCCATGGCAAAGGCGCC | | GCATAGCGGCAGAGA |
| 191 | TGCATGATGGTCCGATGCT | 192 | GGATGAGGCCGAGGCAGGCAGCA |
| | GCCTGCCTCGGCCTCATCC | | TCGGACCATCATGCA |
| 193 | TGCGTCGACGCTTGCGCAT | 194 | GACCGCCCGTTCTGCATGTATGCG |
| | ACATGCAGAACGGGCGGTC | | CAAGCGTCGACGCA |
| 195 | TCAGCTCTGTCGAGGCCCA | 196 | GTCACCAAGGACCACGGCTTGGG |
| | AGCCGTGGTCCTTGGTGAC | | CCTCGACAGAGCTGA |
| 197 | TCTTCCGGTACGCGTCTCCT | 198 | TCGGTTCGGGCGAAACCCAGGAG |
| | GGGTTTCGCCCGAACCGA | | ACGCGTACCGGAAGA |
| 199 | TGGCGCACCGCGGCGGTAT | 200 | GTACAGAGCTCGGCCCGAGATAC |
| | CTCGGGCCGAGCTCTGTAC | | CGCCGCGGTGCGCCA |
| 201 | TGCCAATTGAGCCGAACAG | 202 | GCCGGCAGGCCCGCAGGCGCTGT |
| | CGCCTGCGGGCCTGCCGGC | | TCGGCTCAATTGGCA |
| 203 | TGTCACACATGCTCCCGGC | 204 | GCCGTGCGTCGGGCGAGGTGCCG |
| | ACCTCGCCCGACGCACGGC | | GGAGCATGTGTGACA |
| 205 | TGCGGGCGAACGCTGCTTT | 206 | GAGGTGCCCGGTGCCGCTGAAAG |
| | CAGCGGCACCGGGCACCTC | | CAGCGTTCGCCCGCA |
| 207 | TTGCCGCACGCCGAGCCTT | 208 | TTCGCCCGGGCCCGTAGAGAAGG |
| | CTCTACGGGCCCGGGCGAA | | CTCGGCGTGCGGCAA |
| 209 | TGCCCGCCACGCTGCGTCC | 210 | GGTGACGTTTCCCGTCCCTGGACG |
| | AGGGACGGGAAACGTCACC | | CAGCGTGGCGGGCA |
| 211 | TACGGCGGCCGGACCTCTC | 212 | GCCATGCAGGCGTAACGCGGAGA |
| | CGCGTTACGCCTGCATGGC | | GGTCCGGCCGCCGTA |
| 213 | TGCTGCGAGAACCGCCTAT | 214 | GGCAGCGTGCGCCCGGCTGATAG |
| | CAGCCGGGCGCACGCTGCC | | GCGGTTCTCGCAGCA |
| 215 | TGTGCCCAGGCTTTATCGG | 216 | GGCAACGCGCGGCAGGCGGCCGA |
| | CCGCCTGCCGCGCGTTGCC | | TAAAGCCTGGGCACA |
| 217 | TGCATCGCGGTGCGGCCCG | 218 | GGAAGGCCCGCCGACGGCTCGGG |
| | AGCCGTCGGCGGGCCTTCC | | CCGCACCGCGATGCA |
| 219 | TACGCACACCCGGCCCGGG | 220 | GCCACGCGGCCGAGTCGCTCCCG |
| | AGCGACTCGGCCGCGTGGC | | GGCCGGGTGTGCGTA |
| 221 | TGGCCGACTCCGGACCGTC | 222 | GCCGCGCTCCGCGCGAGGTGACG |
| | ACCTCGCGCGGAGCGCGGC | | GTCCGGAGTCGGCCA |
| 223 | TGCGCGCCACCACGCGCGC | 224 | GGTCTCGTGCGGAGGCCACGCGC |
| | GTGGCCTCCGCACGAGACC | | GCGTGGTGGCGCGCA |
| 225 | TGGCGGATGAGCCGCCCAG | 226 | GGCCGGCCCTCGCCGCCTTCTGGG |
| | AAGGCGGCGAGGGCCGGC | | CGGCTCATCCGCCA |
| | C | | |
| 227 | TGCCAGGTCGACCGAGGCC | 228 | GCCGTCTTCCCGGGCCCGCGGCCT |
| | GCGGGCCCGGGAAGACGG | | CGGTCGACCTGGCA |
| | C | | |
| 229 | TCGCCCGGCGCCGGGATGT | 230 | TTTCCGGGCTCCGCGGCGCACATC |
| | GCGCCGCGGAGCCCGGAAA | | CCGGCGCCGGGCGA |
| 231 | TCGAAGCCGCGGCTGGCGC | 232 | GTGCTCGCCGGGCACGTCCGCGC |
| | GGACGTGCCCGGCGAGCAC | | CAGCCGCGGCTTCGA |
| 233 | TCCGTACGGGCCCAGGGTA | 234 | GCTGCCCGGCACGCGGGCGTACC |
| | CGCCCGCGTGCCGGGCAGC | | CTGGGCCCGTACGGA |
| 235 | TCCGCCTCTTATCTGTGGGC | 236 | GCGGCCCACAGATAAGAGGCGGA |
| | CGC | | |
| 237 | TGCCAACCGTCGGGTCCAT | 238 | TCGCATGGACCCGACGGTTGGCA |
| | GCGA | | |
| 239 | TTCGAGCGCACCCGCAACA | 240 | GGCTTGTTGCGGGTGCGCTCGAA |
| | AGCC | | |
| 241 | TTCGCGCAGCAGGGTTGCG | 242 | GGTTCGCAACCCTGCTGCGCGAA |
| | AACC | | |
| 243 | TTTGGCCGGTGCGGGACAG | 244 | GTACCTGTCCCGCACCGGCCAAA |
| | GTAC | | |
| 245 | TCGTTTGGCGATGGCCTCCT | 246 | GGGAGGAGGCCATCGCCAAACGA |
| | CCC | | |
| 247 | TCTCGGCGAGCCGTCTACG | 248 | GACACGTAGACGGCTCGCCGAGA |
| | TGTC | | |
| 249 | TCGTGGTCTCGGGCATCAG | 250 | GGGTCTGATGCCCGAGACCACGA |
| | ACCC | | |
| 251 | TGCCCAGTCCGTGTCTCTGC | 252 | TGCGCAGAGACACGGACTGGGCA |
| | GCA | | |
| 253 | TGCGTTTGTGCAATGGGCG | 254 | TGATCTGTGGCGATACATGCGCG |
| | GCGCAGTGGGCCGCGCATG | | GCCCACTGCGCCGCCCATTGCACA |
| | TATCGCCACAGATCA | | AACGCA |
| 255 | TCGGAGGCCACGGTCGCCA | 256 | GCCCGACATCCATAGTGAAACAG |
| | CAGCAACGTGGCTGTTTCA | | CCACGTTGCTGTGGCGACCGTGGC |
| | CTATGGATGTCGGGC | | CTCCGA |
| 257 | TCTCGGTCACGCACGGCGC | 258 | TTGGAGTAGCAATGCGTGTCGGG |
| | AGCTCGGACAACCCGACAC | | TTGTCCGAGCTGCGCCGTGCGTGA |
| | GCATTGCTACTCCAA | | CCGAGA |
| 259 | TTGCTCCGTGGTATCGGTCC | 260 | TCCCTTGCGCGGCGGCAAACCTG |
| | CGAGCCATATCAGGTTTGC | | ATATGGCTCGGGACCGATACCAC |
| | CGCCGCGCAAGGGA | | GGAGCAA |
| 261 | TTGGGTGTGGCGCCAACGG | 262 | GGTCCTCTCCACCACAACGGCCGC |
| | GAGTGCGGCCTGGGTACCT | | AATAATAGGTACCCAGGCCGCAC |
| | ATTATTGCGGCCGTTGTGG | | TCCCGTTGGCGCCACACCCAA |
| | TGGAGAGGACC | | |
| 263 | TCGCTGGCGTCGTACGCGG | 264 | TGGTGCAGCCAGGGTCTGCTTGAC |
| | AGTGTGCAAGAGCTGGGTG | | TTTCGCCACCCAGCTCTTGCACAC |
| | GCGAAAGTCAAGCAGACCC | | TCCGCGTACGACGCCAGCGA |
| | TGGCTGCACCA | | |
| 265 | TCGGCTGTGGCAGGCCGGG | 266 | TGTTCCTGACGGCTGGGTAGCTTC |
| | AAGCTCCATGTACACGCTG | | GCCTTGCAGCGTGTACATGGAGCT |
| | CAAGGCGAAGCTACCCAGC | | TCCCGGCCTGCCACAGCCGA |
| | CGTCAGGAACA | | |
| 267 | TTACCGGGCAGTGTCCTTTC | 268 | GACGCGGAAGCCTTCCTCCTGCGT |
| | TGCGCAGCAGGGTCGTACC | | AGTCCGGTACGACCCTGCTGCGC |
| | GGACTACGCAGGAGGAAG | | AGAAAGGACACTGCCCGGTAA |
| | GCTTCCGCGTC | | |
| 269 | TACAACGCGGATCAGGGCA | 270 | GCATACCACCCATCTCGGGACAC |
| | TCCATCCACAGCGTGCGCG | | CGCCGTTCGCGCACGCTGTGGATG |
| | AACGGCGGTGTCCCGAGAT | | GATGCCCTGATCCGCGTTGTA |
| | GGGTGGTATGC | | |
| 271 | TCCTCTCGCCTGAATCGGA | 272 | GGTAGACCGCCTCCGGAGGCTCTT |
| | CTGGACAGCTTCGTGGGAT | | CGAGCCATCCCACGAAGCTGTCC |
| | GGCTCGAAGAGCCTCCGGA | | AGTCCGATTCAGGCGAGAGGA |
| | GGCGGTCTACC | | |
| 273 | TTGCCGACTGATACTGCGT | 274 | GGCTGCGCGGGCGTCCGCTTGCA |
| | CCTTACGCCCGATTGCGTT | | CCCTTGTAACGCAATCGGGCGTA |
| | ACAAGGGTGCAAGCGGAC | | AGGACGCAGTATCAGTCGGCAA |
| | GCCCGCGCAGCC | | |
| 275 | TCCCGGAGCGAAACCGGCC | 276 | GGAAGACAGCCGCCACATATCCA |
| | AACGAAGCGAAAGGGTGG | | CTGCAGGGCCACCCTTTCGCTTCG |
| | CCCTGCAGTGGATATGTGG | | TTGGCCGGTTTCGCTCCGGGA |
| | CGGCTGTCTTCC | | |
| 277 | TCCAGCAAGAGGCAACCCA | 278 | GAAATCGAGCGAGGTTCCGTCGT |
| | CCCACGCGAAGCCACCACT | | CGGCGCAAGTGGTGGCTTCGCGT |
| | TGCGCCGACGACGGAACCT | | GGGTGGGTTGCCTCTTGCTGGA |
| | CGCTCGATTTC | | |
| 279 | TCCGAGGAGGACAGAGCCA | 280 | GCGTGACCTCGCCGCTTCAAGGCC |
| | AGGACAGCCCTTCCTCAAT | | GCGCACGTCGATCGTGAGTAGAT |
| | CTACTCACGATCGACGTGC | | TGAGGAAGGGCTGTCCTTGGCTCT |
| | GCGGCCTTGAAGCGGCGAG | | GTCCTCCTCGGA |
| | GTCACGC | | |
| 281 | TCCCGCCTTCACACCCTCG | 282 | TCCCATCCAGTACAGCCCGGTCCG |
| | AGATACTGCGCGCTCAGCC | | ACTGTGCGCGATAGGTGTGAAGG |
| | TTCACACCTATCGCGCACA | | CTGAGCGCGCAGTATCTCGAGGG |
| | GTCGGACCGGGCTGTACTG | | TGTGAAGGCGGGA |
| | GATGGGA | | |
| 283 | TTGCAGCCAATTGTGGCAA | \|284 | GATAGCTTGGCCAGCAAGTGGCG |
| | CACCGGAAGCCAAGGCACA | | CGCCCACCGGCGATCGGCATCCT |
| | GGATGCCGATCGCCGGTGG | | GTGCCTTGGCTTCCGGTGTTGCCA |
| | GCGCGCCACTTGCTGGCCA | | CAATTGGCTGCAA |
| | AGCTATC | | |
| 285 | TTGGCCAAAGACACCACGA | 286 | GGCTGTGCGCGCCTTCGTCGCAGT |
| | GAAATGCCCAATCAGGGTG | | CCGCTTCGTGGCAGTCCCTGCCAC |
| | GCAGGGACTGCCACGAAGC | | CCTGATTGGGCATTTCTCGTGGTG |
| | GGACTGCGACGAAGGCGCG | | TCTTTGGCCAA |
| | CACAGCC | | |
| 287 | TTACTGAGTGGCCCGTACG | 288 | TGGGATGACCAGAAGACGGACCA |
| | CGCAGGGCCGTCGGCAGAA | | TCTGCCGTCGGTCGACACTGAGTT |
| | CTCAGTGTCGACCGACGGC | | CTGCCGACGGCCCTGCGCGTACG |
| | AGATGGTCCGTCTTCTGGT | | GGCCACTCAGTAA |
| | CATCCCA | | |
| 289 | TCGCGGCCTGCGGTTCGGT | \|290 | GGCTCCGGCGACTTTCGAAGCTCC |
| | ACGTTGCGTTTCAAACCGG | | GCAAGCAGACGTGCAGGAAGACC |
| | TCTTCCTGCACGTCTGCTTG | | GGTTTGAAACGCAACGTACCGAA |
| | CGGAGCTTCGAAAGTCGCC | | CCGCAGGCCGCGA |
| | GGAGCC | | |
| 291 | TGGCGCCTGAGCCGTTTGA | 292 | TCCACACGGGACACCGGAGTGGC |
| | AAGAGGCACTCGTGCCATA | | GTTGCCAGAGGCGTCGAGGCAAT |
| | TTGCCTCGACGCCTCTGGC | | ATGGCACGAGTGCCTCTTTCAAAC |
| | AACGCCACTCCGGTGTCCC | | GGCTCAGGCGCCA |
| | GTGTGGA | | |
| 293 | TCATACGCGGCAATCGCGT | 294 | GGCACGGCTGTGCAACGCCGCCG |
| | CCTTCTGTACGGACCCACT | | TCTCTGACATAGCGGCAACCGTA |
| | ACGGTTGCCGCTATGTCAG | | GTGGGTCCGTACAGAAGGACGCG |
| | AGACGGCGGCGTTGCACAG | | ATTGCCGCGTATGA |
| | CCGTGCC | | |
| 295 | TCCCACTACCGTCAGACTC | 296 | GGGTGTACGCTCCGCTCTCCTTGA |
| | GCTCAGAACAGACCAGATC | | CAAGGCGGATCCCGCCGCCGTGA |
| | ACGGCGGCGGGATCCGCCT | | TCTGGTCTGTTCTGAGCGAGTCTG |
| | TGTCAAGGAGAGCGGAGCG | | ACGGTAGTGGGA |
| | TACACCC | | |
| 297 | TGGGCGACTTACGATTGGA | 298 | TCGCTCCCAGGTGTCCTCCTCCTC |
| | GCATTCTCCTCGGCTTTGTT | | CCAGCGGGTCGCGCTGAGCCAAC |
| | GGCTCAGCGCGACCCGCTG | | AAAGCCGAGGAGAATGCTCCAAT |
| | GGAGGAGGAGGACACCTG | | CGTAAGTCGCCCA |
| | GGAGCGA | | |
| 299 | ACGATGATGGTGATGATGG | 300 | GCACACCATCATCACCATCATCGT |
| | TGTGC | | |

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PERNA may comprise one or more intended nucleotide edits, compared to the TCF4 gene to be edited. Position of the intended nucleotide edit(s) relevant to other components of the PEgRNA, or to particular nucleotides (e.g., mutations) in the TCF4 target gene may vary. In some embodiments, the nucleotide edit is in a region of the PERNA corresponding to or homologous to the protospacer sequence. In some embodiments, the nucleotide edit is in a region of the PERNA corresponding to a region of the TCF4 gene outside of the protospacer sequence.

In some embodiments, incorporation of the one or more intended nucleotide edits results in deletion of the inter-nick duplex (IND) from the double-stranded target DNA, e.g., the TCF4 gene. In some embodiments, the IND comprises a mutation compared to a wild-type gene sequence, e.g., a wild-type TCF4 gene. In some embodiments, the IND comprises a mutation in intron 3 of the TCF4 gene as compared to a wild-type TCF4 gene. In some embodiments, the mutation is expansion of the number of CTG repeats compared to a wild-type TCF4 gene. In some embodiments, the IND is located between positions corresponding to positions 55586156 and 55586227 of human chromosome 18 as set forth in human genome research consortium human build 38 (GRCh38). In some embodiments, the IND is located between positions corresponding to positions 55586056 and 55586327 of human chromosome 18 as set forth in human genome research consortium human build 38 (GRCh38). In some embodiments, the IND is located between positions corresponding to positions 55585956 and 55586427 of human chromosome 18 as set forth in human genome research consortium human build 38 (GRCh38). In some embodiments, the IND is located between positions corresponding to U.S. Pat. Nos. 55,585,856 and 55,586,527 of human chromosome 18 as set forth in human genome research consortium human build 38 (GRCh38). In some embodiments, the IND is located between positions corresponding to positions 55585756 and 55586627 of human chromosome 18 as set forth in human genome research consortium human build 38 (GRCh38). In some embodiments, the IND is located between positions corresponding to positions 55585656 and 55586727 of human chromosome 18 as set forth in human genome research consortium human build 38 (GRCh38).

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises a uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PERNA following 5′-spacer-gRNA core-RTT-PBS-3′ orientation, the 5′ most nucleobase is the “first base”). In some embodiments, the editing template does not comprise a cytosine at the first nucleobase position (e.g., for a PERNA following 5′-spacer-gRNA core-RTT-PBS-3′ orientation, the 5′ most nucleobase is the “first base”).

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a Cas9 nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer and a base paired “upper stem” following the lower stem, where the lower stem and upper stem may be connected by a “bulge” comprising unpaired RNAs. The gRNA core may further comprise a “nexus” distal from the spacer, followed by a hairpin structure, e.g., at the 3′ end, as exemplified in FIG. 3. In some embodiments, the gRNA core comprises modified nucleotides as compared to a wild-type gRNA core in the lower stem, upper stem, and/or the hairpin. For example, nucleotides in the lower stem, upper stem, and/or the hairpin regions may be modified, deleted, or replaced. In some embodiments, RNA nucleotides in the lower stem, upper stem, and/or the hairpin regions may be replaced with one or more DNA sequences. In some embodiments, the gRNA core comprises unmodified or wild-type RNA sequences in the nexus and/or the bulge regions. In some embodiments, the gRNA core does not include long stretches of A-T pairs, for example, a GUUUU-AAAAC pairing element. For all PEgRNA sequences, “T” is used instead of “U” in the sequences for consistency with the ST.26 standard. In some embodiments, the gRNA core is capable of binding to a SpCas9, and comprises the sequence:
GTTTTAGAGCTAGAAATAGCAAGTTAAAATAAGGCTAGTCCGTTATCAACTTG AAAAAGTGGCACCGAGTCGGTGC (SEQ ID NO: 301);
GTTTGAGAGCTAGAAATAGCAAGTTTAAATAAGGCTAGTCCGTTATCAACTTG AAAAAGTGGGACCGAGTCGGTCC (SEQ ID NO: 302), or
GTTTAAGAGCTATGCTGGAAACAGCATAGCAAGTTTAAATAAGGCTAGTCCGT TATCAACTTGAAAAAGTGGCACCGAGTCGGTGC (SEQ ID NO: 303). In some embodiments, the gRNA core comprises the sequence GTTTTAGAGCTAGAAATAGCAAGTTAAAATAAGGCTAGTCCGTTATCAACTTG AAAAAGTGGCACCGAGTCGGTGC (SEQ ID NO: 301). In some embodiments, the gRNA core is capable of binding to a SaCas9 and comprises the sequence GTTTTAGTACTCTGGAAACAGAATCTACTAAAACAAGGCAAAATGCCGTGTTT ATCTCGTCAACTTGTTGGCGAGA (SEQ ID NO: 304). In some embodiments, the gRNA core is capable of binding to a SluCas9 and comprises the sequence GTTTTAGTACTCTGGAAACAGAATCTACTGAAACAAGACAATATGTCGTGTTTA TCCCATCAATTTATTGGTGGGA (SEQ ID NO: 305).

In some embodiments, a PEgRNA comprises a gRNA core that comprises a modified direct repeat compared to the sequence of a naturally occurring CRISPR-Cas guide RNA scaffold, for example, a Cas9 gRNA scaffold. In some embodiments, the PEgRNA comprises a “flip and extension (F+E)” gRNA core, wherein one or more base pairs in a direct repeat is modified. In some embodiments, the PEgRNA comprises a first direct repeat (the first paring element or the lower stem), wherein a uracil is changed to a adenine (such that in the stem region, a U-A base pair is changed to a A-U base pair). In some embodiments, the PEgRNA comprises a first direct repeat wherein the fourth U-A base pair in the stem is changed to a A-U base pair. In some embodiments, the PEgRNA comprises a first direct repeat wherein one or more U-A base pair is changed to a G-C or C-G base pair. For example, in some embodiments, the PEgRNA comprises a first direct repeat comprising a modification to a GUUUU-AAAAC (SEQ ID NO: 380) pairing element, wherein one or more of the U-A base pairs is changed to a A-U base pair, a G-C base pair, or a C-G base pair. In some embodiments, the PEgRNA comprises an extended first direct repeat.

In some embodiments, a PEgRNA comprises a gRNA core comprising the sequence
GTTTTAGAGCTATACGTAGCAAGTTAAAATAAGGCTAGTCCGTTATCAACTTTA CGAAGTGGCACCGAGTCGGTGC (SEQ ID NO: 381) or
GTTTTAGAGCTATACGTAGCAAGTTAAAATAAGGCTAGTCCGTTATCAACTTTA CGAAGTGGGACCGAGTCGGTCC (SEQ ID NO: 382).

In some embodiments, a PEgRNA comprises a gRNA core comprising the sequence
GTTTTAGAGCTAGCTCATGAAAATGAGCTAGCAAGTTAAAATAAGGCTAGTCC GTTATCAACTTGAAAAAGTGGGACCGAGTCGGTCC (SEQ ID NO: 383).

In some embodiments, a PERNA comprises a gRNA core comprising the sequence
GTTTGAGAGCTAGAAATAGCAAGTTTAAATAAGGCTAGTCCGTTATCAACTTG AAAAAGTGGGACCGAGTCGGTCC (SEQ ID NO: 302).

In some embodiments, a PEgRNA comprises a gRNA core comprising the sequence
GTTTAAGAGCTAGAAATAGCAAGTTTAAATAAGGCTAGTCCGTTATCAACTTG AAAAAGTGGCACCGAGTCGGTGC (SEQ ID NO: 384).

In some embodiments, a PEgRNA comprise a gRNA core comprising the sequence GTTTTAGAGCTATGCTGGAAACAGCATAGCAAGTTAAAATAAGGCTAGTCCGT TATCAACTTGAAAAAGTGGCACCGAGTCGGTGC (SEQ ID NO: 385). Table 13 lists gRNA core exemplary sequence for use in PEgRNAs. Any gRNA core sequences known in the art are also contemplated in the prime editing compositions described herein. In some embodiments, one or more nucleotides in the gRNA core is DNA.

TABLE 13
Exemplary gRNA core sequence

| SEQ ID NO: | Scaffold sequence |
|---|---|
| 301 | GTTTTAGAGCTAGAAATAGCAAGTTAAAATAAGGCTAGT |
| | CCGTTATCAACTTGAAAAAGTGGCACCGAGTCGGTGC |
| 302 | GTTTGAGAGCTAGAAATAGCAAGTTTAAATAAGGCT |
| | AGTCCGTTATCAACTTGAAAAAGTGGGACCGAGTCG |
| | GTCC |
| 303 | GTTTAAGAGCTATGCTGGAAACAGCATAGCAAGTTT |
| | AAATAAGGCTAGTCCGTTATCAACTTGAAAAAGTGG |
| | CACCGAGTCGGTGC |
| 304 | GTTTTAGTACTCTGGAAACAGAATCTACTAAAACAA |
| | GGCAAAATGCCGTGTTTATCTCGTCAACTTGTTGGC |
| | GAGA |
| 305 | GTTTTAGTACTCTGGAAACAGAATCTACTGAAACAA |
| | GACAATATGTCGTGTTTATCCCATCAATTTATTGGTG |
| | GGA |
| 381 | GTTTTAGAGCTATACGTAGCAAGTTAAAATAAGGCT |
| | AGTCCGTTATCAACTTTACGAAGTGGCACCGAGTCG |
| | GTGC |
| 382 | GTTTTAGAGCTATACGTAGCAAGTTAAAATAAGGCT |
| | AGTCCGTTATCAACTTTACGAAGTGGGACCGAGTCG |
| | GTCC |
| 383 | GTTTTAGAGCTAGCTCATGAAAATGAGCTAGCAAGT |
| | TAAAATAAGGCTAGTCCGTTATCAACTTGAAAAAGT |
| | GGGACCGAGTCGGTCC |
| 384 | GTTTAAGAGCTAGAAATAGCAAGTTTAAATAAGGCT |
| | AGTCCGTTATCAACTTGAAAAAGTGGCACCGAGTCG |
| | GTGC |
| 385 | GTTTTAGAGCTATGCTGGAAACAGCATAGCAAGTTA |
| | AAATAAGGCTAGTCCGTTATCAACTTGAAAAAGTGG |
| | CACCGAGTCGGTGC |

Exemplary sequences for the first PEgRNA and the second PEgRNA are provided in Tables 14 and 15 (SEQ ID NOs: 306-335). The sequences provided are for use when PEgRNA is produced by transcribing it from a plasmid. If the PEgRNA is chemically synthesized, a 5′ G, if added (as explained above for Tables 6 and 7), and any 3′ A in the PBS sequence, if added (as explained above for Tables 8 and 9), should be omitted, as well as the “ttttttt” 3′ tail. T residues also should be replaced by U residues.

TABLE 14
Exemplary 5′ PEgRNA (first PEgRNA) sequences

| SEQ | |
|---|---|
| ID | |
| NO: | TCF4 5′ PEgRNA Sequence |
| 306 | GTCCAAACCGCCTTCCAAGTGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATTTGGAAGGttttttt |
| 307 | GTCCAAACCGCCTTCCAAGTGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATTTGGAAGGCGttttttt |
| 308 | GTCCAAACCGCCTTCCAAGTGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATTTGGAAGGCGGTAttttttt |
| 309 | GATCCAAACCGCCTTCCAAGTgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATTGGAAGGCttttttt |
| 310 | GATCCAAACCGCCTTCCAAGTgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATTGGAAGGCGGAttttttt |
| 311 | GATCCAAACCGCCTTCCAAGTgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATTGGAAGGCGGTTTttttttt |
| 312 | GAATCCAAACCGCCTTCCAAGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATGGAAGGCGttttttt |
| 313 | GAATCCAAACCGCCTTCCAAGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATGGAAGGCGGTAttttttt |
| 314 | GAATCCAAACCGCCTTCCAAGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATGGAAGGCGGTTTGttttttt |
| 315 | GAACCAAGTCCCAGACATGTCgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATATGTCTGGttttttt |
| 316 | GAACCAAGTCCCAGACATGTCgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATATGTCTGGGAttttttt |
| 317 | GAACCAAGTCCCAGACATGTCgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATATGTCTGGGACTAttttttt |
| 318 | GAGCGGAACTTGACACACTTGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATGTGTGTCAttttttt |
| 319 | GAGCGGAACTTGACACACTTGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATGTGTGTCAAGAttttttt |
| 320 | GAGCGGAACTTGACACACTTGgttttAGAGCTAGAAATAGCAAG |
| | TTAAAATAAGGCTAGTCCGTTATCAACTTGAAAAAGTGGCAC |
| | CGAGTCGgtgcGGCTTGTCGACGACGGCGGTCTCCGTCGTCAG |
| | GATCATGTGTGTCAAGTTCttttttt |

TABLE 15
Exemplary 3′ PERNA (first PEgRNA) sequences

| SEQ | |
|---|---|
| ID | |
| NO: | TCF4 3′ PEgRNA Sequence |
| 321 | GAAAAGAAGGTCTAGAAGAGGgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCCTTCTAGAttttttt |
| 322 | GAAAAGAAGGTCTAGAAGAGGgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCCTTCTAGACCAttttttt |
| 323 | GAAAAGAAGGTCTAGAAGAGGgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCCTTCTAGACCTTCAttttttt |
| 324 | GAAAGCCATTTCTCCAAAAGAgttttAGAGCTAGAAATAGCAA |
| | GTTAAAATAAGGCTAGTCCGTTATCAACTTGAAAAAGTGGC |
| | ACCGAGTCGgtgcATGATCCTGACGACGGAGACCGCCGTCGT |
| | CGACAAGCCTTTGGAGAttttttt |
| 325 | GAAAGCCATTTCTCCAAAAGAgttttAGAGCTAGAAATAGCAA |
| | GTTAAAATAAGGCTAGTCCGTTATCAACTTGAAAAAGTGGC |
| | ACCGAGTCGgtgcATGATCCTGACGACGGAGACCGCCGTCGT |
| | CGACAAGCCTTTGGAGAAAAttttttt |
| 326 | GAAAGCCATTTCTCCAAAAGAgttttAGAGCTAGAAATAGCAA |
| | GTTAAAATAAGGCTAGTCCGTTATCAACTTGAAAAAGTGGC |
| | ACCGAGTCGgtgcATGATCCTGACGACGGAGACCGCCGTCGT |
| | CGACAAGCCTTTGGAGAAATGGttttttt |
| 327 | GAAGTGCAACAAGCAGAAAGGgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCTTCTGCTTttttttt |
| 328 | GAAGTGCAACAAGCAGAAAGGgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCTTCTGCTTGTAttttttt |
| 329 | GAAGTGCAACAAGCAGAAAGGgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCTTCTGCTTGTTGCttttttt |
| 330 | GAGAAAGTGCAACAAGCAGAAgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCTGCTTGTTttttttt |
| 331 | GAGAAAGTGCAACAAGCAGAAgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCTGCTTGTTGCttttttt |
| 332 | GAGAAAGTGCAACAAGCAGAAgttttAGAGCTAGAAATAGCA |
| | AGTTAAAATAAGGCTAGTCCGTTATCAACTTGAAAAAGTGG |
| | CACCGAGTCGgtgcATGATCCTGACGACGGAGACCGCCGTCG |
| | TCGACAAGCCTGCTTGTTGCACAttttttt |
| 333 | GTCAGAGCCTGCAAAAAGCAAgttttAGAGCTAGAAATAGCAA |
| | GTTAAAATAAGGCTAGTCCGTTATCAACTTGAAAAAGTGGC |
| | ACCGAGTCGgtgcATGATCCTGACGACGGAGACCGCCGTCGT |
| | CGACAAGCCCTTTTTGCttttttt |
| 334 | GTCAGAGCCTGCAAAAAGCAAgttttAGAGCTAGAAATAGCAA |
| | GTTAAAATAAGGCTAGTCCGTTATCAACTTGAAAAAGTGGC |
| | ACCGAGTCGgtgcATGATCCTGACGACGGAGACCGCCGTCGT |
| | CGACAAGCCCTTTTTGCAGttttttt |
| 335 | GTCAGAGCCTGCAAAAAGCAAgttttAGAGCTAGAAATAGCAA |
| | GTTAAAATAAGGCTAGTCCGTTATCAACTTGAAAAAGTGGC |
| | ACCGAGTCGgtgcATGATCCTGACGACGGAGACCGCCGTCGT |
| | CGACAAGCCCTTTTTGCAGGCAttttttt |

In some embodiments, a PEgRNA is produced by transcription from a template nucleotide, for example, a template plasmid. In some embodiments, a polynucleotide encoding the PEgRNA is appended with one or more additional nucleotides that improves PEgRNA expression, e.g., expression from a plasmid that encodes the PERNA or ngRNA. In some embodiments, a polynucleotide encoding a PEgRNA is appended with one or more additional nucleotides at the 5′ end or at the 3′ end. In some embodiments, the polynucleotide encoding the PEgRNA is appended with a guanine at the 5′ end, for example, if the first nucleotide at the 5′ end of the spacer is not a guanine. In some embodiments, a polynucleotide encoding the PEgRNA is appended with nucleotide sequence CACC at the 5′ end. In some embodiments, the polynucleotide encoding the PEgRNA is appended with additional nucleotide sequence TTTTTT, TTTTTTT, TTTTT, or TTTT at the 3′ end. In some embodiments, the PEgRNA comprises the appended nucleotides from the transcription template. In some embodiments, the PEgRNA or ngRNA further comprises one or more nucleotides at the 5′ end or the 3′ end in addition to spacer, PBS, and RTT sequences. in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises nucleotide sequence UUUUUUU, UUUUUU, UUUUU, or UUUU at the 3′ end.

A PEgRNA may also comprise optional modifiers, e.g., 3′ end modifier region and/or a 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to aptamers, hairpins, stem/loops, toeloops, and/or RNA-binding protein recruitment domains (e.g., the MS2 aptamer which recruits and binds to the MS2 coat protein (MS2cp)). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PERNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In some embodiments, a PEgRNA comprises up to 50 nucleotides, up to 40 nucleotides, up to 30 nucleotides, up to 20 nucleotides, or up to 10 nucleotides of optional sequence modifiers at either or both of the 3′ and 5′ ends of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a PERNA comprises an additional secondary structure at the 5′ end. In some embodiments, a PEgRNA comprises an additional secondary structure at the 3′ end.

In some embodiments, the secondary structure comprises a pseudoknot. In some embodiments, the secondary structure comprises a pseudoknot derived from a virus. In some embodiments, the secondary structure comprises a pseudoknot of a Moloney murine leukemia virus (M-MLV) genome (a mpknot). In some embodiments, the secondary structure comprises a nucleotide sequence selected from the group consisting of
GGGTCAGGAGCCCCCCCCCTGAACCCAGGATAACCCTCAAAGTCGGGGGGCAA CC, (SEQ ID NO: 337),
GTCAGGGTCAGGAGCCCCCCCCCTGAACCCAGGATAACCCTCAAAGTCGGGGG GCAACCC (SEQ ID NO: 389),
GGGTCAGGAGCCCCCCCCCTGAACCCAGGAAAACCCTCAAAGTCGGGGGGCA ACCC (SEQ ID NO: 338),
GGGTCAGGAGCCCCCCCCCTGCACCCAGGAAAACCCTCAAAGTCGGGGGGCAA CCC (SEQ ID NO: 339),
GGGTCAGGAGCCCCCCCCCTGCACCCAGGATAACCCTCAAAGTCGGGGGGCAA CCC (SEQ ID NO: 340),
GTCAGGGTCAGGAGCCCCCCCCCTGAACCCAGGAAAACCCTCAAAGTCGGGGG GCAACCC (SEQ ID NO: 341),
GTCAGGGTCAGGAGCCCCCCCCCTGCACCCAGGAAAACCCTCAAAGTCGGGGG GCAACCC (SEQ ID NO: 342), and
GGGTCAGGAGCCCCCCCCCTGAACCCAGGATAACCCTCAAAGTCGGGGGGC (SEQ ID NO: 343), or a nucleotide sequence having at least 80%, 85%, 90%, 95%, 96%, 97%, 98%, or 99% sequence identity therewith. In some embodiments, the secondary structure comprises a nucleotide sequence of
GGGTCAGGAGCCCCCCCCCTGAACCCAGGATAACCCTCAAAGTCGGGGGGC (SEQ ID NO: 343), or a nucleotide sequence having at least 80%, 85%, 90%, 95%, 96%, 97%, 98%, or 99% sequence identity therewith.

In some embodiments, the secondary structure comprises a quadruplex. In some embodiments, the secondary structure comprises a G-quadruplex. In some embodiments, the secondary structure comprises a nucleotide sequence selected from the group consisting of gq2 (TGGTGGTGGTGGT (SEQ ID NO:344), stk40 (GGGACAGGGCAGGGACAGGG (SEQ ID NO: 345), apc2 (GGGTCCGGGTCTGGGTCTGGG (SEQ ID NO: 346), stard3 (GGGCAGGGTCTGGGCTGGG (SEQ ID NO: 347), Uns1 (GGGCTGGGATGGGAAAGGG (SEQ ID NO: 348), ceacam4 (GGGCTCTGGGTGGGCCGGG (SEQ ID NO: 349), erc1 (GGGCTGGGCTGGGCAGGG (SEQ ID NO: 350), pitpnm3 (GGGTGGGCTGGGAAGGG (SEQ ID NO:351), rlf (GGGAGGGAGGGCTAGGG (SEQ ID NO: 352), ube3c (GGGCAGGGCTGGGAGGG (SEQ ID NO: 353), taf15 (GGGTGGGAGGGCTGGG (SEQ ID NO: 354), and xrn1 (GCGTAACCTCCATCCGAGTTGCAAGAGAGGGAAACGCAGTCTC (SEQ ID NO: 355), or a nucleotide sequence having at least 80%, 85%, 90%, 95%, 96%, 97%, 98%, or 99% sequence identity therewith.

In some embodiments, the secondary structure comprises a P4-P6 domain of a Group I intron. In some embodiments, the secondary structure comprises the nucleotide sequence of GGAATTGCGGGAAAGGGGTCAACAGCCGTTCAGTACCAAGTCTCAGGGGAAA CTTTGAGATGGCCTTGCAAAGGGTATGGTAATAAGCTGACGGACATGGTCCTA ACCACGCAGCCAAGTCCTAAGTCAACAGATCTTCTGTTGATATGGATGCAGTTC A (SEQ ID NO: 356), or a nucleotide sequence having at least 80%, 85%, 90%, 95%, 96%, 97%, 98%, or 99% sequence identity therewith.

In some embodiments, the secondary structure comprises a riboswitch aptamer. In some embodiments, the secondary structure comprises a riboswitch aptamer derived from a prequeosine-1 riboswitch aptamer. In some embodiments, the secondary structure comprises a modified prequeosine-1 riboswitch aptamer. In some embodiments, the secondary structure comprises a nucleotide sequence selected from the group consisting of TTGACGCGGTTCTATCTAGTTACGCGTTAAACCAACTAGAAA (SEQ ID NO:357), TTGACGCGGTTCTATCTACTTACGCGTTAAACCAACTAGAAA (SEQ ID NO: 358), CGCGAGTCTAGGGGATAACGCGTTAAACTTCCTAGAAGGCGGTT (SEQ ID NO: 359), CGCGGATCTAGATTGTAACGCGTTAAACCATCTAGAAGGCGGTT (SEQ ID NO: 360), CGCGTCGCTACCGCCCGGCGCGTTAAACACACTAGAAGGCGGTT (SEQ ID NO: 361), and CGCGGTTCTATCTAGTTACGCGTTAAACCAACTAGAA (SEQ ID NO: 362), or a nucleotide sequence having at least 80%, 85%, 90%, 95%, 96%, 97%, 98%, or 99% sequence identity therewith. In some embodiments, the secondary structure comprises a nucleotide sequence selected from the group consisting of TTGACGCGGTTCTATCTAGTTACGCGTTAAACCAACTAGAAA (SEQ ID NO: 357), CGCGAGTCTAGGGGATAACGCGTTAAACTTCCTAGAAGGCGGTT (SEQ ID NO: 359), CGCGGATCTAGATTGTAACGCGTTAAACCATCTAGAAGGCGGTT (SEQ ID NO: 360), CGCGTCGCTACCGCCCGGCGCGTTAAACACACTAGAAGGCGGTT (SEQ ID NO: 361), and CGCGGTTCTATCTAGTTACGCGTTAAACCAACTAGAA (SEQ ID NO: 362), or a nucleotide sequence having at least 80%, 85%, 90%, 95%, 96%, 97%, 98%, or 99% sequence identity therewith. In some embodiments, the secondary structure comprises a nucleotide sequence of and CGCGGTTCTATCTAGTTACGCGTTAAACCAACTAGAA (SEQ ID NO: 362), or a nucleotide sequence having at least 80%, 85%, 90%, 95%, 96%, 97%, 98%, or 99% sequence identity therewith.

In some embodiments, the secondary structure is linked to one or more other component of a PEgRNA via a linker. For example, in some embodiments, the secondary structure is at the 3′ end of the PEgRNA and is linked to the 3′ end of a PBS via a linker. In some embodiments, the secondary structure is at the 5′ end of the PEgRNA and is linked to the 5′ end of a spacer via a linker. In some embodiments, the linker is a nucleotide linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the linker is 5 to 10 nucleotides in length. In some embodiments, the linker is 10 to 20 nucleotides in length. In some embodiments, the linker is 15 to 25 nucleotides in length. In some embodiments, the linker is 8 nucleotides in length.

In some embodiments, the linker is designed to minimize base pairing between the linker and another component of the PERNA. In some embodiments, the linker is designed to minimize base pairing between the linker and the spacer. In some embodiments, the linker is designed to minimize base pairing between the linker and the PBS. In some embodiments, the linker is designed to minimize base pairing between the linker and the editing template. In some embodiments, the linker is designed to minimize base pairing between the linker and the sequence of the RNA secondary structure. In some embodiments, the linker is optimized to minimize base pairing between the linker and another component of the PEgRNA, in order of the following priority: spacer, PBS, editing template and then scaffold. In some embodiments, base paring probability is calculated using ViennaRNA 2.0 under standard parameters (37° C., 1 M NaCl, 0.05 M MgCl2).

In some embodiments, the PEgRNA comprises a RNA secondary structure and/or a linker disclosed in Nelson et al. Engineered pegRNAs improve prime editing efficiency. Nat Biotechnol. (2021), the entirety of which is incorporated herein by reference.

In some embodiments, a PEgRNA is transcribed from a nucleotide encoding the PEgRNA, for example, a DNA plasmid encoding the PEgRNA. In some embodiments, the PEgRNA comprises a self-cleaving element. In some embodiments, the self-cleaving element improves transcription and/or processing of the PEgRNA when transcribed form the nucleotide encoding the PEgRNA. In some embodiments, the PEgRNA comprises a hairpin or a RNA quadruplex. In some embodiments, the PEgRNA comprises a self-cleaving ribozyme element, for example, a hammerhead, a pistol, a hatchet, a hairpin, a VS, a twister, or a twister sister ribozyme. In some embodiments, the PEgRNA comprises a HDV ribozyme. In some embodiments, the PEgRNA comprises a hairpin recognized by Csy4. In some embodiments, the PEgRNA comprises an ENE motif. In some embodiments, the PEgRNA comprises an element for nuclear expression (ENE) from MALAT1 Inc RNA. In some embodiments, the PEgRNA comprises an ENE element from Kaposi's sarcoma-associated herpesvirus (KSHV). In some embodiments, the PEgRNA comprises a 3′ box of a U1 snRNA. In some embodiments, the PEgRNA forms a circular RNA.

In some embodiments, the PEgRNA comprises a RNA secondary structure or a motif that improves binding to the DNA-RNA duple or enhances PEgRNA activity. In some embodiments, the PEgRNA comprises a sequence derived from a native nucleotide element involved in reverse transcription, e.g., initiation of retroviral transcription. In some embodiments, the PEgRNA comprises a sequence of, or derived from, a primer binding site of a substrate of a reverse transcriptase, a polypurine tract (PPT), or a kissing loop. In some embodiments, the PEgRNA comprises a dimerization motif, a kissing loop, or a GNRA tetraloop-tetraloop receptor pair that results in circularization of the PEgRNA. In some embodiments, the PEgRNA comprises a RNA secondary structure of a motif that results in physical separation of the spacer and the PBS of the PEgRNA, thereby prevents occlusion of the spacer and improves PEgRNA activity. In some embodiments, the PEgRNA comprises a secondary structure or motif, for example, a 5′ or 3′ extension in the spacer region that form a toehold or hairpin, wherein the secondary structure or motif competes favorably against annealing between the spacer and the PBS of the PEgRNA, thereby prevents occlusion of the spacer and improves PEgRNA activity.

In some embodiments, a PEgRNA comprises the sequence GGCCGGCATGGTCCCAGCCTCCTCGCTGGCGCCGGCTGGGCAACATGCTTCGGCA TGGCGAATGGGAC at the 3′ end (SEQ ID NO: 368). In some embodiments, a PEgRNA comprises the structure [spacer]-[gRNA core]-[editing template]-[PBS]-GGCCGGCATGGTCCCAGCCTCCTCGCTGGCGCCGGCTGGGCAACATGCTTCGGCA TGGCGAATGGGAC (SEQ ID NO: 368), or [spacer]-[gRNA core]-[editing template]-[PBS]-GGCCGGCATGGTCCCAGCCTCCTCGCTGGCGCCGGCTGGGCAACATGCTTCGGCA TGGCGAATGGGAC-(T) n (SEQ ID NO: 369), wherein n is an integer between 3 and 7. The structure derived from hepatitis D virus (HDV) is italicized.

In some embodiments, the PEgRNA comprises the sequence GGTGGGAGACGTCCCACC (SEQ ID NO: 370) at the 5′ end and/or the sequence TGGGAGACGTCCCACC (SEQ ID NO: 371) at the 3′ end. In some embodiments, the PEgRNA comprises the following structure (M-MLV kissing loop): GGTGGGAGACGTCCCACC (SEQ ID NO: 370)-[spacer]-[gRNA core]-[editing template]-[PBS]-TGGGAGACGTCCCACC (SEQ ID NO: 371), or GGTGGGAGACGTCCCACC (SEQ ID NO: 370)-[spacer]-[gRNA core]-[editing template]-[PBS]-TGGGAGACGTCCCACC-(T) n (SEQ ID NO: 372), wherein n is an integer between 3 and 7. The kissing loop structure is italicized. In some embodiments, the PERNA comprises the sequence GAGCAGCATGGCGTCGCTGCTCAC (SEQ ID NO: 373) at the 5′ end and/or the sequence CCATCAGTTGACACCCTGAGG (SEQ ID NO: 374) at the 3′ end. In some embodiments, the PEgRNA comprises the following structure (VS ribozyme kissing loop): GAGCAGCATGGCGTCGCTGCTCAC (SEQ ID NO: 373)-[spacer]-[gRNA core]-[editing template]-[PBS]-CCATCAGTTGACACCCTGAGG (SEQ ID NO: 374), or GAGCAGCATGGCGTCGCTGCTCAC (SEQ ID NO: 373)-[spacer]-[gRNA core]-[editing template]-[PBS]-CCATCAGTTGACACCCTGAGG-(T) n (SEQ ID NO: 375), wherein n is an integer between 3 and 7.

In some embodiments, the PEgRNA comprises the sequence GCAGACCTAAGTGGTGACATATGGTCTG (SEQ ID NO: 376) at the 5′ end and/or the sequence CATGCGATTAGAAATAATCGCATG (SEQ ID NO: 377) at the 3′ end. In some embodiments, the PEgRNA comprises the following structure (tetraloop and receptor): GCAGACCTAAGTGGTGACATATGGTCTG (SEQ ID NO: 376)-[spacer]-[gRNA core]-[editing template]-[PBS]-CATGCGATTAGAAATAATCGCATG (SEQ ID NO: 377), or GCAGACCTAAGTGGTGACATATGGTCTG (SEQ ID NO: 376)-[spacer]-[gRNA core]-[editing template]-[PBS]-CATGCGATTAGAAATAATCGCATG-(T) n (SEQ ID NO: 378), wherein n is an integer between 3 and 7. The tetraloop/tetraloop receptor structure is italicized.

In some embodiments, the PEgRNA comprises the sequence TCTGCCATCAAAGCTGCGACCGTGCTCAGTCTGGTGGGAGACGTCCCACCGGC CGGCATGGTCCCAGCCTCCTCGCTGGCGCCGGCTGGGCAACATGCTTCGGCAT GGCGAATGGGAC at the 3′ end (SEQ ID NO: 379).

A PEgRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in this disclosure may have undergone a chemical or biological modifications. Modifications may be made at any position within a PEgRNA, and may include modification to a nucleobase or to a phosphate backbone of the PEgRNA. In some embodiments, chemical modifications can be structure guided modifications. In some embodiments, a chemical modification is at the 5′ end and/or the 3′ end of a PEgRNA. In some embodiments, a chemical modification may be within the spacer, the extension arm, the editing template sequence, or the primer binding site of a PERNA. In some embodiments, a chemical modification may be within the spacer or the gRNA core of a PEgRNA. In some embodiments, a chemical modification may be within the 3′ most nucleotides of a PEgRNA. In some embodiments, a chemical modification may be within the 3′ most end of a PEgRNA. In some embodiments, a chemical modification may be within the 5′ most end of a PEgRNA. In some embodiments, a PEgRNA comprises 1, 2, 3, 4, 5, 6, 7, 8, 9, 10 or more chemically modified nucleotides at the 3′ end. In some embodiments, a PEgRNA comprises 1, 2, 3, 4, 5, 6, 7, 8, 9, 10 or more chemically modified nucleotides at the 5′ end. In some embodiments, a PEgRNA comprises 1, 2, 3, 4, or 5 or more chemically modified nucleotides at the 3′ end. In some embodiments, a PEgRNA comprises 1, 2, 3, 4, 5 or more chemically modified nucleotides at the 5′ end. In some embodiments, a PERNA comprises 1, 2, or 3 or more chemically modified nucleotides at the 3′ end. In some embodiments, a PEgRNA comprises 1, 2, 3 or more chemically modified nucleotides at the 5′ end.

In some embodiments, a PEgRNA comprises 1, 2, 3, 4, 5, 6, 7, 8, 9, 10 or more contiguous chemically modified nucleotides at the 3′ end. In some embodiments, a PEgRNA comprises 1, 2, 3, 4, 5, 6, 7, 8, 9, 10 or more contiguous chemically modified nucleotides at the 5′ end. In some embodiments, a PERNA comprises 1, 2, 3, 4, or 5 contiguous chemically modified nucleotides at the 3′ end. In some embodiments, a PEgRNA comprises 1, 2, 3, 4, or 5 contiguous chemically modified nucleotides at the 5′ end. In some embodiments, a PERNA comprises 1, 2, or 3 contiguous chemically modified nucleotides at the 3′ end. In some embodiments, a PEgRNA comprises 1, 2, or 3 contiguous chemically modified nucleotides at the 5′ end. In some embodiments, a PEgRNA comprises 3 contiguous chemically modified nucleotides at the 3′ end.

In some embodiments, a PEgRNA comprises 1, 2, 3, 4, 5, or more chemically modified nucleotides near the 3′ end. In some embodiments, a PERNA comprises 3 contiguous chemically modified nucleotides at the 3′ end. In some embodiments, a PEgRNA comprises 3 contiguous chemically modified nucleotides at the 5′ end. In some embodiments, a PEgRNA comprises 1, 2, 3, 4, 5, or more chemically modified nucleotides near the 3′ end. In some embodiments, a PEgRNA comprises 1, 2, 3, 4, 5, or more contiguous chemically modified nucleotides near the 3′ end. In some embodiments, a PEgRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA comprises one or more chemically modified nucleotides in the gRNA core. In some embodiments, the gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA can comprise a ‘-O-methyl (M) RNA, a 2’-O-methyl 3′phosphorothioate (MS) RNA, a 2′-O-methyl 3′thioPACE (MSP) RNA, a 2′-F RNA, a phosphorothioate bond modification, any other chemical modifications known in the art, or combination thereof. A chemical modification may also include, for example, the incorporation of non-nucleotide linkages or modified nucleotides into the PEgRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements. Components of a prime editing composition may be combined to form a complex for prime editing, or may be kept separately, e.g., for administration purposes.

In some embodiments, a prime editing composition comprises a first prime editing guide RNA (PEgRNA), a second PEgRNA, and a prime editor. In some embodiments, a prime editing composition comprises a first prime editing guide RNA (PEgRNA), a second PEgRNA, and a prime editor fusion protein complexed with the first PEgRNA and a prime editor fusion protein complexed with the second PEgRNA. In some embodiments, the prime editor fusion protein complexed with the first PEgRNA and the prime editor fusion protein complexed with the second PERNA are the identical prime editor fusion protein. In some embodiments, the prime editor fusion protein complexed with the first PEgRNA and the prime editor fusion protein complexed with the second PEgRNA are different prime editor fusion proteins.

In some embodiments, a prime editing composition comprises a first prime editing guide RNA (PEgRNA), a second PEgRNA, and a prime editor comprising a DNA binding domain and a DNA polymerase domain associated with each other through the first PEgRNA and/or the second PEgRNA. For example, the prime editing composition may comprise a prime editor comprising a DNA binding domain and a DNA polymerase domain linked to each other by an RNA-protein recruitment aptamer RNA sequence, which is linked to either or both of the first and second PEgRNAs. In some embodiments, the prime editing composition comprises a first PEgRNA, a second PEgRNA, and a prime editor comprising a DNA binding domain and a DNA polymerase domain, wherein the prime editor for both the first PEgRNA and the second PEgRNA are the same. In some embodiments, the prime editing composition comprises a first PEgRNA, a second PEgRNA, and a prime editor comprising a DNA binding domain and a DNA polymerase domain, wherein the prime editor for both the first PEgRNA and the second PERNA are different.

In some embodiments, a prime editing composition comprises a first PEgRNA, a second PEgRNA, and a polynucleotide, a polynucleotide construct, or a vector that encodes a prime editor fusion protein. In some embodiments, a prime editing composition comprises a first PEgRNA, a second PEgRNA, and one or more polynucleotides, one or more polynucleotide constructs, or one or more vectors that encode a prime editor comprising a DNA binding domain and a DNA polymerase domain. In some embodiments, a prime editing composition comprises a first PEgRNA, a second PERNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the first PEgRNA and/or the second PEgRNA of a prime editing composition is associated with the DNA binding domain, e.g., a Cas9 nickase, of the prime editor. In some embodiments, the first PEgRNA and/or the second PEgRNA of a prime editing composition complexes with the DNA binding domain of a prime editor and directs the prime editor to the double-stranded target DNA.

In some embodiments, a prime editing composition comprises one or more polynucleotides that encode prime editor components and/or the first PEgRNA and/or the second PEgRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, (ii) a first PEgRNA or a polynucleotide encoding the first PERNA, and (iii) a second PEgRNA or a polynucleotide encoding the second PEgRNA. In some embodiments, a prime editing composition consists of (i) a polynucleotide encoding a fusion protein comprising a DNA binding domain and a DNA polymerase domain, (ii) a first PEgRNA or a polynucleotide encoding the first PEgRNA, and (iii) a second PEgRNA or a polynucleotide encoding the second PERNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a first PEgRNA or a polynucleotide encoding the first PEgRNA, and (iv) a second PEgRNA or a polynucleotide encoding the second PEgRNA. In some embodiments, a prime editing composition consists of (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a first PEgRNA or a polynucleotide encoding the first PEgRNA, and (iv) a second PEgRNA or a polynucleotide encoding the second PEgRNA.

In some embodiments, the polynucleotide encoding the DNA binding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N (ii) a polynucleotide encoding a C-terminal half of a prime editor fusion protein and an intein-C, (iii) a first PEgRNA or a polynucleotide encoding the first PEgRNA, and (iv) a second PEgRNA or a polynucleotide encoding the second PEgRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a first PEgRNA or a polynucleotide encoding the first PEgRNA, and (iv) a second PEgRNA or a polynucleotide encoding the second PEgRNA.

The editing template of the first PERNA (the “first editing template”) and the editing template of the second PEgRNA (the “second editing template”) of a prime editing system may or may not have sequence complementarity to each other. In some embodiments, the first editing template has a region of complementarity or substantial complementarity to the second editing template. In some embodiments, the region of complementarity or substantial complementarity to the second editing template is 2, 3, 4, 5, 6, 7, 8, 9, 10, 11, 12, 13, 14, 15, 16, 17, 18, 19, 20, 21, 22, 23, 24, 25, 30, 35, 40, 45, 50 or more nucleotides in length. In some embodiments, the region of complementarity or substantial complementarity to the second editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 25 to 30, 25 to 35, 25 to 40, 25 to 45, 25 to 50, 25 to 55, 25 to 60, 25 to 65, 25 to 70, 25 to 75, 25 to 80, 25 to 85, 25 to 90, 25 to 95, 25 to 100, 25 to 110, 25 to 120, 25 to 130, 25 to 140, 25 to 150, 35 to 40, 35 to 45, 35 to 50, 35 to 55, 35 to 60, 35 to 65, 35 to 70, 35 to 75, 35 to 80, 35 to 85, 35 to 90, 35 to 95, 35 to 100, 35 to 110, 35 to 120, 35 to 130, 35 to 140, 35 to 150, 45 to 50, 45 to 55, 45 to 60, 45 to 65, 45 to 70, 45 to 75, 45 to 80, 45 to 85, 45 to 90, 45 to 95, 45 to 100, 45 to 110, 45 to 120, 45 to 130, 45 to 140, 045 to 150, 55 to 60, 55 to 65, 55 to 70, 55 to 75, 55 to 80, 55 to 85, 55 to 90, 55 to 95, 55 to 100, 55 to 110, 55 to 120, 55 to 130, 55 to 140, 55 to 150, 65 to 70, 65 to 75, 65 to 80, 65 to 85, 65 to 90, 65 to 95, 65 to 100, 65 to 110, 65 to 120, 65 to 130, 65 to 140, 65 to 150, 75 to 80, 75 to 85, 75 to 90, 75 to 95, 75 to 100, 75 to 110, 75 to 120, 75 to 130, 75 to 140, 75 to 150, 85 to 90, 85 to 95, 85 to 100, 85 to 110, 85 to 120, 85 to 130, 85 to 140, 85 to 150, 95 to 100, 95 to 110, 95 to 120, 95 to 130, 95 to 140, 95 to 150, 105 to 110, 105 to 120, 105 to 130, 105 to 140, 105 to 150, 115 to 120, 115 to 130, 115 to 140, 115 to 150, 125 to 130, 125 to 140, 125 to 150, 135 to 140, 135 to 150, or 145 to 150 nucleotides in length. In some embodiments, the region of complementarity or substantial complementarity to the second editing template is 10, 11, 12, 13, 14, 15, 16, 17, 18, 19, 20, 21, 22, 23, 24 or 25 nucleotides in length. In some embodiments, the region of complementarity or substantial complementarity to the second editing template is 30, 35, 40, 50, 60, 70, 80, 90, or 100 nucleotides in length. In some embodiments, the region of complementarity or substantial complementarity to the second editing template is at most 20, 25, 30, 35, 40, 50, 60, 70, 80, 90, or 100 nucleotides in length. In some embodiments, the region of complementarity or substantial complementarity to the second editing template is at least 10 nucleotides in length. In some embodiments, the region of complementarity or substantial complementarity to the second editing template is at least 15 nucleotides in length. In some embodiments, the region of complementarity or substantial complementarity to the second editing template is at least 20 nucleotides in length.

In some embodiments, the first editing template has a region of complementarity to the second editing template and does not have a region of complementarity or substantial complementarity to the target double-stranded DNA sequence. In some embodiments, the second editing template has a region of complementarity to the first editing template and does not have a region of complementarity or substantial complementarity to the target double-stranded DNA sequence.

In some embodiments, the first editing template comprises a region of complementarity or substantial complementarity to the target double-stranded DNA sequence. In some embodiments, the first editing template has a region of complementarity or substantial complementarity to the second editing template and has a region of complementarity or substantial complementarity to the target double-stranded DNA sequence.

In some embodiments, the second editing template comprises a region of complementarity or substantial complementarity to the target double-stranded DNA sequence. In some embodiments, the second editing template has a region of complementarity to the first editing template and has a region of complementarity or substantial complementarity to the target double-stranded DNA sequence.

The region of complementarity or substantial complementarity of the first editing template to the double-stranded target DNA sequence may or may not have the same length as the region of complementarity or substantial complementarity of the second editing template to the double-stranded target DNA sequence.

In some embodiments, the region of complementarity or substantial complementarity of the first editing template to the target double-stranded DNA sequence is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of complementarity or substantial complementarity of the first editing template to the target double-stranded DNA sequence is about 10 nucleotides in length. In some embodiments, the region of complementarity or substantial complementarity of the first editing template to the target double-stranded DNA sequence is about 15 nucleotides in length. In some embodiments, the region of complementarity or substantial complementarity of the first editing template to the target double-stranded DNA sequence is about 20 nucleotides in length. In some embodiments, the region of complementarity or substantial complementarity of the first editing template to the target double-stranded DNA sequence is about 21, 22, 23, 24, or 25 nucleotides in length.

In some embodiments, the region of complementarity or substantial complementarity of the second editing template to the target double-stranded DNA sequence is about 5 to 10, 5 to 15, 5 to 20, 5 to 25, 5 to 30, 5 to 35, 5 to 40, 5 to 45, 5 to 50, to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of complementarity or substantial complementarity of the second editing template to the target double-stranded DNA sequence is about 10 nucleotides in length. In some embodiments, the region of complementarity or substantial complementarity of the second editing template to the target double-stranded DNA sequence is about 15 nucleotides in length. In some embodiments, the region of complementarity or substantial complementarity of the second editing template to the target double-stranded DNA sequence is about 20 nucleotides in length. In some embodiments, the region of complementarity or substantial complementarity of the second editing template to the target double-stranded DNA sequence is about 21, 22, 23, 24, or 25 nucleotides in length.

In some embodiments, the first editing template comprises a region that does not have complementarity or substantial complementarity (the non-complementarity region) to the second editing template. In some embodiments, the first editing template comprises a region of complementarity or substantial complementarity to the second editing template, and further comprises a non-complementarity region to the second editing template. In some embodiments, the first editing template comprises a region of complementarity or substantial complementarity to the second editing template, a non-complementarity region to the second editing template, and a region of complementarity or substantial complementarity to the target double-stranded DNA sequence.

In some embodiments, the second editing template comprises a region that does not have complementarity or substantial complementarity (the non-complementarity region) to the first editing template. In some embodiments, the second editing template comprises a region of complementarity or substantial complementarity to the first editing template, and further comprises a non-complementarity region to the first editing template. In some embodiments, the second editing template comprises a region of complementarity or substantial complementarity to the first editing template, a non-complementarity region to the first editing template, and a region of complementarity or substantial complementarity to the target double-stranded DNA sequence.

In some embodiments, the region of non-complementarity of the first editing template to the second editing template and the region of non-complementarity of the second editing template to the first editing template are of the same length. In some embodiments, the region of non-complementarity of the first editing template to the second editing template and the region of non-complementarity of the second editing template to the first editing template are of different lengths. In some embodiments, the first editing template and the second editing template both comprise a region of non-complementarity to each other. In some embodiments, the first editing template comprises a region of non-complementarity to the second editing template, and the second editing template does not comprise a region of non-complementarity to the first editing template. In some embodiments, the second editing template comprises a region of non-complementarity to the first editing template, and the first editing template does not comprise a region of non-complementarity to the second editing template. For example, the first editing template may be complementary or substantially complementary to the second editing template through its entire length, while the second editing template comprises a region that does not have complementarity to the first editing template, or vice versa.

In some embodiments, the region of non-complementarity of the first editing template to the second editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of non-complementarity of the first editing template to the second editing template is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the region of non-complementarity of the second editing template to the first editing template is about 5 to 10, 5 to 15, 5 to 20, 5 to 25, 5 to 30, 5 to 35, 5 to 40, 5 to 45, 5 to 50, 5 to 55, 5 to 60, 5 to 65, 5 to 70, 5 to 75, 5 to 80, 5 to 85, 5 to 90, 5 to 95, 5 to 100, 5 to 110, 5 to 120, 5 to 130, 5 to 140, 5 to 150, 5 to 175, 5 to 200, 5 to 225, 5 to 250, 5 to 275, 5 to 300, 5 to 325, 5 to 350, 5 to 375, 5 to 400, 5 to 425, 5 to 450, 5 to 475, 5 to 500, 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, 10 to 80, 10 to 85, 10 to 90, 10 to 95, 10 to 100, 10 to 110, 10 to 120, 10 to 130, 10 to 140, 10 to 150, 10 to 175, 10 to 200, 10 to 225, 10 to 250, 10 to 275, 10 to 300, 10 to 325, 10 to 350, 10 to 375, 10 to 400, 10 to 425, 10 to 450, 10 to 475, 10 to 500, 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, 15 to 95, 15 to 100, 15 to 110, 15 to 120, 15 to 130, 15 to 140, 15 to 150, 15 to 175, 15 to 200, 15 to 225, 15 to 250, 15 to 275, 15 to 300, 15 to 325, 15 to 350, 15 to 375, 15 to 400, 15 to 425, 15 to 450, 15 to 475, 15 to 500, 20 to 25, 20 to 30, 20 to 35, 20 to 40, 20 to 45, 20 to 50, 20 to 55, 20 to 60, 20 to 65, 20 to 70, 20 to 75, 20 to 80, 20 to 85, 20 to 90, 20 to 95, 20 to 100, 20 to 110, 20 to 120, 20 to 130, 20 to 140, 20 to 150, 20 to 175, 20 to 200, 20 to 225, 20 to 250, 20 to 275, 20 to 300, 20 to 325, 20 to 350, 20 to 375, 20 to 400, 20 to 425, 20 to 450, 20 to 475, 20 to 500, 30 to 35, 30 to 40, 30 to 45, 30 to 50, 30 to 55, 30 to 60, 30 to 65, 30 to 70, 30 to 75, 30 to 80, 30 to 85, 30 to 90, 30 to 95, 30 to 100, 30 to 110, 30 to 120, 30 to 130, 30 to 140, 30 to 150, 30 to 175, 30 to 200, 30 to 225, 30 to 250, 30 to 275, 30 to 300, 30 to 325, 30 to 350, 30 to 375, 30 to 400, 30 to 425, 30 to 450, 30 to 475, 30 to 500, 40 to 45, 40 to 50, 40 to 55, 40 to 60, 40 to 65, 40 to 70, 40 to 75, 40 to 80, 40 to 85, 40 to 90, 40 to 95, 40 to 100, 40 to 110, 40 to 120, 40 to 130, 40 to 140, 40 to 150, 40 to 175, 40 to 200, 40 to 225, 40 to 250, 40 to 275, 40 to 300, 40 to 325, 40 to 350, 40 to 375, 40 to 400, 40 to 425, 40 to 450, 40 to 475, 40 to 500, 50 to 55, 50 to 60, 50 to 65, 50 to 70, 50 to 75, 50 to 80, 50 to 85, 50 to 90, 50 to 95, 50 to 100, 50 to 110, 50 to 120, 50 to 130, 50 to 140, 50 to 150, 50 to 175, 50 to 200, 50 to 225, 50 to 250, 50 to 275, 50 to 300, 50 to 325, 50 to 350, 50 to 375, 50 to 400, 50 to 425, 50 to 450, 50 to 475, 50 to 500, 75 to 80, 75 to 85, 75 to 90, 75 to 95, 75 to 100, 75 to 110, 75 to 120, 75 to 130, 75 to 140, 75 to 150, 75 to 175, 75 to 200, 75 to 225, 75 to 250, 75 to 275, 75 to 300, 75 to 325, 75 to 350, 75 to 375, 75 to 400, 75 to 425, 75 to 450, 75 to 475, 75 to 500, 100 to 110, 100 to 120, 100 to 130, 100 to 140, 100 to 150, 100 to 175, 100 to 200, 100 to 225, 100 to 250, 100 to 275, 100 to 300, 100 to 325, 100 to 350, 100 to 375, 100 to 400, 100 to 425, 100 to 450, 100 to 475, 100 to 500, 125 to 150, 125 to 175, 125 to 200, 125 to 225, 125 to 250, 125 to 275, 125 to 300, 125 to 325, 125 to 350, 125 to 375, 125 to 400, 125 to 425, 125 to 450, 125 to 475, 125 to 500, 150 to 175, 150 to 200, 150 to 225, 150 to 250, 150 to 275, 150 to 300, 150 to 325, 150 to 350, 150 to 375, 150 to 400, 150 to 425, 150 to 450, 150 to 475, 150 to 500, 175 to 200, 175 to 225, 175 to 250, 175 to 275, 175 to 300, 175 to 325, 175 to 350, 175 to 375, 175 to 400, 175 to 425, 175 to 450, 175 to 475, 175 to 500, 200 to 250, 200 to 275, 200 to 300, 200 to 325, 200 to 350, 200 to 375, 200 to 400, 200 to 425, 200 to 450, 200 to 475, 200 to 500, 225 to 250, 225 to 275, 225 to 300, 225 to 325, 225 to 350, 225 to 375, 225 to 400, 225 to 425, 225 to 450, 225 to 475, 225 to 500, 250 to 275, 250 to 300, 275 to 300, 275 to 325, 275 to 350, 275 to 375, 275 to 400, 275 to 425, 275 to 450, 275 to 475, 275 to 500, 300 to 325, 300 to 350, 300 to 375, 300 to 400, 300 to 425, 300 to 450, 300 to 475, 300 to 500, 325 to 350, 325 to 375, 325 to 400, 325 to 425, 325 to 450, 325 to 475, 325 to 500, 350 to 375, 350 to 400, 350 to 425, 350 to 450, 350 to 475, 350 to 500, 375 to 400, 375 to 425, 375 to 450, 375 to 475, 375 to 500, 400 to 425, 400 to 450, 400 to 475, 400 to 500, 425 to 450, 425 to 475, 425 to 500, 450 to 475, 450 to 500, or 475 to 500 nucleotides in length. In some embodiments, the region of non-complementarity of the second editing template to the first editing template is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, 200, 210, 220,230, 240, 250, 260, 270, 280, 290, 300, 310, 320, 330, 340, 350, 360, 370, 380, 390, 400, 410, 420, 430, 440, 450, 460, 470, 480, 490, or 500 or more nucleotides in length.

In some embodiments, the first PEgRNA and the second PEgRNA have the same gRNA cores. In some embodiments, the first PEgRNA and the second PEgRNA have different gRNA cores.

In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PERNA or both PEgRNAs may be delivered simultaneously. For example, in some embodiments, a polynucleotide encoding the prime editor and a polynucleotide encoding a PEgRNA or both PEgRNAs may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

Provided herein in some embodiments are example sequences for PERNAs, including PEgRNA spacers, PBS, and editing templates for a prime editing system comprising a nuclease that recognizes the PAM sequence “NG.” In some embodiments, a PAM motif on the edit strand comprises an “NG” motif, wherein N is any nucleotide.

In some embodiments, a PEgRNA of this disclosure is part of a prime editing system that recognizes the PAM motif GG and comprises a spacer as set forth in SEQ ID NOs: 65--74, a PBS sequence selected from the group consisting of SEQ ID NOs: 75-104, and an RTT sequence selected from the group consisting of SEQ ID NOs: 105-144. In some embodiments, a PEgRNA is part of a prime editing system that recognizes the PAM motif GG and comprises a spacer as set forth in SEQ ID NOs: 65-74, a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386, e.g., SEQ ID NO: 301. In some embodiments, the PEgRNA is part of a prime editing system that recognizes the PAM motif GG and comprises, contiguously from 5′ to 3′: a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386.

Provided herein in some embodiments are example sequences for PEgRNA spacers, PBS, and editing templates for a prime editing system comprising a nuclease that recognizes the PAM sequence “NAG.” In some embodiments, a PAM motif on the edit strand comprises an “NAG” motif, wherein N is any nucleotide.

In some embodiments, a PERNA of this disclosure is part of a prime editing system that recognizes the PAM motif GAG and comprises a spacer as set forth in SEQ ID NOs: 65-74, a PBS sequence selected from the group consisting of SEQ ID NOs: 75-104, and an RTT sequence selected from the group consisting of SEQ ID NOs: 105-144. In some embodiments, a PEgRNA is part of a prime editing system that recognizes the PAM motif GAG and comprises a spacer as set forth in SEQ ID NOs: 65-74, a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386, e.g., SEQ ID NO: 301. In some embodiments, the PEgRNA is part of a prime editing system that recognizes the PAM motif GAG and comprises, contiguously from 5′ to 3′: a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386.

Provided herein in some embodiments are example sequences for PERNA spacers, PBS, and editing templates for a prime editing system comprising a nuclease that recognizes the PAM sequence “NGA.” In some embodiments, a PAM motif on the edit strand comprises an “NGA” motif, wherein N is any nucleotide.

In some embodiments, a PEgRNA of this disclosure is part of a prime editing system that recognizes the PAM motif GGA and comprises a spacer as set forth in SEQ ID NOs: 65-74, a PBS sequence selected from the group consisting of SEQ ID NOs: 75-104, and an RTT sequence selected from the group consisting of SEQ ID NOs: 105-144. In some embodiments, a PEgRNA is part of a prime editing system that recognizes the PAM motif GGA and comprises a spacer as set forth in SEQ ID NOs: 65-74, a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386, e.g., SEQ ID NO: 301. In some embodiments, the PEgRNA is part of a prime editing system that recognizes the PAM motif GGA and comprises, contiguously from 5′ to 3′: a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386.

Provided herein in some embodiments are example sequences for PERNA spacers, PBS, and editing templates for a prime editing system comprising a nuclease that recognizes the PAM sequence “NGG.” In some embodiments, a PAM motif on the edit strand comprises an “NGG” motif, wherein N is any nucleotide.

In some embodiments, a PEgRNA of this disclosure is part of a prime editing system that recognizes the PAM motif CGG and comprises a spacer as set forth in SEQ ID NOs: 65-74, a PBS sequence selected from the group consisting of SEQ ID NOs: 75-104, and an RTT sequence selected from the group consisting of SEQ ID NOs: 105-144. In some embodiments, a PEgRNA is part of a prime editing system that recognizes the PAM motif CGG and comprises a spacer as set forth in SEQ ID NOs: 65-74, a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386, e.g., SEQ ID NO: 301. In some embodiments, the PERNA is part of a prime editing system that recognizes the PAM motif CGG and comprises, contiguously from 5′ to 3′: a PBS sequence selected from the group consisting of SEQ ID NOs: 75-104, an RTT sequence selected from the group consisting of SEQ ID NOs: 105-144, and a gRNA core sequence selected from the group consisting of SEQ ID NOs: 301-305 and 381-386.

Provided herein in some embodiments are example sequences for PEgRNA spacers, PBS, and editing templates for a prime editor complex comprising a nuclease that recognizes the PAM sequence “NNGG.” In some embodiments, a PAM motif on the edit strand comprises an “NNGG” motif, wherein N is any nucleotide.

Provided herein in some embodiments are example sequences for PEgRNA spacers, PBS, and editing templates for a prime editing system comprising a nuclease that recognizes the PAM sequence “NNGRRT.” In some embodiments, a PAM motif on the edit strand comprises an “NNGRRT” motif, wherein N is any nucleotide and R is A or G.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, and physiologic pH)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by dual prime editing.

In some embodiments, the dual prime editing method comprises contacting a target gene, e.g., a TCF4 gene, with a first PEgRNA, a second PEgRNA and a prime editor (PE) polypeptide described herein. In some embodiments, the target gene is double-stranded, and comprises two strands of DNA complementary to each other. In some embodiments, the contacting with the two PEgRNAs and the contacting with a prime editor are performed sequentially. In some embodiments, the contacting with a prime editor is performed after the contacting with the two PEgRNAs. In some embodiments, the contacting with the two PEgRNAs is performed after the contacting with a prime editor. In some embodiments, the contacting with the two PEgRNAs is performed simultaneously either prior to or after contacting with a prime editor. In some embodiments, the contacting with the two PERNAs is performed sequentially either prior to or after contacting with a prime editor. In some embodiments, the contacting with the two PEgRNAs, and the contacting with a prime editor are performed simultaneously. In some embodiments, the two PEgRNAs and the prime editor are associated in complexes prior to contacting a target gene.

In some embodiments, contacting the target gene with the prime editing composition results in binding of the first PEgRNA to a first strand of the target gene, e.g., a TCF4 gene. In some embodiments, contacting the target gene with the prime editing composition results in binding of the second PEgRNA to a second strand of the target gene, e.g., a TCF4 gene. In some embodiments, contacting the target gene with the prime editing composition results in binding of the first PEgRNA to a first strand of the target gene and binding of the second PEgRNA to a second strand of the target gene, e.g., a TCF4 gene. In some embodiments, contacting the target gene with the prime editing composition results in binding of the first PEgRNA to a first search target sequence on the first strand of the target gene upon contacting with the first PEgRNA. In some embodiments, contacting the target gene with the prime editing composition results in binding of the second PEgRNA to a second search target sequence on the second strand of the target gene upon contacting with the second PEgRNA. In some embodiments, contacting the target gene with the prime editing composition results in binding of the first PEgRNA to a first search target sequence on the first strand of the target gene upon contacting with the first PERNA and binding of the second PEgRNA to a second search target sequence on the second strand of the target gene upon contacting with the second PERNA. In some embodiments, contacting the target gene with the prime editing composition results in binding of a first spacer of the first PEgRNA to a first search target sequence on the first strand of the target gene upon said contacting of the first PEgRNA. In some embodiments, contacting the target gene with the prime editing composition results in binding of a second spacer of the second PEgRNA to a second search target sequence on the second strand of the target gene upon said contacting of the second PEgRNA. In some embodiments, contacting the target gene with the prime editing composition results in binding of a first spacer of the first PEgRNA to a first search target sequence on the first strand of the target gene upon said contacting of the first PEgRNA and binding of a second spacer of the second PEgRNA to a second search target sequence on the second strand of the target gene upon said contacting of the second PEgRNA.

In some embodiments, contacting the target gene with the prime editing composition results in binding of the prime editor to the target gene, e.g., the target TCF4 gene, upon the contacting of the PE composition with the target gene. In some embodiments, a DNA binding domain of a prime editor associates with either a first PERNA and/or a second PEgRNA. In some embodiments, a prime editor associated with a first PEgRNA binds the first strand of a target gene, e.g., a TCF4 gene, as directed by the first PEgRNA. In some embodiments, a prime editor associated with a second PEgRNA binds the second strand of a target gene, e.g., a TCF4 gene, as directed by the second PEgRNA. In some embodiments, a prime editor associated with a first PEgRNA binds the first strand of a target gene as directed by the first PERNA, and a prime editor associated with a second PEgRNA binds the second strand of the target gene as directed by the second PEgRNA.

In some embodiments, a first PEgRNA directs a prime editor to generate a nick on the second strand of a target gene. In some embodiments, a second PEgRNA directs a prime editor to generate a nick on the first strand of a target gene. In some embodiments, a first PEgRNA directs a prime editor to generate a first nick on the second strand of a target gene, and a second PEgRNA directs a prime editor to generate a second nick on the first strand of a target gene, thereby generating an inter-nick duplex (IND) between the position of the first nick and the position of the second nick on the target gene. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA (e.g., the first PEgRNA and/or the second PEgRNA) with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an overlap duplex (OD) or replacement duplex (RD) that replaces the IND. In some embodiments, the OD or RD comprises one or more intended nucleotide edits compared to the endogenous sequence of the target gene, e.g., a TCF4 gene. In some embodiments, the intended nucleotide edits are incorporated in the target gene by replacement of the IND by the OD or RD. In some embodiments, the intended nucleotide edits are incorporated in the target gene by excision of the IND and DNA repair. In some embodiments, excision of the 5′ single-stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, the target gene, e.g., a TCF4 gene, is in a cell. Accordingly, also provided herein are methods of modifying a cell.

In some embodiments, the prime editing method comprises introducing a first PEgRNA, a second PEgRNA, and a prime editor into the cell that has the target gene. In some embodiments, the prime editing method comprises introducing into the cell that has the target gene with a prime editing composition comprising a first PEgRNA, a second PEgRNA, and a prime editor polypeptide. In some embodiments, the first PEgRNA, the second PEgRNA, and the prime editor polypeptides form complexes prior to the introduction into the cell. In some embodiments, the first PERNA, the second PEgRNA, and the prime editor polypeptides form complexes after the introduction into the cell. The prime editors, PEgRNAs and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNAs, and prime editing complexes may be introduced into the cell simultaneously or sequentially.

In some embodiments, the prime editing method comprises introducing into the cell a first PEgRNA and a second PEgRNA, or polynucleotides encoding the first PEgRNA and the second PEgRNA, and a prime editor polynucleotide encoding a prime editor polypeptide. In some embodiments, the method comprises introducing the first PEgRNA and the second PEgRNA or the polynucleotides encoding the first PEgRNA and the second PEgRNA, and the polynucleotide encoding the prime editor polypeptide into the cell simultaneously. In some embodiments, the method comprises introducing the first PEgRNA and the second PEgRNA or the polynucleotides encoding the first PEgRNA and the second PEgRNA, and the polynucleotide encoding the prime editor polypeptide into the cell sequentially. In some embodiments, the method comprises introducing the polynucleotide encoding the prime editor polypeptide into the cell before introduction of the first PEgRNA and the second PEgRNA or the polynucleotides encoding the first PEgRNA and the second PEgRNA. In some embodiments, the polynucleotide encoding the prime editor polypeptide is introduced into and expressed in the cell before introduction of the first PEgRNA and the second PEgRNA or the polynucleotides encoding the first PEgRNA and the second PEgRNA into the cell. In some embodiments, the polynucleotide encoding the prime editor polypeptide is introduced into the cell after the first PEgRNA and the second PEgRNA or the polynucleotides encoding the first PEgRNA and the second PEgRNA are introduced into the cell. The polynucleotide encoding the prime editor polypeptide, the first PEgRNA and the second PEgRNA or the polynucleotides encoding the first PEgRNA and the second PERNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide and the polynucleotides encoding the first PEgRNA and the second PEgRNA integrate into the genome of the cell after being introduced into the cell. In some embodiments, the polynucleotide encoding the prime editor polypeptide and the polynucleotides encoding the first PEgRNA and the second PEgRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent, or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a corneal endothelial cell. In some embodiments, the cell is a primary corneal endothelial cell. In some embodiments, the cell is a human corneal endothelial cell. In some embodiments, the cell is a primary human corneal endothelial cell. In some embodiments, the cell is a primary human corneal endothelial cell derived from an induced human pluripotent stem cell (iPSC). In some embodiments, the cell is a corneal epithelial cell. In some embodiments, the cell is a primary corneal epithelial cell. In some embodiments, the cell is a human corneal epithelial cell. In some embodiments, the cell is a primary human corneal epithelial cell. In some embodiments, the cell is a primary human corneal epithelial cell derived from an induced human pluripotent stem cell (iPSC). In some embodiments, the cell is a fibroblast. In some embodiments, the cell is a human corneal endothelial progenitor cell. In some embodiments, the cell is a human corneal endothelial progenitor cell. In some embodiments, the cell is a differentiated corneal endothelial cell. In some embodiments, the differentiated corneal endothelial cell is differentiated from an iPSC, ESC or a corneal endothelial cell progenitor cell. In some embodiments, the cell is a human corneal epithelial progenitor cell or stem cell. In some embodiments, the cell is a human corneal epithelial progenitor cell. In some embodiments, the cell is a differentiated corneal epithelial cell. In some embodiments, the differentiated corneal epithelial cell is differentiated from an iPSC, ESC or a corneal epithelial cell progenitor cell.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, and the PEgRNAs (e.g., the first PEgRNA and the second PEgRNA) or the polynucleotides encoding the PEgRNAs, into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, and the PERNAs (e.g., the first PEgRNA and the second PEgRNA) or the polynucleotides encoding the PEgRNAs, results in incorporation of one or more intended nucleotide edits in the target gene in at least one of the cells in the population of cells. In some embodiments, introduction of the prime editor polypeptide or the polynucleotide encoding the prime editor polypeptide, and the PEgRNAs or the polynucleotides encoding the PEgRNAs, results in incorporation of the one or more intended nucleotide edits in the target gene in a plurality of the population of cells. In some embodiments, introduction of the prime editor polypeptide or the polynucleotide encoding the prime editor polypeptide, and the PEgRNAs or the polynucleotide encoding the PEgRNAs, results in incorporation of the one or more intended nucleotide edits in the target gene in each cell of the population of cells. In some embodiments, introduction of the prime editor polypeptide or the polynucleotide encoding the prime editor polypeptide, and the PEgRNAs or the polynucleotides encoding the PEgRNAs, results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. The percentage of edited target genes can be assessed in any method known in the art, for example, with a next generation sequencing platform (e.g. Miseq) and suitable primers, by the percentage of edited reads over total sequenced reads. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a TCF4 gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the dual prime editing compositions and methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99%. In some embodiments, the prime editing methods disclosed herein have an editing efficiency of at least 25%. In some embodiments, the prime editing method disclosed herein has an editing efficiency of at least 30%. In some embodiments, the prime editing methods disclosed herein have an editing efficiency of at least 35%. In some embodiments, the prime editing methods disclosed herein have an editing efficiency of at least 45%. In some embodiments, the prime editing methods disclosed herein have an editing efficiency of at least 50%.

In some embodiments, the dual prime editing compositions and methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, at least about 95%, or at least about 99% of editing a primary cell.

In some embodiments, the dual primer editing compositions and methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, at least about 95%, or at least about 99% of editing, for example, a corneal endothelial cell relative to a corresponding control corneal endothelial cell. In some embodiments, the corneal endothelial cell is a human corneal endothelial cell.

In some embodiments, the dual prime editing compositions and methods provided herein are capable of incorporating one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37 (3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a TCF4 gene within the genome of a cell) to a prime editing composition.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 5% and an indel frequency of less about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 7.5% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 10% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 15% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 20% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 30% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 40% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 50% and an indel frequency of less than about 5%, less than about 2.5%, less than about 1%, less than about 0.5%, or less than about 0.1% in a target cell, e.g., a human primary cell or corneal endothelial cell. In some embodiments, the prime editing methods disclosed herein have an editing efficiency of at least about 10% and an indel frequency of less than 0.5% in a target cell, e.g., a human primary cell or corneal endothelial cell.

In some embodiments, the prime editing methods disclosed herein have an editing efficiency of at least about 60% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 70% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 80% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 90% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing methods disclosed herein have an editing efficiency of at least about 95% and an indel frequency of less than about 5%, less than about 2.5%, less than about 1%, less than about 0.5%, or less than about 0.1% in a target cell, e.g., a human primary cell or corneal endothelial cell.

In some embodiments, the prime editing composition described herein results in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., the PEgRNAs and prime editors as described herein) and dual prime editing methods disclosed herein can be used to edit a target TCF4 gene. In some embodiments, the target TCF4 gene comprises a mutation compared to a wild-type TCF4 gene. In some embodiments, the mutation is associated with FECD. In some embodiments, the target TCF4 gene comprises an IND sequence that contains the mutation associated with FECD. In some embodiments, the PEgRNAs of the prime editing compositions direct replacement of an edited portion of a TCF4 gene into the TCF4 gene. In some embodiments, the mutation is associated with FECD. In some embodiments, the mutation is in intron 3 of the TCF4 gene. In some embodiments, the mutation is expansion of the number of CTG repeats in intron 3 of the TCF4 gene. In some embodiments, the mutation is an increased number of tri-nucleotide repeats in the array of tri-nucleotide repeats compared to a wild-type TCF4 gene. In some embodiments, the mutation is an array of tri-nucleotide repeats comprising the sequence (CTG) n or a complementary sequence thereof, wherein n is any integer greater than 39. In some embodiments, n is an integer greater than 49. In some embodiments, n is an integer greater than 99. In some embodiments, n is an integer greater than 200, 300, 400, 500, 600, 700, 800, 900, or 1000. In some embodiments, n is an integer greater than 1000. In some embodiments, the prime editing method comprises contacting a target TCF4 gene with a prime editing composition comprising a prime editor, a first PEgRNA and a second PEgRNA. In some embodiments, contacting the target TCF4 gene with the prime editing composition results in incorporation of one or more intended nucleotide edits in the target TCF4 gene. In some embodiments, the incorporation is in a region of the target TCF4 gene that corresponds to an IND in the TCF4 gene. In some embodiments, the one or more intended nucleotide edits comprises a nucleotide substitution, an insertion, a deletion, or any combination thereof, compared to the endogenous sequence of the target TCF4 gene. In some embodiments, incorporation of the one or more intended nucleotide edits results in replacement of the one or more mutations with the corresponding sequence in a wild-type TCF4 gene. In some embodiments, incorporation of the one or more intended nucleotide edits results in correction of a mutation in the target TCF4 gene. In some embodiments, the target TCF4 gene comprises an IND sequence that contains the mutation. In some embodiments, contacting the target TCF4 gene with the prime editing composition results in incorporation of one or more intended nucleotide edits in the target TCF4 gene, which corrects the mutation in the IND in the target TCF4 gene.

In some embodiments, a population of patients with mutations in the target TCF4 gene may be treated with a prime editing composition (e.g., the pair of PEgRNAs and a prime editor as described herein) disclosed herein. In some embodiments, a population of patients with different distinct mutations in the target TCF4 gene can be treated with a single prime editing composition comprising the same pair of PEgRNAs and a prime editor. In some embodiments, a single prime editing composition comprising the same pair of PEgRNAs and a prime editor can be used to correct one or more mutations in the target TCF4 gene in a populations of patients, wherein one or more patients in the population have different mutations from one another. In some embodiments, the prime editing composition comprising the same pair of PEgRNAs and a prime editor can be used to correct two or more mutations in the target TCF4 gene in a populations of patients, wherein one or more patients in the population have different mutations from one another. In some embodiments, the prime editing composition comprising the same pair of PEgRNAs and a prime editor can be used to correct 2, 3, 4, 5, 6, 7, 8, 9, 10, 15, 20, 25 or more mutations in the target TCF4 gene in a populations of patients, wherein one or more patients in the population have different mutations from one another. In some embodiments, the prime editing composition comprising the same pair of PEgRNAs and a prime editor can be used to correct 30, 35, 40, 45, 50, 60 more mutations in the target TCF4 gene in a populations of patients, wherein one or more patients in the population have different mutations from one another. In some embodiments, the first PEgRNA in the pair of PEgRNAs comprises a first editing template comprising a wild-type sequence of the TCF4 gene. In some embodiments, the second PEgRNA in the pair of PEgRNAs comprises a second editing template comprising a wild-type sequence of the TCF4 gene.

In some embodiments, a patient with multiple mutations in the target TCF4 gene may be treated with a prime editing composition (e.g., the pair of PEgRNAs and a prime editor as described herein) disclosed herein. For example, in some embodiments, a subject may comprise two copies of the TCF4 gene, each comprising one or more different mutations. In some embodiments, a patient with one or more different mutations in the target TCF4 gene can be treated with a single prime editing composition comprising a pair of PEgRNAs and a prime editor.

In some embodiments, the dual prime editing composition can be used to correct all of the mutations in a portion of the TCF4 gene. In some embodiments, the dual prime editing composition can be used to correct all of the mutations in the entire TCF4 gene.

In some embodiments, incorporation of the one or more intended nucleotide edits results in correction of a mutation in intron 3 of the TCF4 gene. In some embodiments, the mutation is associated with FECD. In some embodiments, the mutation is expansion of the number of CTG repeats in intron 3 of the TCF4 gene. In some embodiments, the mutation is an increased number of tri-nucleotide repeats in the array of tri-nucleotide repeats compared to a wild-type TCF4 gene. In some embodiments, the mutation is an array of tri-nucleotide repeats comprising the sequence (CTG), or a complementary sequence thereof, wherein n is any integer greater than 39. In some embodiments, n is an integer greater than 49. In some embodiments, n is an integer greater than 99. In some embodiments, n is an integer greater than 200, 300, 400, 500, 600, 700, 800, 900, or 1000. In some embodiments, n is an integer greater than 1000. In some embodiments, incorporation of the one or more intended nucleotide edits results in deletion of the CTG repeats in intron 3 of the TCF4 gene entirely. In some embodiments, incorporation of the one or more intended nucleotide edits results in reduced number of CTG repeats in intron 3 of the TCF4 gene. In some embodiments, incorporation of the one or more intended nucleotide edits results in the number of CTG repeats in intron 3 of the TCF4 gene to less than 39. In some embodiments, incorporation of the one or more intended nucleotide edits results in the number of CTG repeats in intron 3 of the TCF4 gene to less than 34. In some embodiments, incorporation of the one or more intended nucleotide edits results in the number of CTG repeats in intron 3 of the TCF4 gene to less than 30, 20, or 10. In some embodiments, incorporation of the one or more intended nucleotide edits results in the number of CTG repeats in intron 3 of the TCF4 gene to 5. In some embodiments, incorporation of the one or more intended nucleotide edits results in correction of a TCF4 gene sequence and restores expression level of wild-type TCF4 transcripts.

In some embodiments, the target TCF4 gene is in a target cell. Accordingly, in one aspect provided herein is a method of editing a target cell comprising a target TCF4 gene that encodes a polypeptide that comprises one or more mutations relative to a wild-type TCF4 gene. In some embodiments, the methods of the present disclosure comprise introducing a prime editing composition comprising a pair of PEgRNAs (i.e., first PEgRNA and a second PEgRNA), and a prime editor polypeptide into the target cell that has the target TCF4 gene to edit the target TCF4 gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a corneal endothelial cell. In some embodiments, the target cell is a human corneal endothelial cell. In some embodiments, the target cell is a primary human corneal endothelial cell derived from an induced human pluripotent stem cell (iPSC). In some embodiments, the cell is an epithelial cell. In some embodiments, the cell is aneuron epithelial cell from a subject. In some embodiments, the cell an epithelial stem cell of a subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target TCF4 gene that comprises one or more mutations restores wild-type expression and function of the TCF4 protein encoded by the TCF4 gene. In some embodiments, the target TCF4 gene comprises an expansion of the number of CTG repeats as compared to the wild-type TCF4 gene prior to incorporation of the one or more intended nucleotide edits. In some embodiments, expression of a TCF4 transcript with reduced number of CTG repeats compared to a TCF4 transcript encoded by the endogenous TCF4 gene may be measured when expressed in a target cell. In some embodiments, a change in the level of TCF4 mRNA expression comprises a decrease in the amount of TCF4 transcripts having 35 or more CTG repeats. In some embodiments, a change in the level of TCF4 mRNA expression can comprise a fold change of, e.g., at least about 2-fold decrease, about 3-fold decrease, about 4-fold decrease, about 5-fold decrease, about 6-fold decrease, about 7-fold decrease, about 8-fold decrease, about 9-fold decrease, about 10-fold decrease, about 25-fold decrease, about 50-fold decrease, about 100-fold decrease, about 200-fold decrease, about 500-fold decrease, about 700-fold decrease, about 1000-fold decrease, about 5000-fold decrease, or about 10,000-fold decrease in the amount of TCF4 transcripts having 35 or more CTG repeats.

In some embodiments, increase in expression and/or function of proteins affected by expansion of CGT repeats in the TCF4 gene can be measured by a functional assay. For example, in some embodiments, function of proteins of the Muscleblind-like (MBNL) family can be measured to examine the editing efficiency of the TCF4 gene.

### Methods of Treating Fuchs' Endothelial Corneal Dystrophy

In some embodiments, provided herein are methods for treatment of a subject diagnosed with a disease associated with or caused by one or more pathogenic mutations that can be corrected by prime editing. In some embodiments, provided herein are methods for treating FECD that comprise administering to a subject a therapeutically effective amount of a prime editing composition, or a pharmaceutical composition comprising a prime editing composition as described herein.

In some embodiments, administration of the prime editing composition results in incorporation of one or more intended nucleotide edits in the target gene in the subject. In some embodiments, administration of the prime editing composition results in correction of one or more pathogenic mutations, e.g., point mutations, insertions, or deletions, associated with FECD in the subject. In some embodiments, the target gene comprises a sequence, e.g., the IND sequence that contains the pathogenic mutation. In some embodiments, administration of the prime editing composition results in incorporation of one or more intended nucleotide edits in the target gene that corrects the pathogenic mutation, or reduces the pathogenic effect of the mutation by deleting the sequence of the IND and optionally replacing the IND sequence with one or more endogenous or exogenous sequence that has a reduced number of CTG repeats or does not comprise a CTG repeat in the target gene, thereby treating FECD in the subject.

In some embodiments, the method provided herein comprises administering to a subject an effective amount of a prime editing composition, for example, a pair of PEgRNAs (i.e., a first PEgRNA and a second PEgRNA) and a prime editor. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the TCF4 gene in a subject, e.g., a human subject, suffering from, having susceptibility to, or at risk for FECD. Identifying a subject in need of such treatment can be in the judgment of a subject or a health care professional and can be subjective (e.g., opinion) or objective (e.g., measurable by a test or diagnostic method). In some embodiments, the subject has FECD.

In some embodiments, the subject has been diagnosed with FECD by sequencing of a TCF4 gene in the subject. In some embodiments, the subject comprises at least a copy of the TCF4 gene that comprises one or more mutations compared to a wild-type TCF4 gene. In some embodiments, the subject comprises at least a copy of the TCF4 gene that comprises a mutation in a non-coding region of the TCF4 gene. In some embodiments, the subject comprises at least a copy of the TCF4 gene that comprises a mutation in intron 3 of the TCF4 gene, as compared to a wild-type TCF4 gene. In some embodiments, the subject comprises two copies of the TCF4 gene, wherein each of the two copies comprises a mutation in intron 3 of the TCF4 gene as compared to a wild-type TCF4 gene. In some embodiments, the mutation is increased number of CTG repeats in intron 3 as compared to a wild-type TCF4 gene.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising prime editor complexes that comprises (i) a prime editor fusion protein and a first PEgRNA and (ii) a prime editor fusion protein and a second PEgRNA to a subject. In some embodiments, the method comprises administering a polynucleotide or vector encoding a prime editor to a subject simultaneously with the two PEgRNAs. In some embodiments, the method comprises administering a polynucleotide or vector encoding a prime editor to a subject before administration of the two PEgRNAs. In some embodiments, the two PEgRNAs are administered simultaneously. In some embodiments, the two PEgRNAs are administered sequentially. In some embodiments, a first PEgRNA is administered with a prime editor and a second PEgRNA is administered after administration of the first PEgRNA and prime editor. In some embodiments, a first PEgRNA is administered with a prime editor and a second PEgRNA is administered before administration of the first PEgRNA and prime editor.

Suitable routes of administrating the prime editing compositions to a subject include, without limitation: topical, topical by eyedrops, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection into the cornea of a subject. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with the PEgRNAs, or polynucleotides encoding the PEgRNAs.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are corneal endothelial cells. In some embodiments, the edited cells are primary human cells. In some embodiments, the edited cells are human progenitor cells. In some embodiments, the edited cells are human stem cells. In some embodiments, the edited cells are human corneal endothelial cells. In some embodiments, the cell is a fibroblast. In some embodiments, the cell is a human fibroblast. In some embodiments, the edited cell is a corneal endothelial progenitor cell. In some embodiments, the edited cell is a human corneal endothelial progenitor cell. In some embodiments, the edited cell is a human myoblast. In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in the TCF4 gene, or diagnostic measurement associated with FECD, in a subject suffering from FECD symptoms and has been administered an effective amount of a prime editing composition described herein. The level of the diagnostic marker determined in the method can be compared to known levels of the marker in either healthy normal controls or in other afflicted subjects to establish the subject's disease status.

Various aspects of this disclosure provide kits comprising a prime editing composition. In one embodiment, a kit comprises a prime editing composition comprising a pair of PERNAs (i.e., a first PEgRNA and a second PEgRNA) and a prime editor. In one embodiment, a kit comprises a prime editing composition comprising a pair of PEgRNAs (i.e., a first PEgRNA and a second PEgRNA) and a prime editor fusion protein. In some embodiments, the kit comprises a pair of PEgRNAs (i.e., a first PEgRNA and a second PEgRNA) and a polynucleotide encoding a prime editor. In some embodiments, the kit comprises a pair of PEgRNAs (i.e., a first PEgRNA and a second PEgRNA) and a polynucleotide encoding a prime editor fusion protein. In some embodiments, the kit further provides components for delivery of the PEgRNAs and/or the prime editor. In some embodiments, the kit further provides components for delivery of the PEgRNAs and/or the prime editor fusion protein. In some embodiments, the kit further provides components for delivery of the PEgRNAs and/or the polynucleotide encoding the prime editor. In some embodiments, the kit further provides components for delivery of the PERNAs and/or the polynucleotide encoding the prime editor fusion protein.

In some embodiments, the kit provides instructions for using the components of the kit for prime editing. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide and PEgRNAs is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA binding domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PERNA. In some embodiments, the polypeptide encodes two or more components of a prime editing composition, for example, a prime editor fusion protein and a PEgRNA.

In some embodiments, the polynucleotide encoding one or more prime editing composition components that is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from DNA. In some embodiments, a mRNA that encodes a prime editor polypeptide is generated using in vitro transcription. Guide polynucleotides (e.g., PEgRNA) can also be transcribed using in vitro transcription from a cassette containing a T7 promoter, followed by the sequence “GG”, and guide polynucleotide sequence. In some embodiments, the prime editor encoding mRNA and/or PEgRNA(s) are synthesized in vitro using an RNA polymerase enzyme (e.g., T7 polymerase, T3 polymerase, or SP6 polymerase). Once synthesized, the RNA can directly contact a target TCF4 gene or can be introduced into a cell using any suitable technique for introducing nucleic acids into cells (e.g., microinjection, electroporation, or transfection). In some embodiments, the prime editor-coding sequences and/or the PEgRNAs are modified to include one or more modified nucleoside e.g., using pseudo-U or 5-Methyl-C.

Methods of non-viral delivery of nucleic acids can include lipofection, nucleofection, microinjection, biolistics, virosomes, liposomes, immunoliposomes, polycation or lipid-nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid-nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and Psi-2 (ψ2) cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. The intein, nuclease and capsid protein can be fused together in any arrangement (e.g., nuclease-intein-capsid, intein-nuclease-capsid, or capsid-intein-nuclease). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

A target cell can be transiently or non-transiently transfected with one or more vectors described herein. A cell can be transfected as it naturally occurs in a subject. A cell can be taken from a subject and transfected. A cell can be taken from a subject and immortalized to make a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 387). As another example, the permeant peptide can comprise the HIV-1 tat basic region amino acid sequence, which may include, for example, amino acids 49-57 of naturally-occurring tat protein, YGRKKRRQRRR (SEQ ID NO: 388). Other permeant domains can include poly-arginine motifs, for example, the region of amino acids 34-56 of HIV-1 rev protein, nona-arginine (SEQ ID NO: 404), and octa-arginine (SEQ ID NO: 405). The nona-arginine (R9) sequence can be used (SEQ ID NO: 404). The site at which the fusion can be made may be selected in order to optimize the biological activity, entry, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PERNA(s) are introduced to a target cell by nanoparticles. In some embodiments, the prime editor polypeptide components and the PEgRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a corneal endothelial cell, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids and polymers used to produce nanoparticle formulations for polynucleotide delivery are provided in Tables 16 and 17 below, respectively.

TABLE 16
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy1-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethylpyridinium | 2Oc | Cationic |
| 2,3-Dioleyloxy-N-P(spenninecarboxamido-ethylJ- | DOSPA | Cationic |
| N,Ndimethyl- | | |
| 1-propanatninium trifluoroacetate | | |
| 1,2-Dioley1-3-trimethylamtnonium-propane | DOPA | Cationic |
| N-(2-Hydroxyethyl)-N,N-dimethy1-2,3-bis(tetradecyloxy)- | MDRIE | Cationic |
| 1- | | |
| propanaminium bromide | | |
| Dimyristooxypropyl dimethyl hydroxyethyl ammonium | DMRI | Cationic |
| bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecyl-3- | diCl4- | Cationic |
| tetradecylaminopropionamidine | amidine | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9- | CDAN | Cationic |
| diamine | | |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

TABLE 17
Exemplary polymers for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

In some embodiments, components of a prime editing composition form a complex prior to delivery to a target cell. For example, a prime editor fusion protein and a PEgRNA can form a complex prior to delivery to the target cell. In some embodiments, a prime editing polypeptide (e.g., a prime editor fusion protein) and a guide polynucleotide (e.g., a PEgRNA) form a ribonucleoprotein (RNP) for delivery to a target cell. In some embodiments, the RNP comprises a prime editor fusion protein in complex with a PERNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary delivery methods for polynucleotides encoding prime editing composition components are shown in Table 18 below.

TABLE 18
Exemplary polynucleotide delivery methods

| | | Delivery | | | |
|---|---|---|---|---|---|
| | | into Non- | | | Type of |
| | | Dividing | Duration of | Genome | Molecule |
| Delivery | Vector/Mode | Cells | Expression | Integration | Delivered |
| Physical | (e.g., | YES | Transient | NO | Nucleic |
| | electroporation, | | | | Acids and |
| | particle gun, | | | | Proteins |
| | Calcium | | | | |
| | phosphate | | | | |
| | transfection) | | | | |
| Viral | Retrovirus | NO | Stable | YES | RNA |
| | Lentivirus | YES | Stable | YES/NO | RNA |
| | | | | with | |
| | | | | modification | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic |
| | | | | what is | acids and |
| | | | | delivered | Proteins |
| | Polymeric | YES | Transient | NO | Nucleic |
| | Nanoparticles | | | | Acids |
| Biological | Attenuated | YES | Transient | NO | Nucleic |
| | Bacteria | | | | Acids |
| Non-Viral | Engineered | YES | Transient | NO | Nucleic |
| Delivery | Bacteriophages | | | | Acids |
| Vehicles | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, for example, one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prime editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., twice, three times or more. In cases in which two or more different prime editing system components, e.g., two different polynucleotide constructs are administered to the subject (e.g., different components of the same prime editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

The following examples are provided for illustrative purposes only and are not intended to limit the scope of the claims provided herein.

### Example 1—General Methods for Producing Prime Editing Components

PEgRNA assembly: PEgRNA libraries are assembled by one of three methods: in the first method, pooled synthesized DNA oligos encoding the PEgRNA and flanking U6 expression plasmid homology regions are cloned into U6 expression plasmids via Gibson cloning and sequencing of bacterial colonies via Sanger or Next-generation sequencing. In the second method, double-stranded linear DNA fragments encoding PEgRNA and homology sequences as above are individually Gibson-cloned into U6 expression plasmids. In the third method, for each PEgRNA, separate oligos encoding a protospacer, a gRNA scaffold, and PEgRNA extension (PBS and RTT) are ligated, and then cloned into a U6 expression plasmid as described in Anzalone et al., Nature. 2019 December; 576 (7785): 149-157. Bacterial colonies carrying sequence-verified plasmids are propagated in TB. Plasmid DNA is purified by minipreps for mammalian transfection.

HEK cell culture and transfection: HEK293T cells are propagated in DMEM with 10% FBS. Prior to transfection, cells are seeded in 96-well plates and then transfected with Lipofectamine 2000 according to the manufacturer's directions with DNA encoding a prime editor fusion protein and a pair of PERNA. Three days after transfection, gDNA is harvested in lysis buffer for high throughput sequencing using the Miseq system.

iPSC transfection and editing analysis. iPSC cells are plated in 96-well plate on Matrigel-coated plates in E8 media with 10 μM of ROCK inhibitor. Cells are transfected on the following day with Lipofectamine STEM according to the manufacturer's directions with polynucleotides encoding a pair of PEgRNAs and a prime editor fusion protein. Three days after transfection, gDNA is harvested in lysis buffer for sequencing and analysis with droplet digital PCR (ddPCR).

### Example 2—Screening of PERNA for Excising CTG Repeats Associated with Fuchs' Endothelial Corneal Dystrophy

PEgRNA design: PEgRNA libraries were designed to replace CTG repeats in the TCF4 gene by the following method. PEgRNAs spacer sequences and binding regions flanking the 5′ and 3′ repeat regions of the TCF4 gene locus (target site) were developed (see Tables 6 and 7).

Corresponding 5′ and 3′ prime binding site (PBS) sequences directed to the complementary strands were developed based on the 5′ and 3′ flanking spacer locations and advantageous nicking locations (see Tables 8 and 9). For the initial studies, 5′ and 3′ homologous sequences of Bbx1 attB were used as the editing template (RTT) sequence for detection and validation.

PEgRNA assembly: PEgRNA libraries containing 5′ PEgRNAs (PEgRNAs that recognize search target sequences 5′ of the CTG repeats) and 3′ PEgRNAs (PEgRNAs that recognize search target sequences 3′ of the CTG repeats) were assembled by the following method: double-stranded linear DNA fragments encoding PEgRNA and homology sequences were individually Gibson-cloned into U6 expression plasmids as described in Anzalone et al., Nature. 2019 December; 576 (7785): 149-157. Bacterial colonies carrying sequence-verified plasmids were propagated in TB medium. Plasmid DNA was purified by minipreps for mammalian transfection.

Preparation of a PERNA matrix: Sequence-verified PEgRNA plasmids were assembled in a matrix of combinations, such that each 5′ PEgRNA of the repeat region was paired with a 3′ PERNA of the repeat region (see Tables 14 and 15).

Wild-type HEK 293T cells were transfected with polynucleotides encoding a prime editor fusion protein a pair of PEgRNAs as described in Example 1. Editing efficiency of dual prime editing is reflected by excision of the 5 CTG repeats (SEQ ID NO: 390) in wild-type HEK293T cells and integration of the attB sequence encoded by the RTTs of the PEgRNA pair.

TCF4 CTG repeat excision and attB integration with the pair of PERNAs-PE system: The HEK 293T cell line as described was transiently transfected with a PE and PEgRNAs in arrayed 96-well plates for assessment of editing by high-throughput sequencing. A total of 225 PEgRNAs combinations (sequences shown in Tables 14 and 15) were tested for CTG repeat excision using a dual PEgRNA-PE editing system in wild-type HEK293T cells. A perfect edit was expected to replace the repeat sequence with the 38 nt attB sequence, flanked by non-repeat endogenous sequence. Sequencing in this region was expected to yield, in the case of a perfect edit, a 72nt sequence consisting of the 38nt attB sequence flanked by 17nt upstream and downstream of non-repeat endogenous sequence, which was used as a marker sequence for attB integration at the position of CTG repeat excision to assess dual prime editing efficiency. Sequencing read numbers, including (a) total editing reads, (b) reads of sequences that have exact match to the 72 bp sequence including the attB sequence and the flanking sequences on both sides, and (c) reads of sequences that match the 38 bp attB sequence (but not match the 72 bp sequence) were calculated. Editing % and indel % were estimated by version 1 CRISPResso2 dual-flap analysis pipeline. Editing efficiency was calculated using the following formula: editing %=(a)/(b) %, where (a) is the number of reads aligned to a perfect edit (amplicon1) and (b) is the total number of reads (i.e., reads aligned to both the edited genome and non-edited (reference) genome). Indel frequency is represented by 1) indel rates of the average indel % observed at each nucleotide position between the 5′ nick and the 3′ nick (“indel_N2N %”); 2) the indel % at the position one nucleotide downstream of the 5′ nick (“indel_5N %”); and 3) the indel % at the position one nucleotide upstream of the 3′ nick (“indel_3N %”). The results are shown in Table 19 below. The following scale was used: PEgRNA pairs exhibiting 0% editing efficiency or indel rate are indicated with “−”, PEgRNA pairs exhibiting editing efficiency between 0% to 1% are indicated with “+”, PEgRNA pairs exhibiting editing efficiency of 1% to 10% are indicated with “++”, and PEgRNA pairs exhibiting editing efficiency of 10% to 100% are indicated with “+++”. Indel rates, as reflected by the largest of N2N %, 5N %, or 3N % for the particular PEgRNA pair, are reported on the same scale.

TABLE 19
PEgRNA pair editing efficiency and indel level

| 5′ | 3′ | | |
|---|---|---|---|
| PEgRNA | PEgRNA | | |
| SEQ ID | SEQ | | |
| NO | ID NO | Edit % | Indel % |
| 306 | 321 | +++ | ++ |
| 306 | 322 | +++ | ++ |
| 306 | 323 | +++ | ++ |
| 306 | 324 | +++ | + |
| 306 | 325 | +++ | ++ |
| 306 | 326 | +++ | ++ |
| 306 | 327 | + | +++ |
| 306 | 328 | +++ | ++ |
| 306 | 329 | +++ | + |
| 306 | 330 | + | +++ |
| 306 | 331 | +++ | + |
| 306 | 332 | +++ | + |
| 306 | 333 | + | +++ |
| 306 | 334 | + | ++ |
| 306 | 335 | ++ | ++ |
| 307 | 321 | +++ | ++ |
| 307 | 322 | +++ | + |
| 307 | 323 | +++ | + |
| 307 | 324 | +++ | ++ |
| 307 | 325 | +++ | ++ |
| 307 | 326 | +++ | + |
| 307 | 327 | − | +++ |
| 307 | 328 | +++ | ++ |
| 307 | 329 | +++ | + |
| 307 | 330 | − | +++ |
| 307 | 331 | +++ | + |
| 307 | 332 | +++ | + |
| 307 | 333 | + | +++ |
| 307 | 334 | + | ++ |
| 307 | 335 | ++ | + |
| 308 | 321 | +++ | ++ |
| 308 | 322 | +++ | ++ |
| 308 | 323 | +++ | + |
| 308 | 324 | +++ | ++ |
| 308 | 325 | +++ | ++ |
| 308 | 326 | +++ | + |
| 308 | 327 | − | +++ |
| 308 | 328 | +++ | ++ |
| 308 | 329 | +++ | ++ |
| 308 | 330 | − | +++ |
| 308 | 331 | +++ | + |
| 308 | 332 | +++ | + |
| 308 | 333 | + | ++ |
| 308 | 334 | + | +++ |
| 308 | 335 | ++ | ++ |
| 309 | 321 | +++ | ++ |
| 309 | 322 | +++ | + |
| 309 | 323 | +++ | + |
| 309 | 324 | +++ | + |
| 309 | 20 | +++ | + |
| 309 | 21 | +++ | + |
| 309 | 22 | − | +++ |
| 309 | 23 | +++ | ++ |
| 309 | 24 | +++ | + |
| 309 | 25 | − | +++ |
| 309 | 26 | +++ | + |
| 309 | 27 | +++ | + |
| 309 | 333 | + | +++ |
| 309 | 334 | − | +++ |
| 309 | 335 | ++ | ++ |
| 310 | 32 | +++ | ++ |
| 310 | 322 | +++ | + |
| 310 | 323 | +++ | + |
| 310 | 324 | +++ | ++ |
| 310 | 325 | +++ | + |
| 310 | 326 | +++ | + |
| 310 | 327 | − | +++ |
| 310 | 328 | +++ | ++ |
| 310 | 329 | +++ | ++ |
| 310 | 330 | − | +++ |
| 310 | 331 | +++ | + |
| 310 | 332 | +++ | + |
| 310 | 333 | + | ++ |
| 310 | 334 | ++ | +++ |
| 310 | 335 | ++ | + |
| 311 | 321 | +++ | ++ |
| 311 | 322 | +++ | + |
| 311 | 323 | +++ | + |
| 311 | 324 | +++ | ++ |
| 311 | 325 | +++ | ++ |
| 311 | 326 | +++ | ++ |
| 311 | 327 | − | +++ |
| 311 | 328 | +++ | ++ |
| 311 | 329 | +++ | + |
| 311 | 330 | − | +++ |
| 311 | 331 | +++ | + |
| 311 | 332 | +++ | + |
| 311 | 333 | ++ | +++ |
| 311 | 334 | + | +++ |
| 311 | 335 | ++ | ++ |
| 312 | 321 | +++ | ++ |
| 312 | 322 | +++ | + |
| 312 | 323 | +++ | + |
| 312 | 324 | +++ | ++ |
| 312 | 325 | +++ | ++ |
| 312 | 326 | +++ | + |
| 312 | 327 | − | +++ |
| 312 | 328 | +++ | ++ |
| 312 | 329 | +++ | + |
| 312 | 330 | − | +++ |
| 312 | 331 | +++ | + |
| 312 | 332 | +++ | + |
| 312 | 333 | + | ++ |
| 312 | 334 | ++ | +++ |
| 312 | 335 | ++ | ++ |
| 313 | 321 | +++ | ++ |
| 313 | 322 | +++ | + |
| 313 | 323 | +++ | + |
| 313 | 324 | +++ | ++ |
| 313 | 325 | +++ | ++ |
| 313 | 326 | +++ | ++ |
| 313 | 327 | − | +++ |
| 313 | 328 | +++ | ++ |
| 313 | 329 | +++ | ++ |
| 313 | 330 | − | +++ |
| 313 | 331 | +++ | ++ |
| 313 | 332 | +++ | + |
| 313 | 333 | + | ++ |
| 313 | 334 | ++ | +++ |
| 313 | 335 | ++ | ++ |
| 314 | 321 | +++ | ++ |
| 314 | 322 | +++ | ++ |
| 314 | 323 | +++ | ++ |
| 314 | 324 | +++ | ++ |
| 314 | 325 | +++ | ++ |
| 314 | 326 | +++ | + |
| 314 | 327 | − | +++ |
| 314 | 328 | +++ | +++ |
| 314 | 329 | +++ | ++ |
| 314 | 330 | − | +++ |
| 314 | 331 | +++ | ++ |
| 314 | 332 | +++ | + |
| 314 | 333 | + | ++ |
| 314 | 334 | + | +++ |
| 314 | 335 | ++ | +++ |
| 315 | 321 | +++ | − |
| 315 | 322 | +++ | + |
| 315 | 323 | +++ | + |
| 315 | 324 | +++ | ++ |
| 315 | 325 | +++ | + |
| 315 | 326 | +++ | + |
| 315 | 327 | − | +++ |
| 315 | 328 | +++ | + |
| 315 | 329 | +++ | + |
| 315 | 330 | − | +++ |
| 315 | 331 | +++ | − |
| 315 | 332 | +++ | − |
| 315 | 333 | − | ++ |
| 315 | 334 | − | +++ |
| 315 | 335 | ++ | − |
| 316 | 321 | +++ | ++ |
| 316 | 322 | +++ | + |
| 316 | 323 | +++ | + |
| 316 | 324 | +++ | ++ |
| 316 | 325 | +++ | + |
| 316 | 326 | +++ | + |
| 316 | 327 | − | − |
| 316 | 328 | +++ | + |
| 316 | 329 | +++ | − |
| 316 | 330 | − | +++ |
| 316 | 331 | +++ | ++ |
| 316 | 332 | +++ | − |
| 316 | | + | + |
| 316 | 334 | + | + |
| 316 | 335 | ++ | − |
| 317 | 321 | +++ | + |
| 317 | 322 | +++ | + |
| 317 | 323 | +++ | + |
| 317 | 324 | +++ | − |
| 317 | 325 | +++ | + |
| 317 | 326 | +++ | + |
| 17 | 327 | − | +++ |
| 317 | 328 | +++ | ++ |
| 317 | 329 | +++ | + |
| 317 | 330 | − | +++ |
| 317 | 331 | +++ | + |
| 317 | 332 | +++ | + |
| 317 | 333 | − | ++ |
| 317 | 334 | ++ | +++ |
| 317 | 335 | +++ | − |
| 318 | 321 | +++ | − |
| 318 | 322 | +++ | − |
| 318 | 323 | +++ | + |
| 318 | 324 | +++ | ++ |
| 318 | 325 | +++ | ++ |
| 318 | 326 | +++ | + |
| 318 | 327 | − | +++ |
| 318 | 328 | +++ | ++ |
| 318 | 329 | +++ | + |
| 318 | 330 | − | ++ |
| 318 | 331 | +++ | + |
| 318 | 332 | +++ | + |
| 318 | 333 | − | ++ |
| 318 | 334 | ++ | + |
| 318 | 335 | +++ | + |
| 319 | 321 | +++ | − |
| 319 | 322 | +++ | + |
| 319 | 323 | +++ | − |
| 319 | 324 | +++ | − |
| 319 | 325 | +++ | + |
| 319 | 326 | +++ | − |
| 319 | 327 | − | +++ |
| 319 | 328 | +++ | + |
| 319 | 329 | +++ | − |
| 319 | 330 | − | +++ |
| 319 | 331 | +++ | + |
| 319 | 332 | +++ | + |
| 319 | 333 | ++ | +++ |
| 319 | 334 | ++ | − |
| 319 | 335 | +++ | − |
| 320 | 321 | +++ | ++ |
| 320 | 322 | +++ | + |
| 320 | 323 | +++ | + |
| 320 | 324 | +++ | − |
| 320 | 325 | +++ | − |
| 320 | 326 | +++ | + |
| 320 | 327 | − | − |
| 320 | 328 | +++ | ++ |
| 320 | 329 | +++ | + |
| 320 | 330 | − | − |
| 15320 | 331 | +++ | + |
| 15320 | 332 | +++ | + |
| 15320 | 333 | ++ | − |
| 15320 | 334 | + | + |
| 15320 | 335 | ++ | − |

Table 20 provides sequences referenced throughout the present disclosure.

TABLE 20
Amino acid and nucleic acid sequences for dual prime editing components

| SEQ | | |
|---|---|---|
| ID NO: | Description | Sequence |
| 1 | WT-M- | TLNIEDEHRLHETSKEPDVSLGSTWLSDFPQAWAETGG |
| | MLV RT | MGLAVRQAPLIIPLKATSTPVSIKQYPMSQEARLGIKPHI |
| | | QRLLDQGILVPCQSPWNTPLLPVKKPGTNDYRPVQDLR |
| | | EVNKRVEDIHPTVPNPYNLLSGLPPSHQWYTVLDLKDA |
| | | FFCLRLHPTSQPLFAFEWRDPEMGISGQLTWTRLPQGFK |
| | | NSPTLFDEALHRDLADFRIQHPDLILLQYVDDLLLAATS |
| | | ELDCQQGTRALLQTLGNLGYRASAKKAQICQKQVKYL |
| | | GYLLKEGQRWLTEARKETVMGQPTPKTPRQLREFLGT |
| | | AGFCRLWIPGFAEMAAPLYPLTKTGTLFNWGPDQQKA |
| | | YQEIKQALLTAPALGLPDLTKPFELFVDEKQGYAKGVL |
| | | TQKLGPWRRPVAYLSKKLDPVAAGWPPCLRMVAAIAV |
| | | LTKDAGKLTMGQPLVILAPHAVEALVKQPPDRWLSNA |
| | | RMTHYQALLLDTDRVQFGPVVALNPATLLPLPEEGLQH |
| | | NCLDILAEAHGTRPDLTDQPLPDADHTWYTDGSSLLQE |
| | | GQRKAGAAVTTETEVIWAKALPAGTSAQRAELIALTQA |
| | | LKMAEGKKLNVYTDSRYAFATAHIHGEIYRRRGLLTSE |
| | | GKEIKNKDEILALLKALFLPKRLSIIHCPGHQKGHSAEAR |
| | | GNRMADQAARKAAITETPDTSTLLIENSSP |
| 2 | PEmax- | TLNIEDEYRLHETSKEPDVSLGSTWLSDFPQAWAETGG |
| | MMLV_RT | MGLAVRQAPLIIPLKATSTPVSIKQYPMSQEARLGIKPHI |
| | D200N | QRLLDQGILVPCQSPWNTPLLPVKKPGTNDYRPVQDLR |
| | T330P | EVNKRVEDIHPTVPNPYNLLSGLPPSHQWYTVLDLKDA |
| | L603W | FFCLRLHPTSQPLFAFEWRDPEMGISGQLTWTRLPQGFK |
| | T306K | NSPTLFNEALHRDLADFRIQHPDLILLQYVDDLLLAATS |
| | W313F | ELDCQQGTRALLQTLGNLGYRASAKKAQICQKQVKYL |
| | (same | GYLLKEGQRWLTEARKETVMGQPTPKTPRQLREFLGK |
| | sequence as | AGFCRLFIPGFAEMAAPLYPLTKPGTLFNWGPDQQKAY |
| | MMLV | QEIKQALLTAPALGLPDLTKPFELFVDEKQGYAKGVLT |
| | variant) | QKLGPWRRPVAYLSKKLDPVAAGWPPCLRMVAAIAVL |
| | pentamutant | TKDAGKLTMGQPLVILAPHAVEALVKQPPDRWLSNAR |
| | | MTHYQALLLDTDRVQFGPVVALNPATLLPLPEEGLQHN |
| | | CLDILAEAHGTRPDLTDQPLPDADHTWYTDGSSLLQEG |
| | | QRKAGAAVTTETEVIWAKALPAGTSAQRAELIALTQAL |
| | | KMAEGKKLNVYTDSRYAFATAHIHGEIYRRRGWLTSE |
| | | GKEIKNKDEILALLKALFLPKRLSIIHCPGHQKGHSAEAR |
| | | GNRMADQAARKAAITETPDTSTLLIENSSP |
| 3 | Wild-type | MDKKYSIGLDIGTNSVGWAVITDEYKVPSKKFKVLGNT |
| | Streptococcus | DRHSIKKNLIGALLFDSGETAEATRLKRTARRRYTRRKN |
| | pyogenes | RICYLQEIFSNEMAKVDDSFFHRLEESFLVEEDKKHERH |
| | Cas9 | PIFGNIVDEVAYHEKYPTIYHLRKKLVDSTDKADLRLIY |
| | (SpCas9) | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDHIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFDSPTVAYSVLVVAKVEKGKSK |
| | | KLKSVKELLGITIMERSSFEKNPIDFLEAKGYKEVKKDLI |
| | | IKLPKYSLFELENGRKRMLASAGELQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKRYTSTKEVLDATLIHQSI |
| | | TGLYETRIDLSQLGGD |
| 4 | WT sluCas9 | MNQKFILGLDIGITSVGYGLIDYETKNIIDAGVRLFPEAN |
| | (recognized | VENNEGRRSKRGSRRLKRRRIHRLERVKKLLEDYNLLD |
| | NNGG | QSQIPQSTNPYAIRVKGLSEALSKDELVIALLHIAKRRGI |
| | PAM) | HKIDVIDSNDDVGNELSTKEQLNKNSKLLKDKFVCQIQ |
| | | LERMNEGQVRGEKNRFKTADIIKEIIQLLNVQKNFHQLD |
| | | ENFINKYIELVEMRREYFEGPGKGSPYGWEGDPKAWYE |
| | | TLMGHCTYFPDELRSVKYAYSADLFNALNDLNNLVIQR |
| | | DGLSKLEYHEKYHIIENVFKQKKKPTLKQIANEINVNPE |
| | | DIKGYRITKSGKPQFTEFKLYHDLKSVLFDQSILENEDV |
| | | LDQIAEILTIYQDKDSIKSKLTELDILLNEEDKENIAQLTG |
| | | YTGTHRLSLKCIRLVLEEQWYSSRNQMEIFTHLNIKPKK |
| | | INLTAANKIPKAMIDEFILSPVVKRTFGQAINLINKIIEKY |
| | | GVPEDIIIELARENNSKDKQKFINEMQKKNENTRKRINEI |
| | | IGKYGNQNAKRLVEKIRLHDEQEGKCLYSLESIPLEDLL |
| | | NNPNHYEVDHIIPRSVSFDNSYHNKVLVKQSENSKKSN |
| | | LTPYQYFNSGKSKLSYNQFKQHILNLSKSQDRISKKKKE |
| | | YLLEERDINKFEVQKEFINRNLVDTRYATRELTNYLKA |
| | | YFSANNMNVKVKTINGSFTDYLRKVWKFKKERNHGYK |
| | | HHAEDALIIANADFLFKENKKLKAVNSVLEKPEIESKQL |
| | | DIQVDSEDNYSEMFIIPKQVQDIKDFRNFKYSHRVDKKP |
| | | NRQLINDTLYSTRKKDNSTYIVQTIKDIYAKDNTTLKKQ |
| | | FDKSPEKFLMYQHDPRTFEKLEVIMKQYANEKNPLAKY |
| | | HEETGEYLTKYSKKNNGPIVKSLKYIGNKLGSHLDVTH |
| | | QFKSSTKKLVKLSIKPYRFDVYLTDKGYKFITISYLDVL |
| | | KKDNYYYIPEQKYDKLKLGKAIDKNAKFIASFYKNDLI |
| | | KLDGEIYKIIGVNSDTRNMIELDLPDIRYKEYCELNNIKG |
| | | EPRIKKTIGKKVNSIEKLTTDVLGNVFTNTQYTKPQLLF |
| | | KRGN |
| 5 | SpCas9 | MDKKYSIGLDIGTNSVGWAVITDEYKVPSKKFKVLGNT |
| | (H840A) | DRHSIKKNLIGALLFDSGETAEATRLKRTARRRYTRRKN |
| | Nickase | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDAIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFDSPTVAYSVLVVAKVEKGKSK |
| | | KLKSVKELLGITIMERSSFEKNPIDFLEAKGYKEVKKDLI |
| | | IKLPKYSLFELENGRKRMLASAGELQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKRYTSTKEVLDATLIHQSI |
| | | TGLYETRIDLSQLGGD |
| 6 | Met(−) | DKKYSIGLDIGTNSVGWAVITDEYKVPSKKFKVLGNTD |
| | SpCas9 | RHSIKKNLIGALLFDSGETAEATRLKRTARRRYTRRKNR |
| | (H840A) | ICYLQEIFSNEMAKVDDSFFHRLEESFLVEEDKKHERHPI |
| | Nickase | FGNIVDEVAYHEKYPTIYHLRKKLVDSTDKADLRLIYL |
| | | ALAHMIKFRGHFLIEGDLNPDNSDVDKLFIQLVQTYNQ |
| | | LFEENPINASGVDAKAILSARLSKSRRLENLIAQLPGEKK |
| | | NGLFGNLIALSLGLTPNFKSNFDLAEDAKLQLSKDTYD |
| | | DDLDNLLAQIGDQYADLFLAAKNLSDAILLSDILRVNTE |
| | | ITKAPLSASMIKRYDEHHQDLTLLKALVRQQLPEKYKEI |
| | | FFDQSKNGYAGYIDGGASQEEFYKFIKPILEKMDGTEEL |
| | | LVKLNREDLLRKQRTFDNGSIPHQIHLGELHAILRRQED |
| | | FYPFLKDNREKIEKILTFRIPYYVGPLARGNSRFAWMTR |
| | | KSEETITPWNFEEVVDKGASAQSFIERMTNFDKNLPNEK |
| | | VLPKHSLLYEYFTVYNELTKVKYVTEGMRKPAFLSGEQ |
| | | KKAIVDLLFKTNRKVTVKQLKEDYFKKIECFDSVEISGV |
| | | EDRFNASLGTYHDLLKIIKDKDFLDNEENEDILEDIVLTL |
| | | TLFEDREMIEERLKTYAHLFDDKVMKQLKRRRYTGWG |
| | | RLSRKLINGIRDKQSGKTILDFLKSDGFANRNFMQLIHD |
| | | DSLTFKEDIQKAQVSGQGDSLHEHIANLAGSPAIKKGIL |
| | | QTVKVVDELVKVMGRHKPENIVIEMARENQTTQKGQK |
| | | NSRERMKRIEEGIKELGSQILKEHPVENTQLQNEKLYLY |
| | | YLQNGRDMYVDQELDINRLSDYDVDAIVPQSFLKDDSI |
| | | DNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFDSPTVAYSVLVVAKVEKGKSK |
| | | KLKSVKELLGITIMERSSFEKNPIDFLEAKGYKEVKKDLI |
| | | IKLPKYSLFELENGRKRMLASAGELQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKRYTSTKEVLDATLIHQSI |
| | | TGLYETRIDLSQLGGD |
| 7 | sluCas9 | MNQKFILGLDIGITSVGYGLIDYETKNIIDAGVRLFPEAN |
| | (N582A) | VENNEGRRSKRGSRRLKRRRIHRLERVKKLLEDYNLLD |
| | Nickase | QSQIPQSTNPYAIRVKGLSEALSKDELVIALLHIAKRRGI |
| | | HKIDVIDSNDDVGNELSTKEQLNKNSKLLKDKFVCQIQ |
| | | LERMNEGQVRGEKNRFKTADIIKEIIQLLNVQKNFHQLD |
| | | ENFINKYIELVEMRREYFEGPGKGSPYGWEGDPKAWYE |
| | | TLMGHCTYFPDELRSVKYAYSADLFNALNDLNNLVIQR |
| | | DGLSKLEYHEKYHIIENVFKQKKKPTLKQIANEINVNPE |
| | | DIKGYRITKSGKPQFTEFKLYHDLKSVLFDQSILENEDV |
| | | LDQIAEILTIYQDKDSIKSKLTELDILLNEEDKENIAQLTG |
| | | YTGTHRLSLKCIRLVLEEQWYSSRNQMEIFTHLNIKPKK |
| | | INLTAANKIPKAMIDEFILSPVVKRTFGQAINLINKIIEKY |
| | | GVPEDIIIELARENNSKDKQKFINEMQKKNENTRKRINEI |
| | | IGKYGNQNAKRLVEKIRLHDEQEGKCLYSLESIPLEDLL |
| | | NNPNHYEVDHIIPRSVSFDNSYHNKVLVKQSEASKKSN |
| | | LTPYQYFNSGKSKLSYNQFKQHILNLSKSQDRISKKKKE |
| | | YLLEERDINKFEVQKEFINRNLVDTRYATRELTNYLKA |
| | | YFSANNMNVKVKTINGSFTDYLRKVWKFKKERNHGYK |
| | | HHAEDALIIANADFLFKENKKLKAVNSVLEKPEIESKQL |
| | | DIQVDSEDNYSEMFIIPKQVQDIKDFRNFKYSHRVDKKP |
| | | NRQLINDTLYSTRKKDNSTYIVQTIKDIYAKDNTTLKKQ |
| | | FDKSPEKFLMYQHDPRTFEKLEVIMKQYANEKNPLAKY |
| | | HEETGEYLTKYSKKNNGPIVKSLKYIGNKLGSHLDVTH |
| | | QFKSSTKKLVKLSIKPYRFDVYLTDKGYKFITISYLDVL |
| | | KKDNYYYIPEQKYDKLKLGKAIDKNAKFIASFYKNDLI |
| | | KLDGEIYKIIGVNSDTRNMIELDLPDIRYKEYCELNNIKG |
| | | EPRIKKTIGKKVNSIEKLTTDVLGNVFTNTQYTKPQLLF |
| | | KRGN |
| 8 | PEmax- | DKKYSIGLDIGTNSVGWAVITDEYKVPSKKFKVLGNTD |
| | CAS9 | RHSIKKNLIGALLFDSGETAEATRLKRTARRRYTRRKNR |
| | (R221K | ICYLQEIFSNEMAKVDDSFFHRLEESFLVEEDKKHERHPI |
| | N394K | FGNIVDEVAYHEKYPTIYHLRKKLVDSTDKADLRLIYL |
| | H840A) | ALAHMIKFRGHFLIEGDLNPDNSDVDKLFIQLVQTYNQ |
| | (not | LFEENPINASGVDAKAILSARLSKSRKLENLIAQLPGEKK |
| | including N- | NGLFGNLIALSLGLTPNFKSNFDLAEDAKLQLSKDTYD |
| | terminal | DDLDNLLAQIGDQYADLFLAAKNLSDAILLSDILRVNTE |
| | Met in | ITKAPLSASMIKRYDEHHQDLTLLKALVRQQLPEKYKEI |
| | Cas9) | FFDQSKNGYAGYIDGGASQEEFYKFIKPILEKMDGTEEL |
| | | LVKLKREDLLRKQRTFDNGSIPHQIHLGELHAILRRQED |
| | | FYPFLKDNREKIEKILTFRIPYYVGPLARGNSRFAWMTR |
| | | KSEETITPWNFEEVVDKGASAQSFIERMTNFDKNLPNEK |
| | | VLPKHSLLYEYFTVYNELTKVKYVTEGMRKPAFLSGEQ |
| | | KKAIVDLLFKTNRKVTVKQLKEDYFKKIECFDSVEISGV |
| | | EDRFNASLGTYHDLLKIIKDKDFLDNEENEDILEDIVLTL |
| | | TLFEDREMIEERLKTYAHLFDDKVMKQLKRRRYTGWG |
| | | RLSRKLINGIRDKQSGKTILDFLKSDGFANRNFMQLIHD |
| | | DSLTFKEDIQKAQVSGQGDSLHEHIANLAGSPAIKKGIL |
| | | QTVKVVDELVKVMGRHKPENIVIEMARENQTTQKGQK |
| | | NSRERMKRIEEGIKELGSQILKEHPVENTQLQNEKLYLY |
| | | YLQNGRDMYVDQELDINRLSDYDVDAIVPQSFLKDDSI |
| | | DNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFDSPTVAYSVLVVAKVEKGKSK |
| | | KLKSVKELLGITIMERSSFEKNPIDFLEAKGYKEVKKDLI |
| | | IKLPKYSLFELENGRKRMLASAGELQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKRYTSTKEVLDATLIHQSI |
| | | TGLYETRIDLSQLGGD |
| 9 | Met(+) | MNQKFILGLDIGITSVGYGLIDYETKNIIDAGVRLFPEAN |
| | sluCas9 | VENNEGRRSKRGSRRLKRRRIHRLERVKKLLEDYNLLD |
| | ( )Nickase | QSQIPQSTNPYAIRVKGLSEALSKDELVIALLHIAKRRGI |
| | | HKIDVIDSNDDVGNELSTKEQLNKNSKLLKDKFVCQIQ |
| | | LERMNEGQVRGEKNRFKTADIIKEIIQLLNVQKNFHQLD |
| | | ENFINKYIELVEMRREYFEGPGKGSPYGWEGDPKAWYE |
| | | TLMGHCTYFPDELRSVKYAYSADLFNALNDLNNLVIQR |
| | | DGLSKLEYHEKYHIIENVFKQKKKPTLKQIANEINVNPE |
| | | DIKGYRITKSGKPQFTEFKLYHDLKSVLFDQSILENEDV |
| | | LDQIAEILTIYQDKDSIKSKLTELDILLNEEDKENIAQLTG |
| | | YTGTHRLSLKCIRLVLEEQWYSSRNQMEIFTHLNIKPKK |
| | | INLTAANKIPKAMIDEFILSPVVKRTFGQAINLINKIIEKY |
| | | GVPEDIIIELARENNSKDKQKFINEMQKKNENTRKRINEI |
| | | IGKYGNQNAKRLVEKIRLHDEQEGKCLYSLESIPLEDLL |
| | | NNPNHYEVDHIIPRSVSFDNSYHNKVLVKQSEASKKSN |
| | | LTPYQYFNSGKSKLSYNQFKQHILNLSKSQDRISKKKKE |
| | | YLLEERDINKFEVQKEFINRNLVDTRYATRELTNYLKA |
| | | YFSANNMNVKVKTINGSFTDYLRKVWKFKKERNHGYK |
| | | HHAEDALIIANADFLFKENKKLKAVNSVLEKPEIESKQL |
| | | DIQVDSEDNYSEMFIIPKQVQDIKDFRNFKYSHRVDKKP |
| | | NRQLINDTLYSTRKKDNSTYIVQTIKDIYAKDNTTLKKQ |
| | | FDKSPEKFLMYQHDPRTFEKLEVIMKQYANEKNPLAKY |
| | | HEETGEYLTKYSKKNNGPIVKSLKYIGNKLGSHLDVTH |
| | | QFKSSTKKLVKLSIKPYRFDVYLTDKGYKFITISYLDVL |
| | | KKDNYYYIPEQKYDKLKLGKAIDKNAKFIASFYKNDLI |
| | | KLDGEIYKIIGVNSDTRNMIELDLPDIRYKEYCELNNIKG |
| | | EPRIKKTIGKKVNSIEKLTTDVLGNVFTNTQYTKPQLLF |
| | | KRGN |
| 10 | Met(−) | NQKFILGLDIGITSVGYGLIDYETKNIIDAGVRLFPEANV |
| | sluCas9 | ENNEGRRSKRGSRRLKRRRIHRLERVKKLLEDYNLLDQ |
| | ( )Nickase | SQIPQSTNPYAIRVKGLSEALSKDELVIALLHIAKRRGIH |
| | | KIDVIDSNDDVGNELSTKEQLNKNSKLLKDKFVCQIQLE |
| | | RMNEGQVRGEKNRFKTADIIKEIIQLLNVQKNFHQLDE |
| | | NFINKYIELVEMRREYFEGPGKGSPYGWEGDPKAWYET |
| | | LMGHCTYFPDELRSVKYAYSADLFNALNDLNNLVIQRD |
| | | GLSKLEYHEKYHIIENVFKQKKKPTLKQIANEINVNPEDI |
| | | KGYRITKSGKPQFTEFKLYHDLKSVLFDQSILENEDVLD |
| | | QIAEILTIYQDKDSIKSKLTELDILLNEEDKENIAQLTGYT |
| | | GTHRLSLKCIRLVLEEQWYSSRNQMEIFTHLNIKPKKIN |
| | | LTAANKIPKAMIDEFILSPVVKRTFGQAINLINKIIEKYG |
| | | VPEDIIIELARENNSKDKQKFINEMQKKNENTRKRINEII |
| | | GKYGNQNAKRLVEKIRLHDEQEGKCLYSLESIPLEDLL |
| | | NNPNHYEVDHIIPRSVSFDNSYHNKVLVKQSEASKKSN |
| | | LTPYQYFNSGKSKLSYNQFKQHILNLSKSQDRISKKKKE |
| | | YLLEERDINKFEVQKEFINRNLVDTRYATRELTNYLKA |
| | | YFSANNMNVKVKTINGSFTDYLRKVWKFKKERNHGYK |
| | | HHAEDALIIANADFLFKENKKLKAVNSVLEKPEIESKQL |
| | | DIQVDSEDNYSEMFIIPKQVQDIKDFRNFKYSHRVDKKP |
| | | NRQLINDTLYSTRKKDNSTYIVQTIKDIYAKDNTTLKKQ |
| | | FDKSPEKFLMYQHDPRTFEKLEVIMKQYANEKNPLAKY |
| | | HEETGEYLTKYSKKNNGPIVKSLKYIGNKLGSHLDVTH |
| | | QFKSSTKKLVKLSIKPYRFDVYLTDKGYKFITISYLDVL |
| | | KKDNYYYIPEQKYDKLKLGKAIDKNAKFIASFYKNDLI |
| | | KLDGEIYKIIGVNSDTRNMIELDLPDIRYKEYCELNNIKG |
| | | EPRIKKTIGKKVNSIEKLTTDVLGNVFTNTQYTKPQLLF |
| | | KRGN |
| 11 | spCas9-NG | MDKKYSIGLDIGTNSVGWAVITDEYKVPSKKFKVLGNT |
| | | DRHSIKKNLIGALLFDSGETAEATRLKRTARRRYTRRKN |
| | | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDHIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESIRPKRNSDK |
| | | LIARKKDWDPKKYGGFVSPTVAYSVLVVAKVEKGKSK |
| | | KLKSVKELLGITIMERSSFEKNPIDFLEAKGYKEVKKDLI |
| | | IKLPKYSLFELENGRKRMLASARFLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPRAFKYFDTTIDRKVYRSTKEVLDATLIHQSI |
| | | TGLYETRIDLSQLGGD |
| 12 | SpCas9-NG | MDKKYSIGLDIGTNSVGWAVITDEYKVPSKKFKVLGNT |
| | (H840A) | DRHSIKKNLIGALLFDSGETAEATRLKRTARRRYTRRKN |
| | Nickase | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDAIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESIRPKRNSDK |
| | | LIARKKDWDPKKYGGFVSPTVAYSVLVVAKVEKGKSK |
| | | KLKSVKELLGITIMERSSFEKNPIDFLEAKGYKEVKKDLI |
| | | IKLPKYSLFELENGRKRMLASARFLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPRAFKYFDTTIDRKVYRSTKEVLDATLIHQSI |
| | | TGLYETRIDLSQLGGD |
| 13 | SpCas9-NG | DKKYSIGLDIGTNSVGWAVITDEYKVPSKKFKVLGNTD |
| | (H839A) | RHSIKKNLIGALLFDSGETAEATRLKRTARRRYTRRKNR |
| | Nickase | ICYLQEIFSNEMAKVDDSFFHRLEESFLVEEDKKHERHPI |
| | | FGNIVDEVAYHEKYPTIYHLRKKLVDSTDKADLRLIYL |
| | | ALAHMIKFRGHFLIEGDLNPDNSDVDKLFIQLVQTYNQ |
| | | LFEENPINASGVDAKAILSARLSKSRRLENLIAQLPGEKK |
| | | NGLFGNLIALSLGLTPNFKSNFDLAEDAKLQLSKDTYD |
| | | DDLDNLLAQIGDQYADLFLAAKNLSDAILLSDILRVNTE |
| | | ITKAPLSASMIKRYDEHHQDLTLLKALVRQQLPEKYKEI |
| | | FFDQSKNGYAGYIDGGASQEEFYKFIKPILEKMDGTEEL |
| | | LVKLNREDLLRKQRTFDNGSIPHQIHLGELHAILRRQED |
| | | FYPFLKDNREKIEKILTFRIPYYVGPLARGNSRFAWMTR |
| | | KSEETITPWNFEEVVDKGASAQSFIERMTNFDKNLPNEK |
| | | VLPKHSLLYEYFTVYNELTKVKYVTEGMRKPAFLSGEQ |
| | | KKAIVDLLFKTNRKVTVKQLKEDYFKKIECFDSVEISGV |
| | | EDRFNASLGTYHDLLKIIKDKDFLDNEENEDILEDIVLTL |
| | | TLFEDREMIEERLKTYAHLFDDKVMKQLKRRRYTGWG |
| | | RLSRKLINGIRDKQSGKTILDFLKSDGFANRNFMQLIHD |
| | | DSLTFKEDIQKAQVSGQGDSLHEHIANLAGSPAIKKGIL |
| | | QTVKVVDELVKVMGRHKPENIVIEMARENQTTQKGQK |
| | | NSRERMKRIEEGIKELGSQILKEHPVENTQLQNEKLYLY |
| | | YLQNGRDMYVDQELDINRLSDYDVDAIVPQSFLKDDSI |
| | | DNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESIRPKRNSDK |
| | | LIARKKDWDPKKYGGFVSPTVAYSVLVVAKVEKGKSK |
| | | KLKSVKELLGITIMERSSFEKNPIDFLEAKGYKEVKKDLI |
| | | IKLPKYSLFELENGRKRMLASARFLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPRAFKYFDTTIDRKVYRSTKEVLDATLIHQSI |
| | | TGLYETRIDLSQLGGD |
| 14 | spCas9- | MDKKYSIGLDIGTNSVGWAVITDEYKVPSKKFKVLGNT |
| | VRQR | DRHSIKKNLIGALLFDSGETAEATRLKRTARRRYTRRKN |
| | | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDHIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFVSPTVAYSVLVVAKVEKGKSK |
| | | KLKSVKELLGITIMERSSFEKNPIDFLEAKGYKEVKKDLI |
| | | IKLPKYSLFELENGRKRMLASARELQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKQYRSTKEVLDATLIHQS |
| | | ITGLYETRIDLSQLGGD |
| 15 | spCas9- | MDKKYSIGLDIGTNSVGWAVITDEYKVPSKKFKVLGNT |
| | VRQR | DRHSIKKNLIGALLFDSGETAEATRLKRTARRRYTRRKN |
| | (H840A) | RICYLQEIFSNEMAKVDDSFFHRLEESFLVEEDKKHERH |
| | Nickase | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDAIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFVSPTVAYSVLVVAKVEKGKSK |
| | | KLKSVKELLGITIMERSSFEKNPIDFLEAKGYKEVKKDLI |
| | | IKLPKYSLFELENGRKRMLASARELQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKQYRSTKEVLDATLIHQS |
| | | ITGLYETRIDLSQLGGD |
| 16 | Met(−) | DKKYSIGLDIGTNSVGWAVITDEYKVPSKKFKVLGNTD |
| | SpCas9- | RHSIKKNLIGALLFDSGETAEATRLKRTARRRYTRRKNR |
| | VRQR | ICYLQEIFSNEMAKVDDSFFHRLEESFLVEEDKKHERHPI |
| | (H839A) | FGNIVDEVAYHEKYPTIYHLRKKLVDSTDKADLRLIYL |
| | Nickase | ALAHMIKFRGHFLIEGDLNPDNSDVDKLFIQLVQTYNQ |
| | | LFEENPINASGVDAKAILSARLSKSRRLENLIAQLPGEKK |
| | | NGLFGNLIALSLGLTPNFKSNFDLAEDAKLQLSKDTYD |
| | | DDLDNLLAQIGDQYADLFLAAKNLSDAILLSDILRVNTE |
| | | ITKAPLSASMIKRYDEHHQDLTLLKALVRQQLPEKYKEI |
| | | FFDQSKNGYAGYIDGGASQEEFYKFIKPILEKMDGTEEL |
| | | LVKLNREDLLRKQRTFDNGSIPHQIHLGELHAILRRQED |
| | | FYPFLKDNREKIEKILTFRIPYYVGPLARGNSRFAWMTR |
| | | KSEETITPWNFEEVVDKGASAQSFIERMTNFDKNLPNEK |
| | | VLPKHSLLYEYFTVYNELTKVKYVTEGMRKPAFLSGEQ |
| | | KKAIVDLLFKTNRKVTVKQLKEDYFKKIECFDSVEISGV |
| | | EDRFNASLGTYHDLLKIIKDKDFLDNEENEDILEDIVLTL |
| | | TLFEDREMIEERLKTYAHLFDDKVMKQLKRRRYTGWG |
| | | RLSRKLINGIRDKQSGKTILDFLKSDGFANRNFMQLIHD |
| | | DSLTFKEDIQKAQVSGQGDSLHEHIANLAGSPAIKKGIL |
| | | QTVKVVDELVKVMGRHKPENIVIEMARENQTTQKGQK |
| | | NSRERMKRIEEGIKELGSQILKEHPVENTQLQNEKLYLY |
| | | YLQNGRDMYVDQELDINRLSDYDVDAIVPQSFLKDDSI |
| | | DNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFVSPTVAYSVLVVAKVEKGKSK |
| | | KLKSVKELLGITIMERSSFEKNPIDFLEAKGYKEVKKDLI |
| | | IKLPKYSLFELENGRKRMLASARELQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKQYRSTKEVLDATLIHQS |
| | | ITGLYETRIDLSQLGGD |
| 17 | SpRY Cas9 | MDKKYSIGLDIGTNSVGWAVITDEYKVPSKKFKVLGNT |
| | | DRHSIKKNLIGALLFDSGETAERTRLKRTARRRYTRRKN |
| | | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDHIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESIRPKRNSDK |
| | | LIARKKDWDPKKYGGFLWPTVAYSVLVVAKVEKGKSK |
| | | KLKSVKELLGITIMERSSFEKNPIDFLEAKGYKEVKKDLI |
| | | IKLPKYSLFELENGRKRMLASAKQLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTRLGAPRAFKYFDTTIDPKQYRSTKEVLDATLIHQSI |
| | | TGLYETRIDLSQLGGD |
| 18 | SpRY | MDKKYSIGLDIGTNSVGWAVITDEYKVPSKKFKVLGNT |
| | (H840A) Nickase | DRHSIKKNLIGALLFDSGETAERTRLKRTARRRYTRRKN |
| | | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDAIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESIRPKRNSDK |
| | | LIARKKDWDPKKYGGFLWPTVAYSVLVVAKVEKGKSK |
| | | KLKSVKELLGITIMERSSFEKNPIDFLEAKGYKEVKKDLI |
| | | IKLPKYSLFELENGRKRMLASAKQLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTRLGAPRAFKYFDTTIDPKQYRSTKEVLDATLIHQSI |
| | | TGLYETRIDLSQLGGD |
| 19 | Met(−) | DKKYSIGLDIGTNSVGWAVITDEYKVPSKKFKVLGNTD |
| | SpRY | RHSIKKNLIGALLFDSGETAERTRLKRTARRRYTRRKNR |
| | (H839A) Nickase | ICYLQEIFSNEMAKVDDSFFHRLEESFLVEEDKKHERHPI |
| | | FGNIVDEVAYHEKYPTIYHLRKKLVDSTDKADLRLIYL |
| | | ALAHMIKFRGHFLIEGDLNPDNSDVDKLFIQLVQTYNQ |
| | | LFEENPINASGVDAKAILSARLSKSRRLENLIAQLPGEKK |
| | | NGLFGNLIALSLGLTPNFKSNFDLAEDAKLQLSKDTYD |
| | | DDLDNLLAQIGDQYADLFLAAKNLSDAILLSDILRVNTE |
| | | ITKAPLSASMIKRYDEHHQDLTLLKALVRQQLPEKYKEI |
| | | FFDQSKNGYAGYIDGGASQEEFYKFIKPILEKMDGTEEL |
| | | LVKLNREDLLRKQRTFDNGSIPHQIHLGELHAILRRQED |
| | | FYPFLKDNREKIEKILTFRIPYYVGPLARGNSRFAWMTR |
| | | KSEETITPWNFEEVVDKGASAQSFIERMTNFDKNLPNEK |
| | | VLPKHSLLYEYFTVYNELTKVKYVTEGMRKPAFLSGEQ |
| | | KKAIVDLLFKTNRKVTVKQLKEDYFKKIECFDSVEISGV |
| | | EDRFNASLGTYHDLLKIIKDKDFLDNEENEDILEDIVLTL |
| | | TLFEDREMIEERLKTYAHLFDDKVMKQLKRRRYTGWG |
| | | RLSRKLINGIRDKQSGKTILDFLKSDGFANRNFMQLIHD |
| | | DSLTFKEDIQKAQVSGQGDSLHEHIANLAGSPAIKKGIL |
| | | QTVKVVDELVKVMGRHKPENIVIEMARENQTTQKGQK |
| | | NSRERMKRIEEGIKELGSQILKEHPVENTQLQNEKLYLY |
| | | YLQNGRDMYVDQELDINRLSDYDVDAIVPQSFLKDDSI |
| | | DNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESIRPKRNSDK |
| | | LIARKKDWDPKKYGGFLWPTVAYSVLVVAKVEKGKSK |
| | | KLKSVKELLGITIMERSSFEKNPIDFLEAKGYKEVKKDLI |
| | | IKLPKYSLFELENGRKRMLASAKQLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTRLGAPRAFKYFDTTIDPKQYRSTKEVLDATLIHQSI |
| | | TGLYETRIDLSQLGGD |
| 20 | SRGN3.1 | MNQKFILGLDIGITSVGYGLIDYETKNIIDAGVRLFPEAN |
| | | VENNEGRRSKRGSRRLKRRRIHRLERVKLLLTEYDLINK |
| | | EQIPTSNNPYQIRVKGLSEILSKDELAIALLHLAKRRGIH |
| | | NVDVAADKEETASDSLSTKDQINKNAKFLESRYVCELQ |
| | | KERLENEGHVRGVENRFLTKDIVREAKKIIDTQMQYYP |
| | | EIDETFKEKYISLVETRREYFEGPGQGSPFGWNGDLKK |
| | | WYEMLMGHCTYFPQELRSVKYAYSADLFNALNDLNNL |
| | | IIQRDNSEKLEYHEKYHIIENVFKQKKKPTLKQIAKEIGV |
| | | NPEDIKGYRITKSGTPEFTSFKLFHDLKKVVKDHAILDDI |
| | | DLLNQIAEILTIYQDKDSIVAELGQLEYLMSEADKQSISE |
| | | LTGYTGTHSLSLKCMNMIIDELWHSSMNQMEVFTYLN |
| | | MRPKKYELKGYQRIPTDMIDDAILSPVVKRTFIQSINVIN |
| | | KVIEKYGIPEDIIIELARENNSDDRKKFINNLQKKNEATR |
| | | KRINEIIGQTGNQNAKRIVEKIRLHDQQEGKCLYSLESIP |
| | | LEDLLNNPNHYEVDHIIPRSVSFDNSYHNKVLVKQSENS |
| | | KKSNLTPYQYFNSGKSKLSYNQFKQHILNLSKSQDRISK |
| | | KKKEYLLEERDINKFEVQKEFINRNLVDTRYATRELTN |
| | | YLKAYFSANNMNVKVKTINGSFTDYLRKVWKFKKERN |
| | | HGYKHHAEDALIIANADFLFKENKKLKAVNSVLEKPEIE |
| | | TKQLDIQVDSEDNYSEMFIIPKQVQDIKDFRNFKYSHRV |
| | | DKKPNRQLINDTLYSTRKKDNSTYIVQTIKDIYAKDNTT |
| | | LKKQFDKSPEKFLMYQHDPRTFEKLEVIMKQYANEKNP |
| | | LAKYHEETGEYLTKYSKKNNGPIVKSLKYIGNKLGSHL |
| | | DVTHQFKSSTKKLVKLSIKNYRFDVYLTEKGYKFVTIA |
| | | YLNVFKKDNYYYIPKDKYQELKEKKKIKDTDQFIASFY |
| | | KNDLIKLNGDLYKIIGVNSDDRNIIELDYYDIKYKDYCEI |
| | | NNIKGEPRIKKTIGKKTESIEKFTTDVLGNLYLHSTEKAP |
| | | QLIFKRGL |
| 21 | SRGN3.1 | MNQKFILGLDIGITSVGYGLIDYETKNIIDAGVRLFPEAN |
| | (N585A) | VENNEGRRSKRGSRRLKRRRIHRLERVKLLLTEYDLINK |
| | Nickase | EQIPTSNNPYQIRVKGLSEILSKDELAIALLHLAKRRGIH |
| | | NVDVAADKEETASDSLSTKDQINKNAKFLESRYVCELQ |
| | | KERLENEGHVRGVENRFLTKDIVREAKKIIDTQMQYYP |
| | | EIDETFKEKYISLVETRREYFEGPGQGSPFGWNGDLKK |
| | | WYEMLMGHCTYFPQELRSVKYAYSADLFNALNDLNNL |
| | | IIQRDNSEKLEYHEKYHIIENVFKQKKKPTLKQIAKEIGV |
| | | NPEDIKGYRITKSGTPEFTSFKLFHDLKKVVKDHAILDDI |
| | | DLLNQIAEILTIYQDKDSIVAELGQLEYLMSEADKQSISE |
| | | LTGYTGTHSLSLKCMNMIIDELWHSSMNQMEVFTYLN |
| | | MRPKKYELKGYQRIPTDMIDDAILSPVVKRTFIQSINVIN |
| | | KVIEKYGIPEDIIIELARENNSDDRKKFINNLQKKNEATR |
| | | KRINEIIGQTGNQNAKRIVEKIRLHDQQEGKCLYSLESIP |
| | | LEDLLNNPNHYEVDHIIPRSVSFDNSYHNKVLVKQSEAS |
| | | KKSNLTPYQYFNSGKSKLSYNQFKQHILNLSKSQDRISK |
| | | KKKEYLLEERDINKFEVQKEFINRNLVDTRYATRELTN |
| | | YLKAYFSANNMNVKVKTINGSFTDYLRKVWKFKKERN |
| | | HGYKHHAEDALIIANADFLFKENKKLKAVNSVLEKPEIE |
| | | TKQLDIQVDSEDNYSEMFIIPKQVQDIKDFRNFKYSHRV |
| | | DKKPNRQLINDTLYSTRKKDNSTYIVQTIKDIYAKDNTT |
| | | LKKQFDKSPEKFLMYQHDPRTFEKLEVIMKQYANEKNP |
| | | LAKYHEETGEYLTKYSKKNNGPIVKSLKYIGNKLGSHL |
| | | DVTHQFKSSTKKLVKLSIKNYRFDVYLTEKGYKFVTIA |
| | | YLNVFKKDNYYYIPKDKYQELKEKKKIKDTDQFIASFY |
| | | KNDLIKLNGDLYKIIGVNSDDRNIIELDYYDIKYKDYCEI |
| | | NNIKGEPRIKKTIGKKTESIEKFTTDVLGNLYLHSTEKAP |
| | | QLIFKRGL |
| 22 | Met(−) | NQKFILGLDIGITSVGYGLIDYETKNIIDAGVRLFPEANV |
| | SRGN3.1 | ENNEGRRSKRGSRRLKRRRIHRLERVKLLLTEYDLINKE |
| | (N584A) | QIPTSNNPYQIRVKGLSEILSKDELAIALLHLAKRRGIHN |
| | Nickase | VDVAADKEETASDSLSTKDQINKNAKFLESRYVCELQK |
| | | ERLENEGHVRGVENRFLTKDIVREAKKIIDTQMQYYPEI |
| | | DETFKEKYISLVETRREYFEGPGQGSPFGWNGDLKKWY |
| | | EMLMGHCTYFPQELRSVKYAYSADLFNALNDLNNLIIQ |
| | | RDNSEKLEYHEKYHIIENVFKQKKKPTLKQIAKEIGVNP |
| | | EDIKGYRITKSGTPEFTSFKLFHDLKKVVKDHAILDDIDL |
| | | LNQIAEILTIYQDKDSIVAELGQLEYLMSEADKQSISELT |
| | | GYTGTHSLSLKCMNMIIDELWHSSMNQMEVFTYLNMR |
| | | PKKYELKGYQRIPTDMIDDAILSPVVKRTFIQSINVINKV |
| | | IEKYGIPEDIIIELARENNSDDRKKFINNLQKKNEATRKRI |
| | | NEIIGQTGNQNAKRIVEKIRLHDQQEGKCLYSLESIPLED |
| | | LLNNPNHYEVDHIIPRSVSFDNSYHNKVLVKQSEASKKS |
| | | NLTPYQYFNSGKSKLSYNQFKQHILNLSKSQDRISKKKK |
| | | EYLLEERDINKFEVQKEFINRNLVDTRYATRELTNYLKA |
| | | YFSANNMNVKVKTINGSFTDYLRKVWKFKKERNHGYK |
| | | HHAEDALIIANADFLFKENKKLKAVNSVLEKPEIETKQL |
| | | DIQVDSEDNYSEMFIIPKQVQDIKDFRNFKYSHRVDKKP |
| | | NRQLINDTLYSTRKKDNSTYIVQTIKDIYAKDNTTLKKQ |
| | | FDKSPEKFLMYQHDPRTFEKLEVIMKQYANEKNPLAKY |
| | | HEETGEYLTKYSKKNNGPIVKSLKYIGNKLGSHLDVTH |
| | | QFKSSTKKLVKLSIKNYRFDVYLTEKGYKFVTIAYLNVF |
| | | KKDNYYYIPKDKYQELKEKKKIKDTDQFIASFYKNDLI |
| | | KLNGDLYKIIGVNSDDRNIIELDYYDIKYKDYCEINNIKG |
| | | EPRIKKTIGKKTESIEKFTTDVLGNLYLHSTEKAPQLIFK |
| | | RGL |
| 23 | sRGN3.3 | MNQKFILGLDIGITSVGYGLIDYETKNIIDAGVRLFPEAN |
| | Nickase | VENNEGRRSKRGSRRLKRRRIHRLERVKLLLTEYDLINK |
| | | EQIPTSNNPYQIRVKGLSEILSKDELAIALLHLAKRRGIH |
| | | NVDVAADKEETASDSLSTKDQINKNAKFLESRYVCELQ |
| | | KERLENEGHVRGVENRFLTKDIVREAKKIIDTQMQYYP |
| | | EIDETFKEKYISLVETRREYFEGPGQGSPFGWNGDLKK |
| | | WYEMLMGHCTYFPQELRSVKYAYSADLFNALNDLNNL |
| | | IIQRDNSEKLEYHEKYHIIENVFKQKKKPTLKQIAKEIGV |
| | | NPEDIKGYRITKSGTPEFTSFKLFHDLKKVVKDHAILDDI |
| | | DLLNQIAEILTIYQDKDSIVAELGQLEYLMSEADKQSISE |
| | | LTGYTGTHSLSLKCMNMIIDELWHSSMNQMEVFTYLN |
| | | MRPKKYELKGYQRIPTDMIDDAILSPVVKRTFIQSINVIN |
| | | KVIEKYGIPEDIIIELARENNSDDRKKFINNLQKKNEATR |
| | | KRINEIIGQTGNQNAKRIVEKIRLHDQQEGKCLYSLESIP |
| | | LEDLLNNPNHYEVDHIIPRSVSFDNSYHNKVLVKQSENS |
| | | KKSNLTPYQYFNSGKSKLSYNQFKQHILNLSKSQDRISK |
| | | KKKEYLLEERDINKFEVQKEFINRNLVDTRYATRELTSY |
| | | LKAYFSANNMDVKVKTINGSFTNHLRKVWRFDKYRNH |
| | | GYKHHAEDALIIANADFLFKENKKLQNTNKILEKPTIEN |
| | | NTKKVTVEKEEDYNNVFETPKLVEDIKQYRDYKFSHRV |
| | | DKKPNRQLINDTLYSTRMKDEHDYIVQTITDIYGKDNT |
| | | NLKKQFNKNPEKFLMYQNDPKTFEKLSIIMKQYSDEKN |
| | | PLAKYYEETGEYLTKYSKKNNGPIVKKIKLLGNKVGNH |
| | | LDVTNKYENSTKKLVKLSIKNYRFDVYLTEKGYKFVTI |
| | | AYLNVFKKDNYYYIPKDKYQELKEKKKIKDTDQFIASF |
| | | YKNDLIKLNGDLYKIIGVNSDDRNIIELDYYDIKYKDYC |
| | | EINNIKGEPRIKKTIGKKTESIEKFTTDVLGNLYLHSTEK |
| | | APQLIFKRGL |
| 24 | SRGN3.3 | MNQKFILGLDIGITSVGYGLIDYETKNIIDAGVRLFPEAN |
| | (N585A) | VENNEGRRSKRGSRRLKRRRIHRLERVKLLLTEYDLINK |
| | Nickase | EQIPTSNNPYQIRVKGLSEILSKDELAIALLHLAKRRGIH |
| | | NVDVAADKEETASDSLSTKDQINKNAKFLESRYVCELQ |
| | | KERLENEGHVRGVENRFLTKDIVREAKKIIDTQMQYYP |
| | | EIDETFKEKYISLVETRREYFEGPGQGSPFGWNGDLKK |
| | | WYEMLMGHCTYFPQELRSVKYAYSADLFNALNDLNNL |
| | | IIQRDNSEKLEYHEKYHIIENVFKQKKKPTLKQIAKEIGV |
| | | NPEDIKGYRITKSGTPEFTSFKLFHDLKKVVKDHAILDDI |
| | | DLLNQIAEILTIYQDKDSIVAELGQLEYLMSEADKQSISE |
| | | LTGYTGTHSLSLKCMNMIIDELWHSSMNQMEVFTYLN |
| | | MRPKKYELKGYQRIPTDMIDDAILSPVVKRTFIQSINVIN |
| | | KVIEKYGIPEDIIIELARENNSDDRKKFINNLQKKNEATR |
| | | KRINEIIGQTGNQNAKRIVEKIRLHDQQEGKCLYSLESIP |
| | | LEDLLNNPNHYEVDHIIPRSVSFDNSYHNKVLVKQSEAS |
| | | KKSNLTPYQYFNSGKSKLSYNQFKQHILNLSKSQDRISK |
| | | KKKEYLLEERDINKFEVQKEFINRNLVDTRYATRELTSY |
| | | LKAYFSANNMDVKVKTINGSFTNHLRKVWRFDKYRNH |
| | | GYKHHAEDALIIANADFLFKENKKLQNTNKILEKPTIEN |
| | | NTKKVTVEKEEDYNNVFETPKLVEDIKQYRDYKFSHRV |
| | | DKKPNRQLINDTLYSTRMKDEHDYIVQTITDIYGKDNT |
| | | NLKKQFNKNPEKFLMYQNDPKTFEKLSIIMKQYSDEKN |
| | | PLAKYYEETGEYLTKYSKKNNGPIVKKIKLLGNKVGNH |
| | | LDVTNKYENSTKKLVKLSIKNYRFDVYLTEKGYKFVTI |
| | | AYLNVFKKDNYYYIPKDKYQELKEKKKIKDTDQFIASF |
| | | YKNDLIKLNGDLYKIIGVNSDDRNIIELDYYDIKYKDYC |
| | | EINNIKGEPRIKKTIGKKTESIEKFTTDVLGNLYLHSTEK |
| | | APQLIFKRGL |
| 25 | Met(−) | NQKFILGLDIGITSVGYGLIDYETKNIIDAGVRLFPEANV |
| | SRGN3.3 | ENNEGRRSKRGSRRLKRRRIHRLERVKLLLTEYDLINKE |
| | (N584A) | QIPTSNNPYQIRVKGLSEILSKDELAIALLHLAKRRGIHN |
| | Nickase | VDVAADKEETASDSLSTKDQINKNAKFLESRYVCELQK |
| | | ERLENEGHVRGVENRFLTKDIVREAKKIIDTQMQYYPEI |
| | | DETFKEKYISLVETRREYFEGPGQGSPFGWNGDLKKWY |
| | | EMLMGHCTYFPQELRSVKYAYSADLFNALNDLNNLIIQ |
| | | RDNSEKLEYHEKYHIIENVFKQKKKPTLKQIAKEIGVNP |
| | | EDIKGYRITKSGTPEFTSFKLFHDLKKVVKDHAILDDIDL |
| | | LNQIAEILTIYQDKDSIVAELGQLEYLMSEADKQSISELT |
| | | GYTGTHSLSLKCMNMIIDELWHSSMNQMEVFTYLNMR |
| | | PKKYELKGYQRIPTDMIDDAILSPVVKRTFIQSINVINKV |
| | | IEKYGIPEDIIIELARENNSDDRKKFINNLQKKNEATRKRI |
| | | NEIIGQTGNQNAKRIVEKIRLHDQQEGKCLYSLESIPLED |
| | | LLNNPNHYEVDHIIPRSVSFDNSYHNKVLVKQSEASKKS |
| | | NLTPYQYFNSGKSKLSYNQFKQHILNLSKSQDRISKKKK |
| | | EYLLEERDINKFEVQKEFINRNLVDTRYATRELTSYLKA |
| | | YFSANNMDVKVKTINGSFTNHLRKVWRFDKYRNHGY |
| | | KHHAEDALIIANADFLFKENKKLQNTNKILEKPTIENNT |
| | | KKVTVEKEEDYNNVFETPKLVEDIKQYRDYKFSHRVD |
| | | KKPNRQLINDTLYSTRMKDEHDYIVQTITDIYGKDNTNL |
| | | KKQFNKNPEKFLMYQNDPKTFEKLSIIMKQYSDEKNPL |
| | | AKYYEETGEYLTKYSKKNNGPIVKKIKLLGNKVGNHLD |
| | | VTNKYENSTKKLVKLSIKNYRFDVYLTEKGYKFVTIAY |
| | | LNVFKKDNYYYIPKDKYQELKEKKKIKDTDQFIASFYK |
| | | NDLIKLNGDLYKIIGVNSDDRNIIELDYYDIKYKDYCEIN |
| | | NIKGEPRIKKTIGKKTESIEKFTTDVLGNLYLHSTEKAPQ |
| | | LIFKRGL |
| 26 | SpG Cas9 | MDKKYSIGLDIGTNSVGWAVITDEYKVPSKKFKVLGNT |
| | | DRHSIKKNLIGALLFDSGETAEATRLKRTARRRYTRRKN |
| | | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDHIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFLWPTVAYSVLVVAKVEKGKSK |
| | | KLKSVKELLGITIMERSSFEKNPIDFLEAKGYKEVKKDLI |
| | | IKLPKYSLFELENGRKRMLASAKQLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKQYRSTKEVLDATLIHQS |
| | | ITGLYETRIDLSQLGGD |
| 27 | SpG | MDKKYSIGLDIGTNSVGWAVITDEYKVPSKKFKVLGNT |
| | (H840A) | DRHSIKKNLIGALLFDSGETAEATRLKRTARRRYTRRKN |
| | Nickase | RICYLQEIFSNEMAKVDDSFFHRLEESFLVEEDKKHERH |
| | | PIFGNIVDEVAYHEKYPTIYHLRKKLVDSTDKADLRLIY |
| | | LALAHMIKFRGHFLIEGDLNPDNSDVDKLFIQLVQTYN |
| | | QLFEENPINASGVDAKAILSARLSKSRRLENLIAQLPGEK |
| | | KNGLFGNLIALSLGLTPNFKSNFDLAEDAKLQLSKDTY |
| | | DDDLDNLLAQIGDQYADLFLAAKNLSDAILLSDILRVNT |
| | | EITKAPLSASMIKRYDEHHQDLTLLKALVRQQLPEKYK |
| | | EIFFDQSKNGYAGYIDGGASQEEFYKFIKPILEKMDGTE |
| | | ELLVKLNREDLLRKQRTFDNGSIPHQIHLGELHAILRRQ |
| | | EDFYPFLKDNREKIEKILTFRIPYYVGPLARGNSRFAWM |
| | | TRKSEETITPWNFEEVVDKGASAQSFIERMTNFDKNLPN |
| | | EKVLPKHSLLYEYFTVYNELTKVKYVTEGMRKPAFLSG |
| | | EQKKAIVDLLFKTNRKVTVKQLKEDYFKKIECFDSVEIS |
| | | GVEDRFNASLGTYHDLLKIIKDKDFLDNEENEDILEDIV |
| | | LTLTLFEDREMIEERLKTYAHLFDDKVMKQLKRRRYTG |
| | | WGRLSRKLINGIRDKQSGKTILDFLKSDGFANRNFMQLI |
| | | HDDSLTFKEDIQKAQVSGQGDSLHEHIANLAGSPAIKKG |
| | | ILQTVKVVDELVKVMGRHKPENIVIEMARENQTTQKGQ |
| | | KNSRERMKRIEEGIKELGSQILKEHPVENTQLQNEKLYL |
| | | YYLQNGRDMYVDQELDINRLSDYDVDAIVPQSFLKDDS |
| | | IDNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFLWPTVAYSVLVVAKVEKGKSK |
| | | KLKSVKELLGITIMERSSFEKNPIDFLEAKGYKEVKKDLI |
| | | IKLPKYSLFELENGRKRMLASAKQLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKQYRSTKEVLDATLIHQS |
| | | ITGLYETRIDLSQLGGD |
| 28 | Met(−) SpG | DKKYSIGLDIGTNSVGWAVITDEYKVPSKKFKVLGNTD |
| | (H839A) | RHSIKKNLIGALLFDSGETAEATRLKRTARRRYTRRKNR |
| | Nickase | ICYLQEIFSNEMAKVDDSFFHRLEESFLVEEDKKHERHPI |
| | | FGNIVDEVAYHEKYPTIYHLRKKLVDSTDKADLRLIYL |
| | | ALAHMIKFRGHFLIEGDLNPDNSDVDKLFIQLVQTYNQ |
| | | LFEENPINASGVDAKAILSARLSKSRRLENLIAQLPGEKK |
| | | NGLFGNLIALSLGLTPNFKSNFDLAEDAKLQLSKDTYD |
| | | DDLDNLLAQIGDQYADLFLAAKNLSDAILLSDILRVNTE |
| | | ITKAPLSASMIKRYDEHHQDLTLLKALVRQQLPEKYKEI |
| | | FFDQSKNGYAGYIDGGASQEEFYKFIKPILEKMDGTEEL |
| | | LVKLNREDLLRKQRTFDNGSIPHQIHLGELHAILRRQED |
| | | FYPFLKDNREKIEKILTFRIPYYVGPLARGNSRFAWMTR |
| | | KSEETITPWNFEEVVDKGASAQSFIERMTNFDKNLPNEK |
| | | VLPKHSLLYEYFTVYNELTKVKYVTEGMRKPAFLSGEQ |
| | | KKAIVDLLFKTNRKVTVKQLKEDYFKKIECFDSVEISGV |
| | | EDRFNASLGTYHDLLKIIKDKDFLDNEENEDILEDIVLTL |
| | | TLFEDREMIEERLKTYAHLFDDKVMKQLKRRRYTGWG |
| | | RLSRKLINGIRDKQSGKTILDFLKSDGFANRNFMQLIHD |
| | | DSLTFKEDIQKAQVSGQGDSLHEHIANLAGSPAIKKGIL |
| | | QTVKVVDELVKVMGRHKPENIVIEMARENQTTQKGQK |
| | | NSRERMKRIEEGIKELGSQILKEHPVENTQLQNEKLYLY |
| | | YLQNGRDMYVDQELDINRLSDYDVDAIVPQSFLKDDSI |
| | | DNKVLTRSDKNRGKSDNVPSEEVVKKMKNYWRQLLN |
| | | AKLITQRKFDNLTKAERGGLSELDKAGFIKRQLVETRQI |
| | | TKHVAQILDSRMNTKYDENDKLIREVKVITLKSKLVSD |
| | | FRKDFQFYKVREINNYHHAHDAYLNAVVGTALIKKYP |
| | | KLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDK |
| | | LIARKKDWDPKKYGGFLWPTVAYSVLVVAKVEKGKSK |
| | | KLKSVKELLGITIMERSSFEKNPIDFLEAKGYKEVKKDLI |
| | | IKLPKYSLFELENGRKRMLASAKQLQKGNELALPSKYV |
| | | NFLYLASHYEKLKGSPEDNEQKQLFVEQHKHYLDEIIEQ |
| | | ISEFSKRVILADANLDKVLSAYNKHRDKPIREQAENIIHL |
| | | FTLTNLGAPAAFKYFDTTIDRKQYRSTKEVLDATLIHQS |
| | | ITGLYETRIDLSQLGGD |
| NA | saCas9 | NNGRRT |
| | PAM | |
| NA | saCas9 | NNGRRN |
| | PAM | |
| NA | saCas9- | NNNRRT |
| | KKH | |
| | (E782K, | |
| | N968K, | |
| | R1015H) | |
| | PAM | |
| NA | NmCas9 | NNNNGATT |
| | PAM | |
| NA | StCas9 | NNAGAAW |
| | PAM | |
| NA | TdCas9 | NAAAAC |
| | PAM | |
| 29 | NLS | MDSLLMNRRKFLYQFKNVRWAKGRRETYLC |
| 30 | N-terminal | MKRTADGSEFESPKKKRKV |
| | NLS and | |
| | Linker for | |
| | Exem. PE | |
| 35 | NLS of | PKKKRKV |
| | SV40 Large | |
| | T-AG | |
| 38 | NLS of | AVKRPAATKKAGQAKKKKLD |
| | Nucleoplas | |
| | min | |
| 39 | NLS of | MSRRRKANPTKLSENAKKLAKEVEN |
| | EGL-13 | |
| 40 | NLS of C- | PAAKRVKLD |
| | Myc | |
| 41 | NLS of Tus- | KLKIKRPVK |
| | protein | |
| 42 | NLS of | VSRKRPRP |
| | polyoma | |
| | large T-AG | |
| 43 | NLS of | EGAPPAKRAR |
| | Hepatitis D | |
| | virus | |
| | antigen | |
| 44 | NLS of | PPQPKKKPLDGE |
| | murine p53 | |
| 45 | C-Terminal | SGGSKRTADGSEFEPKKKRKV |
| | linker and | |
| | NLS for | |
| | Exemplary | |
| | Prime | |
| | Editor | |
| 46 | MS2 | GCCAACATGAGGATCACCCATGTCTGCAGGGCC |
| | aptamer | |
| 47 | MS2cp | GSASNFTQFVLVDNGGTGDVTVAPSNFANGVAEWISSN |
| | | SRSQAYKVTCSVRQSSAQNRKYTIKVEVPKVATQTVGG |
| | | EELPVAGWRSYLNMELTIPIFATNSDCELIVKAMQGLLK |
| | | DGNPIPSAIAANSGIY |
| 48 | Linker | (GGGGS)n |
| 49 | Linker | (G)n |
| 50 | Linker | (EAAAK)n |
| 51 | Linker | (GGS)n |
| 52 | Linker | (SGGS)n |
| 53 | Linker | (XP)n |
| 54 | Linker | SGSETPGTSESATPES |
| 55 | Linker | SGGSSGGSSGSETPGTSESATPESSGGSSGGS |
| 56 | Linker | SGGSSGGSSGSETPGTSESATPESSGGSSGGSS |
| | between | |
| | Cas9 | |
| | domain and | |
| | RT domain- | |
| | Exemplary | |
| | PE | |
| 57 | Linker | SGGSGGSGGS |
| 58 | Linker | SGGS |
| 59 | Linker | SGGSSGGSSGSETPGTSESATPESAGSYPYDVPDYAGSA |
| | | APAAKKKKLDGSGSGGSSGGS |
| 60 | Exemplary | MKRTADGSEFESPKKKRKVDKKYSIGLDIGTNSVGWA |
| | Prime | VITDEYKVPSKKFKVLGNTDRHSIKKNLIGALLFDSGET |
| | Editor | AEATRLKRTARRRYTRRKNRICYLQEIFSNEMAKVDDS |
| | [NLS]-[Cas9 | FFHRLEESFLVEEDKKHERHPIFGNIVDEVAYHEKYPTIY |
| | (H840A)]- | HLRKKLVDSTDKADLRLIYLALAHMIKFRGHFLIEGDL |
| | [linker]- | NPDNSDVDKLFIQLVQTYNQLFEENPINASGVDAKAILS |
| | [MMLV_RT | ARLSKSRRLENLIAQLPGEKKNGLFGNLIALSLGLTPNF |
| | (D200N) | KSNFDLAEDAKLQLSKDTYDDDLDNLLAQIGDQYADL |
| | (T330P) | FLAAKNLSDAILLSDILRVNTEITKAPLSASMIKRYDEHH |
| | (L603W) | QDLTLLKALVRQQLPEKYKEIFFDQSKNGYAGYIDGGA |
| | (T306K) | SQEEFYKFIKPILEKMDGTEELLVKLNREDLLRKQRTFD |
| | (W313F)]- | NGSIPHQIHLGELHAILRRQEDFYPFLKDNREKIEKILTFR |
| | [NLS] | IPYYVGPLARGNSRFAWMTRKSEETITPWNFEEVVDKG |
| | | ASAQSFIERMTNFDKNLPNEKVLPKHSLLYEYFTVYNEL |
| | | TKVKYVTEGMRKPAFLSGEQKKAIVDLLFKTNRKVTV |
| | | KQLKEDYFKKIECFDSVEISGVEDRFNASLGTYHDLLKII |
| | | KDKDFLDNEENEDILEDIVLTLTLFEDREMIEERLKTYA |
| | | HLFDDKVMKQLKRRRYTGWGRLSRKLINGIRDKQSGK |
| | | TILDFLKSDGFANRNFMQLIHDDSLTFKEDIQKAQVSGQ |
| | | GDSLHEHIANLAGSPAIKKGILQTVKVVDELVKVMGRH |
| | | KPENIVIEMARENQTTQKGQKNSRERMKRIEEGIKELGS |
| | | QILKEHPVENTQLQNEKLYLYYLQNGRDMYVDQELDI |
| | | NRLSDYDVDAIVPQSFLKDDSIDNKVLTRSDKNRGKSD |
| | | NVPSEEVVKKMKNYWRQLLNAKLITQRKFDNLTKAER |
| | | GGLSELDKAGFIKRQLVETRQITKHVAQILDSRMNTKY |
| | | DENDKLIREVKVITLKSKLVSDFRKDFQFYKVREINNYH |
| | | HAHDAYLNAVVGTALIKKYPKLESEFVYGDYKVYDVR |
| | | KMIAKSEQEIGKATAKYFFYSNIMNFFKTEITLANGEIRK |
| | | RPLIETNGETGEIVWDKGRDFATVRKVLSMPQVNIVKK |
| | | TEVQTGGFSKESILPKRNSDKLIARKKDWDPKKYGGFD |
| | | SPTVAYSVLVVAKVEKGKSKKLKSVKELLGITIMERSSF |
| | | EKNPIDFLEAKGYKEVKKDLIIKLPKYSLFELENGRKRM |
| | | LASAGELQKGNELALPSKYVNFLYLASHYEKLKGSPED |
| | | NEQKQLFVEQHKHYLDEIIEQISEFSKRVILADANLDKV |
| | | LSAYNKHRDKPIREQAENIIHLFTLTNLGAPAAFKYFDT |
| | | TIDRKRYTSTKEVLDATLIHQSITGLYETRIDLSQLGGDS |
| | | GGSSGGSSGSETPGTSESATPESSGGSSGGSSTLNIEDEY |
| | | RLHETSKEPDVSLGSTWLSDFPQAWAETGGMGLAVRQ |
| | | APLIIPLKATSTPVSIKQYPMSQEARLGIKPHIQRLLDQGI |
| | | LVPCQSPWNTPLLPVKKPGTNDYRPVQDLREVNKRVE |
| | | DIHPTVPNPYNLLSGLPPSHQWYTVLDLKDAFFCLRLHP |
| | | TSQPLFAFEWRDPEMGISGQLTWTRLPQGFKNSPTLFNE |
| | | ALHRDLADFRIQHPDLILLQYVDDLLLAATSELDCQQG |
| | | TRALLQTLGNLGYRASAKKAQICQKQVKYLGYLLKEG |
| | | QRWLTEARKETVMGQPTPKTPRQLREFLGKAGFCRLFI |
| | | PGFAEMAAPLYPLTKPGTLFNWGPDQQKAYQEIKQALL |
| | | TAPALGLPDLTKPFELFVDEKQGYAKGVLTQKLGPWRR |
| | | PVAYLSKKLDPVAAGWPPCLRMVAAIAVLTKDAGKLT |
| | | MGQPLVILAPHAVEALVKQPPDRWLSNARMTHYQALL |
| | | LDTDRVQFGPVVALNPATLLPLPEEGLQHNCLDILAEA |
| | | HGTRPDLTDQPLPDADHTWYTDGSSLLQEGQRKAGAA |
| | | VTTETEVIWAKALPAGTSAQRAELIALTQALKMAEGKK |
| | | LNVYTDSRYAFATAHIHGEIYRRRGWLTSEGKEIKNKD |
| | | EILALLKALFLPKRLSIIHCPGHQKGHSAEARGNRMADQ |
| | | AARKAAITETPDTSTLLIENSSPSGGSKRTADGSEFEPKK |
| | | KRKV |
| 61 | PEmax-C- | SGGSKRTADGSEFESPKKKRKV |
| | terminal | |
| | linker-NLS | |
| 62 | PEmax | MKRTADGSEFESPKKKRKVDKKYSIGLDIGTNSVGWA |
| | Prime | VITDEYKVPSKKFKVLGNTDRHSIKKNLIGALLFDSGET |
| | Editor + | AEATRLKRTARRRYTRRKNRICYLQEIFSNEMAKVDDS |
| | NLS | FFHRLEESFLVEEDKKHERHPIFGNIVDEVAYHEKYPTIY |
| | | HLRKKLVDSTDKADLRLIYLALAHMIKFRGHFLIEGDL |
| | | NPDNSDVDKLFIQLVQTYNQLFEENPINASGVDAKAILS |
| | | ARLSKSRKLENLIAQLPGEKKNGLFGNLIALSLGLTPNF |
| | | KSNFDLAEDAKLQLSKDTYDDDLDNLLAQIGDQYADL |
| | | FLAAKNLSDAILLSDILRVNTEITKAPLSASMIKRYDEHH |
| | | QDLTLLKALVRQQLPEKYKEIFFDQSKNGYAGYIDGGA |
| | | SQEEFYKFIKPILEKMDGTEELLVKLKREDLLRKQRTFD |
| | | NGSIPHQIHLGELHAILRRQEDFYPFLKDNREKIEKILTFR |
| | | IPYYVGPLARGNSRFAWMTRKSEETITPWNFEEVVDKG |
| | | ASAQSFIERMTNFDKNLPNEKVLPKHSLLYEYFTVYNEL |
| | | TKVKYVTEGMRKPAFLSGEQKKAIVDLLFKTNRKVTV |
| | | KQLKEDYFKKIECFDSVEISGVEDRFNASLGTYHDLLKII |
| | | KDKDFLDNEENEDILEDIVLTLTLFEDREMIEERLKTYA |
| | | HLFDDKVMKQLKRRRYTGWGRLSRKLINGIRDKQSGK |
| | | TILDFLKSDGFANRNFMQLIHDDSLTFKEDIQKAQVSGQ |
| | | GDSLHEHIANLAGSPAIKKGILQTVKVVDELVKVMGRH |
| | | KPENIVIEMARENQTTQKGQKNSRERMKRIEEGIKELGS |
| | | QILKEHPVENTQLQNEKLYLYYLQNGRDMYVDQELDI |
| | | NRLSDYDVDAIVPQSFLKDDSIDNKVLTRSDKNRGKSD |
| | | NVPSEEVVKKMKNYWRQLLNAKLITQRKFDNLTKAER |
| | | GGLSELDKAGFIKRQLVETRQITKHVAQILDSRMNTKY |
| | | DENDKLIREVKVITLKSKLVSDFRKDFQFYKVREINNYH |
| | | HAHDAYLNAVVGTALIKKYPKLESEFVYGDYKVYDVR |
| | | KMIAKSEQEIGKATAKYFFYSNIMNFFKTEITLANGEIRK |
| | | RPLIETNGETGEIVWDKGRDFATVRKVLSMPQVNIVKK |
| | | TEVQTGGFSKESILPKRNSDKLIARKKDWDPKKYGGFD |
| | | SPTVAYSVLVVAKVEKGKSKKLKSVKELLGITIMERSSF |
| | | EKNPIDFLEAKGYKEVKKDLIIKLPKYSLFELENGRKRM |
| | | LASAGELQKGNELALPSKYVNFLYLASHYEKLKGSPED |
| | | NEQKQLFVEQHKHYLDEIIEQISEFSKRVILADANLDKV |
| | | LSAYNKHRDKPIREQAENIIHLFTLTNLGAPAAFKYFDT |
| | | TIDRKRYTSTKEVLDATLIHQSITGLYETRIDLSQLGGDS |
| | | GGSSGGSKRTADGSEFESPKKKRKVSGGSSGGSTLNIED |
| | | EYRLHETSKEPDVSLGSTWLSDFPQAWAETGGMGLAV |
| | | RQAPLIIPLKATSTPVSIKQYPMSQEARLGIKPHIQRLLD |
| | | QGILVPCQSPWNTPLLPVKKPGTNDYRPVQDLREVNKR |
| | | VEDIHPTVPNPYNLLSGLPPSHQWYTVLDLKDAFFCLRL |
| | | HPTSQPLFAFEWRDPEMGISGQLTWTRLPQGFKNSPTLF |
| | | NEALHRDLADFRIQHPDLILLQYVDDLLLAATSELDCQ |
| | | QGTRALLQTLGNLGYRASAKKAQICQKQVKYLGYLLK |
| | | EGQRWLTEARKETVMGQPTPKTPRQLREFLGKAGFCRL |
| | | FIPGFAEMAAPLYPLTKPGTLFNWGPDQQKAYQEIKQA |
| | | LLTAPALGLPDLTKPFELFVDEKQGYAKGVLTQKLGPW |
| | | RRPVAYLSKKLDPVAAGWPPCLRMVAAIAVLTKDAGK |
| | | LTMGQPLVILAPHAVEALVKQPPDRWLSNARMTHYQA |
| | | LLLDTDRVQFGPVVALNPATLLPLPEEGLQHNCLDILAE |
| | | AHGTRPDLTDQPLPDADHTWYTDGSSLLQEGQRKAGA |
| | | AVTTETEVIWAKALPAGTSAQRAELIALTQALKMAEGK |
| | | KLNVYTDSRYAFATAHIHGEIYRRRGWLTSEGKEIKNK |
| | | DEILALLKALFLPKRLSIIHCPGHQKGHSAEARGNRMAD |
| | | QAARKAAITETPDTSTLLIENSSPSGGSKRTADGSEFESP |
| | | KKKRKVGSGPAAKRVKLD |
| 63 | PEmax- | SGGSSGGSKRTADGSEFESPKKKRKVSGGSSGGS |
| | SGGSx2- | |
| | bpSV40NLS- | |
| | SGGSx2 | |
| | linker | |
| 64 | PEmax-C- | GSGPAAKRVKLD |
| | terminal | |
| | linker-NLS2 | |
| 301 | gRNA core | GTTTTAGAGCTAGAAATAGCAAGTTAAAATAAGGCT |
| | | AGTCCGTTATCAACTTGAAAAAGTGGCACCGAGTCG |
| | | GTGC |
| 302 | gRNA core | GTTTGAGAGCTAGAAATAGCAAGTTTAAATAAGGCT |
| | | AGTCCGTTATCAACTTGAAAAAGTGGGACCGAGTCG |
| | | GTCC |
| 303 | gRNA core | GTTTAAGAGCTATGCTGGAAACAGCATAGCAAGTTTA |
| | | AATAAGGCTAGTCCGTTATCAACTTGAAAAAGTGGC |
| | | ACCGAGTCGGTGC |
| 304 | SaCas9 | GTTTTAGTACTCTGGAAACAGAATCTACTAAAACAAG |
| | gRNA core | GCAAAATGCCGTGTTTATCTCGTCAACTTGTTGGCGA |
| | | GA |
| 305 | SluCas9 | GTTTTAGTACTCTGGAAACAGAATCTACTGAAACAAG |
| | gRNA core | ACAATATGTCGTGTTTATCCCATCAATTTATTGGTGG |
| | | GA |
| 363 | Staphylococcus | MKRNYILGLDIGITSVGYGIIDYETRDVIDAGVRLFKEA |
| | aureus | NVENNEGRRSKRGARRLKRRRRHRIQRVKKLLFDYNLL |
| | Cas9 | TDHSELSGINPYEARVKGLSQKLSEEEFSAALLHLAKRR |
| | (saCas9) | GVHNVNEVEEDTGNELSTKEQISRNSKALEEKYVAELQ |
| | | LERLKKDGEVRGSINRFKTSDYVKEAKQLLKVQKAYH |
| | | QLDQSFIDTYIDLLETRRTYYEGPGEGSPFGWKDIKEWY |
| | | EMLMGHCTYFPEELRSVKYAYNADLYNALNDLNNLVI |
| | | TRDENEKLEYYEKFQIIENVFKQKKKPTLKQIAKEILVN |
| | | EEDIKGYRVTSTGKPEFTNLKVYHDIKDITARKEIIENAE |
| | | LLDQIAKILTIYQSSEDIQEELTNLNSELTQEEIEQISNLK |
| | | GYTGTHNLSLKAINLILDELWHTNDNQIAIFNRLKLVPK |
| | | KVDLSQQKEIPTTLVDDFILSPVVKRSFIQSIKVINAIIKK |
| | | YGLPNDIIIELAREKNSKDAQKMINEMQKRNRQTNERIE |
| | | EIIRTTGKENAKYLIEKIKLHDMQEGKCLYSLEAIPLEDL |
| | | LNNPFNYEVDHIIPRSVSFDNSFNNKVLVKQEENSKKGN |
| | | RTPFQYLSSSDSKISYETFKKHILNLAKGKGRISKTKKEY |
| | | LLEERDINRFSVQKDFINRNLVDTRYATRGLMNLLRSYF |
| | | RVNNLDVKVKSINGGFTSFLRRKWKFKKERNKGYKHH |
| | | AEDALIIANADFIFKEWKKLDKAKKVMENQMFEEKQA |
| | | ESMPEIETEQEYKEIFITPHQIKHIKDFKDYKYSHRVDKK |
| | | PNRELINDTLYSTRKDDKGNTLIVNNLNGLYDKDNDKL |
| | | KKLINKSPEKLLMYHHDPQTYQKLKLIMEQYGDEKNPL |
| | | YKYYEETGNYLTKYSKKDNGPVIKKIKYYGNKLNAHL |
| | | DITDDYPNSRNKVVKLSLKPYRFDVYLDNGVYKFVTV |
| | | KNLDVIKKENYYEVNSKCYEEAKKLKKISNQAEFIASFY |
| | | NNDLIKINGELYRVIGVNNDLLNRIEVNMIDITYREYLE |
| | | NMNDKRPPRIIKTIASKTQSIKKYSTDILGNLYEVKSKKH |
| | | PQIIKKG |
| 364 | saCas9 | MKRNYILGLDIGITSVGYGIIDYETRDVIDAGVRLFKEA |
| | (N580A) | NVENNEGRRSKRGARRLKRRRRHRIQRVKKLLFDYNLL |
| | Nickase | TDHSELSGINPYEARVKGLSQKLSEEEFSAALLHLAKRR |
| | | GVHNVNEVEEDTGNELSTKEQISRNSKALEEKYVAELQ |
| | | LERLKKDGEVRGSINRFKTSDYVKEAKQLLKVQKAYH |
| | | QLDQSFIDTYIDLLETRRTYYEGPGEGSPFGWKDIKEWY |
| | | EMLMGHCTYFPEELRSVKYAYNADLYNALNDLNNLVI |
| | | TRDENEKLEYYEKFQIIENVFKQKKKPTLKQIAKEILVN |
| | | EEDIKGYRVTSTGKPEFTNLKVYHDIKDITARKEIIENAE |
| | | LLDQIAKILTIYQSSEDIQEELTNLNSELTQEEIEQISNLK |
| | | GYTGTHNLSLKAINLILDELWHTNDNQIAIFNRLKLVPK |
| | | KVDLSQQKEIPTTLVDDFILSPVVKRSFIQSIKVINAIIKK |
| | | YGLPNDIIIELAREKNSKDAQKMINEMQKRNRQTNERIE |
| | | EIIRTTGKENAKYLIEKIKLHDMQEGKCLYSLEAIPLEDL |
| | | LNNPFNYEVDHIIPRSVSFDNSFNNKVLVKQEEASKKGN |
| | | RTPFQYLSSSDSKISYETFKKHILNLAKGKGRISKTKKEY |
| | | LLEERDINRFSVQKDFINRNLVDTRYATRGLMNLLRSYF |
| | | RVNNLDVKVKSINGGFTSFLRRKWKFKKERNKGYKHH |
| | | AEDALIIANADFIFKEWKKLDKAKKVMENQMFEEKQA |
| | | ESMPEIETEQEYKEIFITPHQIKHIKDFKDYKYSHRVDKK |
| | | PNRELINDTLYSTRKDDKGNTLIVNNLNGLYDKDNDKL |
| | | KKLINKSPEKLLMYHHDPQTYQKLKLIMEQYGDEKNPL |
| | | YKYYEETGNYLTKYSKKDNGPVIKKIKYYGNKLNAHL |
| | | DITDDYPNSRNKVVKLSLKPYRFDVYLDNGVYKFVTV |
| | | KNLDVIKKENYYEVNSKCYEEAKKLKKISNQAEFIASFY |
| | | NNDLIKINGELYRVIGVNNDLLNRIEVNMIDITYREYLE |
| | | NMNDKRPPRIIKTIASKTQSIKKYSTDILGNLYEVKSKKH |
| | | PQIIKKG |
| 365 | Met(−) | KRNYILGLDIGITSVGYGIIDYETRDVIDAGVRLFKEANV |
| | saCas9 | ENNEGRRSKRGARRLKRRRRHRIQRVKKLLFDYNLLTD |
| | (N579A) Nickase | HSELSGINPYEARVKGLSQKLSEEEFSAALLHLAKRRGV |
| | | HNVNEVEEDTGNELSTKEQISRNSKALEEKYVAELQLE |
| | | RLKKDGEVRGSINRFKTSDYVKEAKQLLKVQKAYHQL |
| | | DQSFIDTYIDLLETRRTYYEGPGEGSPFGWKDIKEWYE |
| | | MLMGHCTYFPEELRSVKYAYNADLYNALNDLNNLVIT |
| | | RDENEKLEYYEKFQIIENVFKQKKKPTLKQIAKEILVNE |
| | | EDIKGYRVTSTGKPEFTNLKVYHDIKDITARKEIIENAEL |
| | | LDQIAKILTIYQSSEDIQEELTNLNSELTQEEIEQISNLKG |
| | | YTGTHNLSLKAINLILDELWHTNDNQIAIFNRLKLVPKK |
| | | VDLSQQKEIPTTLVDDFILSPVVKRSFIQSIKVINAIIKKY |
| | | GLPNDIIIELAREKNSKDAQKMINEMQKRNRQTNERIEEI |
| | | IRTTGKENAKYLIEKIKLHDMQEGKCLYSLEAIPLEDLL |
| | | NNPFNYEVDHIIPRSVSFDNSFNNKVLVKQEEASKKGNR |
| | | TPFQYLSSSDSKISYETFKKHILNLAKGKGRISKTKKEYL |
| | | LEERDINRFSVQKDFINRNLVDTRYATRGLMNLLRSYFR |
| | | VNNLDVKVKSINGGFTSFLRRKWKFKKERNKGYKHHA |
| | | EDALIIANADFIFKEWKKLDKAKKVMENQMFEEKQAES |
| | | MPEIETEQEYKEIFITPHQIKHIKDFKDYKYSHRVDKKPN |
| | | RELINDTLYSTRKDDKGNTLIVNNLNGLYDKDNDKLKK |
| | | LINKSPEKLLMYHHDPQTYQKLKLIMEQYGDEKNPLYK |
| | | YYEETGNYLTKYSKKDNGPVIKKIKYYGNKLNAHLDIT |
| | | DDYPNSRNKVVKLSLKPYRFDVYLDNGVYKFVTVKNL |
| | | DVIKKENYYEVNSKCYEEAKKLKKISNQAEFIASFYNN |
| | | DLIKINGELYRVIGVNNDLLNRIEVNMIDITYREYLENM |
| | | NDKRPPRIIKTIASKTQSIKKYSTDILGNLYEVKSKKHPQI |
| | | IKKG |
| 366 | Reference | TLNIEDEYRLHETSKEPDVSLGSTWLSDFPQAWAETGG |
| | M-MLV | MGLAVRQAPLIIPLKATSTPVSIKQYPMSQEARLGIKPHI |
| | RT; M- | QRLLDQGILVPCQSPWNTPLLPVKKPGTNDYRPVQDLR |
| | MLV (H8Y) | EVNKRVEDIHPTVPNPYNLLSGLPPSHQWYTVLDLKDA |
| | RT | FFCLRLHPTSQPLFAFEWRDPEMGISGQLTWTRLPQGFK |
| | | NSPTLFDEALHRDLADFRIQHPDLILLQYVDDLLLAATS |
| | | ELDCQQGTRALLQTLGNLGYRASAKKAQICQKQVKYL |
| | | GYLLKEGQRWLTEARKETVMGQPTPKTPRQLREFLGT |
| | | AGFCRLWIPGFAEMAAPLYPLTKTGTLFNWGPDQQKA |
| | | YQEIKQALLTAPALGLPDLTKPFELFVDEKQGYAKGVL |
| | | TQKLGPWRRPVAYLSKKLDPVAAGWPPCLRMVAAIAV |
| | | LTKDAGKLTMGQPLVILAPHAVEALVKQPPDRWLSNA |
| | | RMTHYQALLLDTDRVQFGPVVALNPATLLPLPEEGLQH |
| | | NCLDILAEAHGTRPDLTDQPLPDADHTWYTDGSSLLQE |
| | | GQRKAGAAVTTETEVIWAKALPAGTSAQRAELIALTQA |
| | | LKMAEGKKLNVYTDSRYAFATAHIHGEIYRRRGLLTSE |
| | | GKEIKNKDEILALLKALFLPKRLSIIHCPGHQKGHSAEAR |
| | | GNRMADQAARKAAITETPDTSTLLIENSSP |
| 367 | Linker | GGSGGS |
| 386 | Linker | GGSGGSGGS |

1. A prime editing composition comprising (A) a first prime editing guide RNA (PEgRNA) or one or more polynucleotides encoding the first PERNA and (B) a second PEgRNA or one or more polynucleotides encoding the second PEgRNA,, wherein:
the first PEgRNA comprises
(i) a first spacer that is complementary to a first search target sequence on a first strand of a TCF4 gene,
(ii) a first gRNA core capable of binding to a Cas9 protein; and
(iii) a first extension arm comprising a first editing template and a first primer binding site (PBS),
wherein the second PEgRNA comprises
(i) a second spacer that is complementary to a second search target sequence on a second strand of the TCF4 gene,
(ii) a second gRNA core capable of binding to a Cas9 protein; and
(iii) a second extension arm comprising a second editing template and a second PBS and
wherein the first strand and the second strand of the double-stranded TCF4 gene are complementary to each other, wherein the first editing template comprises a region of identity to a sequence on the first strand of the TCF4 gene, and wherein the second editing template comprises a region of identity to a sequence on the second strand of the double-stranded TCF4 gene.

2. The prime editing composition of claim 1, wherein the first PEgRNA comprises a sequence selected from the group consisting of SEQ ID NOs: 306-320 and the second PEgRNA comprises a sequence selected from group consisting of SEQ ID NOs: 321-335.

3. The prime editing composition of claim 1, wherein the selected sequence for the first spacer is SEQ ID NOs: 65, 66, 67, 68 and 69, or wherein the selected sequence for the second spacer is SEQ ID NOs: 70, 71, 72, 73 and 74.

4. (canceled)

5. The composition of claim 2, wherein the editing pair is selected from the group comprising; SEQ ID NOs: 317 and 335, SEQ ID NOs: 318 and 321, SEQ ID NOs: 318 and 322, SEQ ID NOs: 315 and 321, SEQ ID NOs: 315 and 331, SEQ ID NOs: 319 and 321, SEQ ID NOs: 319 and 323, SEQ ID NOs: 319 and 324, SEQ ID NOs: 319 and 326, SEQ ID NOs: 315 and 332, SEQ ID NOs: 316 and 329, SEQ ID NOs: 316 and 332, SEQ ID NOs: 319 and 329, SEQ ID NOs: 319 and 335, SEQ ID NOs: 320 and 324, SEQ ID NOs: 320 and 325, SEQ ID NOs: 317 and 324, or combination thereof.

6. The composition of claim 2, wherein the editing pair is selected from the group comprising SEQ ID NOs: 306 and 324, SEQ ID NOs: 306 and 329, SEQ ID NOs: 306 and 331, SEQ ID NOs: 306 and 332, SEQ ID NOs: 310 and 322, SEQ ID NOs: 310 and 323, SEQ ID NOs: 310 and 325, SEQ ID NOs: 310 and 326, SEQ ID NOs: 310 and 331, SEQ ID NOs: 310 and 332, SEQ ID NOs: 313 and 332, SEQ ID NOs: 314 and 326, SEQ ID NOs: 317 and 329, SEQ ID NOs: 317 and 331, SEQ ID NOs: 317 and 332, SEQ ID NOs: 318 and 323, SEQ ID NOs: 318 and 326, SEQ ID NOs: 307 and 322, SEQ ID NOs: 307 and 323, SEQ ID NOs: 307 and 326, SEQ ID NOs: 307 and 329, SEQ ID NOs: 307 and 331, SEQ ID NOs: 307 and 332, SEQ ID NOs: 311 and 322, SEQ ID NOs: 311 and 323, SEQ ID NOs: 311 and 329, SEQ ID NOs: 311 and 331, SEQ ID NOs: 311 and 332, SEQ ID NOs: 314 and 332, SEQ ID NOs: 315 and 322, SEQ ID NOs: 315 and 323, SEQ ID NOs: 315 and 325, SEQ ID NOs: 315 and 326, SEQ ID NOs: 315 and 328, SEQ ID NOs: 315 and 329, SEQ ID NOs: 318 and 329, SEQ ID NOs: 318 and 331, SEQ ID NOs: 318 and 332, SEQ ID NOs: 318 and 335, SEQ ID NOs: 319 and 322, SEQ ID NOs: 319 and 325, SEQ ID NOs: 319 and 328, SEQ ID NOs: 308 and 323, SEQ ID NOs: 308 and 326, SEQ ID NOs: 308 and 331, SEQ ID NOs: 308 and 332, SEQ ID NOs: 309 and 322, SEQ ID NOs: 309 and 323, SEQ ID NOs: 309 and 324, SEQ ID NOs: 312 and 322, SEQ ID NOs: 312 and 323, SEQ ID NOs: 312 and 326, SEQ ID NOs: 312 and 329, SEQ ID NOs: 312 and 331, SEQ ID NOs: 312 and 332, SEQ ID NOs: 316 and 322, SEQ ID NOs: 316 and 323, SEQ ID NOs: 316 and 325, SEQ ID NOs: 316 and 326, SEQ ID NOs: 316 and 328, SEQ ID NOs: 319 and 331, SEQ ID NOs: 319 and 332, SEQ ID NOs: 320 and 322, SEQ ID NOs: 320 and 323, SEQ ID NOs: 320 and 326, SEQ ID NOs: 320 and 329, SEQ ID NOs: 309 and 325, SEQ ID NOs: 309 and 326, SEQ ID NOs: 309 and 329, SEQ ID NOs: 309 and 331, SEQ ID NOs: 309 and 332, SEQ ID NOs: 313 and 322, SEQ ID NOs: 313 and 323, SEQ ID NOs: 317 and 321, SEQ ID NOs: 317 and 322, SEQ ID NOs: 317 and 323, SEQ ID NOs: 317 and 325, SEQ ID NOs: 317 and 326, SEQ ID NOs: 320 and 331, SEQ ID NOs: 320 and 332, or combination thereof.

7. (canceled)

8. The prime editing composition of claim 1, wherein the first gRNA core and the second gRNA core each comprises a sequence from the group consisting of SEQ ID NOs: 301, 302, 303, 304, 305, 381, 382, 383, 384, and 385.

9-12. (canceled)

13. The composition of claim 1, wherein the first PEgRNA directs the first prime editor to generate a first nick on the second strand of the TCF4 gene, wherein the second PEgRNA directs the second prime editor to generate a second nick on the first strand of the TCF4 gene, and wherein the TCF4 gene comprises an inter-nick duplex (IND) between the position of the first nick and the position of the second nick.

14-22. (canceled)

23. The composition of claim 1, wherein the region of complementarity between the first editing template and the second editing template comprises an exogenous sequence compared to the TCF4 gene.

24. (canceled)

25. The composition of claim 13 wherein the first editing template comprises a region of complementarity to the IND on the second strand of the TCF4 gene, or wherein the second editing template comprises a region of complementarity to the IND on the first strand of the TCF4 gene.

26-27. (canceled)

28. The composition of claim 1, wherein the first editing template comprises the sequence (CUG)ₙ, wherein n is any integer between 0 and 38 (SEQ ID NO: 402).

29-30. (canceled)

31. The composition of claim 1, wherein the second editing template comprises the sequence (CAG)ₘ, wherein m is any integer between 0 and 38 (SEQ ID NO: 403).

32-33. (canceled)

34. The composition of claim 1, wherein a region of complementarity between the first editing template and the second editing template comprises the sequence (CUG)_{w}, wherein w is any integer between 0 and 38 (SEQ ID NO: 402).

35-54. (canceled)

55. The composition of claim 1, wherein the first editing template comprises at its 5′ end, a sequence selected from the group consisting of: nucleotides 1-100 of SEQ ID NO: 115; nucleotides 1-90 of SEQ ID NO: 116; nucleotides 1-80 of SEQ ID NO: 117; nucleotides 1-70 of SEQ ID NO: 118; nucleotides 1-60 of SEQ ID NO: 119; nucleotides 1-50 of SEQ ID NO: 120; nucleotides 1-40 of SEQ ID NO: 121; 30 nucleotides 1-30 of SEQ ID NO: 122; nucleotides 1-20 of SEQ ID NO: 123, nucleotides 1-10 of SEQ ID NO: 124, and SEQ ID NOs: 105-114 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, and 299.

56. The composition of claim 1, wherein the first editing template comprises at its 3′ end, a sequence selected from the group consisting of: the last 100 nucleotides of SEQ ID NO: 115; the last90 nucleotides of SEQ ID NO: 116; the last 80 nucleotides of SEQ ID NO: 117; the last 70 nucleotides of SEQ ID NO: 118; the last60 nucleotides of SEQ ID NO: 119; the last 50 nucleotides of SEQ ID NO: 120; the last 40 nucleotides of SEQ ID NO: 121; the last 30 nucleotides of SEQ ID NO: 122; the last 20 nucleotides of SEQ ID NO: 123; the last10 nucleotides of SEQ ID NO: 124, and SEQ ID NOs: 104-114 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, and 299.

57. The prime editing composition of claim 13, wherein the second editing template further comprises a region of complementarity to the sequence of the IND downstream of the array of tri-nucleotide repeats.

58-61. (canceled)

62. The composition of claim 1, wherein the second editing template comprises at its 5′ end, a sequence selected from the group consisting of: nucleotides 1-100 of SEQ ID NO: 135; nucleotides 1-90 of SEQ ID NO: 136; nucleotides 1-80 of SEQ ID NO: 137; nucleotides 1-70 of SEQ ID NO: 138; nucleotides 1-60 of SEQ ID NO: 139; nucleotides 1-50 of SEQ ID NO: 140; nucleotides 1-40 of SEQ ID NO: 141; nucleotides 1-30 of SEQ ID NO: 142; nucleotides 1-20 of SEQ ID NO: 143, nucleotides 1-10 of SEQ ID NO: 144, and SEQ ID NOs: 125-134; and 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

63. The composition of claim 1, wherein the second editing template comprises at its 3′ end, a sequence selected from the group consisting of: the last 100 nucleotides of SEQ ID NO: 135; the last 90 nucleotides of SEQ ID NO: 136; the last 80 nucleotides of SEQ ID NO: 137; the last 70 nucleotides of SEQ ID NO: 138; the last 60 nucleotides of SEQ ID NO: 139; the last 50 nucleotides of SEQ ID NO: 140; the last 40 nucleotides of SEQ ID NO: 141; the last 30 nucleotides of SEQ ID NO: 142; the last 20 nucleotides of SEQ ID NO: 143, the last 10 nucleotides of SEQ ID NO: 144, and SEQ ID NOs: 125-134; 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

64. The composition of claim 1, wherein the first editing template and the second editing template are not complementary to each other, or wherein the first editing template and the second editing template comprise a region of complementarity to each other.

65. (canceled)

66. The composition of claim 64, wherein the region of complementarity between the first editing template and the second editing template comprises an exogenous sequence compared to the double-stranded TCF4 gene, and wherein the exogenous sequence comprises a marker, an expression tag, a barcode, or a regulatory sequence.

67-73. (canceled)

74. The composition of claim 1, wherein the first PEgRNA comprises a first primer binding site (PBS) sequence that comprises a region of complementarity to the second strand of the double-stranded TCF4 gene.

75. The composition of claim 74, wherein the second PEgRNA comprises a second PBS sequence that comprises a region of complementarity to the first strand of the double-stranded TCF4 gene.

76. The composition of claim 74, wherein the first PEgRNA comprises a structure selected from:
(i) 5′-[first spacer]-[first gRNA core]-[first editing template]-[first primer binding site sequence]-3′; or
(ii) 5′-[first editing template]-[first primer binding site sequence]-[first spacer]-[first gRNA core]-3′.

77. (canceled)

78. The composition of claim 75, wherein the second PEgRNA comprises a structure: 5′-[second spacer sequence]-[second gRNA core]-[second editing template]-[second primer binding site]-3′; or wherein the second PEgRNA comprises a structure: 5′-[second editing template]-[second primer binding site sequence]-[second spacer]-[second gRNA core]-3′.

79-94. (canceled)

95. The composition of claim 1, (i) wherein the first spacer comprises a sequence selected from the group consisting of SEQ ID NOs: 65-69; (ii) wherein the second spacer comprises a sequence selected from the group consisting of SEQ ID NOs: 70-74; wherein the first PBS comprises a sequence selected from the group consisting of SEQ ID NOs: 75-89, or (iii) wherein the second PBS comprises a sequence selected from the group consisting of SEQ ID NOs: 90-104.

96-98. (canceled)

99. The composition of claim 1, wherein the first editing template comprises a sequence selected from the group consisting of SEQ ID NOs: 105-124 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, and 299.

100-104. (canceled)

105. The composition of claim 1, wherein the second editing template comprises a sequence selected from the group consisting of SEQ ID NOs: 125-144 and 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

106-112. (canceled)

113. A composition comprising a first prime editing guide RNA (PEgRNA) and a second PEgRNA, wherein the first PEgRNA comprises a first spacer comprising a sequence selected from the group consisting of SEQ ID NOs: 65-69, a guide RNA core comprising a sequence selected from the group consisting of SEQ ID NOs: 301-305; 381-385, a first PBS comprising a sequence selected from the group consisting of SEQ ID NOs: 75-89, and a first editing template comprising a sequence selected from the group consisting of SEQ ID NOs: 105-124 and 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, and 299, and wherein the second PEgRNA comprises a second spacer comprising a sequence selected from the group consisting of SEQ ID NOs: 70-74, a guide RNA core comprising a sequence selected from the group consisting of SEQ ID NOs: 301-305; 381-385, a second PBS comprising a sequence selected from the group consisting of SEQ ID NOs: 90-104, and a second editing template comprising a sequence selected from the group consisting of SEQ ID NOs: 125-144 and 146, 148, 150, 152 152,154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, and 300.

114. The composition of claim 113, further comprising a first prime editor that comprises a DNA binding domain and a DNA polymerase domain and associates with the first PEgRNA, and a second prime editor that comprises a DNA binding domain and a DNA polymerase domain and associates with the second PEgRNA.

115. (canceled)

116. The composition of claim 114;
(i) wherein the DNA binding domain is a CRISPR associated (Cas) protein domain;
(ii) wherein the DNA binding domain is a Cas protein domain with a nickase activity;
(iii) wherein the DNA binding domain is a Cas protein domain comprising Cas9;
(iv) wherein DNA binding domain is a Cas9 comprising a mutation in an HNH domain;
(v) wherein the DNA binding domain is a Cas9 comprising a H840A mutation in the HNH domain; or
(vi) wherein Cas12a, Cas12b, Cas12c, Cas12d, Cas12e, Cas14a, Cas14b, Cas14c, Cas14d, Cas14e, Cas14f, Cas14g, Cas14h, Cas14u, or a Casφ.

117-122. (canceled)

123. The composition of claim 114:
(i) wherein the DNA polymerase domain is a reverse transcriptase;
(ii) wherein the reverse transcriptase is a retrovirus reverse transcriptase; or
(iii) wherein the reverse transcriptase is a Moloney murine leukemia virus (M-MLV) reverse transcriptase.

124-126. (canceled)

127. A lipid nanoparticle (LNP) or ribonucleoprotein (RNP) comprising the prime editing composition of claim 1, or a component thereof.

128. A polynucleotide encoding the first PEgRNA and second PEgRNA of claim 1, the dual prime editing system claim 114, or a component thereof.

129-131. (canceled)

132. A vector comprising the polynucleotide of claim 128.

133. (canceled)

134. An isolated cell comprising the first PEgRNA and second PEgRNA of claim 1.

135. (canceled)

136. The cell of claim 134, wherein the cell is a corneal endothelial cell, a corneal endothelial progenitor cell, or a differentiated corneal endothelial cell.

137. A pharmaceutical composition comprising the composition of claim 1.

138. A method for editing a TCF4 gene, the method comprising contacting the TCF4 gene with (i) the composition of claim 1, (ii) a first prime editor comprising a DNA binding domain and a DNA polymerase domain that associates with the first PEgRNA, and (iii) a second prime editor comprising a DNA binding domain and a DNA polymerase domain that associates with the second PEgRNA, wherein the first PEgRNA directs the first prime editor to generate a first nick on the second strand of the TCF4 gene, wherein the second PEgRNA directs the second prime editor to generate a second nick on the first strand of the TCF4 gene, and wherein the contacting results in excision of an inter-nick duplex (IND) between the position of the first nick and the position of the second nick of the TCF4 gene, thereby editing the TCF4 gene.

139-144. (canceled)

145. The method of claim 138 wherein the contacting results in deletion of the sequence (CTG) x in the TCF4 gene, wherein x is an integer no less than 1.

146-158. (canceled)

159. A method for treating Fuchs endothelial corneal dystrophy type 3 in a subject in need thereof, the method comprising administering to the subject the composition of claim 1, the dual prime editing system of claim 114, the LNP or RNP of claim 127, the polynucleotide of claim 128, the vector of claim 132, or the pharmaceutical composition of claim 137, wherein the administration results in a reduced number of an array of CTG repeats in the TCF4 gene in the subject, thereby treating Fuchs endothelial corneal dystrophy type 3 in the subject.

160-162. (canceled)
